SEQUENCE LISTING (89 sequences)

SEQ ID NO: 1 - DNA, 8450 bp, Triticum aestivum
  source 1..8450: mol_type = genomic DNA
  variation 1208..1491: note = N can be any nucleotide selected from adenine, guanine, cytosine and thymine.
catcactcactcgggtacagagcgcagcgggccgcgtgactctcgcgtcgtcgcatcgcgcgcgctgcacaccaccgtcagcgcgaccactgccgccaatggcgaccgcctcgccgccgctcgccacccccttccgcccgctcgccgtcgccggaggtagcggcggactcgtcgtgggcgccagggccgtggtaccgccgaggcgggggcggcgggggttcgtggtgcggagcgtggcgagcgatcgggaggtgcgggggcccgccccgaccgaggaaggtaagcggccgcgcgctcccattggctgccacgacctggaatgctggtcggctgtaatgtggttcggattttcgctggtgcgacctgaatttgtgatcctctgactgacggcctatgtgttccccgggtttcgggctgttgattttgtgcgcgcgcattgctaatctacttctctaggatttgttagggctcagagcaatcttaaattcgcgaatacacttcgacgaatgtagtctgaatttactgaaactagcttagtttggcgcgtcaagatagactgatgtatttatggtagcaaattctgaaaacttcttctgtgcgtggacagccacccctcgtggatgatgcagcaagtgctaacacagtgtttgctagtacttgctagtatctgtagctgtaacaaaatccttcgaatctgaactagtgagataaatcgtttgagatttttgctgaagattacacagtatcaatatttctgaaggtgattggaacagtatagcatttcaaactggcaaccaagcaggatcagtatactgacgcaatttttttctgaaggtggttggaataccggtatgaattttataatcattgaattaaggcgtaagatttggtaacaaaaactgcttttcacgggaaacgatgaaacatggcacaattcaacctgactttcttttatgaaaaggctctcaagctgccttgtactccctccgtcccataatataagaacgtttttgacattagtgtagtaccaaaaacgttcttatattatgggacagagggagtatttattaatggataaaaagagaggtacagggttctagtagtaacatatgcaaatggtgatacagtatattatctacgattttgtgcaagaattttcacacgaaaacaatattccatgcagtttttagttttgacttttttttttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgaacccaacatacagtacaatagtacaaatgccggggcggcagcagagagagaggcagggggcgacccccccatgctatgttacatgcagctttgttctacccacgcagagcctgtagccagttttgactctttttgaattgtttggattgcttaccacatgtgtcacgtggttttagttttgactcttgactgtttaatacctactctttttaagattttggattctaatgtcatctgtcttgcaactcagagctttcagccgtgctaacttccattgattcatccgccatcgcatcaaacatccagcaccatgcagacttcacaccgttattctcgccagagcactcttcacccctaaaggcttaccatgcaactgccaaaagtgtttttgattctctgatattgaattggaatgcaacatatgactattacaacaaagtgaatgcaaagcaagcttattacctatccatggaatttttgcaggtctcgcaatatactttcttcagccttatctcataccttttggtaactgcgttatgatgttatatgtttttttttgtactgcagggaagagctcttacaaatgctattggcaacctagagctaacaggacaatatgcagaagcgttaaaacaacttggacagaacctagaggatgttgctagtcaggtaagccatttttgtcagagaatggtttttagtccatatcaaaattattgccgagccatttttctaaataacattttattattattcatgaacttccatatttttttcaaatctgcaaaaggagaagtttttttcctgggggttccaccatctgacttatatttgttatgcagttttcagagtgattgactatgaaattacgccaaaacacgagcattgcctagagtgccctgtagttttagaaatgttaagattggatatgttacttttctattacaatgaaacaggaaccagatcctgcccttggcaatggtggactaggccgtctagcgtcctgttttctggattctatggcaaccctaaattatccagcatggggatatggacttcggtacagatatggcctctttaagcaaatcataacaaaggatggtcaggaggaggtagctgagaattggctagaggtagtgatatgtcttctacttgtttactgtttggaagcgttgattatgttggtttctgttttaatgatctgcatgttagcttcttttgccatttataactgatgctgtatctccagatgggaaatccatgggagattgtaagaaatgatgtctcttatcctctgaaattctatggcaaagtggttgaaggcactgatgggagaaaacactggattggaggagagaatatcaaggctgtggcacatgatgttcctattcctggctacaagactaaaactactaataatcttcgtctttggtcaacaacagtaccatcacaaaactttgatttgggagcttttaatgctggggatcatgtcaaggccaacgaagcacatctaaatgctgaaaaggtattcctttgtctgcatttattttgaatataaatcattaaatctcaataactattgatttaaatggtaaacactaaacattgtctagctagtagtcactgtggtgtccctcaatgtcattttagaatgtgcacagtcaaatgtttgataactttgattgtaataatatgataatatacagaaactttgattgacactagatttttcataaacatacttattgtatttccatgtaaatagaaaagctgatgttcagaggtttgtgtgtcaaaatgaacagagtttgtgcacacataatatcttcatctggtgtttgcagttctctaggttgtgtttgcgttttctcctgctctgccttaactaatctcagcatatgcatcaggctacattgctcctctgtgtctataaaaaataagtctgatgatatatggttgcagtccagtagttcgtagatggcacagcatggaagaacttttcacatggaagccttacccttataattttaattataccttttgagtgtttgtgcattgtgtacatacaccattaagtatgggtgctcaaaaatggtttatcccaaactgtgcaaaagccgccactgtttgtgtattaatttttttgaactagctaaacaacttgaccgaacaagatacatacaattgcactcacttctgaagcagctcagttggacggatttgttccaatcccgtgcatgggttaaattgtcgagttgtcctcgcttgttcagtagtaggagtactatctattatatttatactaaagttttctttttgtgatacagtacaaacttttagtaatacttacatttgcgaccactgtggacaaaatttattatgtctaacttaactttatcactggtcttctcatcatgccagatatgccacgtattgtatccaggggacgaatcatcagaggggaaaattcttcgcctgaaacaacaatatacactatgctcggcctccctgcaggacattatttctcgttttgagtcgagggctggtgattctctcaactgggaggacttcccctctaaagttgcagttcagatgaatgacacccatccaacactgtgcattcccgagttaatgagaatactgatggatgttaagggattgagctggaatgagtcttggagtatcacggaaaggtatcacagaagggtactgcagtttgatgtgttatgctgttcctccagttccagttaactaattttatttcctcatctgtcttccattcaatcatgtactgattttctgatatccacatcatttgtagaaccgttgcatacactaaccacacagtacttcctgaagctctggagaagtggagcttggacattatgcagaaacttctacctcgacatgttgagattatagaaagaatagatgaggaggtatcaaatgtgcagataatattatacaattaatttcatgctctgtagttcaacttatggtttacttgtcttcatcgttgattttaaacagctgatgaacaccatcgtctcaaaatatggaacggcagatatttcactgttaaaacagaagcttaaagatatgaggatcttagacaacgttgatcttccagcctctgttgctaaactgtttattaaaccaaaagagaaaaaggggaagttgcttgttgaatctttggagtctattgctgaagctgacgagaaaactgagtcagaagaggaggaaaacattctatccgagacaacagagaaaaagggcgaatctgactctgaagaagctcctgatgcagaaaaggaggatcctgagtatgagttagatccatttacaaaatacgatcctcagttacctagagttgttcgaatggcgaacctctgcgttgttggtggacattcagttaatggtgtggctgagattcacagtgaaattgttaagcaagatgtgttcaatagcttttatgaggtatttgcatgatgatttggaaaccgtgatcttattttccaatggctgctataatcgtcaggtgatatggtattcaccatctgccattttcttgtgttctttgccagatgtggcctactaagttccaaaacaaaacaaatggagtgacccccaggcgttggatccggttttgtaatcctgaattaagtacaataatttcaaaatggataggctctgatgactggattctgaacactgataaacttgcaggactgaagaaggtacttccagtagttttattccccaataatcagtaatctaatgcttccttcacttcaatgttaactgtgcattgtttttacattgatgtgtgttcatcctgtaacagtttgctgatgatgaggatctgcaatcagaatggcgtactgctaaaaggaataacaagatgaaggtagtttcgctgataagagacaagactggatatgttgtcagcccagatgcaatgtttgatgtgcaggtatattttgtactagaaaacatgtgtcttcttataaatatggtttaactcagctcttggtactggacacttaggtgaaaagaatacatgaatataagcggcagcttctaaatatccttggtatcatttaccgctacaagaagatgaaagaaatgagtgcaaaagatagaataaagagctttgttccaagggtatgcatatttggcgggaaagcttttgccacttacgtacaggcaaagaggattgtgaagtttatcacagatgtcgcagctactgtaaattatgatcctgatattggagatctactgaaggtaaatttgtgtctttgatgccacttatcagtcttaattttcatgttctgtcactggcaagttgactccgactgaaaagcattgaatgttcagagtttcaaaggaaaacagagcagtgaatgttcttaatgtcggtgcttgtaaaactgagcaactgaatagccacaggttttttagagaacaatatagccacaaatttttttgacacttttattgctatcagcatctgatgctgatattactgataactagatgaggtaaatataaggtgacaggttaagtctgaggatttttgtttgttatccaggttgtatttgtcccagattataatgttagcgttgctgagacgctcattcctgccagtgaattgtcccagcatatcaggtaatattctgcctactcttatatagtatgtgaatacataaaaaaagaatattacctggagttggctgacatagaatttttcatgtgcctttttgtttgctataatatgtacctggagtcttactgggatagtgccatccagaagtgccaccccagtttctatttctgtataagacgtgcatttttaacagtattactatccattcagtaccgctggaatggaagctagtggaaccagcaacatgaagtttgcaatgaatggctgtattcttattggaactttggatggtgcgaatgtggagatcagagaagaggttggagaggagaactttttcctctttggtgcacatgcacctgaaatcgctggtttgaggcaagagagagctgaagggaaggtaccacacttgtttatatagctgtgcctactatcaaactagtaaagatctctcccatatgtgctgtccaaatagtttagctgaaggaaaatcataactttacaagtttcctttgcacttgtagtttgtgcctgacccgagatttgaagaggttaaggaatacgtccgcagtggcatctttgggactggcaactatgatgaattgatgggttctttggaaggaaatgaaggttatggacgtgcagattattttcttgttggcaaggattttcccagttacattgaatgccaggaaaaagttgatgaagcataccgagatcagaaggtaagttgtataactgacgctgtttgttttctggctgaaatgctcacagtgattatgcaaactttctgatatgtgatggctgacatcagtgtttcactgtgaacgacttcattgattcgtaggaaggaatatttcttagtttaaagtttcaaactgaagtatctatcggtgtgttggaagatcataaacaatcagagaatatagttgctagatgtgctatattactcatctgttggctgttgagtgttgatacatgagctttgatgtagctagaccttcaggaaagtgcttctgtgcattccaataaaaaaataacttataacttaacaaaacaaaacaattaggttgaacctcaccagggggccttttgttatctgttttctgaaatgaaaatcagaagtggggagctcctcaattcgaaaaagaaaaagaaaatcagggggtaactgagggtctggtttccctctgttggagcccccaagaattctgtggtcttatttttgtaagagtataagatgcactgctccgttaagtttctccttcacccatttgttttcattgcttcgtgacatttcgtgtgacagctcagatcaccatatgcatcccatttgtacacattattttgctaccgtggatatttccactctgtttcatgtgtgacatttttaattcgttacattgttactctaaacattagtctaatagattttcagagttttgaccctcctaatcacataatatccgtttcgacctgcagctatggacaagaatgtctatcctcaacacagcgggttcccccaagttcagcagtgaccggacgatccacgagtatgccaaggacatatggggtatcagccccgtcatcatgccctgaataattcaagcagcttccaggaaccttgagagtctatgaggcgcgattggtcgctgatttgccttgattcagcgagaatgtattgctccccccagttttgtataggcatatgcaagttctgacatggacctgagtgctgagagcatgcagggtagttcatccttagcaatattgcatgtcagttagccagtattacccaggcttatggtgtgtgtatttcgctgaataaagttggagcagagtcatcaaacaaacatactttggtgtatctgtaatgataaataaatggtacatgtgactgaacaaaaagtgtcgttggcgtgcacatgtagtagaagcatgtttcttcagagagggtgcaaagctgaaagcaccccagctttggagtgagaatgcagaagttacgaaaacatttgtttacatccaaagtgcaacgggattcgtcaagagttcagagtcttccatcacggtcatagagcatgtccaatagcagccctataaacctataacagagcagtagcagtggtccctcacattttgaaccaaaaatgcagctccaacggctgccctatttgtaaaatttacttcaaaatttgttggacagccctaaaatgcaacaccaactgatgtaaatgtacaactattcggctgctgccctaaaatttgctccaagtggcgaaccactcaggaccac

SEQ ID NO: 2 - DNA, 2907 bp, Triticum aestivum
  source 1..2907: mol_type = genomic DNA
atggcgaccgcctcgccgccgctcgccacccccttccgcccgctcgccgtcgccggaggtagcggcggactcgtcgtgggcgccagggccgtggtaccgccgaggcgggggcggcgggggttcgtggtgcggagcgtggcgagcgatcgggaggtgcgggggcccgccccgaccgaggaagagctttcagccgtgctaacttccattgattcatccgccatcgcatcaaacatccagcaccatgcagacttcacaccgttattctcgccagagcactcttcacccctaaaggcttaccatgcaactgccaaaagtgtttttgattctctgatattgaattggaatgcaacatatgactattacaacaaagtgaatgcaaagcaagcttattacctatccatggaatttttgcagggaagagctcttacaaatgctattggcaacctagagctaacaggacaatatgcagaagcgttaaaacaacttggacagaacctagaggatgttgctagtcaggaaccagatcctgcccttggcaatggtggactaggccgtctagcgtcctgttttctggattctatggcaaccctaaattatccagcatggggatatggacttcggtacagatatggcctctttaagcaaatcataacaaaggatggtcaggaggaggtagctgagaattggctagagatgggaaatccatgggagattgtaagaaatgatgtctcttatcctctgaaattctatggcaaagtggttgaaggcactgatgggagaaaacactggattggaggagagaatatcaaggctgtggcacatgatgttcctattcctggctacaagactaaaactactaataatcttcgtctttggtcaacaacagtaccatcacaaaactttgatttgggagcttttaatgctggggatcatgtcaaggccaacgaagcacatctaaatgctgaaaagatatgccacgtattgtatccaggggacgaatcatcagaggggaaaattcttcgcctgaaacaacaatatacactatgctcggcctccctgcaggacattatttctcgttttgagtcgagggctggtgattctctcaactgggaggacttcccctctaaagttgcagttcagatgaatgacacccatccaacactgtgcattcccgagttaatgagaatactgatggatgttaagggattgagctggaatgagtcttggagtatcacggaaagaaccgttgcatacactaaccacacagtacttcctgaagctctggagaagtggagcttggacattatgcagaaacttctacctcgacatgttgagattatagaaagaatagatgaggagctgatgaacaccatcgtctcaaaatatggaacggcagatatttcactgttaaaacagaagcttaaagatatgaggatcttagacaacgttgatcttccagcctctgttgctaaactgtttattaaaccaaaagagaaaaaggggaagttgcttgttgaatctttggagtctattgctgaagctgacgagaaaactgagtcagaagaggaggaaaacattctatccgagacaacagagaaaaagggcgaatctgactctgaagaagctcctgatgcagaaaaggaggatcctgagtatgagttagatccatttacaaaatacgatcctcagttacctagagttgttcgaatggcgaacctctgcgttgttggtggacattcagttaatggtgtggctgagattcacagtgaaattgttaagcaagatgtgttcaatagcttttatgagatgtggcctactaagttccaaaacaaaacaaatggagtgacccccaggcgttggatccggttttgtaatcctgaattaagtacaataatttcaaaatggataggctctgatgactggattctgaacactgataaacttgcaggactgaagaagtttgctgatgatgaggatctgcaatcagaatggcgtactgctaaaaggaataacaagatgaaggtagtttcgctgataagagacaagactggatatgttgtcagcccagatgcaatgtttgatgtgcaggtgaaaagaatacatgaatataagcggcagcttctaaatatccttggtatcatttaccgctacaagaagatgaaagaaatgagtgcaaaagatagaataaagagctttgttccaagggtatgcatatttggcgggaaagcttttgccacttacgtacaggcaaagaggattgtgaagtttatcacagatgtcgcagctactgtaaattatgatcctgatattggagatctactgaaggttgtatttgtcccagattataatgttagcgttgctgagacgctcattcctgccagtgaattgtcccagcatatcagtaccgctggaatggaagctagtggaaccagcaacatgaagtttgcaatgaatggctgtattcttattggaactttggatggtgcgaatgtggagatcagagaagaggttggagaggagaactttttcctctttggtgcacatgcacctgaaatcgctggtttgaggcaagagagagctgaagggaagtttgtgcctgacccgagatttgaagaggttaaggaatacgtccgcagtggcatctttgggactggcaactatgatgaattgatgggttctttggaaggaaatgaaggttatggacgtgcagattattttcttgttggcaaggattttcccagttacattgaatgccaggaaaaagttgatgaagcataccgagatcagaagctatggacaagaatgtctatcctcaacacagcgggttcccccaagttcagcagtgaccggacgatccacgagtatgccaaggacatatggggtatcagccccgtcatcatgccctga

SEQ ID NO: 3 - protein, 968 aa, Triticum aestivum
MATASPPLATPFRPLAVAGGSGGLVVGARAVVPPRRGRRGFVVRSVASDREVRGPAPTEEELSAVLTSIDSSAIASNIQHHADFTPLFSPEHSSPLKAYHATAKSVFDSLILNWNATYDYYNKVNAKQAYYLSMEFLQGRALTNAIGNLELTGQYAEALKQLGQNLEDVASQEPDPALGNGGLGRLASCFLDSMATLNYPAWGYGLRYRYGLFKQIITKDGQEEVAENWLEMGNPWEIVRNDVSYPLKFYGKVVEGTDGRKHWIGGENIKAVAHDVPIPGYKTKTTNNLRLWSTTVPSQNFDLGAFNAGDHVKANEAHLNAEKICHVLYPGDESSEGKILRLKQQYTLCSASLQDIISRFESRAGDSLNWEDFPSKVAVQMNDTHPTLCIPELMRILMDVKGLSWNESWSITERTVAYTNHTVLPEALEKWSLDIMQKLLPRHVEIIERIDEELMNTIVSKYGTADISLLKQKLKDMRILDNVDLPASVAKLFIKPKEKKGKLLVESLESIAEADEKTESEEEENILSETTEKKGESDSEEAPDAEKEDPEYELDPFTKYDPQLPRVVRMANLCVVGGHSVNGVAEIHSEIVKQDVFNSFYEMWPTKFQNKTNGVTPRRWIRFCNPELSTIISKWIGSDDWILNTDKLAGLKKFADDEDLQSEWRTAKRNNKMKVVSLIRDKTGYVVSPDAMFDVQVKRIHEYKRQLLNILGIIYRYKKMKEMSAKDRIKSFVPRVCIFGGKAFATYVQAKRIVKFITDVAATVNYDPDIGDLLKVVFVPDYNVSVAETLIPASELSQHISTAGMEASGTSNMKFAMNGCILIGTLDGANVEIREEVGEENFFLFGAHAPEIAGLRQERAEGKFVPDPRFEEVKEYVRSGIFGTGNYDELMGSLEGNEGYGRADYFLVGKDFPSYIECQEKVDEAYRDQKLWTRMSILNTAGSPKFSSDRTIHEYAKDIWGISPVIMP

SEQ ID NO: 4 - DNA, 2000 bp, Triticum aestivum
  source 1..2000: mol_type = genomic DNA
caccacgcagcctaggggcaccgtctggccatctggagccccggtggctcccttggaccccatcttctcgctagtggggccttggtccaaaataatccatgtaatttttcatgttttccggcttcgtatttttttggtaaaaacccaaaaacaatagaaaagaagaattggcctctgacactagattgataggttagtccaaatcaatattatcaaaaattgtgcccaaaccatatacaaatgatataaatataactagaatactttataaatttagatacgtttgataaagtgccaagaatggaagagaagtgatcatatgccgccctgtccatatcgggctcatcagaatgttcccaccatcagactccagccccaggattattaacttatgtcggcggtgcgctgaatggatcttgaagaaggcggaatttgtgtcgccctcacgtaaccaggagaacttggcaggttgacgcgcaatcgacctctccagcaaagcaagtcctaggtaggcgcgcttcagctcgcagagtaaccgtacttctggaggagcaagcgggtggtgatcttgagccgccgagacgagcaatcaattaccgagcgagaagcatctgagtggagatgttaccgagcgttctggaactccaactctgccgtcagtgcattgtcagcttgtgctttgcgtagattcgccggaaaggatcggggtcatgcacgccagaattccaggtgtcagagaccacctaatgaaaaccatcgagctttgatcaaaatagctcaagatggaatctccgatcaccttgggagcgaaaatcttactagtttcgttcaaggccaaggtagttatatatttgtgtgtgcaattactaatcattggccgcttagcaaactcttattggttcgataaacgttaaagtcatccactttgagaaaaaatgctacttgctacaaaccctttcactttgggaggagggggggtgggtggggcaacatgtatcaggatgatggtgccacccttggcaaggaataaatccttgccgcctcgtccttgtttggatgctgcttcttgtcatctcaaggaggcgtgtggagggtggtgtgtccccggatcttgttctctcggttgggttttttctatggcaggtttatctcagtggtagtgtcacgtgaagcagatgacgcgactatgcgtcttggtcctctgcgctctaattccggcagatgaaggttgaccagcttcagcctcgtcggggagcgtatgagatttgtgttttttgactccatctggtcggacctgtggcattatgtagaccctctcatgtatttttttctcctggacagtagtctgttacacaaatcgtggtcatcggatcttctagtccacatcggcgactttttcgaccgccacttctacaaactccttattttcaacaagtttccttcattgagatggaagtccaggcaatggctttgctcacggcacactgccggcggatgcaagagaaagaagacattcgacacccctaaaactatgaagatgtaatttttaatctttgtaagtgtttttgtgaggatttgtgctacttttaatatattgccttttgccttttgcaagggaaaaaaactaaaccattacacgaagagaagagccattcccagccgtccaataaacacgacaaaacgctttacagcacgggggcaattcccagccgtcgaaatgcgctgcaacggaaccagcgctccccgacacggcggcacatgagccacaactcgtaccatgcaccgtgcaccggcggtgggcccactggccagcgtccccacacgtgtaccggcggccacaattacacggcacggcacggcgacggagagatattttaacgccttccaattcagaagctcccccatcactcactcgggtacagagcgcagcgggccgcgtgactctcgcgtcgtcgcatcgcgcgcgctgcacaccaccgtcagcgcgaccactgccgcca

SEQ ID NO: 5 - DNA, 7692 bp, Triticum aestivum
  source 1..7692: mol_type = genomic DNA
ttgcacggcacggcgacggagagatatttttaacgccttccaattcagaagctccccatcactcgctcgggtatcagagcgggccgcgtgactctcgcgtcgtcgcatcgcgcgcgctgcacaccaccgtcagcgcgaccactggcgccaatggcgaccgcctcgccgccgctcgccaccgccttccgcccgctcgccgccgccggcggcgccggaggaggaggagcgcacgccgtgggcgccgcgggccgggtcgcgccgaggcgggggcgccggggcttcgtggtgcggagcgtggcgagcgatcgggaggtgcgggggcccgcctcgaccgaggaaggtaagcggccgcgcgctcccgttggctacgacgacccggaatgctggtcggctgtaatgtggttcggattttcgctggtgcgacctgaatttgtgatcctctgactgatggtctatgtgttcccgggtttcgggctgttgattttgtgcgcgcgcattgctaatctactgctctaggatctgctagggttcagaggaatcttaaattcgtgaataagcttcgacgaatgtattctgaatttactgaaactggcttagtttggggcgtcaagttagaatgatgtatttatggcacagtagcaaattctgacaaattcttctgtgcgtggacagccaccccttgtggatgatgcagcaagtgtcaacatactgtcgctactatctgtagctgtaacaaaatcttacgccttgttttctggtggtacagtatattatcattttacaaagagtagctgtaacaaaatcttacgccttgttttctggtgggttgccattgaggagtaagtagaatgcaaatggtgatacagtatattatctaccattttgtgcaagaattttcacacggaaacaatatcccatgcaatttttagttttgactctcgactgtttatacctactctttgtaaaattttggatcctaatattgttgttcttgcaactcagagctttcagccgtgctaacttccattgattcttccgccatcgcgtcaaatatccagcaccatgcagacttcacaccgttgttctcaccagagcactcttcacccctaaaggcttaccatgcaactgctaaaagtgtttttgattctctgatcatcaattggaatgcaacatatgactattacaacaaagtgaatgcaaagcaagcttattacctgtccatggagtttttgcaggtctcacaatatacttcctttagccttatctcaataccttttggtaactgcgttatgatgttatatggttgttatctgtgtactgcagggaagagctctcacaaatgctattggcaatctagagctaacaggacaatatgcagaagcgttaaaacaacttggacagaacctagaggatgttgctagtcaggtaagtgatttttgtcggagattggtttttagtccgtatcaaaattatcgcagggccattgtttttattttgaaaacgcattttattattaatcatcaagttccctttgtttaaaaactatctgaaaagaagaagtttttttcctgagggttccaccgtctgacttatgatctttatgcagttttcagagtgactatgaaattacgccaaaacacgatcttattgatgcctagagtgccctgtagtttagaaatctaagattggatatgttacttttctattacaatgaaacaggaaccagatcctgcccttggcaatggtggtctaggccgtctagcgtcctgttttctggattctatggcaaccctaaattatccagcatggggatatggacttcggtacagatatggcctctttaagcaaatcatagcaaaggatggtcaggaggaggtagctgagaattggctagaggtagtgatatgtcttctacttgtttactgtttggaagtgttgattatgttggtttatttatgttctaatgacctgcatgttagcttcttttctatttataactgatgctgtatctccagatgggaaatccatgggagatcgtaagaaatgatgtctcttatcctgtgaaattctatggcaaagtggttgaaggcactgatgggagaaagcactggattggaggagagaatatcaaggctgtggcacatgatgttcctattcctggctacaagactaaaactactaataatcttcgtctttggtcaacaacagtaccatcacaaaattttgatttgggtgcctttaatgctggggatcatgccaaggccaatgaagcacatctaaatgctgaaaaggtattctttcgtctactttattttgaatataaaccattaactctcaacaactactgattcaaaggctaaacagtgctttgctagtagttactgtgttttctcttagatgtcattttataatgtgtgcagtcaatcgtttgatatacacaaacctatttggatttctcctgtaaaaatgttgtcactagatttttcatgaaaaaacttattgtatatgtaaatagaaaagctgaggctcaggtgctttgtgttcaaatgacaagtaaaaatgaacagagtttgtgcacccataacttcttcacctggtgtgtgcaattctttcggttgtatttatgcgtgttcttattctgcctgaatgattctcagcatatgcatcaggctacatttctcctctgtgtcaagaaaaaataagtctgaccagatgcagtccagtagtctttagatcttttcacatggaagcgttactctatgatttttattatacctttagagtgtttgtgcattgtgcacatataccatgaagtatgggtgttcaaatgtggttctctcaaactgtgctaaatccaccactgtttgccagtattcatttttctgaactagcaacaacttgactggacaagatacattgtactcacatctgaagctgctgggttggacagatttgtccgacccgtctttgaacatctctactatttagtgtgcatgggttaaattgttgacttttcctcgtttgttcagtagtaggagcactatctaattatatttatattcaagttttctctgtgtgttacgttataaacttttagtcatacttacatttgtggacaaaagttatcatgtctagcttaagtttgtgactgatcttctcatcatgccagatatgccacgtattgtatccaggggacgaatcatcagaggggaaaattcttcgcttgaaacaacaatatacattatgctcggcctccctgcaggacattatttctcgttttgagtcgagggctggtgattctctcaactgggaggacttcccctctaaagttgcagttcagatgaatgacactcatccaacactgtgcattccggagttaatgagaatactgatggatataaagggattgagctggaatgaggcttggagtatcacagaaaggtaccacagaagggtactgcagtttgatgtgttatgctgttcctccagctccaattaactaattgtattgcctcatctgtcttccattcaattatgtactgattctctgatatcatttgtagaaccgttgcatacactaaccacacggtacttcctgaagctctggagaagtggagcttggacattatgcagaaacttctaccccgacatgttgagatcatagaaacaatagatgagaaggtaacaaatgtgcagataatattatgcaataaatttcattctctgtagttcaacttatggtttacttatcaacatcgttgattttaaaacagctgatgaacaacatcgtctcaaaatatggaacggcagatatttcactgttaaaacagaagcttaaagatatgaggatcttagacaacgttgatcttccagcctctgttgctaaactgtttattaaacctaaagagaaaacggggaagttgcttgttcaatctttggagtctattgctgaaggtgacgagaaaactgagtcacaagaggaggaaaacattctatccgagacagcagagaaaaagggcggatctgactctgaagaagctcctgatgcagaaaaggaggatcctgtgtatgagttagatccatttgcaaaatacgatcctcagttacctagagttgttcgaatggcaaatctctgtgttgttggtggacattcagttaatggtgtggctgagattcacagtgaaattgttaagcaagatgtgttcaatagcttttatgaggtatttgcacgatgatttggaaaccatgatcttattttctaacggctgctataatcgccaggtaatatggtatcaccatctgccattttcttatgttatttgccagatgtggcctactaagttccaaaacaaaacaaacggagtaactcccaggcgttggatccggttttgtaatcctgaattaagtgcaatcatttcaaaatggataggctctgatgactggattctgaacactgataaacttgcaggactgaagaaggtacttccagtaattttaatttccaaatatcagtaatctaatgcttccttcacttccatgttacctgtgccttgtttttacattgatgtgtgttcatcccgtaatagtttgctgatgatgaggatctgcaatcagaatggcgtactgctaaaaggaataacaagatgaaggtagtttcgctgataagagacaagactggatatgttgtcagcccagatgcgatgtttgatgtgcaggtatattttggactagaaacatgtgtctcctgttaaatatggtttaactcggatcttggtacttgacacttaggtgaaaagaatacatgaatataagcggcagctgctaaatatccttggtatcgtttaccgatacaagaagatgaaagaaatgagtgccaaagatagaataaagagctttgttccaagggtatgcatatttggtgggaaagcttttgccacttacgtacaggcaaagaggattgtgaagtttatcacagatgtcgcagctactgtaaattatgaccctgatgttggagatttactgaaggtaaatttgtgtcttcaatggcacttatcattctaaattttcatgatctgtcactagcaatttgactccgactaggggttactgaaaagcactgaatgttccgagtttcaaaagaaaacagagcagtgaatgttcttaatgtcggtgcttgtagaactgagaaactgaatagccacagtttttttttagagaacaatagccacaattgttctgatgctgattttgctgataactagatgaggtaaatacaaggtgacaggttaagttctgaggatttttgtttgttatccaggttgtatttgtcccagattataatgttagcgttgctgagaagctcattcctgccagtgaattgtcccagcatatcaggtaatattctggctactcttatatagtgctccctccgtcccaaaattcttgtcttagatttgtccaaatacggatgtatctagttacattttagtgttagatacatccgtatctagacaaatgtaagagaagaattttgggacggagggagtatgtgaatacaaagaaaaggagtcttatctggaggtgactgatgtggaaattttcatgtgcctttttgtttgctactccctccgtcccataatataagagcgtttttgacagtacactattttcaaaaatgctcttataatatgggacggagggagtataatatatacctggagtcttgctgggatagtgacatccagaagtgccacgccagcttctatttctgtacaagacgtgcatttttaacactattactatccattcagtaccgctggaatggaagctagtggaaccagcaacatgaagtttgcaatgaatggttgtattcttattggaactttggatggtgcgaatgtggagatcagagaagaggttggagaggagaactttttcctctttggtgcacatgcacctgaaatcgctggtttgaggcaagagagggctgaaggaaaggtaccacacttgtttatatagctgtgcctactatcaaactagtaaagatatctcccatatgtgtgctgtccaaatagtttagctgaaggaaaatcataactttatcattttcctttgcacttgtagtttgtgcctgacccgagatttgaagaggttaaggaatacgtccgcagtggcgtctttgggactagcaactatgatgaattgatgggttctttggaaggaaatgaaggttatggacgtgcagattattttcttgttggcaaggattttcccagttacattgaatgccagcaaaaagttgatgaagcataccgagatcagaaggtaagttgtataactgacgctgtttgttttctggctgaaatgcatacggtgattatgcaaactttctgatatgtgatggctgacatcagtgtttcactgttaatgactttactgattcgtaggaaggaatatttcttaattcaaagtttcaaaccggagtatctatcgatgtgttggaacttggaagatcataaacaatcagagaatatagttgctagatgtgctatactactcatctgttggctgttgagtgttgatacatgagctttgataaacctagctagaccttcaggaaagtgcttctatgcattcaataaaaaaataactttataacttaacaaaataattaggttgaacctcgccaggggggcccttttgtaatctgttttctgaaatgaaaatcagaagcagggagctcctcgattctaaaatgaaaaataaaatcagggataactgagggtctggtttccctctgttggagccccagagaattctgcggtcttatttttgtaagagtataagatgcactgctccgttaagtttctccttcatccatttgttttctttgcttcgtgacatttggtgtaacagagcatcccatttgtacacattattttgctatcgtggatatttccactctgtttcatgtgcgacattttaaattcgttacatcgttactaactacacttagtctgatagattttcagagttttggtcctcctaataacataatatccgtttctacctgcagctatggacaagaatgtctatcctcaacacagcgggttcccccaagttcagcagtgaccggacgatccatgagtacgccaaggacatatgggatatcagccccgtcatcatgccctgaataattcaagcagcttccaggaaccttgagagtctatgaggcgcgattggtcgcatatttgcctcgattcagcgagaatgcattgctcccccccagttttgtataggcatatgcaagttctgacatggacctgagtgctgagagcatgcagggtagttaatccttagcaatcttgcgtgtcagttggccagtattacccaggcttatggtgtgtgtatttcgccgaataaagttggagcagaatcatcaaacaaacatactttggtgtatctgtaatgataaataaatggtaaatgactgaacaaaaagtgtcgttggcgcacacatgtagtagaagcatgtttcttcagagagggtgcaaagctgaaagcaccccaactttgaagtgagaatgcagaagtcacgaaaacatttgtttacatccaaagtgcaatgggattcgtcaagagttcagaatcttccctataatagagtagtagcagtggtccctcacattttga

SEQ ID NO: 6 - DNA, 2916 bp, Triticum aestivum
  source 1..2916: mol_type = genomic DNA
atggcgaccgcctcgccgccgctcgccaccgccttccgcccgctcgccgccgccggcggcgccggaggaggaggagcgcacgccgtgggcgccgcgggccgggtcgcgccgaggcgggggcgccggggcttcgtggtgcggagcgtggcgagcgatcgggaggtgcgggggcccgcctcgaccgaggaagagctttcagccgtgctaacttccattgattcttccgccatcgcgtcaaatatccagcaccatgcagacttcacaccgttgttctcaccagagcactcttcacccctaaaggcttaccatgcaactgctaaaagtgtttttgattctctgatcatcaattggaatgcaacatatgactattacaacaaagtgaatgcaaagcaagcttattacctgtccatggagtttttgcagggaagagctctcacaaatgctattggcaatctagagctaacaggacaatatgcagaagcgttaaaacaacttggacagaacctagaggatgttgctagtcaggaaccagatcctgcccttggcaatggtggtctaggccgtctagcgtcctgttttctggattctatggcaaccctaaattatccagcatggggatatggacttcggtacagatatggcctctttaagcaaatcatagcaaaggatggtcaggaggaggtagctgagaattggctagagatgggaaatccatgggagatcgtaagaaatgatgtctcttatcctgtgaaattctatggcaaagtggttgaaggcactgatgggagaaagcactggattggaggagagaatatcaaggctgtggcacatgatgttcctattcctggctacaagactaaaactactaataatcttcgtctttggtcaacaacagtaccatcacaaaattttgatttgggtgcctttaatgctggggatcatgccaaggccaatgaagcacatctaaatgctgaaaagatatgccacgtattgtatccaggggacgaatcatcagaggggaaaattcttcgcttgaaacaacaatatacattatgctcggcctccctgcaggacattatttctcgttttgagtcgagggctggtgattctctcaactgggaggacttcccctctaaagttgcagttcagatgaatgacactcatccaacactgtgcattccggagttaatgagaatactgatggatataaagggattgagctggaatgaggcttggagtatcacagaaagaaccgttgcatacactaaccacacggtacttcctgaagctctggagaagtggagcttggacattatgcagaaacttctaccccgacatgttgagatcatagaaacaatagatgagaagctgatgaacaacatcgtctcaaaatatggaacggcagatatttcactgttaaaacagaagcttaaagatatgaggatcttagacaacgttgatcttccagcctctgttgctaaactgtttattaaacctaaagagaaaacggggaagttgcttgttcaatctttggagtctattgctgaaggtgacgagaaaactgagtcacaagaggaggaaaacattctatccgagacagcagagaaaaagggcggatctgactctgaagaagctcctgatgcagaaaaggaggatcctgtgtatgagttagatccatttgcaaaatacgatcctcagttacctagagttgttcgaatggcaaatctctgtgttgttggtggacattcagttaatggtgtggctgagattcacagtgaaattgttaagcaagatgtgttcaatagcttttatgagatgtggcctactaagttccaaaacaaaacaaacggagtaactcccaggcgttggatccggttttgtaatcctgaattaagtgcaatcatttcaaaatggataggctctgatgactggattctgaacactgataaacttgcaggactgaagaagtttgctgatgatgaggatctgcaatcagaatggcgtactgctaaaaggaataacaagatgaaggtagtttcgctgataagagacaagactggatatgttgtcagcccagatgcgatgtttgatgtgcaggtgaaaagaatacatgaatataagcggcagctgctaaatatccttggtatcgtttaccgatacaagaagatgaaagaaatgagtgccaaagatagaataaagagctttgttccaagggtatgcatatttggtgggaaagcttttgccacttacgtacaggcaaagaggattgtgaagtttatcacagatgtcgcagctactgtaaattatgaccctgatgttggagatttactgaaggttgtatttgtcccagattataatgttagcgttgctgagaagctcattcctgccagtgaattgtcccagcatatcagtaccgctggaatggaagctagtggaaccagcaacatgaagtttgcaatgaatggttgtattcttattggaactttggatggtgcgaatgtggagatcagagaagaggttggagaggagaactttttcctctttggtgcacatgcacctgaaatcgctggtttgaggcaagagagggctgaaggaaagtttgtgcctgacccgagatttgaagaggttaaggaatacgtccgcagtggcgtctttgggactagcaactatgatgaattgatgggttctttggaaggaaatgaaggttatggacgtgcagattattttcttgttggcaaggattttcccagttacattgaatgccagcaaaaagttgatgaagcataccgagatcagaagctatggacaagaatgtctatcctcaacacagcgggttcccccaagttcagcagtgaccggacgatccatgagtacgccaaggacatatgggatatcagccccgtcatcatgccctga

SEQ ID NO: 7 - protein, 971 aa, Triticum aestivum
MATASPPLATAFRPLAAAGGAGGGGAHAVGAAGRVAPRRGRRGFVVRSVASDREVRGPASTEEELSAVLTSIDSSAIASNIQHHADFTPLFSPEHSSPLKAYHATAKSVFDSLIINWNATYDYYNKVNAKQAYYLSMEFLQGRALTNAIGNLELTGQYAEALKQLGQNLEDVASQEPDPALGNGGLGRLASCFLDSMATLNYPAWGYGLRYRYGLFKQIIAKDGQEEVAENWLEMGNPWEIVRNDVSYPVKFYGKVVEGTDGRKHWIGGENIKAVAHDVPIPGYKTKTTNNLRLWSTTVPSQNFDLGAFNAGDHAKANEAHLNAEKICHVLYPGDESSEGKILRLKQQYTLCSASLQDIISRFESRAGDSLNWEDFPSKVAVQMNDTHPTLCIPELMRILMDIKGLSWNEAWSITERTVAYTNHTVLPEALEKWSLDIMQKLLPRHVEIIETIDEKLMNNIVSKYGTADISLLKQKLKDMRILDNVDLPASVAKLFIKPKEKTGKLLVQSLESIAEGDEKTESQEEENILSETAEKKGGSDSEEAPDAEKEDPVYELDPFAKYDPQLPRVVRMANLCVVGGHSVNGVAEIHSEIVKQDVFNSFYEMWPTKFQNKTNGVTPRRWIRFCNPELSAIISKWIGSDDWILNTDKLAGLKKFADDEDLQSEWRTAKRNNKMKVVSLIRDKTGYVVSPDAMFDVQVKRIHEYKRQLLNILGIVYRYKKMKEMSAKDRIKSFVPRVCIFGGKAFATYVQAKRIVKFITDVAATVNYDPDVGDLLKVVFVPDYNVSVAEKLIPASELSQHISTAGMEASGTSNMKFAMNGCILIGTLDGANVEIREEVGEENFFLFGAHAPEIAGLRQERAEGKFVPDPRFEEVKEYVRSGVFGTSNYDELMGSLEGNEGYGRADYFLVGKDFPSYIECQQKVDEAYRDQKLWTRMSILNTAGSPKFSSDRTIHEYAKDIWDISPVIMP

SEQ ID NO: 8 - DNA, 2000 bp, Triticum aestivum
  source 1..2000: mol_type = genomic DNA
  variation 749..966: note = N can be any nucleotide selected from adenine, guanine, cytosine and thymine.
tcatcctcggtgaagggctgctcaagtgcagggaggtcaaagtgatgtgggtcaatgctatggggatcaagggagagggtcggtgctcaaaagtgccaagaatggaagagaagtgatcatataccgccttggccatattgggctcgctggaatgtgcccaccatcaggctgcagccctagaatcattaacttctgtcgctagtgcgtggaatggatcttgaagaaggcggaatttgtgtcgccctcacgtagccaggagaacttgacagggacctctccagtgaagcaagtcctaggtaggcgcgcttcagctcgcggagtaactagacttctggaggagctaccaggtggtgatcttgagccgcatcgagacgagcggtctactcccgagcgagaagcatctgagtggacatgttactgagcgttctggggatccaactctatagtcagcgcattgtcagcttgagctttgcgtagatccatcggaaaggatcggggtcatgcacgctagaattccaggtgtcagagaccacctgactaaaaccatcgagctttcgccaaaatagctgaagatggaatctttgatcaccttgggagcaaaaatttcattggaggccaagttagttctatatttgtgtctgcaattactgatcattggccgcttagcaaactcctattggttcggtaaagcttaaggtcatccactttggaaaaatgctacttgctacaaaccctctcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntggcttttctagttgagaggaagcaaagacgtatcaagacgacaatatcaccctcggcaaggaataagtccttcccgcctcgtcctcgtttcgatgctgcttcttgtcattgcggtgaggcgcgtggaggttggcgtgtccccaaatctagttatctgggtcggggtttttctttggcaggtttatctcagtggtagtgtcccgtgaaggggatgacacgactatgtgtcttggtcctccatgctctagttttgacatgtgaaggttggccaacttcagcgttgtcggggagcatatgagatttgtgttcctttacttcgcctggtcagacctgcggtattatataagccctctcatgtgttttcttctcttgggcagcagtcagttacgcaaatcgtggtcgctgacatcttctagtccacattggcgacttcccgaccgccgcttctacaagctccttatttttcaacaagttttctccaccgagatggaaatcaagaggcggcaacggctttgctcatggcgcactgcccatggatgcaggagaaggaagacattccacacccctcaaactatgaagatgtaatttttagtttttgtaagtatttttgtgaggatttgtgctacttttaatatatatggccttttgcctttttccaggaaaggaaaaactaaaccattacatgaggccattcccatccgtccaataaatacgacaaaacgctttacagcacgggggcaattcccagccgtcgaaatgcgctgcaacggaactagctctcccagacagacacagcggcacatgagccacgcctcccatcgcaaccgtgcaccggcggtgggcccaccggccagcgcccccacacgtgtaccggcggccagcattgcacggcacggcgacggagagatatttttaacgccttccaattcagaagctccccatcactcgctcgggtatcagagcgggccgcgtgactctcgcgtcgtcgcatcgcgcgcgctgcacaccaccgtcagcgcgaccactggcgcca

SEQ ID NO: 9 - DNA, 8733 bp, Triticum aestivum
  source 1..8733: mol_type = genomic DNA
agtggtagtgtctcgtgaaagcggatgacgcgactatgtgtcttggtcctccacgctctagtttcgacaggtgaaggttggccagcttcggcctcgtcggggagcgtatgagatttgtgttcctttacttcgtctggccagacttcccgaccgccgcttctacaagctccttatttcaagcaagttttcttcaccgagatggaagtccagaggcggcaacagctttgctcacggcgcagtggcggtggatgcaggagaaagaagacattcaacacccctaaactatgaagatgtaattttttatttttgtaagtgcttttgtgaggacttgtgctacttttaatatgtggccttttgcctttttccagaaaaaaaaaactaaaccattacacgaggagaagagccattcccagccgtccaataaacacgacaaaacgctttacagcacgggggcagttcccagccgtcgaaatgcgctgcaacggaactagtagctctccctgacacggcggcacatgagccacgccttgcgccgtgcaccggcggtgggcccaccggccagtgcccccacacgtgtaccggcggccagcattgcacggcacggcgacggagagatatttttaacgccttccaattcagaagctcccccatcactcactcgggtacagagcgggccgcgtgactctcgcgtcgtcgcatcgcgcgcgctgcacaccaccgtcagcgcgaccactgccaccaatggcgaccgcctcgccgccgctcgccaccgccttccgcccgctcgccgccggcggcggcaccggaggaggaggagcgcacgccgcgggctgggtcgcgccgaggcgggggcgccgggggttcgtggtgcggagcgtggcgagcgatcgggaggtgcgggggccggcctcgaccgaggaaggtaagcgccgcgcgctcccgttggctacgacgacccggaatgctggtcggctgtaatgtggttcggattttcgctggtgcgacctgaatttgtgatcctctgactgacggtctctgtgttccccggtttcgggctattgattttgtgcgcgcgcattgctaatctactgctctaggatttgttagggttcagaggaatcttaaattcgtgcataaacttcgacgaatgtattctgaatttactgaaactggcttagtttggcgcatcaagttagaatgatgtatttatggtacagtagcaaattctgagaaattcttctgtgcctgcacagccaccccttgtggatgatgcagcaagtgtcaacatactgtcgctactatctgtagctgtaacaaaatcttacgccttgttttctggtggtacagtagtattatcattttacaaagagtagctgtaacaaaatcttacgccttgttttctggtggcttgccattggggagtagaatgcaaatggtgatacagtatattatctacgattttgtgcaagaattttcacacgaaaacaatgtcccatgcagtttttagttttgactcttgactgtttaatacctactctttgtaagatttcggatcctaatattgttgttcttgcaactcagagctttcagccgtgctaacttccattgattcttccgccatcgcgtcaaatatccagcaccatgcagacttcacaccgttgttctcgccagagcactcttcacccctaaaggcttaccatgcaactgctaaaagtgtttttgattctctgatcatcaattggaatgcaacatatgactattacaacaaagtgaatgcaaagcaagcttattacctgtccatggagtttttgcaggtctcacaatatacttcctttagccttatctcaatactttttggtaactgcgttatgatgttatatggttattatctgtgtactgcagggaagagctctcacaaatgctattggcaatctagagctaacaggacaatatgcagaagcgttaaaacaacttggacataacctagaggatgttgctagtcaggtaagtgatttttgtcagagattggtttttagtctgtatcaaaattatcgcagagccattttttttttctaaaaaacacattttattattaatcatcaagttccttttgtttaaaaactatctgaaaagaagaagtattttcccctgggggttccagcatctgacttatattctttatgcagttttcagagtgagtgactatgaaattacgccaaaacacgagcttattgatgcctagagtgccctgtagtttagaaatctattattggatatgttacttttctattacaatgaaacaggaaccagatcctgcccttggcaatggtggtctaggccgtctagcgtcctgttttctggattctatggcaaccctaaattatccagcatggggatatggacttcggtacagatatggcctctttaagcaaatcataacaaaggatggtcaggaggaggtagctgagaattggctagaggtagtgatatgtcttctacttgtttactgtttcgaagtgttgactatgttggtttatgttttaatgacctgcatgttagcttcttttgctgttataactgatgctgtatctccagatgggaaatccatgggagattgtaagaaatgatgtctcttatcctgtgaaattctacggcaaagtggttgaagggactgatgggagaaaacactggattggaggagagaatattaaggctgtggcacatgatgttcctattcctggctacaagactaaaactaccaataatcttcgtctttggtcaacaacagtaccatcacaaaattttgatctgggagctttcaatgctggggatcatgccaaggccaatgaagcacatctaaatgctgaaaaggtattcttccgtctacatttattttgaacgtaaaccattaactctcaacaactattgattcaaaggctaaacactgtcttgctagtagttactgtgttttctcttagatgtcattttagaatgtgcgcggtcaatcgtttgataactttgatagtaataatatactggtatacacaaacctatttgggtttctcatgtaaaaatgtcgtcaccaaatttttcatgaaaatacttattgtattttcatgtaaatagaaaagctgatggtcaggtgttttgtgtgtcaaatgacaagtaaaaatgaacagagtttgtgcaccaataacttcttcacctggtgtttgtaattctttcggttgtatttatgtgttttcttattctgccttaattaactcagcatatgcatcaggttacattactcctctgtgtcaagaaaaaagaagtctgatgagatgcagtccagtagtctgtagatggcacattgtagaagaacttttcacatggaaacgttactctgtgatttttgttatgccttttgagtgtttgtgcattgtgcacatataccattaagtataagtgttcaaaagtggttctctcatactgtgcaaactagctaaacaacttgactggacaagataaattgtactcacatctgaagctgccgggttggacagatttgtccgaaccatctttgaacatccctactagtgtgcatgggttaaattgttgacttttcctcgtttgttcagtagtaggactatctaaatatatttatattcaagttttctttgtgtgttacgttataaacttttagtcataattacatttgtgaccattgtggacaaaatttatcatgtctaacttaagtttgtgactggtcttctcatcatgccagatatgccacgtattgtatccaggggacgaatcatcagaggggaaaattcttcgcttgaaacaacaatatacattatgctcggcctccctgcaggacattatttctcgttttgagtcgagggctggtgattctctcaactgggaggacttcccctctaaagttgcagttcagatgaatgacactcatccaacactgtgcattcccgagttaatgagaatactgatggatataaagggattgagctggaatgaggcttggagtatcacagaaaggtaccacagaagggtactgcagtttgatgtgttctgctgttcctccagtttcaattaactaattttatccctcatatgtcttccattcaataataaactgattgtctgatatcatttgtagaaccgttgcatacactaaccacacggtacttcctgaagccctggagaagtggagcttggacattatgcagaaacttctacctcgacatgttgagatcatagaaacaatagatgaggaggtaacaaatgtgcagataatattatacaattaatttcatgctctgtaggtcaacttatggtttacttatcttcatcgttgattttaaaacagctgatgaacaacatcgtctcaaaatatggaacggcagatatttcactgttaaaacagaagcttaaagatatgaggatcttagacaacgttgatcttccagcctctgtggctaaactgtttgttaaaacaaaaatgaaaaaggggaagttgcttgttgaatctttggagtctattgctgaagctgacgagaaaactgagccagaagaagaggaaaacattctatccgagacaacagagaagaagggcgagtctgactctgaagaagctcctgatgcagaaaaggaggatcctgagtatgagttagatccatttgcaaaatacgatcctcagttacctagagttgttcgaatggcaaacctctgtgttgttggtggacattcagttaatggtgtggctgagattcacagtgaaattgttaagcaagatgtgttcaatagcttttatgaggtatttgcacgatgatttggaaaccatgatcttatcttctaacggctgctataatcgccaggtaatatggtattcaccatctgccattttcttgtgttctttgccagatgtggcctactaagttccaaaacaaaacaaacggagtaactcccaggcgttggatccggttttgtaatcctgaattaagtgcaatcatttcaaaatggataggctctgatgactggattctgaacactgataaacttgcaggactgaagaaggtacttccagtaattttaatttccaataatcagtaatctaatgcttccttcacttccatgttacccgtgccttgtttttacattgatatgtgttcatcccgtaatagtttgctgatgatgaggatctgcaatcagaatggcgtactgctaaaaggaataacaagatgaaagtagtttcactgataagagataagactggatatgttgtcagcccagatgcgatgtttgatgtgcaggtatattttggacaagaaacatctgtcttctgttaaatatggtttaactcggatcttggtacttgacacttaggtgaaaagaatacatgaatataagcggcagctgctaaatatccttggtatcgtttaccggtacaagaagatgaaagaaatgagtgcaaaagatagaataaagagctttgttccaagggtatgcatatttggtgggaaagcttttgccacttatgtacaggcaaagaggattgtgaagtttatcacagatgtcgcagctactgtaaattatgatcctgatattggagatttactgaaggtaaatttgtgtcttcaatggcacttattattcttaattttcatgttctgtcactgggaatttgactccgactaggggttactgaaaagcagtgaatgttccgagtttcaaaagaaaacagagcagtgaatgttcttaatgtcggtgcttgtagaaccgagaaactgaatagccacagtattttttagagaacaatagccacaattgttttgacccttctattgctatcaagcatctgatgccgattttactgataactagatgaggtaaatacaaggtgacaggttaagttctgaggatttttgtttgttatacaggttgtatttgtcccagattataatgttagcgttgctgagaagctcattcctgccagtgaattgtctcagcatatcaggtaatattctggctactcttatatagtatgtgaatacatagaaaaggagtcttacctggagttggctgatgtggaaattttcatgtgccgttttgtttgctataatatatacctggagtcttgctgggatagtgccatccagaagtgccacgccagcttctatttctgtacaggatgtgcatttttaacactattactatccattcagtaccgctggaatggaggctagtggaaccagcaacatgaagtttgcaatgaatggttgtattcttattggaactttggatggtgcgaatgtggagatcagagaagaggttggagaggagaactttttcctctttggtgcacatgcacctgaaatcgctggtttgaggcaagagagggctgaaggaaaggtaccatcactttgcaaacatctccaaattcgttcctccagtatgtgaaacaaatatccacttgtttatatagctgtgcctactatcaaactactccctccgtccataatataagagtgttttttacactacactagtgtcaaaaacgctcttatattatgggacggagggagtagtaaagatctctcccaaatgtgtgctgtccaaatagtttagctgaaggaaaattataaatcattttcctttgcacttgtagtttgtgcctgacccgagatttgaagaggttaaggaatacgtccgcagtggcgtctttgggactagcaactatgatgaattgatgggttctttggaaggaaatgaaggttatggacgtgcagattattttcttgttggcaaggattttcccagttacattgaatgccaggaaaaagttgatgaagcataccgagatcagaaggtaagttgtataactgactctgtttgttttctggctgaaatgcacacagtgattatgcaaactttctgatatgtgatggctgacatcagtgtttcactgtgaacgacttcactgattcgtaggaaggaatatttcttagttcaaagtttcaaactgaagtatctatcggtgtgttggaagatcataaacaatcagagaatatagttgctcgatgtgctatattactcatctgttggctgttgagtgttgatacatgagctttgatgaacctagctagaccttcgggaaagtgcttctatgcattccaataaaaaaaacttatatcttaacaaaacaattaggttgaacctcgccagggggctttttgttatctgtttcctgaaatgaaaatcagaagcggggagctcctcaatttgaaaaaaaaatcaagggtaactgagggtctggtttccctctgttggagcccccaagaattctgtggtcttatttttgtaatagtataagatgcactgctccgttaagtttctccttcatccatttgcttttattgcttcgtgacatttcgtgtaacagctcagattaccatgcatcccattacacattattttgctactgtggatatttccactctgtttcatgcttgacatttttaattcgttacatcgtgaccaactaaacatagtctgatagattttcagagttttgatcctcctaatcacataatatccgtttctacctgcagctatggacaagaatgtctatcctcaacacagcgggttcccccaagttcagcagtgaccggacgatccacgagtacgccaaggacatatgggatatcagccctgtcatcatgccctgaataattcaagcagcttccaggaaccttgagagtctatgaggcgcgattggtcgcagatttgcctcgattcagcgagaatgtattgctccccccagttttgtataggcatatgcaagttctggcatggacctgagtgctgagagcatgcagggtagttcatccttagcaatcttgcatgtcagttggccagtatacccaggcttatggtgtgtatttcgccgaataaagttggagcagaatcatcaaacaaacatactttggtgtatctgtaatgataaataaagggtaaatgactgaacaaaaagtgtcgttggcgcacacatgtagtagaagcatgtttcttcagagagggtgcaaagctgaaagcacccccagctttgaaatgagaatgcagaagttgggaaaacatttgtttacatccaaagtgcaacgggattcgtcaagagttcagagtcttccatcacggccatagagcatgtccaatagcagtcctataatagagcagtagcagtggtccctcatattttgaaccaaaaacgcagctccaacggctgccctatttgtaaaatttacttcaaaatttgttggacagcccgaaaatgcaacaccagctgatgtaaatgtacaacacttcggctgctgccctaaaatttgcttcaagtggcgaaccactcaagaccacaagaaaacagttgttttacgttactttgtgccaaaaacgttgctccaacaactgcgctataataggccaccctaatgtattttagggctgttgttggagatacatattttcgggtgcctaaaaataaaatcattttttggtgccttacgttttacgccaaccactcttttgtgccctattatagggctgctattggagatactctaacagttccatttttccataaaagtttctgatttggagggagttgagcaaaaaacaaagtttttgtaaacgtgaatttttagtggcctttatagttagcccaactccccctccacctaa

SEQ ID NO: 10 - DNA, 2907 bp, Triticum aestivum
  source 1..2907: mol_type = genomic DNA
atggcgaccgcctcgccgccgctcgccaccgccttccgcccgctcgccgccggcggcggcaccggaggaggaggagcgcacgccgcgggctgggtcgcgccgaggcgggggcgccgggggttcgtggtgcggagcgtggcgagcgatcgggaggtgcgggggccggcctcgaccgaggaagagctttcagccgtgctaacttccattgattcttccgccatcgcgtcaaatatccagcaccatgcagacttcacaccgttgttctcgccagagcactcttcacccctaaaggcttaccatgcaactgctaaaagtgtttttgattctctgatcatcaattggaatgcaacatatgactattacaacaaagtgaatgcaaagcaagcttattacctgtccatggagtttttgcagggaagagctctcacaaatgctattggcaatctagagctaacaggacaatatgcagaagcgttaaaacaacttggacataacctagaggatgttgctagtcaggaaccagatcctgcccttggcaatggtggtctaggccgtctagcgtcctgttttctggattctatggcaaccctaaattatccagcatggggatatggacttcggtacagatatggcctctttaagcaaatcataacaaaggatggtcaggaggaggtagctgagaattggctagagatgggaaatccatgggagattgtaagaaatgatgtctcttatcctgtgaaattctacggcaaagtggttgaagggactgatgggagaaaacactggattggaggagagaatattaaggctgtggcacatgatgttcctattcctggctacaagactaaaactaccaataatcttcgtctttggtcaacaacagtaccatcacaaaattttgatctgggagctttcaatgctggggatcatgccaaggccaatgaagcacatctaaatgctgaaaagatatgccacgtattgtatccaggggacgaatcatcagaggggaaaattcttcgcttgaaacaacaatatacattatgctcggcctccctgcaggacattatttctcgttttgagtcgagggctggtgattctctcaactgggaggacttcccctctaaagttgcagttcagatgaatgacactcatccaacactgtgcattcccgagttaatgagaatactgatggatataaagggattgagctggaatgaggcttggagtatcacagaaagaaccgttgcatacactaaccacacggtacttcctgaagccctggagaagtggagcttggacattatgcagaaacttctacctcgacatgttgagatcatagaaacaatagatgaggagctgatgaacaacatcgtctcaaaatatggaacggcagatatttcactgttaaaacagaagcttaaagatatgaggatcttagacaacgttgatcttccagcctctgtggctaaactgtttgttaaaacaaaaatgaaaaaggggaagttgcttgttgaatctttggagtctattgctgaagctgacgagaaaactgagccagaagaagaggaaaacattctatccgagacaacagagaagaagggcgagtctgactctgaagaagctcctgatgcagaaaaggaggatcctgagtatgagttagatccatttgcaaaatacgatcctcagttacctagagttgttcgaatggcaaacctctgtgttgttggtggacattcagttaatggtgtggctgagattcacagtgaaattgttaagcaagatgtgttcaatagcttttatgagatgtggcctactaagttccaaaacaaaacaaacggagtaactcccaggcgttggatccggttttgtaatcctgaattaagtgcaatcatttcaaaatggataggctctgatgactggattctgaacactgataaacttgcaggactgaagaagtttgctgatgatgaggatctgcaatcagaatggcgtactgctaaaaggaataacaagatgaaagtagtttcactgataagagataagactggatatgttgtcagcccagatgcgatgtttgatgtgcaggtgaaaagaatacatgaatataagcggcagctgctaaatatccttggtatcgtttaccggtacaagaagatgaaagaaatgagtgcaaaagatagaataaagagctttgttccaagggtatgcatatttggtgggaaagcttttgccacttatgtacaggcaaagaggattgtgaagtttatcacagatgtcgcagctactgtaaattatgatcctgatattggagatttactgaaggttgtatttgtcccagattataatgttagcgttgctgagaagctcattcctgccagtgaattgtctcagcatatcagtaccgctggaatggaggctagtggaaccagcaacatgaagtttgcaatgaatggttgtattcttattggaactttggatggtgcgaatgtggagatcagagaagaggttggagaggagaactttttcctctttggtgcacatgcacctgaaatcgctggtttgaggcaagagagggctgaaggaaagtttgtgcctgacccgagatttgaagaggttaaggaatacgtccgcagtggcgtctttgggactagcaactatgatgaattgatgggttctttggaaggaaatgaaggttatggacgtgcagattattttcttgttggcaaggattttcccagttacattgaatgccaggaaaaagttgatgaagcataccgagatcagaagctatggacaagaatgtctatcctcaacacagcgggttcccccaagttcagcagtgaccggacgatccacgagtacgccaaggacatatgggatatcagccctgtcatcatgccctga

SEQ ID NO: 11 - protein, 968 aa, Triticum aestivum
MATASPPLATAFRPLAAGGGTGGGGAHAAGWVAPRRGRRGFVVRSVASDREVRGPASTEEELSAVLTSIDSSAIASNIQHHADFTPLFSPEHSSPLKAYHATAKSVFDSLIINWNATYDYYNKVNAKQAYYLSMEFLQGRALTNAIGNLELTGQYAEALKQLGHNLEDVASQEPDPALGNGGLGRLASCFLDSMATLNYPAWGYGLRYRYGLFKQIITKDGQEEVAENWLEMGNPWEIVRNDVSYPVKFYGKVVEGTDGRKHWIGGENIKAVAHDVPIPGYKTKTTNNLRLWSTTVPSQNFDLGAFNAGDHAKANEAHLNAEKICHVLYPGDESSEGKILRLKQQYTLCSASLQDIISRFESRAGDSLNWEDFPSKVAVQMNDTHPTLCIPELMRILMDIKGLSWNEAWSITERTVAYTNHTVLPEALEKWSLDIMQKLLPRHVEIIETIDEELMNNIVSKYGTADISLLKQKLKDMRILDNVDLPASVAKLFVKTKMKKGKLLVESLESIAEADEKTEPEEEENILSETTEKKGESDSEEAPDAEKEDPEYELDPFAKYDPQLPRVVRMANLCVVGGHSVNGVAEIHSEIVKQDVFNSFYEMWPTKFQNKTNGVTPRRWIRFCNPELSAIISKWIGSDDWILNTDKLAGLKKFADDEDLQSEWRTAKRNNKMKVVSLIRDKTGYVVSPDAMFDVQVKRIHEYKRQLLNILGIVYRYKKMKEMSAKDRIKSFVPRVCIFGGKAFATYVQAKRIVKFITDVAATVNYDPDIGDLLKVVFVPDYNVSVAEKLIPASELSQHISTAGMEASGTSNMKFAMNGCILIGTLDGANVEIREEVGEENFFLFGAHAPEIAGLRQERAEGKFVPDPRFEEVKEYVRSGVFGTSNYDELMGSLEGNEGYGRADYFLVGKDFPSYIECQEKVDEAYRDQKLWTRMSILNTAGSPKFSSDRTIHEYAKDIWDISPVIMP

SEQ ID NO: 12 - DNA, 2000 bp, Triticum aestivum
  source 1..2000: mol_type = genomic DNA
tatgttagtccaaataaatatcatctaaaattgtgcctaaaccatatacaaatgatgtaaatgtggacataatacttcataaagtatagatatgtttgagacgtatcaggagcgtcgacctttttgggaagccaagtgagcaaagcctcatcaagtcttgggaatccacggtcattgagggagaaaaatttatcaaaggtctcacacataccccctttgatgatggaccagcaggaccgcagtaatttggccgtgaagccatctagccccgtcaacttgccggaagggaacctcttgaccgcctcctaaatttcatcctcagtgaagggttgctcaagtgcagggaggtcaaagtgatgcgggtcggtactatggagatcaagggagagagtcagtgctcaaaagtgtcaagaatggaagagaagtgatcatatgccgccctggccatatcgggctcgccagaatgtgcccaccatcagactgcacccctaggattattaacttctgtcgtcggtgcacagaatggatcttgaagaaggtggaatttgtgtcgccctcacgtagccaggagaacctggcaggcagatgtgcaatcgacctctccaccgaagcaaaccctaggtaggcgcgcttcagctcgcggagtaaccatacttctggaggagcaagcaggcggtgatcttgagccgcatcgagatgagcggtcaactcccgagcgagaagcatctgagtggagatgttaccaagcgttctagacctccaactctacagtcagcgcattgtcagcttgagcttggcgtagatcccctgaaaatgattggggtcatgcacgccagaattccaggtgtcagagaccacctaatgaaaacaatcgagctttggccaaaatagctcaaaatggaatctccgatcaccttgggagtgaaaatgttactagtttcgttggaggccaaggtagttatgtatttgtgtgtgcaattactaatcattggcccttagcaaactcctattggttcaaaccgtttcactttgaaaaaaatgctacttgctacaaaccgtttcacttggaggggcgtcttttctagttgagagggagcaaaaggcgtatcaagacgacaacgccaccctcggcaatgtacaagtccttccccgtcctcgtttcgatgctgctttttgtcatcgcagcgaggcgtgtagagggtggtgtgtccccagatctagttatctgggttggggtttttctttggcaggtttatctcagtggtagtgtctcgtgaaagcggatgacgcgactatgtgtcttggtcctccacgctctagtttcgacaggtgaaggttggccagcttcggcctcgtcggggagcgtatgagatttgtgttcctttacttcgtctggccagacttcccgaccgccgcttctacaagctccttatttcaagcaagttttcttcaccgagatggaagtccagaggcggcaacagctttgctcacggcgcagtggcggtggatgcaggagaaagaagacattcaacacccctaaactatgaagatgtaattttttatttttgtaagtgcttttgtgaggacttgtgctacttttaatatgtggccttttgcctttttccagaaaaaaaaaactaaaccattacacgaggagaagagccattcccagccgtccaataaacacgacaaaacgctttacagcacgggggcagttcccagccgtcgaaatgcgctgcaacggaactagtagctctccctgacacggcggcacatgagccacgccttgcgccgtgcaccggcggtgggcccaccggccagtgcccccacacgtgtaccggcggccagcattgcacggcacggcgacggagagatatttttaacgccttccaattcagaagctcccccatcactcactcgggtacagagcgggccgcgtgactctcgcgtcgtcgcatcgcgcgcgctgcacaccaccgtcagcgcgaccactgccacca

SEQ ID NO: 13 - DNA, 8314 bp, Triticum aestivum
  source 1..8314: mol_type = genomic DNA
ggcacggcgacggagagatattttaacgccttccaattcagaagctcccccatcactcactcgggtacagagcgcagcgggccgcgtgactctcgcgtcgtcgcatcgcgcgcgctgcacaccaccgtcagcgcgaccactgccgccaatggcgaccgcctcgccgccgctcgccacccccttccgcccgctcgccgtcgccggaggtagcggcggactcgtcgtgggcgccagggccgtggtaccgccgaggcgggggcggcgggggttcgtggtgcggagcgtggcgagcgatcgggaggtgcgggggcccgccccgaccgaggaaggtaagcggccgcgcgctcccattggctgccacgacctggaatgctggtcggctgtaatgtggttcggattttcgctggtgcgacctgaatttgtgatcctctgactgacggcctatgtgttccccgggtttcgggctgttgattttgtgcgcgcgcattgctaatctacttctctaggatttgttagggctcagagcaatcttaaattcgcgaatacacttcgacgaatgtagtctgaatttactgaaactagcttagtttggcgcgtcaagatagactgatgtatttatggtagcaaattctgaaaacttcttctgtgcgtggacagccacccctcgtggatgatgcagcaagtgctaacacagtgtttgctagtacttgctagtatctgtagctgtaacaaaatccttcgaatctgaactagtgagataaatcgtttgagatttttgctgaagattacacagtatcaatatttctgaaggtgattggaacagtatagcatttcaaactggcaaccaagcaggatcagtatactgacgcaatttttttctgaaggtggttggaataccggtatgaattttataatcattgaattaaggcgtaagatttggtaacaaaaactgcttttcacgggaaacgatgaaacatggcacaattcaacctgactttcttttatgaaaaggctctcaagctgccttgtactccctccgtcccataatataagaacgtttttgacattagtgtagtaccaaaaacgttcttatattatgggacagagggagtatttattaatggataaaaagagaggtacagggttctagtagtaacatatgcaaatggtgatacagtatattatctacgattttgtgcaagaattttcacacgaaaacaatattccatgcagtttttagttttgacttttttttttgaattttcacgcgcgcggggggggggggggggggggggggnnnnnnnnnnnnnnnnnnnnnnnnnnnnnnnnnnnnnnnnnnnnnnnnnnnncagagagagaggcagggggcgacccccccatgctatgttacatgcagctttgttctacccacgcagagcctgtagccagttttgactctttttgaattgtttggattgcttaccacatgtgtcacgtggttttagttttgactcttgactgtttaatacctactctttttaagattttggattctaatgtcatctgtcttgcaactcagagctttcagccgtgctaacttccattgattcatccgccatcgcatcaaacatccagcaccatgcagacttcacaccgttattctcgccagagcactcttcacccctaaaggcttaccatgcaactgccaaaagtgtttttgattctctgatattgaattggaatgcaacatatgactattacaacaaagtgaatgcaaagcaagcttattacctatccatggaatttttgcaggtctcgcaatatactttcttcagccttatctcataccttttggtaactgcgttatgatgttatatgtttttttttgtactgcagggaagagctcttacaaatgctattggcaacctagagctaacaggacaatatgcagaagcgttaaaacaacttggacagaacctagaggatgttgctagtcaggtaagccatttttgtcagagaatggtttttagtccatatcaaaattattgccgagccatttttctaaataacattttattattattcatgaacttccatatttttttcaaatctgcaaaaggagaagtttttttcctgggggttccaccatctgacttatatttgttatgcagttttcagagtgattgactatgaaattacgccaaaacacgagcattgcctagagtgccctgtagttttagaaatgttaagattggatatgttacttttctattacaatgaaacaggaaccagatcctgcccttggcaatggtggactaggccgtctagcgtcctgttttctggattctatggcaaccctaaattatccagcatggggatatggacttcggtacagatatggcctctttaagcaaatcataacaaaggatggtcaggaggaggtagctgagaattggctagaggtagtgatatgtcttctacttgtttactgtttggaagcgttgattatgttggtttctgttttaatgatctgcatgttagcttcttttgccatttataactgatgctgtatctccagatgggaaatccatgggagattgtaagaaatgatgtctcttatcctctgaaattctatggcaaagtggttgaaggcactgatgggagaaaacactggattggaggagagaatatcaaggctgtggcacatgatgttcctattcctggctacaagactaaaactactaataatcttcgtctttggtcaacaacagtaccatcacaaaactttgatttgggagcttttaatgctggggatcatgtcaaggccaacgaagcacatctaaatgctgaaaaggtattcctttgtctgcatttattttgaatataaatcattaaatctcaataactattgatttaaatggtaaacactaaacattgtctagctagtagtcactgtggtgtccctcaatgtcattttagaatgtgcacagtcaaatgtttgataactttgattgtaataatatgataatatacagaaactttgattgacactagatttttcataaacatacttattgtatttccatgtaaatagaaaagctgatgttcagaggtttgtgtgtcaaaatgaacagagtttgtgcacacataatatcttcatctggtgtttgcagttctctaggttgtgtttgcgttttctcctgctctgccttaactaatctcagcatatgcatcaggctacattgctcctctgtgtctataaaaaataagtctgatgatatatggttgcagtccagtagttcgtagatggcacagcatggaagaacttttcacatggaagccttacccttataattttaattataccttttgagtgtttgtgcattgtgtacatacaccattaagtatgggtgctcaaaaatggtttatcccaaactgtgcaaaagccgccactgtttgtgtattaatttttttgaactagctaaacaacttgaccgaacaagatacatacaattgcactcacttctgaagcagctcagttggacggatttgttccaatcccgtgcatgggttaaattgtcgagttgtcctcgcttgttcagtagtaggagtactatctattatatttatactaaagttttctttttgtgatacagtacaaacttttagtaatacttacatttgcgaccactgtggacaaaatttattatgtctaacttaactttatcactggtcttctcatcatgccagatatgccacgtattgtatccaggggacgaatcatcagaggggaaaattcttcgcctgaaacaacaatatacactatgctcggcctccctgcaggacattatttctcgttttgagtcgagggctggtgattctctcaactgggaggacttcccctctaaagttgcagttcagatgaatgacacccatccaacactgtgcattcccgagttaatgagaatactgatggatgttaagggattgagctggaatgagtcttggagtatcacggaaaggtatcacagaagggtactgcagtttgatgtgttatgctgttcctccagttccagttaactaattttatttcctcatctgtcttccattcaatcatgtactgattttctgatatccacatcatttgtagaaccgttgcatacactaaccacacagtacttcctgaagctctggagaagtggagcttggacattatgcagaaacttctacctcgacatgttgagattatagaaagaatagatgaggaggtatcaaatgtgcagataatattatacaattaatttcatgctctgtagttcaacttatggtttacttgtcttcatcgttgattttaaacagctgatgaacaccatcgtctcaaaatatggaacggcagatatttcactgttaaaacagaagcttaaagatatgaggatcttagacaacgttgatcttccagcctctgttgctaaactgtttattaaaccaaaagagaaaaaggggaagttgcttgttgaatctttggagtctattgctgaagctgacgagaaaactgagtcagaagaggaggaaaacattctatccgagacaacagagaaaaagggcgaatctgactctgaagaagctcctgatgcagaaaaggaggatcctgagtatgagttagatccatttacaaaatacgatcctcagttacctagagttgttcgaatggcgaacctctgcgttgttggtggacattcagttaatggtgtggctgagattcacagtgaaattgttaagcaagatgtgttcaatagcttttatgaggtatttgcatgatgatttggaaaccgtgatcttattttccaatggctgctataatcgtcaggtgatatggtattcaccatctgccattttcttgtgttctttgccagatgtggcctactaagttccaaaacaaaacaaatggagtgacccccaggcgttggatccggttttgtaatcctgaattaagtacaataatttcaaaatggataggctctgatgactggattctgaacactgataaacttgcaggactgaagaaggtacttccagtagttttattccccaataatcagtaatctaatgcttccttcacttcaatgttaactgtgcattgtttttacattgatgtgtgttcatcctgtaacagtttgctgatgatgaggatctgcaatcagaatggcgtactgctaaaaggaataacaagatgaaggtagtttcgctgataagagacaagactggatatgttgtcagcccagatgcaatgtttgatgtgcaggtatattttgtactagaaaacatgtgtcttcttataaatatggtttaactcagctcttggtactggacacttaggtgaaaagaatacatgaatataagcggcagcttctaaatatccttggtatcatttaccgctacaagaagatgaaagaaatgagtgcaaaagatagaataaagagctttgttccaagggtatgcatatttggcgggaaagcttttgccacttacgtacaggcaaagaggattgtgaagtttatcacagatgtcgcagctactgtaaattatgatcctgatattggagatctactgaaggtaaatttgtgtctttgatgccacttatcagtcttaattttcatgttctgtcactggcaagttgactccgactgaaaagcattgaatgttcagagtttcaaaggaaaacagagcagtgaatgttcttaatgtcggtgcttgtaaaactgagcaactgaatagccacaggttttttagagaacaatatagccacaaatttttttgacacttttattgctatcagcatctgatgctgatattactgataactagatgaggtaaatataaggtgacaggttaagtctgaggatttttgtttgttatccaggttgtatttgtcccagattataatgttagcgttgctgagacgctcattcctgccagtgaattgtcccagcatatcaggtaatattctgcctactcttatatagtatgtgaatacataaaaaaagaatattacctggagttggctgacatagaatttttcatgtgcctttttgtttgctataatatgtacctggagtcttactgggatagtgccatccagaagtgccaccccagtttctatttctgtataagacgtgcatttttaacagtattactatccattcagtaccgctggaatggaagctagtggaaccagcaacatgaagtttgcaatgaatggctgtattcttattggaactttggatggtgcgaatgtggagatcagagaagaggttggagaggagaactttttcctctttggtgcacatgcacctgaaatcgctggtttgaggcaagagagagctgaagggaaggtaccacacttgtttatatagctgtgcctactatcaaactagtaaagatctctcccatatgtgctgtccaaatagtttagctgaaggaaaatcataactttacaagtttcctttgcacttgtagtttgtgcctgacccgagatttgaagaggttaaggaatacgtccgcagtggcatctttgggactggcaactatgatgaattgatgggttctttggaaggaaatgaaggttatggacgtgcagattattttcttgttggcaaggattttcccagttacattgaatgccaggaaaaagttgatgaagcataccgagatcagaaggtaagttgtataactgacgctgtttgttttctggctgaaatgctcacagtgattatgcaaactttctgatatgtgatggctgacatcagtgtttcactgtgaacgacttcattgattcgtaggaaggaatatttcttagtttaaagtttcaaactgaagtatctatcggtgtgttggaagatcataaacaatcagagaatatagttgctagatgtgctatattactcatctgttggctgttgagtgttgatacatgagctttgatgtagctagaccttcaggaaagtgcttctgtgcattccaataaaaaaataacttataacttaacaaaacaaaacaattaggttgaacctcaccagggggccttttgttatctgttttctgaaatgaaaatcagaagtggggagctcctcaattcgaaaaagaaaaagaaaatcagggggtaactgagggtctggtttccctctgttggagcccccaagaattctgtggtcttatttttgtaagagtataagatgcactgctccgttaagtttctccttcacccatttgttttcattgcttcgtgacatttcgtgtgacagctcagatcaccatatgcatcccatttgtacacattattttgctaccgtggatatttccactctgtttcatgtgtgacatttttaattcgttacattgttactctaaacattagtctaatagattttcagagttttgaccctcctaatcacataatatccgtttcgacctgcagctatggacaagaatgtctatcctcaacacagcgggttcccccaagttcagcagtgaccggacgatccacgagtatgccaaggacatatggggtatcagccccgtcatcatgccctgaataattcaagcagcttccaggaaccttgagagtctatgaggcgcgattggtcgctgatttgccttgattcagcgagaatgtattgctccccccagttttgtataggcatatgcaagttctgacatggacctgagtgctgagagcatgcagggtagttcatccttagcaatattgcatgtcagttagccagtattacccaggcttatggtgtgtgtatttcgctgaataaagttggagcagagtcatcaaacaaacatactttggtgtatctgtaatgataaataaatggtacatgtgactgaacaaaaagtgtcgttggcgtgcacatgtagtagaagcatgtttcttcagagagggtgcaaagctgaaagcaccccagctttggagtgagaatgcagaagttacgaaaacatttgtttacatccaaagtgcaacgggattcgtcaagagttcagagtcttccatcacggtcatagagcatgtccaatagcagccctataaacctataacagagcagtagcagtggtccctcacattttgaaccaaaaatgcagctccaacggctgccctatttgtaaaatttacttcaaaatttgttggacagccctaaaatgcaacaccaactgatgtaaatgtacaactattcggctgctgccctaaaatttgctccaagtggcgaaccactcaggaccacaagaaaacagttgttttacgttgtgccaaaaatgttgcttcaactactgc

SEQ ID NO: 14 - DNA, 2907 bp, Triticum aestivum; identical to SEQ ID NO: 2
  source 1..2907: mol_type = genomic DNA

SEQ ID NO: 15 - protein, 968 aa, Triticum aestivum; identical to SEQ ID NO: 3

SEQ ID NO: 16 - DNA, 2000 bp, Triticum aestivum; identical to SEQ ID NO: 4
  source 1..2000: mol_type = genomic DNA

SEQ ID NO: 17 - DNA, 7692 bp, Triticum aestivum; identical to SEQ ID NO: 5
  source 1..7692: mol_type = genomic DNA

SEQ ID NO: 18 - DNA, 2916 bp, Triticum aestivum; identical to SEQ ID NO: 6
  source 1..2916: mol_type = genomic DNA

SEQ ID NO: 19 - protein, 971 aa, Triticum aestivum; identical to SEQ ID NO: 7

SEQ ID NO: 20 - DNA, 1456 bp, Triticum aestivum
  source 1..1456: mol_type = genomic DNA
atgcattaccgagtgggcccagagatacctctccgtcatacggagtgacaaatcccagtctcgatccgtgtcaacccaacagacactttcggagatacccgtagtatacctttatagtcacccagttatgttgtgacgtttggtacacccaaagcactcctacggtgtccgggagttacacgatctcatggtctaaggaaaagatacttgacattggaaaagctctagcaaacgaactacacgatcttgtgctatgcttaggattgggtcttgtccatcacatcattctcctaatgatgtgatcccgttatcaacgacatccaatgtccatagtcaagaaaccatgactatcatttgatcaacgagctagtcaactagaggcttactagggacatgttgtggtctatgtattcacacatgtattacgatttccggataacacaattataacatgaataacagacgattatcatgaacaaggaaatataataataatcttttattattgcctctagggcatatttccaacaattgcggtgaggcgcgtggaggttggcgtgtccccaaatctagttatctgggttggggtttttctttggaaggtttatctcagtggtagtgtcccgtgaagcggatgacacggctatgtgtcttggtcctccatgctctagttttgacatgtgaaggttggccaacttcggcgttgtcagggagcatatgagatttgtgttcctttacttcgcctggtcagacctacggtattatataagccctctcatgtgttttcttctcttgggcagcagtcagttacgcaaatcgtggtcgctgacatcttctagtccacatcggcgacttcccgaccgccgcttctacaagctccttatttttcaacaagttttctccaccgagatggaagtcaagaggcggcaacggctttgctcatggcgcactgcggatggatgcaggagaaggaagacattccacacccctcaaactatgaagatgtaatttttagtttttgtaagtatttttgtgaggatttgtgctacttttaatatatatggccttttgcctttttccaggaaaggaaaaactaaaccattacatgaggccattcccatccgtccaataaatacgacaaaacgctttacagcacgggggcaattcccagccgtcgaaatgcgctgcaacggaactagctctcccagacagacacagcggcacatgagccacgcctcccatcgcaaccgtgcaccggcggtgggcccaccggccagcgcccccacacgtgtaccggcggccagcattgcacggcacggcgacggagagatatttttaacgccttccaattcagaagctccccatcactcgctcgggtatcagagcgggccgcgtgactctcgcgtcgtcgcatcgcgcgcgctgcacaccaccgtcagcgcgaccactggcgcca

SEQ ID NO: 21 - DNA, 8733 bp, Triticum aestivum; identical to SEQ ID NO: 9
  source 1..8733: mol_type = genomic DNA

SEQ ID NO: 22 - DNA, 2907 bp, Triticum aestivum; identical to SEQ ID NO: 10
  source 1..2907: mol_type = genomic DNA

SEQ ID NO: 23 - protein, 968 aa, Triticum aestivum; identical to SEQ ID NO: 11

SEQ ID NO: 24 - DNA, 2000 bp, Triticum aestivum; identical to SEQ ID NO: 12
  source 1..2000: mol_type = genomic DNA

SEQ ID NO: 25 - DNA, 10610 bp, Triticum durum
  source 1..10610: mol_type = genomic DNA
  variation 1459..1589: note = N can be any nucleotide selected from adenine, guanine, cytosine and thymine.
cgagggcggcggcgaagggatctggtgtggtgtcgctcgagggcaccggactaccggaggcttcgctcgctcgaccgcacgttgcgagtccaagacgattttttttcgctggttttctttcgtcggtttaatttcgttcgatctactttgcctgcgaggggagtgcctgcaggcatcgctccctgataacgtaggtttgttcggtttttttaacgtacgaggggatgtgggaggaaataccaaaaaagtatcaaaaaagaccgggtgaggtgggacgaaaataaaacctggaatgcgacctaccaactgagatattaggagtagagatatatgttttgtatctcctttttttggtataattttttttggtgaaacgccgtttcttttgtaagcgaagatccatatctccgatcaggcagattctgcacattcttttcttattttaggattgctcttgcaaaaagaagatcatcaatcaacttagggatcctccacgcagacccaccacgcagcctaggggcaccgtctggccatctggagccccggtggctcccttggaccccatcttctcgctagtggggccttggtccaaaataatccatgtaatttttcatgttttccggcttcgtattttttggtaaaaacccaaaaacaatagaaaagaagaattggcctctgacactagattgataggttagtccaaatcaatattatcaaaaattgtgcccaaaccatatacaaatgatataaatataactagaatacttcataaatttagatacgtttgataaagtgccaagaatggaagagaagtgatcatatgccgccctgtccatatcgggctcatcagaatgttcccaccatcagactccagccccaggattattaacttatgtcggcggtgcgcggaatggatcttgaagaaagcggaatttgtgtcgccctcacgtaaccaggagaacttggcaggttgacgcgcaatcgacctctccagcaaagcaagtcctaggtaggcgcgcttcagctcgcagagtaaccgtacttctggaggagcaagcgggtggtgatcttgagccgccgagacgagcaatcaattaccgagcgagaagcatctgagtggagatgttaccgagcgttctggagctccaactctgccgtcagtgcattgtcagcttgtgctttgcgtagattcgccggaaaggatcaggatcatgcacgccagaattccaggtgtcagagaccacctaatgaaaaccatcgagctttgatcaaaatagctcaagatggaatctccgatcaccttgggagcgaaaatcttactagtttcgttcaaggccaaggtagttatatatttgtgtgtgcaattattaatcattggccgcttagcaaactcttattggttcgataaacattaaagtcatccccactttgagaaaaaatgctacttgctacaaaccctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcaagtaataaatccttgccgcctcgtccttgtttggatgctgcttcttgtcatctcaaggaggcgtgtggagggtggtgtgtccccggatcttgttctctcggttgggttttttctatggcaggtttatctcagtggtagtgtcacgtgaagcagatgacgcgactatgcgtcttggtcctctgcgctctaattccggcagatgaaggttgaccagcttcagcctcgtcggggagcgtatgagatttgtgttttttgactccatctggtcggacctgtggcattatgtagaccctctcatgtattctttttctcctggacagtagtctgttacacaaatcgtggtcgtcggatcttctagtccacatcggcgacttttccgaccgccacttctacaaactccttattttcaacaagtctccttcattgagatggaagtccaggcaatggctttgctcacggcacactgccggcggatgcaagagaaagaagacattcgacacccctaaaactatgaagatgtaatttttaatctttgtaagtgtttttgtgaggatttgtgctacttttaatatattgccttttgccttttgcaagggaaaaaaaactaaaccattacacgaagagaagagccattcccagccgtccaataaacacgacaaaacgctttacagcacgggggcaattcccagccgtcgaaatgcgctgcaacggaaccagcgctccccgacacggcggcacatgagccacaactcgtaccatgcaccgtgcaccggcggtgggcccactggccagcgtccccacacgtgtaccggcggccacaattacacggcacggcacggcgacggagagatattttaacgccttccaattcagaagctcccccatcactcactcgggtacagagcgcagcgggccgcgtgactctcgcgtcgtcgcatcgcgcgcgctgcacaccaccgtcagcgcgaccactgccgccaatggcgaccgcctcgccgccgctcgccacccccttccgcccgctcgccgtcgccggaggtagcggcggactcgtcgtgggcgccagggccgtggtaccgccgaggcgggggcggcgggggttcgtggtgcggagcgtggcgagcgatcgggaggtgcgggggcccgccccgaccgaggaaggtaagcggccgcgcgctcccattggctgccacgacctggaatgctggtcggctgtaatgtggttcggattttcgctggtgcgacctgaatttgtgatcctctgactgacggcctatgtgttccccgggtttcgggctgttgattttgtgcgcgcgcattgctaatctacttctctaggatttgttagggctcagagcaatcttaaattcgcgaatacacttcgacgaatgtagtctgaatttactgaaactagcttagtttggcgcgtcaagatagactgatgtatttatggtagcaaattctgaaaacttcttctgtgcgtggacagccacccctcgtggatgatgcagcaagtgctaacacagtgtttgctagtacttgctagtatctgtagctgtaacaaaatccttcgaatctgaactagtgagataaatcgtttgagatttttgctgaagattacacagtatcaatatttctgaaggtgattggaacagtatagcatttcaaactggcaaccaagcaggatcagtatactgacgcaatttttttctgaaggtggttggaataccggtatgaattttataatcattgaattaaggcgtaagatttggtaacaaaaactgcttttcacgggaaacgatgaaacatggcacaattcaacctgactttcttttatgaaaaggctctcaagctgccttgtactccctccgtcccataatataagaacgtttttgacattagtgtagtaccaaaaacgttcttatattatgggacagagggagtatttattaatggataaaaagagaggtacagggttctagtagtaacatatgcaaatggtgatacagtatattatctacgattttgtgcaagaattttcacacgaaaacaatattccatgcagtttttagttttgacttttttttttgaattttcacgcgcgcggggggtggggggggggggagagaactcccccacctgaatatatttctcgaacccaacatacagtacaatagtacaaatgccggggcggcagcagagagagaggcagggggcgacccccccatgctatgttacatgcagctttgttctacccacgcagagcctgtagccagttttgactctttttgaattgtttggattgcttaccacatgtgtcacgtggttttagttttgactcttgactgtttaatacctactctttttaagattttggattctaatgtcatctgtcttgcaactcagagctttcagccgtgctaacttccattgattcatccgccatcgcatcaaacatccagcaccatgcagacttcacaccgttattctcgccagagcactcttcacccctaaaggcttaccatgcaactgccaaaagtgtttttgattctctgatattgaattggaatgcaacatatgactattacaacaaagtgaatgcaaagcaagcttattacctatccatggaatttttgcaggtctcgcaatatactttcttcagccttatctcataccttttggtaactgcgttatgatgttatatgtttttttttgtactgcagggaagagctcttacaaatgctattggcaacctagagctaacaggacaatatgcagaagcgttaaaacaacttggacagaacctagaggatgttgctagtcaggtaagccatttttgtcagagaatggtttttagtccatatcaaaattattgccgagccatttttctaaataacattttattattattcatgaacttccatatttttttcaaatctgcaaaaggagaagtttttttcctgggggttccaccatctgacttatatttgttatgcagttttcagagtgattgactatgaaattacgccaaaacacgagcattgcctagagtgccctgtagttttagaaatgttaagattggatatgttacttttctattacaatgaaacaggaaccagatcctgcccttggcaatggtggactaggccgtctagcgtcctgttttctggattctatggcaaccctaaattatccagcatggggatatggacttcggtacagatatggcctctttaagcaaatcataacaaaggatggtcaggaggaggtagctgagaattggctagaggtagtgatatgtcttctacttgtttactgtttggaagcgttgattatgttggtttctgttttaatgatctgcatgttagcttcttttgccatttataactgatgctgtatctccagatgggaaatccatgggagattgtaagaaatgatgtctcttatcctctgaaattctatggcaaagtggttgaaggcactgatgggagaaaacactggattggaggagagaatatcaaggctgtggcacatgatgttcctattcctggctacaagactaaaactactaataatcttcgtctttggtcaacaacagtaccatcacaaaactttgatttgggagcttttaatgctggggatcatgtcaaggccaacgaagcacatctaaatgctgaaaaggtattcctttgtctgcatttattttgaatataaatcattaaatctcaataactattgatttaaatggtaaacactaaacattgtctagctagtagtcactgtggtgtccctcaatgtcattttagaatgtgcacagtcaaatgtttgataactttgattgtaataatatgataatatacagaaactttgattgacactagatttttcataaacatacttattgtatttccatgtaaatagaaaagctgatgttcagaggtttgtgtgtcaaaatgaacagagtttgtgcacacataatatcttcatctggtgtttgcagttctctaggttgtgtttgcgttttctcctgctctgccttaactaatctcagcatatgcatcaggctacattgctcctctgtgtctataaaaaataagtctgatgatatatggttgcagtccagtagttcgtagatggcacagcatggaagaacttttcacatggaagccttacccttataattttaattataccttttgagtgtttgtgcattgtgtacatacaccattaagtatgggtgctcaaaaatggtttatcccaaactgtgcaaaagccgccactgtttgtgtattaatttttttgaactagctaaacaacttgaccgaacaagatacatacaattgcactcacttctgaagcagctcagttggacggatttgttccaatcccgtgcatgggttaaattgtcgagttgtcctcgcttgttcagtagtaggagtactatctattatatttatactaaagttttctttttgtgatacagtacaaacttttagtaatacttacatttgcgaccactgtggacaaaatttattatgtctaacttaactttatcactggtcttctcatcatgccagatatgccacgtattgtatccaggggacgaatcatcagaggggaaaattcttcgcctgaaacaacaatatacactatgctcggcctccctgcaggacattatttctcgttttgagtcgagggctggtgattctctcaactgggaggacttcccctctaaagttgcagttcagatgaatgacacccatccaacactgtgcattcccgagttaatgagaatactgatggatgttaagggattgagctggaatgagtcttggagtatcacggaaaggtatcacagaagggtactgcagtttgatgtgttatgctgttcctccagttccagttaactaattttatttcctcatctgtcttccattcaatcatgtactgattttctgatatccacatcatttgtagaaccgttgcatacactaaccacacagtacttcctgaagctctggagaagtggagcttggacattatgcagaaacttctacctcgacatgttgagattatagaaagaatagatgaggaggtatcaaatgtgcagataatattatacaattaatttcatgctctgtagttcaacttatggtttacttgtcttcatcgttgattttaaacagctgatgaacaccatcgtctcaaaatatggaacggcagatatttcactgttaaaacagaagcttaaagatatgaggatcttagacaacgttgatcttccagcctctgttgctaaactgtttattaaaccaaaagagaaaaaggggaagttgcttgttgaatctttggagtctattgctgaagctgacgagaaaactgagtcagaagaggaggaaaacattctatccgagacaacagagaaaaagggcgaatctgactctgaagaagctcctgatgcagaaaaggaggatcctgagtatgagttagatccatttacaaaatacgatcctcagttacctagagttgttcgaatggcgaacctctgcgttgttggtggacattcagttaatggtgtggctgagattcacagtgaaattgttaagcaagatgtgttcaatagcttttatgaggtatttgcatgatgatttggaaaccgtgatcttattttccaatggctgctataatcgtcaggtgatatggtattcaccatctgccattttcttgtgttctttgccagatgtggcctactaagttccaaaacaaaacaaatggagtgacccccaggcgttggatccggttttgtaatcctgaattaagtacaataatttcaaaatggataggctctgatgactggattctgaacactgataaacttgcaggactgaagaaggtacttccagtagttttattccccaataatcagtaatctaatgcttccttcacttcaatgttaactgtgcattgtttttacattgatgtgtgttcatcctgtaacagtttgctgatgatgaggatctgcaatcagaatggcgtactgctaaaaggaataacaagatgaaggtagtttcgctgataagagacaagactggatatgttgtcagcccagatgcaatgtttgatgtgcaggtatattttgtactagaaaacatgtgtcttcttataaatatggtttaactcagctcttggtactggacacttaggtgaaaagaatacatgaatataagcggcagcttctaaatatccttggtatcgtttaccgctacaagaagatgaaagaaatgagtgcaaaagatagaataaagagctttgttccaagggtatgcatatttggcgggaaagcttttgccacttacgtacaggcaaagaggattgtgaagtttatcacagatgtcgcagctactgtaaattatgatcctgatattggagatctactgaaggtaaatttgtgtctttgatgccacttatcagtcttaattttcatgttctgtcactggcaagttgactccgactgaaaagcattgaatgttcagagtttcaaaggaaaacagagcagtgaatgttcttaatgtcggtgcttgtaaaactgagcaactgaatagccacaggttttttagagaacaatatagccacaaatttttttgacacttttattgctatcagcatctgatgctgatattactgataactagatgaggtaaatataaggtgacaggttaagtctgaggatttttgtttgttatccaggttgtatttgtcccagattataatgttagcgttgctgagacgctcattcctgccagtgaattgtcccagcatatcaggtaatattctgcctactcttatatagtatgtgaatacataaaaaaagaatattacctggagttggctgacatagaatttttcatgtgcctttttgtttgctataatatgtacctggagtcttactgggatagtgccatccagaagtgccaccccagtttctatttctgtataagacgtgcatttttaacagtattactatccattcagtaccgctggaatggaagctagtggaaccagcaacatgaagtttgcaatgaatggctgtattcttattggaactttggatggtgcgaatgtggagatcagagaagaggttggagaggagaactttttcctctttggtgcacatgcacctgaaatcgctggtttgaggcaagagagagctgaagggaaggtaccacacttgtttatatagctgtgcctactatcaaactagtaaagatctctcccatatgtgctgtccaaatagtttagctgaaggaaaatcataactttacaagtttcctttgcacttgtagtttgtgcctgacccgagatttgaagaggttaaggaatacgtccgcagtggcatctttgggactggcaactatgatgaattgatgggttctttggaaggaaatgaaggttatggacgtgcagattattttcttgttggcaaggattttcccagttacattgaatgccaggaaaaagttgatgaagcataccgagatcagaaggtaagttgtataactgacgctgtttgttttctggctgaaatgctcacagtgattatgcaaactttctgatatgtgatggctgacatcagtgtttcactgtgaacgacttcattgattcgtaggaaggaatatttcttagtttaaagtttcaaactgaagtatctatcggtgtgttggaagatcataaacaatcagagaatatagttgctagatgtgctatattactcatctgttggctgttgagtgttgatacatgagctttgatgtagctagaccttcaggaaagtgcttctgtgcattccaataaaaaaataacttataacttaacaaaacaaaacaattaggttgaacctcaccagggggccttttgttatctgttttctgaaatgaaaatcagaagtggggagctcctcaattcgaaaaagaaaaagaaaatcagggggtaactgagggtctggtttccctctgttggagcccccaagaattctgtggtcttatttttgtaagagtataagatgcactgctccgttaagtttctccttcacccatttgttttcattgcttcgtgacatttcgtgtgacagctcagatcaccatatgcatcccatttgtacacattattttgctaccgtggatatttccactctgtttcatgtgtgacatttttaattcgttacattgttactctaaacattagtctaatagattttcagagttttgaccctcctaatcacataatatccgtttcgacctgcagctatggacaagaatgtctatcctcaacacagcgggttcccccaagttcagcagtgaccggacgatccacgagtatgccaaggacatatggggtatcagccccgtcatcatgccctgaataattcaagcagcttccaggaaccttgagagtctatgaggcgcgattggtcgctgatttgccttgattcagcgagaatgtattgctccccccagttttgtataggcatatgcaagttctgacatggacctgagtgctgagagcatgcagggtagttcatccttagcaatattgcatgtcagttagccagtattacccaggcttatggtgtgtgtatttcgctgaataaagttggagcagagtcatcaaacaaacatactttggtgtatctgtaatgataaataaatggtacatgtgactgaacaaaaagtgtcgttggcgtgcacatgtagtagaagcatgtttcttcagagagggtgcaaagctgaaagcaccccagctttggagtgagaatgcagaagttacgaaaacatttgtttacatccaaagtgcaacgggattcgtcaagagttcagagtcttccatcacggtcatagagcatgtccaatagcagccctataaacctataacagagcagtagcagtggtccctcacattttgaaccaaaaatgcagctccaacggctgccctatttgtaaaatttacttcaaaatttgttgg

SEQ ID NO: 26 - DNA, 2907 bp, Triticum durum
  source 1..2907: mol_type = genomic DNA
atggcgaccgcctcgccgccgctcgccacccccttccgcccgctcgccgtcgccggaggtagcggcggactcgtcgtgggcgccagggccgtggtaccgccgaggcgggggcggcgggggttcgtggtgcggagcgtggcgagcgatcgggaggtgcgggggcccgccccgaccgaggaagagctttcagccgtgctaacttccattgattcatccgccatcgcatcaaacatccagcaccatgcagacttcacaccgttattctcgccagagcactcttcacccctaaaggcttaccatgcaactgccaaaagtgtttttgattctctgatattgaattggaatgcaacatatgactattacaacaaagtgaatgcaaagcaagcttattacctatccatggaatttttgcagggaagagctcttacaaatgctattggcaacctagagctaacaggacaatatgcagaagcgttaaaacaacttggacagaacctagaggatgttgctagtcaggaaccagatcctgcccttggcaatggtggactaggccgtctagcgtcctgttttctggattctatggcaaccctaaattatccagcatggggatatggacttcggtacagatatggcctctttaagcaaatcataacaaaggatggtcaggaggaggtagctgagaattggctagagatgggaaatccatgggagattgtaagaaatgatgtctcttatcctctgaaattctatggcaaagtggttgaaggcactgatgggagaaaacactggattggaggagagaatatcaaggctgtggcacatgatgttcctattcctggctacaagactaaaactactaataatcttcgtctttggtcaacaacagtaccatcacaaaactttgatttgggagcttttaatgctggggatcatgtcaaggccaacgaagcacatctaaatgctgaaaagatatgccacgtattgtatccaggggacgaatcatcagaggggaaaattcttcgcctgaaacaacaatatacactatgctcggcctccctgcaggacattatttctcgttttgagtcgagggctggtgattctctcaactgggaggacttcccctctaaagttgcagttcagatgaatgacacccatccaacactgtgcattcccgagttaatgagaatactgatggatgttaagggattgagctggaatgagtcttggagtatcacggaaagaaccgttgcatacactaaccacacagtacttcctgaagctctggagaagtggagcttggacattatgcagaaacttctacctcgacatgttgagattatagaaagaatagatgaggagctgatgaacaccatcgtctcaaaatatggaacggcagatatttcactgttaaaacagaagcttaaagatatgaggatcttagacaacgttgatcttccagcctctgttgctaaactgtttattaaaccaaaagagaaaaaggggaagttgcttgttgaatctttggagtctattgctgaagctgacgagaaaactgagtcagaagaggaggaaaacattctatccgagacaacagagaaaaagggcgaatctgactctgaagaagctcctgatgcagaaaaggaggatcctgagtatgagttagatccatttacaaaatacgatcctcagttacctagagttgttcgaatggcgaacctctgcgttgttggtggacattcagttaatggtgtggctgagattcacagtgaaattgttaagcaagatgtgttcaatagcttttatgagatgtggcctactaagttccaaaacaaaacaaatggagtgacccccaggcgttggatccggttttgtaatcctgaattaagtacaataatttcaaaatggataggctctgatgactggattctgaacactgataaacttgcaggactgaagaagtttgctgatgatgaggatctgcaatcagaatggcgtactgctaaaaggaataacaagatgaaggtagtttcgctgataagagacaagactggatatgttgtcagcccagatgcaatgtttgatgtgcaggtgaaaagaatacatgaatataagcggcagcttctaaatatccttggtatcgtttaccgctacaagaagatgaaagaaatgagtgcaaaagatagaataaagagctttgttccaagggtatgcatatttggcgggaaagcttttgccacttacgtacaggcaaagaggattgtgaagtttatcacagatgtcgcagctactgtaaattatgatcctgatattggagatctactgaaggttgtatttgtcccagattataatgttagcgttgctgagacgctcattcctgccagtgaattgtcccagcatatcagtaccgctggaatggaagctagtggaaccagcaacatgaagtttgcaatgaatggctgtattcttattggaactttggatggtgcgaatgtggagatcagagaagaggttggagaggagaactttttcctctttggtgcacatgcacctgaaatcgctggtttgaggcaagagagagctgaagggaagtttgtgcctgacccgagatttgaagaggttaaggaatacgtccgcagtggcatctttgggactggcaactatgatgaattgatgggttctttggaaggaaatgaaggttatggacgtgcagattattttcttgttggcaaggattttcccagttacattgaatgccaggaaaaagttgatgaagcataccgagatcagaagctatggacaagaatgtctatcctcaacacagcgggttcccccaagttcagcagtgaccggacgatccacgagtatgccaaggacatatggggtatcagccccgtcatcatgccctga

SEQ ID NO: 27 - protein, 968 aa, Triticum durum
MATASPPLATPFRPLAVAGGSGGLVVGARAVVPPRRGRRGFVVRSVASDREVRGPAPTEEELSAVLTSIDSSAIASNIQHHADFTPLFSPEHSSPLKAYHATAKSVFDSLILNWNATYDYYNKVNAKQAYYLSMEFLQGRALTNAIGNLELTGQYAEALKQLGQNLEDVASQEPDPALGNGGLGRLASCFLDSMATLNYPAWGYGLRYRYGLFKQIITKDGQEEVAENWLEMGNPWEIVRNDVSYPLKFYGKVVEGTDGRKHWIGGENIKAVAHDVPIPGYKTKTTNNLRLWSTTVPSQNFDLGAFNAGDHVKANEAHLNAEKICHVLYPGDESSEGKILRLKQQYTLCSASLQDIISRFESRAGDSLNWEDFPSKVAVQMNDTHPTLCIPELMRILMDVKGLSWNESWSITERTVAYTNHTVLPEALEKWSLDIMQKLLPRHVEIIERIDEELMNTIVSKYGTADISLLKQKLKDMRILDNVDLPASVAKLFIKPKEKKGKLLVESLESIAEADEKTESEEEENILSETTEKKGESDSEEAPDAEKEDPEYELDPFTKYDPQLPRVVRMANLCVVGGHSVNGVAEIHSEIVKQDVFNSFYEMWPTKFQNKTNGVTPRRWIRFCNPELSTIISKWIGSDDWILNTDKLAGLKKFADDEDLQSEWRTAKRNNKMKVVSLIRDKTGYVVSPDAMFDVQVKRIHEYKRQLLNILGIVYRYKKMKEMSAKDRIKSFVPRVCIFGGKAFATYVQAKRIVKFITDVAATVNYDPDIGDLLKVVFVPDYNVSVAETLIPASELSQHISTAGMEASGTSNMKFAMNGCILIGTLDGANVEIREEVGEENFFLFGAHAPEIAGLRQERAEGKFVPDPRFEEVKEYVRSGIFGTGNYDELMGSLEGNEGYGRADYFLVGKDFPSYIECQEKVDEAYRDQKLWTRMSILNTAGSPKFSSDRTIHEYAKDIWGISPVIMP

SEQ ID NO: 28 - DNA, 2000 bp, Triticum durum
  source 1..2000: mol_type = genomic DNA
  variation 887..1117: note = N can be any nucleotide selected from adenine, guanine, cytosine and thymine.
ctagtggggccttggtccaaaataatccatgtaatttttcatgttttccggcttcgtattttttggtaaaaacccaaaaacaatagaaaagaagaattggcctctgacactagattgataggttagtccaaatcaatattatcaaaaattgtgcccaaaccatatacaaatgatataaatataactagaatacttcataaatttagatacgtttgataaagtgccaagaatggaagagaagtgatcatatgccgccctgtccatatcgggctcatcagaatgttcccaccatcagactccagccccaggattattaacttatgtcggcggtgcgcggaatggatcttgaagaaagcggaatttgtgtcgccctcacgtaaccaggagaacttggcaggttgacgcgcaatcgacctctccagcaaagcaagtcctaggtaggcgcgcttcagctcgcagagtaaccgtacttctggaggagcaagcgggtggtgatcttgagccgccgagacgagcaatcaattaccgagcgagaagcatctgagtggagatgttaccgagcgttctggagctccaactctgccgtcagtgcattgtcagcttgtgctttgcgtagattcgccggaaaggatcaggatcatgcacgccagaattccaggtgtcagagaccacctaatgaaaaccatcgagctttgatcaaaatagctcaagatggaatctccgatcaccttgggagcgaaaatcttactagtttcgttcaaggccaaggtagttatatatttgtgtgtgcaattattaatcattggccgcttagcaaactcttattggttcgataaacattaaagtcatccccactttgagaaaaaatgctacttgctacaaaccctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcaagtaataaatccttgccgcctcgtccttgtttggatgctgcttcttgtcatctcaaggaggcgtgtggagggtggtgtgtccccggatcttgttctctcggttgggttttttctatggcaggtttatctcagtggtagtgtcacgtgaagcagatgacgcgactatgcgtcttggtcctctgcgctctaattccggcagatgaaggttgaccagcttcagcctcgtcggggagcgtatgagatttgtgttttttgactccatctggtcggacctgtggcattatgtagaccctctcatgtattctttttctcctggacagtagtctgttacacaaatcgtggtcgtcggatcttctagtccacatcggcgacttttccgaccgccacttctacaaactccttattttcaacaagtctccttcattgagatggaagtccaggcaatggctttgctcacggcacactgccggcggatgcaagagaaagaagacattcgacacccctaaaactatgaagatgtaatttttaatctttgtaagtgtttttgtgaggatttgtgctacttttaatatattgccttttgccttttgcaagggaaaaaaaactaaaccattacacgaagagaagagccattcccagccgtccaataaacacgacaaaacgctttacagcacgggggcaattcccagccgtcgaaatgcgctgcaacggaaccagcgctccccgacacggcggcacatgagccacaactcgtaccatgcaccgtgcaccggcggtgggcccactggccagcgtccccacacgtgtaccggcggccacaattacacggcacggcacggcgacggagagatattttaacgccttccaattcagaagctcccccatcactcactcgggtacagagcgcagcgggccgcgtgactctcgcgtcgtcgcatcgcgcgcgctgcacaccaccgtcagcgcgaccactgccgcca

SEQ ID NO: 29 - DNA, 7862 bp, Triticum durum
  source 1..7862: mol_type = genomic DNA
aactaaaccattacatgaggccattcccatccgtccaataaatacgacaaaacgctttacagcacgggggcaattcccagccgtcgaaatgcgctgcaacggaactagctctcccagacagacacagcggcacatgagccacgcctcccatcgcaaccgtgcaccggcggtgggcccaccggccagcgcccccacacgtgtaccggcgcccagcattgcacggcacggcgacggagagatatttttaacgccttccaattcagaagctcccccatcactcactcgggtacagagcgcagcgggccgcgtgactctcgcgtcgtcgcatcgcgcgcgctgcacaccaccgtcagcgcgaccactgccaccaatggcgaccgcctcgccgccgctcgccaccgccttccgcccgctcgcagccggcggcggcgccggaggaggaggagcgcacggcgtgggcgccgcgggccgggtcgcgccgaggcgggggcgccggggcttcgtggtgcggagcgtggcgagcgatcgggaggtgcggggccccgcctcgaccgaggaaggtaagccgtcgcgcgctcccgttggctgcgacgacccggaatgctggtcggctgtaatgtggttcggattttcgctggtgcgacctgaatttctgatcctctgactaatggtctctctgttcccgggtttcgggctgttgattttgtgcgcgcgcattgctaatctactgctctaggatttgttagggttcagaggaatcttaaattcacgaataaacttcgacgagtgtattctgaatttactgaaactggcttagtttggggcgtcaagttagaatggtgtatttatggcacagtagcaaattctgagaaatgcttctgtgcgtgcacagccaccccttgtggatgatgcagcaagtgtcaacataccgtcgctactatctgtagctgtaacaaaatcttacgccttgttttctggtggtacagtaatattatcattttacaaagagtggctgtaacaaaatcttacgccttgttttctggtggcttgccattgaggagtagaatgcaaatggcgatacagtatattatctacgattttgtgcaagaattttcacacgtatcccatgcaatttttagttttgactctcgactgtttatacctactctttgtaaaattttggatcctaatattgttgttcttgcaactcagagctttcagccgtgctaacttccattgattcttccgccatcgcgtcaaatatccagcaccatgcagacttcacaccgttgttctcgccagagcactcttcacccctaaaggcttaccatgcaactgctaaaagtgtttttgattctctgatcatcaattggaatgcaacatatgactattacaacaaagtgaatgcaaagcaagcttattacctgtccatggagtttttgcaggtctcacaatatacttcctttagccttacctcaatactttttggtaactgcgttatgatgttatatggttgttatctgtgtactgcagggaagagctctcacaaatgctattggcaatctagagctaacaggacaatatgcagaagcgttaaaacaacttggacagaacctagaggatgttgctagtcaggtaagtgatttttgtcggagattggtttttagtccgtatcaaaattatcgcagggccattatttttattttgaaaacgcattttattattaatcatcaagttccctttgtttaaaaacaatctgaaaagaagaagtttttttcctgagggttccaccgtctgacttatgatctttatgcagttttcagagtgactatgaaattacgccaaaacacgatcttattgatgcctagagtgccctgtagtttagaaatctaagattggatatgttacttttctattacaatgaaacaggaaccagatcctgcccttggcaatggtggtctaggccgtctagcgtcctgttttctggattctatggcaaccctaaattatccagcatggggatatggacttcggtacagatacggcctctttaagcaaatcataacaaaggatggtcaggaggaggtagctgagaattggctagaggtagtgatgtgtcttctacttgtttactgtttggaagtgttgattatgttggtttatttatgttctaatgacctgcatgttagcttcttttctatttataactgatgctgtatctccagatgggaaatccatgggagatcgtaagaaatgatgtctcttatcctgtgaaattctatggcaaagtggttgaaggcactgatgggagaaaacactggattggaggagagaatatcaaggctgtggcacatgatgttcctattcctggctacaagactaaaactgctaataatcttcgtctttggtcaacaacagtaccatcacaaaattttgatttgggtgcctttaatgctggggatcatgccaaggccaatgaagcacatctaaatgctgaaaaggtattctttcgtctactttattttgaatataaaccattaactctcaacaactactgattcaaaggctaaacagtgctttgctagtagttactgtgttttctcttagatgtcatttcataatgtgtgcagtcaatcgtttgatatacacaaacctatttggatttctcctgtaaaaatgttgtcactagatttttcatgaaaaaacttattgtatatgtaaatagaaaagctgatgctcaggtgctttgtgttcaaatgacaagtaaaaatgaacagagtttgtgcacccataacttcttcacctggtgtgtgcaattcgttcggttgtatttatgcgtgttcttattctgcctgaatgattctcagcatatgcatcaggctacatttctcctctgtgtcaagaaaaaataagtctgaccagatgcagtccagtagtctttagatcttttcacatggaagcgttactctatgatttttattatacctttagagtgtttgtgcattgtgcacatataccatgaagtatgggtgttcaaatgtggttctctcaaactgtgctaaacccaccactgtttgccagtattcatttttctgaactagcaacaacttgactggacaagatacattgtactcacatctgaagctgctgggttggacagatttgtccgacccgtctttgaacatctctactatttagtgtgcatgggttaaattgttgacttttcctcgtttgttcagtagtaggagcactatctaattatatttatattcaagttttctctgtgtgttacgttataaacttttagtcatacttacatttgtggacaaaagttatcatgtctaacttaagtttgtgactgatcttctcatcatgccagatatgccacgtattgtatccaggggacgaatcatcagaggggaaaattcttcgcttgaaacaacaatatacattatgctcggcctccctgcaggacattatttctcgttttgagtcgagggctggtgattctctcaactgggaggacttcccctctaaagttgcagttcagatgaatgacactcatccaacactgtgcattccggagttaatgagaatactgatggatataaagggattgagctggaatgaggcttggagtatcacagaaaggtaccacagaagggtactgcagtttgatgtgttatgctgttcctccagctccaattaactaattgtattgcctcatctgtcttccattcaattatgtactgattctctgatatcatttgtagaaccgttgcatacactaaccacacggtacttcctgaagctctggagaagtggagcttggacattatgcagaaacttctaccccgacatgttgagatcatagaaacaatagatgagaaggtaacaaatgtgcagataatattatgcaataaatttcattctctgtagttcaacttatggtttacttatcaacatcgttgattttaaaacagctgatgaacaacatcgtctcaaaatatggaacggcagatatttcactgttaaaacagaagcttaaagatatgaggatcttagacaacgttgatcttccagcctctgttgctaaactgtttattaaacctaaagagaaaaaggggaagttgcttgttgaatctttggagtctattgctgaaggtgacgagaaaactgagtcacaagaggaggaaaacattctatccgagacagcagagaaaaagggcggatctgactctgaagaagctcctgatgcagaaaaggaggatactgtgtatgagttagatccatttgcaaaatacgatcctcagttacctagagttgttcgaatggcaaatctctgtgttgttggtggacattcagttaatggtgtggctgagattcacagtgaaattgttaagcaagatgtgttcaatagcttttatgaggtatttgcacgatgatttggaaaccatgatcttattttctaacggctgctataatcgccaggtaatatggtatcaccatctgccattttcttatgttatttgccagatgtggcctactaagttccaaaacaaaacaaacggagtaactcccaggcgttggatccggttttgtaatcctgaattaagtgcaatcatttcaaaatggataggctctgatgactggattctgaacactgataaacttgcaggactgaagaaggtacttccagtaattttaatttccaaatatcagtaatctaatgcttccttcacttccatgttacctgtgccttgtttttacattgatgtgtgttcatcccgtaatagtttgctgatgatgaggatctgcaatcagaatggcgtactgctaaaaggaataacaagatgaaggtagtttcgctgataagagacaagactggatatgttgtcagcccagatgcaatgtttgatgtgcaggtatattttggactagaaacatgtgtctcctgttaaatatggtttaactcggatcttggtacttgacacttaggtgaaaagaatacatgaatataagcggcagctgctaaatatccttggtatcgtttaccgatacaagaagatgaaagaaatgagtgccaaagatagaataaagagctttgttccaagggtatgcatatttggtgggaaagcttttgccacttacgtacaggcaaagaggattgtgaagtttatcacagatgtcgcagctactgtaaattatgaccctgatgttggagatttactgaaggtaaatttgtgtcttcaatggcacttatcattctaaattttcatgatctgtcactagcaatttgactccgactaggggttactgaaaagcactgaatgttccgagtttcaaaagaaaacagagcagtgaatgttcttaatgtcggtgcttgtagaactgagaaactgaatagccacagttttttttagagaacaatagccacaattgttctgatgctgattttgctgataactagatgaggtaaatacaaggtgacaggttaagttctgaggatttttgtttgttatccaggttgtatttgtcccagattataatgttagcattgctgagaagctcattcctgccagtgaattgtcccagcatatcaggtaatattctggctactcttatatagtatgtgaatacaaagaaaaggagtcttatctggaggtgactgatgtggaaattttcatgtgcctttttgtttgctactccctctgtcccataatataagagcgcttttgacagtacactagtgtcaaaaacgctcttataatatgggacggagggagtataatatatacctggagtcttgctgggatagtgacatccagaagtgccacgccagcttctatttctgtacaagacgtgcatttttaacactattactatccattcagtaccgctggaatggaagctagtggaacaagcaacatgaagtttgcaatgaatggttgtattcttattggaactttggatggtgcgaatgtggagatcagagaagaggttggagaggagaactttttcctctttggtgcacatgcacctgaaatcgctggtttgaggcaagagagagctgaaggaaaggtaccacacttgtttatagaactgtgcctaactatgaaactagtaaagatctctcccaaatgtgtgttgtccaaatagtttaaaggaagaaaaattataactttaccattttcctttgcacttgtagtttgtgcctgacccgagatttgaagaggttaaggaatacgtccgcagtggcatctttgggactagcaactatgatgaattgatgggttctttggaaggaaatgaaggttatggacgtgcagattattttcttgttggcaaggattttcccagttacattgaatgccagcaaaaagttgatgaagcataccgagatcagaaggtaagttgtataactgacgctgtttgttttctggctgaaatgcatacagtgattatgcaaactttctgatatgtgatggctgccatcagtgtttcactgtgaacgacttcattgattcgtaggaaggaatatttcttagttcaaagtttcaaactggattatctataggtgtgttggaagatcataaacaatcagagaatatagttgctagatgtgctatactatatgttggctgttgagtgttgatacatgagctttgatgaacctagctagaccttcaggaaagtgcttctatgcattccaataaaaaaaacttataacttaacaaaacaattaggttgaacctcgccagggagcgttttgttatctgttttctgaaatgaaaatcagaagcggggagctcctcgattctaaaatgaaaaaaaaatccagggataactgagggcctggtttccctctgttggagccccaaagaattctgtggccttatttttgtaagagtataagatgcactgctccgttaagtttctccttcatccatttgttttcattgcttcgtgacatttcgtgtaacagagcatcccatttgtacacattattttgctatcgtggatatttccactctgtttcatgtgtaacatttttaattcgttacatccttgctaactacacatagtctgatagattttcagagttttggtcctcctaataacataatatccgtttctacctgcagctatggacaagaatgtccatcctcaacacagttggttcccccaagttcagcagtgaccggacgatccacgagtatgccaaggacatatgggatatcagccccgtcatcatgccctgaattattcaagcagctttcaggaacattcagagtctatgaggcgcgattggtcgcaatgtattgctccccccagttttgtataggcacatatgcaagttctgacatggacctgagtgctgagagcatgcagggtagttcatccttagcaatattgcatgtcacttagcccgtattacccaggcttatggtgtgtgtatttcgccgaataaagttggagcagaatcatcaaacaaacatactttggtgtatctgtaatgataaataaatggtaaatgactgaacaaaaagtgtcgttggcgcacacatgtagtagaagcatgtttcttcagagaggctgcaaaactgaaagcaccctagctttgaagtgagaatgaagaagttacggaaacatttgtttacatccaaagtgcaacgggattcgtcaagactcaagagttcagagtcttccgtcacggccatagggcatgtccaacagcagccctataatagagcagtagcagtggtctctcacattttgaaccaaaaacgcagctccaacggctgccctatttgtaaaatttacttcaaaatttgttggacagcccgaaaatgcaacaccag

SEQ ID NO: 30 - DNA, 2916 bp, Triticum durum
  source 1..2916: mol_type = genomic DNA
atggcgaccgcctcgccgccgctcgccaccgccttccgcccgctcgcagccggcggcggcgccggaggaggaggagcgcacggcgtgggcgccgcgggccgggtcgcgccgaggcgggggcgccggggcttcgtggtgcggagcgtggcgagcgatcgggaggtgcggggccccgcctcgaccgaggaagagctttcagccgtgctaacttccattgattcttccgccatcgcgtcaaatatccagcaccatgcagacttcacaccgttgttctcgccagagcactcttcacccctaaaggcttaccatgcaactgctaaaagtgtttttgattctctgatcatcaattggaatgcaacatatgactattacaacaaagtgaatgcaaagcaagcttattacctgtccatggagtttttgcagggaagagctctcacaaatgctattggcaatctagagctaacaggacaatatgcagaagcgttaaaacaacttggacagaacctagaggatgttgctagtcaggaaccagatcctgcccttggcaatggtggtctaggccgtctagcgtcctgttttctggattctatggcaaccctaaattatccagcatggggatatggacttcggtacagatacggcctctttaagcaaatcataacaaaggatggtcaggaggaggtagctgagaattggctagagatgggaaatccatgggagatcgtaagaaatgatgtctcttatcctgtgaaattctatggcaaagtggttgaaggcactgatgggagaaaacactggattggaggagagaatatcaaggctgtggcacatgatgttcctattcctggctacaagactaaaactgctaataatcttcgtctttggtcaacaacagtaccatcacaaaattttgatttgggtgcctttaatgctggggatcatgccaaggccaatgaagcacatctaaatgctgaaaagatatgccacgtattgtatccaggggacgaatcatcagaggggaaaattcttcgcttgaaacaacaatatacattatgctcggcctccctgcaggacattatttctcgttttgagtcgagggctggtgattctctcaactgggaggacttcccctctaaagttgcagttcagatgaatgacactcatccaacactgtgcattccggagttaatgagaatactgatggatataaagggattgagctggaatgaggcttggagtatcacagaaagaaccgttgcatacactaaccacacggtacttcctgaagctctggagaagtggagcttggacattatgcagaaacttctaccccgacatgttgagatcatagaaacaatagatgagaagctgatgaacaacatcgtctcaaaatatggaacggcagatatttcactgttaaaacagaagcttaaagatatgaggatcttagacaacgttgatcttccagcctctgttgctaaactgtttattaaacctaaagagaaaaaggggaagttgcttgttgaatctttggagtctattgctgaaggtgacgagaaaactgagtcacaagaggaggaaaacattctatccgagacagcagagaaaaagggcggatctgactctgaagaagctcctgatgcagaaaaggaggatactgtgtatgagttagatccatttgcaaaatacgatcctcagttacctagagttgttcgaatggcaaatctctgtgttgttggtggacattcagttaatggtgtggctgagattcacagtgaaattgttaagcaagatgtgttcaatagcttttatgagatgtggcctactaagttccaaaacaaaacaaacggagtaactcccaggcgttggatccggttttgtaatcctgaattaagtgcaatcatttcaaaatggataggctctgatgactggattctgaacactgataaacttgcaggactgaagaagtttgctgatgatgaggatctgcaatcagaatggcgtactgctaaaaggaataacaagatgaaggtagtttcgctgataagagacaagactggatatgttgtcagcccagatgcaatgtttgatgtgcaggtgaaaagaatacatgaatataagcggcagctgctaaatatccttggtatcgtttaccgatacaagaagatgaaagaaatgagtgccaaagatagaataaagagctttgttccaagggtatgcatatttggtgggaaagcttttgccacttacgtacaggcaaagaggattgtgaagtttatcacagatgtcgcagctactgtaaattatgaccctgatgttggagatttactgaaggttgtatttgtcccagattataatgttagcattgctgagaagctcattcctgccagtgaattgtcccagcatatcagtaccgctggaatggaagctagtggaacaagcaacatgaagtttgcaatgaatggttgtattcttattggaactttggatggtgcgaatgtggagatcagagaagaggttggagaggagaactttttcctctttggtgcacatgcacctgaaatcgctggtttgaggcaagagagagctgaaggaaagtttgtgcctgacccgagatttgaagaggttaaggaatacgtccgcagtggcatctttgggactagcaactatgatgaattgatgggttctttggaaggaaatgaaggttatggacgtgcagattattttcttgttggcaaggattttcccagttacattgaatgccagcaaaaagttgatgaagcataccgagatcagaagctatggacaagaatgtccatcctcaacacagttggttcccccaagttcagcagtgaccggacgatccacgagtatgccaaggacatatgggatatcagccccgtcatcatgccctga

SEQ ID NO: 31 - protein, 971 aa, Triticum durum
MATASPPLATAFRPLAAGGGAGGGGAHGVGAAGRVAPRRGRRGFVVRSVASDREVRGPASTEEELSAVLTSIDSSAIASNIQHHADFTPLFSPEHSSPLKAYHATAKSVFDSLIINWNATYDYYNKVNAKQAYYLSMEFLQGRALTNAIGNLELTGQYAEALKQLGQNLEDVASQEPDPALGNGGLGRLASCFLDSMATLNYPAWGYGLRYRYGLFKQIITKDGQEEVAENWLEMGNPWEIVRNDVSYPVKFYGKVVEGTDGRKHWIGGENIKAVAHDVPIPGYKTKTANNLRLWSTTVPSQNFDLGAFNAGDHAKANEAHLNAEKICHVLYPGDESSEGKILRLKQQYTLCSASLQDIISRFESRAGDSLNWEDFPSKVAVQMNDTHPTLCIPELMRILMDIKGLSWNEAWSITERTVAYTNHTVLPEALEKWSLDIMQKLLPRHVEIIETIDEKLMNNIVSKYGTADISLLKQKLKDMRILDNVDLPASVAKLFIKPKEKKGKLLVESLESIAEGDEKTESQEEENILSETAEKKGGSDSEEAPDAEKEDTVYELDPFAKYDPQLPRVVRMANLCVVGGHSVNGVAEIHSEIVKQDVFNSFYEMWPTKFQNKTNGVTPRRWIRFCNPELSAIISKWIGSDDWILNTDKLAGLKKFADDEDLQSEWRTAKRNNKMKVVSLIRDKTGYVVSPDAMFDVQVKRIHEYKRQLLNILGIVYRYKKMKEMSAKDRIKSFVPRVCIFGGKAFATYVQAKRIVKFITDVAATVNYDPDVGDLLKVVFVPDYNVSIAEKLIPASELSQHISTAGMEASGTSNMKFAMNGCILIGTLDGANVEIREEVGEENFFLFGAHAPEIAGLRQERAEGKFVPDPRFEEVKEYVRSGIFGTSNYDELMGSLEGNEGYGRADYFLVGKDFPSYIECQQKVDEAYRDQKLWTRMSILNTVGSPKFSSDRTIHEYAKDIWDISPVIMP

SEQ ID NO: 32 - DNA, 2000 bp, Triticum durum
  source 1..2000: mol_type = genomic DNA
  variation 1081..1090: note = N can be any amino acid selected from adenine, guanine, cytosine and thymine.
gttggctcccacatgctcctcgatgatcttcatcggatgaaccacgatgtcgaggattcaagcaaccccgtatacaattccctttgtcaatcgatacgttacttgcccgagattcgatcgtcggtatcccaatacctcgctcaatctcattaccggcaagtcactttactcgtaccgtaatgcatgatcccgtgaccaaacacttggtcactttgagctcattatgatgatgcattaccgagtgggcccagagatacctctccgtcatacggagtgacaaatcccagtctcgatccgtgtcaacccaacagacactttcggagatacccgtagtatacctttatagtcacccagttatgttgtgacgtttggtacacccaaagcactcctacggtgtccgggagttacacgatctcatggtctaaggaaaagatacttgacattggaaaagctctagcaaacgaactacacgatcttgtgctatgcttaggattgggtcttgtccatcacatcattctcctaatgatgtgatcccgttatcaacgacatccaatgtccatagtcaagaaaccatgactatcatttgatcaacgagctagtcaactagaggcttactagggacatgttgtggtctatgtattcacacatgtattacgatttccggataacacaattataacatgaataacagacgattatcatgaacaaggaaatataataataatcttttattattgcctctagggcatatttccaacaattgcggtgaggcgcgtggaggttggcgtgtccccaaatctagttatctgggtcaggttttttctttggtaggtttatctcagtggtagtgtcccgtgaagcggatgacacgactatgtgtcttggtcctccatgctctagttgggatggaatctccgaccacctatcgaaaaccatcaagctttcgctaaaattagtcaagatggaatctccgatcaccttgggagcgaaaatttcattggaggccaaggtagttatgtatttgtgtgtgcaattaccgaccattggccgcttagcaaactcctattggctcggtaaagcnnnnnnnnnngttggcgtgtccccaaatctagttatctgggttggggtttttctttggaaggtttatctcagtggtagtgtcccgtgaagcggatgacacggctatgtgtcttggtcctccatgctctagttttgacatgtgaaggttggccaacttcggcgttgtcagggagcatatgagatttgtgttcctttacttcgcctggtcagacctacggtattatataagccctctcatgtgttttcttctcttgggcagcagtcagttacgcaaatcgtggtcgctgacatcttctagtccacatcggcgacttcccgaccgccgcttctacaagctccttatttttcaacaagttttctccaccgagatggaagtcaagaggcggcaacggctttgctcatggcgcactgcggatggatgcaggagaaggaagacattccacacccctcaaactatgaagatgtaatttttagtttttgtaagtatttttgtgaggatttgtgctacttttaatatatatggccttttgcctttttccaggaaaggaaaaactaaaccattacatgaggccattcccatccgtccaataaatacgacaaaacgctttacagcacgggggcaattcccagccgtcgaaatgcgctgcaacggaactagctctcccagacagacacagcggcacatgagccacgcctcccatcgcaaccgtgcaccggcggtgggcccaccggccagcgcccccacacgtgtaccggcgcccagcattgcacggcacggcgacggagagatatttttaacgccttccaattcagaagctcccccatcactcactcgggtacagagcgcagcgggccgcgtgactctcgcgtcgtcgcatcgcgcgcgctgcacaccaccgtcagcgcgaccactgccacca

SEQ ID NO: 33 - DNA, 8351 bp, Triticum turgidum
  source 1..8351: mol_type = genomic DNA
ggcacggcgacggagagatattttaacgccttccaattcagaagctcccccatcactcactcgggtacagagcgcagcgggccgcgtgactctcgcgtcgtcgcatcgcgcgcgctgcacaccaccgtcagcgcgaccactgccgccaatggcgaccgcctcgccgccgctcgccacccccttccgcccgctcgccgtcgccggaggtagcggcggactcgtcgtgggcgccagggccgtggtaccgccgaggcgggggcggcgggggttcgtggtgcggagcgtggcgagcgatcgggaggtgcgggggcccgccccgaccgaggaaggtaagcggccgcgcgctcccattggctgccacgacctggaatgctggtcggctgtaatgtggttcggattttcgctggtgcgacctgaatttgtgatcctctgactgacggcctatgtgttccccgggtttcgggctgttgattttgtgcgcgcgcattgctaatctacttctctaggatttgttagggctcagagcaatcttaaattcgcgaatacacttcgacgaatgtagtctgaatttactgaaactagcttagtttggcgcgtcaagatagactgatgtatttatggtagcaaattctgaaaacttcttctgtgcgtggacagccacccctcgtggatgatgcagcaagtgctaacacagtgtttgctagtacttgctagtatctgtagctgtaacaaaatccttcgaatctgaactagtgagataaatcgtttgagatttttgctgaagattacacagtatcaatatttctgaaggtgattggaacagtatagcatttcaaactggcaaccaagcaggatcagtatactgacgcaatttttttctgaaggtggttggaataccggtatgaattttataatcattgaattaaggcgtaagatttggtaacaaaaactgcttttcacgggaaacgatgaaacatggcacaattcaacctgactttcttttatgaaaaggctctcaagctgccttgtactccctccgtcccataatataagaacgtttttgacattagtgtagtaccaaaaacgttcttatattatgggacagagggagtatttattaatggataaaaagagaggtacagggttctagtagtaacatatgcaaatggtgatacagtatattatctacgattttgtgcaagaattttcacacgaaaacaatattccatgcagtttttagttttgacttttttttttgaattttcnacttttttttttgaattttcacgcgcgcggggggtggggggggggggagagaactcccccacctgaatatatttctcgaacccaacatacagtacaatagtacaaatgccggggcggcagcagagagagaggcagggggcgacccccccatgctatgttacatgcagctttgttctacccacgcagagcctgtagccagttttgactctttttgaattgtttggattgcttaccacatgtgtcacgtggttttagttttgactcttgactgtttaatacctactctttttaagattttggattctaatgtcatctgtcttgcaactcagagctttcagccgtgctaacttccattgattcatccgccatcgcatcaaacatccagcaccatgcagacttcacaccgttattctcgccagagcactcttcacccctaaaggcttaccatgcaactgccaaaagtgtttttgattctctgatattgaattggaatgcaacatatgactattacaacaaagtgaatgcaaagcaagcttattacctatccatggaatttttgcaggtctcgcaatatactttcttcagccttatctcataccttttggtaactgcgttatgatgttatatgtttttttttgtactgcagggaagagctcttacaaatgctattggcaacctagagctaacaggacaatatgcagaagcgttaaaacaacttggacagaacctagaggatgttgctagtcaggtaagccatttttgtcagagaatggtttttagtccatatcaaaattattgccgagccatttttctaaataacattttattattattcatgaacttccatatttttttcaaatctgcaaaaggagaagtttttttcctgggggttccaccatctgacttatatttgttatgcagttttcagagtgattgactatgaaattacgccaaaacacgagcattgcctagagtgccctgtagttttagaaatgttaagattggatatgttacttttctattacaatgaaacaggaaccagatcctgcccttggcaatggtggactaggccgtctagcgtcctgttttctggattctatggcaaccctaaattatccagcatggggatatggacttcggtacagatatggcctctttaagcaaatcataacaaaggatggtcaggaggaggtagctgagaattggctagaggtagtgatatgtcttctacttgtttactgtttggaagcgttgattatgttggtttctgttttaatgatctgcatgttagcttcttttgccatttataactgatgctgtatctccagatgggaaatccatgggagattgtaagaaatgatgtctcttatcctctgaaattctatggcaaagtggttgaaggcactgatgggagaaaacactggattggaggagagaatatcaaggctgtggcacatgatgttcctattcctggctacaagactaaaactactaataatcttcgtctttggtcaacaacagtaccatcacaaaactttgatttgggagcttttaatgctggggatcatgtcaaggccaacgaagcacatctaaatgctgaaaaggtattcctttgtctgcatttattttgaatataaatcattaaatctcaataactattgatttaaatggtaaacactaaacattgtctagctagtagtcactgtggtgtccctcaatgtcattttagaatgtgcacagtcaaatgtttgataactttgattgtaataatatgataatatacagaaactttgattgacactagatttttcataaacatacttattgtatttccatgtaaatagaaaagctgatgttcagaggtttgtgtgtcaaaatgaacagagtttgtgcacacataatatcttcatctggtgtttgcagttctctaggttgtgtttgcgttttctcctgctctgccttaactaatctcagcatatgcatcaggctacattgctcctctgtgtctataaaaaataagtctgatgatatatggttgcagtccagtagttcgtagatggcacagcatggaagaacttttcacatggaagccttacccttataattttaattataccttttgagtgtttgtgcattgtgtacatacaccattaagtatgggtgctcaaaaatggtttatcccaaactgtgcaaaagccgccactgtttgtgtattaatttttttgaactagctaaacaacttgaccgaacaagatacatacaattgcactcacttctgaagcagctcagttggacggatttgttccaatcccgtgcatgggttaaattgtcgagttgtcctcgcttgttcagtagtaggagtactatctattatatttatactaaagttttctttttgtgatacagtacaaacttttagtaatacttacatttgcgaccactgtggacaaaatttattatgtctaacttaactttatcactggtcttctcatcatgccagatatgccacgtattgtatccaggggacgaatcatcagaggggaaaattcttcgcctgaaacaacaatatacactatgctcggcctccctgcaggacattatttctcgttttgagtcgagggctggtgattctctcaactgggaggacttcccctctaaagttgcagttcagatgaatgacacccatccaacactgtgcattcccgagttaatgagaatactgatggatgttaagggattgagctggaatgagtcttggagtatcacggaaaggtatcacagaagggtactgcagtttgatgtgttatgctgttcctccagttccagttaactaattttatttcctcatctgtcttccattcaatcatgtactgattttctgatatccacatcatttgtagaaccgttgcatacactaaccacacagtacttcctgaagctctggagaagtggagcttggacattatgcagaaacttctacctcgacatgttgagattatagaaagaatagatgaggaggtatcaaatgtgcagataatattatacaattaatttcatgctctgtagttcaacttatggtttacttgtcttcatcgttgattttaaacagctgatgaacaccatcgtctcaaaatatggaacggcagatatttcactgttaaaacagaagcttaaagatatgaggatcttagacaacgttgatcttccagcctctgttgctaaactgtttattaaaccaaaagagaaaaaggggaagttgcttgttgaatctttggagtctattgctgaagctgacgagaaaactgagtcagaagaggaggaaaacattctatccgagacaacagagaaaaagggcgaatctgactctgaagaagctcctgatgcagaaaaggaggatcctgagtatgagttagatccatttacaaaatacgatcctcagttacctagagttgttcgaatggcgaacctctgcgttgttggtggacattcagttaatggtgtggctgagattcacagtgaaattgttaagcaagatgtgttcaatagcttttatgaggtatttgcatgatgatttggaaaccgtgatcttattttccaatggctgctataatcgtcaggtgatatggtattcaccatctgccattttcttgtgttctttgccagatgtggcctactaagttccaaaacaaaacaaatggagtgacccccaggcgttggatccggttttgtaatcctgaattaagtacaataatttcaaaatggataggctctgatgactggattctgaacactgataaacttgcaggactgaagaaggtacttccagtagttttattccccaataatcagtaatctaatgcttccttcacttcaatgttaactgtgcattgtttttacattgatgtgtgttcatcctgtaacagtttgctgatgatgaggatctgcaatcagaatggcgtactgctaaaaggaataacaagatgaaggtagtttcgctgataagagacaagactggatatgttgtcagcccagatgcaatgtttgatgtgcaggtatattttgtactagaaaacatgtgtcttcttataaatatggtttaactcagctcttggtactggacacttaggtgaaaagaatacatgaatataagcggcagcttctaaatatccttggtatcgtttaccgctacaagaagatgaaagaaatgagtgcaaaagatagaataaagagctttgttccaagggtatgcatatttggcgggaaagcttttgccacttacgtacaggcaaagaggattgtgaagtttatcacagatgtcgcagctactgtaaattatgatcctgatattggagatctactgaaggtaaatttgtgtctttgatgccacttatcagtcttaattttcatgttctgtcactggcaagttgactccgactgaaaagcattgaatgttcagagtttcaaaggaaaacagagcagtgaatgttcttaatgtcggtgcttgtaaaactgagcaactgaatagccacaggttttttagagaacaatatagccacaaatttttttgacacttttattgctatcagcatctgatgctgatattactgataactagatgaggtaaatataaggtgacaggttaagtctgaggatttttgtttgttatccaggttgtatttgtcccagattataatgttagcgttgctgagacgctcattcctgccagtgaattgtcccagcatatcaggtaatattctgcctactcttatatagtatgtgaatacataaaaaaagaatattacctggagttggctgacatagaatttttcatgtgcctttttgtttgctataatatgtacctggagtcttactgggatagtgccatccagaagtgccaccccagtttctatttctgtataagacgtgcatttttaacagtattactatccattcagtaccgctggaatggaagctagtggaaccagcaacatgaagtttgcaatgaatggctgtattcttattggaactttggatggtgcgaatgtggagatcagagaagaggttggagaggagaactttttcctctttggtgcacatgcacctgaaatcgctggtttgaggcaagagagagctgaagggaaggtaccacacttgtttatatagctgtgcctactatcaaactagtaaagatctctcccatatgtgctgtccaaatagtttagctgaaggaaaatcataactttacaagtttcctttgcacttgtagtttgtgcctgacccgagatttgaagaggttaaggaatacgtccgcagtggcatctttgggactggcaactatgatgaattgatgggttctttggaaggaaatgaaggttatggacgtgcagattattttcttgttggcaaggattttcccagttacattgaatgccaggaaaaagttgatgaagcataccgagatcagaaggtaagttgtataactgacgctgtttgttttctggctgaaatgctcacagtgattatgcaaactttctgatatgtgatggctgacatcagtgtttcactgtgaacgacttcattgattcgtaggaaggaatatttcttagtttaaagtttcaaactgaagtatctatcggtgtgttggaagatcataaacaatcagagaatatagttgctagatgtgctatattactcatctgttggctgttgagtgttgatacatgagctttgatgtagctagaccttcaggaaagtgcttctgtgcattccaataaaaaaataacttataacttaacaaaacaaaacaattaggttgaacctcaccagggggccttttgttatctgttttctgaaatgaaaatcagaagtggggagctcctcaattcgaaaaagaaaaagaaaatcagggggtaactgagggtctggtttccctctgttggagcccccaagaattctgtggtcttatttttgtaagagtataagatgcactgctccgttaagtttctccttcacccatttgttttcattgcttcgtgacatttcgtgtgacagctcagatcaccatatgcatcccatttgtacacattattttgctaccgtggatatttccactctgtttcatgtgtgacatttttaattcgttacattgttactctaaacattagtctaatagattttcagagttttgaccctcctaatcacataatatccgtttcgacctgcagctatggacaagaatgtctatcctcaacacagcgggttcccccaagttcagcagtgaccggacgatccacgagtatgccaaggacatatggggtatcagccccgtcatcatgccctgaataattcaagcagcttccaggaaccttgagagtctatgaggcgcgattggtcgctgatttgccttgattcagcgagaatgtattgctccccccagttttgtataggcatatgcaagttctgacatggacctgagtgctgagagcatgcagggtagttcatccttagcaatattgcatgtcagttagccagtattacccaggcttatggtgtgtgtatttcgctgaataaagttggagcagagtcatcaaacaaacatactttggtgtatctgtaatgataaataaatggtacatgtgactgaacaaaaagtgtcgttggcgtgcacatgtagtagaagcatgtttcttcagagagggtgcaaagctgaaagcaccccagctttggagtgagaatgcagaagttacgaaaacatttgtttacatccaaagtgcaacgggattcgtcaagagttcagagtcttccatcacggtcatagagcatgtccaatagcagccctataaacctataacagagcagtagcagtggtccctcacattttgaaccaaaaatgcagctccaacggctgccctatttgtaaaatttacttcaaaatttgttggacagccctaaaatgcaacaccaactgatgtaaatgtacaactattcggctgctgccctaaaatttgctccaagtggcgaaccactcaggaccacaagaaaacagttgttttacgttgtgccaaaaatgttgcttcaactactgc

SEQ ID NO: 34 - DNA, 2907 bp, Triticum turgidum; identical to SEQ ID NO: 26
  source 1..2907: mol_type = genomic DNA

SEQ ID NO: 35 - protein, 968 aa, Triticum turgidum; identical to SEQ ID NO: 27

SEQ ID NO: 36 - DNA, 2000 bp, Triticum turgidum
  source 1..2000: mol_type = genomic DNA
  variation 702..977: note = N can be any nucleotide selected from adenine, guanine, cytosine and thymine.
ataaatataactagaatacttcataaatttagatacgtttgataaagtgccaagaatggaagagaagtgatcatatgccgccctgtccatatcgggctcatcagaatgttcccaccatcagactccagccccaggattattaacttatgtcggcggtgcgcggaatggatcttgaagaaagcggaatttgtgtcgccctcacgtaaccaggagaacttggcaggttgacgcgcaatcgacctctccagcaaagcaagtcctaggtaggcgcgcttcagctcgcagagtaaccgtacttctggaggagcaagcgggtggtgatcttgagccgccgagacgagcaatcaattaccgagcgagaagcatctgagtggagatgttaccgagcgttctggagctccaactctgccgtcagtgcattgtcagcttgtgctttgcgtagattcgccggaaaggatcaggatcatgcacgccagaattccaggtgtcagagaccacctaatgaaaaccatcgagctttgatcaaaatagctcaagatggaatctccgatcaccttgggagcgaaaatcttactagtttcgttcaaggccaaggtagttatatatttgtgtgtgcaattattaatcattggccgcttagcaaactcttattggttcgataaacattaaagtcatccccactttgagaaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgggggggggggggggggggggggggcaatgccatccttggcaagtaataaatccttgccgcctcgtccttgtttggatgctgcttcttgtcatctcaaggaggcgtgtggagggtggtgtgtccccggatcttgttctctcggttgggttttttctatggcaggtttatctcagtggtagtgtcacgtgaagcagatgacgcgactatgcgtcttggtcctctgcgctctaattccggcagatgaaggttgaccagcttcagcctcgtcggggagcgtatgagatttgtgttttttgactccatctggtcggacctgtggcattatgtagaccctctcatgtattctttttctcctggacagtagtctgttacacaaatcgtggtcgtcggatcttctagtccacatcggcgacttttccgaccgccacttctacaaactccttattttcaacaagtctccttcattgagatggaagtccaggcaatggctttgctcacggcacactgccggcggatgcaagagaaagaagacattcgacacccctaaaactatgaagatgtaatttttaatctttgtaagtgtttttgtgaggatttgtgctacttttaatatattgccttttgccttttgcaagggaaaaaaaactaaaccattacacgaagagaagagccattcccagccgtccaataaacacgacaaaacgctttacagcacgggggcaattcccagccgtcgaaatgcgctgcaacggaaccagcgctccccgacacggcggcacatgagccacaactcgtaccatgcaccgtgcaccggcggtgggcccactggccagcgtccccacacgtgtaccggcggccacaattacacggcacggcacggcgacggagagatattttaacgccttccaattcagaagctcccccatcactcactcgggtacagagcgcagcgggccgcgtgactctcgcgtcgtcgcatcgcgcgcgctgcacaccaccgtcagcgcgaccactgccgcca

SEQ ID NO: 37 - DNA, 7474 bp, Triticum turgidum
  source 1..7474: mol_type = genomic DNA
ttgcacggcacggcgacggagagatatttttaacgccttccaattcagaagctcccccatcactcactcgggtacagagcgcagcgggccgcgtgactctcgcgtcgtcgcatcgcgcgcgctgcacaccaccgtcagcgcgaccactgccaccaatggcgaccgcctcgccgccgctcgccaccgccttccgcccgctcgcagccggcggcggcgccggaggaggaggagcgcacggcgtgggcgccgcgggccgggtcgcgccgaggcgggggcgccggggcttcgtggtgcggagcgtggcgagcgatcgggaggtgcggggccccgcctcgaccgaggaaggtaagccgtcgcgcgctcccgttggctgcgacgacccggaatgctggtcggctgtaatgtggttcggattttcgctggtgcgacctgaatttctgatcctctgactaatggtctctctgttcccgggtttcgggctgttgattttgtgcgcgcgcattgctaatctactgctctaggatttgttagggttcagaggaatcttaaattcacgaataaacttcgacgagtgtattctgaatttactgaaactggcttagtttggggcgtcaagttagaatggtgtatttatggcacagtagcaaattctgagaaatgcttctgtgcgtgcacagccaccccttgtggatgatgcagcaagtgtcaacataccgtcgctactatctgtagctgtaacaaaatcttacgccttgttttctggtggtacagtaatattatcattttacaaagagtggctgtaacaaaatcttacgccttgttttctggtggcttgccattgaggagtagaatgcaaatggcgatacagtatattatctacgattttgtgcaagaattttcacacgtatcccatgcaatttttagttttgactctcgactgtttatacctactctttgtaaaattttggatcctaatattgttgttcttgcaactcagagctttcagccgtgctaacttccattgattcttccgccatcgcgtcaaatatccagcaccatgcagacttcacaccgttgttctcgccagagcactcttcacccctaaaggcttaccatgcaactgctaaaagtgtttttgattctctgatcatcaattggaatgcaacatatgactattacaacaaagtgaatgcaaagcaagcttattacctgtccatggagtttttgcaggtctcacaatatacttcctttagccttacctcaatactttttggtaactgcgttatgatgttatatggttgttatctgtgtactgcagggaagagctctcacaaatgctattggcaatctagagctaacaggacaatatgcagaagcgttaaaacaacttggacagaacctagaggatgttgctagtcaggtaagtgatttttgtcggagattggtttttagtccgtatcaaaattatcgcagggccattatttttattttgaaaacgcattttattattaatcatcaagttccctttgtttaaaaacaatctgaaaagaagaagtttttttcctgagggttccaccgtctgacttatgatctttatgcagttttcagagtgactatgaaattacgccaaaacacgatcttattgatgcctagagtgccctgtagtttagaaatctaagattggatatgttacttttctattacaatgaaacaggaaccagatcctgcccttggcaatggtggtctaggccgtctagcgtcctgttttctggattctatggcaaccctaaattatccagcatggggatatggacttcggtacagatacggcctctttaagcaaatcataacaaaggatggtcaggaggaggtagctgagaattggctagaggtagtgatgtgtcttctacttgtttactgtttggaagtgttgattatgttggtttatttatgttctaatgacctgcatgttagcttcttttctatttataactgatgctgtatctccagatgggaaatccatgggagatcgtaagaaatgatgtctcttatcctgtgaaattctatggcaaagtggttgaaggcactgatgggagaaagcactggattggaggagagaatatcaaggctgtggcacatgatgttcctattcctggctacaagactaaaactactaataatcttcgtctttggtcaacaacagtaccatcacaaaattttgatttgggtgcctttaatgctggggatcatgccaaggccaatgaagcacatctaaatgctgaaaaggtattctttcgtctactttattttgaatataaaccattaactctcaacaactactgattcaaaggctaaacagtgctttgctagtagttactgtgttttctcttagatgtcatttcataatgtgtgcagtcaatcgtttgatatacacaaacctatttggatttctcctgtaaaaatgttgtcactagatttttcatgaaaaaacttattgtatatgtaaatagaaaagctgatgctcaggtgctttgtgttcaaatgacaagtaaaaatgaacagagtttgtgcacccataacttcttcacctggtgtgtgcaattcgttcggttgtatttatgcgtgttcttattctgcctgaatgattctcagcatatgcatcaggctacatttctcctctgtgtcaagaaaaaataagtctgaccagatgcagtccagtagtctttagatcttttcacatggaagcgttactctatgatttttattatacctttagagtgtttgtgcattgtgcacatataccatgaagtatgggtgttcaaatgtggttctctcaaactgtgctaaacccaccactgtttgccagtattcatttttctgaactagcaacaacttgactggacaagatacattgtactcacatctgaagctgctgggttggacagatttgtccgacccgtctttgaacatctctactatttagtgtgcatgggttaaattgttgacttttcctcgtttgttcagtagtaggagcactatctaattatatttatattcaagttttctctgtgtgttacgttataaacttttagtcatacttacatttgtggacaaaagttatcatgtctaacttaagtttgtgactgatcttctcatcatgccagatatgccacgtattgtatccaggggacgaatcatcagaggggaaaattcttcgcttgaaacaacaatatacattatgctcggcctccctgcaggacattatttctcgttttgagtcgagggctggtgattctctcaactgggaggacttcccctctaaagttgcagttcagatgaatgacactcatccaacactgtgcattccggagttaatgagaatactgatggatataaagggattgagctggaatgaggcttggagtatcacagaaaggtaccacagaagggtactgcagtttgatgtgttatgctgttcctccagctccaattaactaattgtattgcctcatctgtcttccattcaattatgtactgattctctgatatcatttgtagaaccgttgcatacactaaccacacggtacttcctgaagctctggagaagtggagcttggacattatgcagaaacttctaccccgacatgttgagatcatagaaacaatagatgagaaggtaacaaatgtgcagataatattatgcaataaatttcattctctgtagttcaacttatggtttacttatcaacatcgttgattttaaaacagctgatgaacaacatcgtctcaaaatatggaacggcagatatttcactgttaaaacagaagcttaaagatatgaggatcttagacaacgttgatcttccagcctctgttgctaaactgtttattaaacctaaagagaaaaaggggaagttgcttgttgaatctttggagtctattgctgaaggtgacgagaaaactgagtcacaagaggaggaaaacattctatccgagacagcagagaaaaagggcggatctgactctgaagaagctcctgatgcagaaaaggaggatactgtgtatgagttagatccatttgcaaaatacgatcctcagttacctagagttgttcgaatggcaaatctctgtgttgttggtggacattcagttaatggtgtggctgagattcacagtgaaattgttaagcaagatgtgttcaatagcttttatgaggtatttgcacgatgatttggaaaccatgatcttattttctaacggctgctataatcgccaggtaatatggtatcaccatctgccattttcttatgttatttgccagatgtggcctactaagttccaaaacaaaacaaacggagtaactcccaggcgttggatccggttttgtaatcctgaattaagtgcaatcatttcaaaatggataggctctgatgactggattctgaacactgataaacttgcaggactgaagaaggtacttccagtaattttaatttccaaatatcagtaatctaatgcttccttcacttccatgttacctgtgccttgtttttacattgatgtgtgttcatcccgtaatagtttgctgatgatgaggatctgcaatcagaatggcgtactgctaaaaggaataacaagatgaaggtagtttcgctgataagagacaagactggatatgttgtcagcccagatgcaatgtttgatgtgcaggtatattttggactagaaacatgtgtctcctgttaaatatggtttaactcggatcttggtacttgacacttaggtgaaaagaatacatgaatataagcggcagctgctaaatatccttggtatcgtttaccgatacaagaagatgaaagaaatgagtgccaaagatagaataaagagctttgttccaagggtatgcatatttggtgggaaagcttttgccacttacgtacaggcaaagaggattgtgaagtttatcacagatgtcgcagctactgtaaattatgaccctgatgttggagatttactgaaggtaaatttgtgtcttcaatggcacttatcattctaaattttcatgatctgtcactagcaatttgactccgactaggggttactgaaaagcactgaatgttccgagtttcaaaagaaaacagagcagtgaatgttcttaatgtcggtgcttgtagaactgagaaactgaatagccacagttttttttagagaacaatagccacaattgttctgatgctgattttgctgataactagatgaggtaaatacaaggtgacaggttaagttctgaggatttttgtttgttatccaggttgtatttgtcccagattataatgttagcattgctgagaagctcattcctgccagtgaattgtcccagcatatcaggtaatattctggctactcttatatagtatgtgaatacaaagaaaaggagtcttatctggaggtgactgatgtggaaattttcatgtgcctttttgtttgctactccctctgtcccataatataagagcgcttttgacagtacactagtgtcaaaaacgctcttataatatgggacggagggagtataatatatacctggagtcttgctgggatagtgacatccagaagtgccacgccagcttctatttctgtacaagacgtgcatttttaacactattactatccattcagtaccgctggaatggaagctagtggaacaagcaacatgaagtttgcaatgaatggttgtattcttattggaactttggatggtgcgaatgtggagatcagagaagaggttggagaggagaactttttcctctttggtgcacatgcacctgaaatcgctggtttgaggcaagagagagctgaaggaaaggtaccacacttgtttatagaactgtgcctaactatgaaactagtaaagatctctcccaaatgtgtgttgtccaaatagtttaaaggaagaaaaattataactttaccattttcctttgcacttgtagtttgtgcctgacccgagatttgaagaggttaaggaatacgtccgcagtggcatctttgggactagcaactatgatgaattgatgggttctttggaaggaaatgaaggttatggacgtgcagattattttcttgttggcaaggattttcccagttacattgaatgccagcaaaaagttgatgaagcataccgagatcagaaggtaagttgtataactgacgctgtttgttttctggctgaaatgcatacagtgattatgcaaactttctgatatgtgatggctgccatcagtgtttcactgtgaacgacttcattgattcgtaggaaggaatatttcttagttcaaagtttcaaactggattatctataggtgtgttggaagatcataaacaatcagagaatatagttgctagatgtgctatactatatgttggctgttgagtgttgatacatgagctttgatgaacctagctagaccttcaggaaagtgcttctatgcattccaataaaaaaaacttataacttaacaaaacaattaggttgaacctcgccagggagcgttttgttatctgttttctgaaatgaaaatcagaagcggggagctcctcgattctaaaatgaaaaaaaaatccagggataactgagggcctggtttccctctgttggagccccaaagaattctgtggccttatttttgtaagagtataagatgcactgctccgttaagtttctccttcatccatttgttttcattgcttcgtgacatttcgtgtaacagagcatcccatttgtacacattattttgctatcgtggatatttccactctgtttcatgtgtaacatttttaattcgttacatccttgctaactacacatagtctgatagattttcagagttttggtcctcctaataacataatatccgtttctacctgcagctatggacaagaatgtccatcctcaacacagttggttcccccaagttcagcagtgaccggacgatccacgagtatgccaaggacatatgggatatcagccccgtcatcatgccctgaattattcaagcagctttcaggaacattcagagtctatgaggcgcgattggtcgcaatgtattgctccccccagttttgtataggcacatatgcaagttctgacatggacctgagtgctgagagcatgcagggtagttcatccttagcaatattgcatgtcacttagcccgtattacccaggcttatggtgtgtgtatttcgccgaataaagttggagcagaatcatcaaacaaacatactttggtgtatctgtaatgataaataaatggtaaatgactgaacaaaaagtgtcgttggcgcacacatgtagtagaagcatgtttcttcagagaggctgcaaaactgaaagcaccctagctttgaagtgagaatgaagaagttacggaaacatttgtttacatccaaagtgcaacgggattcgtcaaga

SEQ ID NO: 38 - DNA, 2916 bp, Triticum turgidum
  source 1..2916: mol_type = genomic DNA
atggcgaccgcctcgccgccgctcgccaccgccttccgcccgctcgcagccggcggcggcgccggaggaggaggagcgcacggcgtgggcgccgcgggccgggtcgcgccgaggcgggggcgccggggcttcgtggtgcggagcgtggcgagcgatcgggaggtgcggggccccgcctcgaccgaggaagagctttcagccgtgctaacttccattgattcttccgccatcgcgtcaaatatccagcaccatgcagacttcacaccgttgttctcgccagagcactcttcacccctaaaggcttaccatgcaactgctaaaagtgtttttgattctctgatcatcaattggaatgcaacatatgactattacaacaaagtgaatgcaaagcaagcttattacctgtccatggagtttttgcagggaagagctctcacaaatgctattggcaatctagagctaacaggacaatatgcagaagcgttaaaacaacttggacagaacctagaggatgttgctagtcaggaaccagatcctgcccttggcaatggtggtctaggccgtctagcgtcctgttttctggattctatggcaaccctaaattatccagcatggggatatggacttcggtacagatacggcctctttaagcaaatcataacaaaggatggtcaggaggaggtagctgagaattggctagagatgggaaatccatgggagatcgtaagaaatgatgtctcttatcctgtgaaattctatggcaaagtggttgaaggcactgatgggagaaagcactggattggaggagagaatatcaaggctgtggcacatgatgttcctattcctggctacaagactaaaactactaataatcttcgtctttggtcaacaacagtaccatcacaaaattttgatttgggtgcctttaatgctggggatcatgccaaggccaatgaagcacatctaaatgctgaaaagatatgccacgtattgtatccaggggacgaatcatcagaggggaaaattcttcgcttgaaacaacaatatacattatgctcggcctccctgcaggacattatttctcgttttgagtcgagggctggtgattctctcaactgggaggacttcccctctaaagttgcagttcagatgaatgacactcatccaacactgtgcattccggagttaatgagaatactgatggatataaagggattgagctggaatgaggcttggagtatcacagaaagaaccgttgcatacactaaccacacggtacttcctgaagctctggagaagtggagcttggacattatgcagaaacttctaccccgacatgttgagatcatagaaacaatagatgagaagctgatgaacaacatcgtctcaaaatatggaacggcagatatttcactgttaaaacagaagcttaaagatatgaggatcttagacaacgttgatcttccagcctctgttgctaaactgtttattaaacctaaagagaaaaaggggaagttgcttgttgaatctttggagtctattgctgaaggtgacgagaaaactgagtcacaagaggaggaaaacattctatccgagacagcagagaaaaagggcggatctgactctgaagaagctcctgatgcagaaaaggaggatactgtgtatgagttagatccatttgcaaaatacgatcctcagttacctagagttgttcgaatggcaaatctctgtgttgttggtggacattcagttaatggtgtggctgagattcacagtgaaattgttaagcaagatgtgttcaatagcttttatgagatgtggcctactaagttccaaaacaaaacaaacggagtaactcccaggcgttggatccggttttgtaatcctgaattaagtgcaatcatttcaaaatggataggctctgatgactggattctgaacactgataaacttgcaggactgaagaagtttgctgatgatgaggatctgcaatcagaatggcgtactgctaaaaggaataacaagatgaaggtagtttcgctgataagagacaagactggatatgttgtcagcccagatgcaatgtttgatgtgcaggtgaaaagaatacatgaatataagcggcagctgctaaatatccttggtatcgtttaccgatacaagaagatgaaagaaatgagtgccaaagatagaataaagagctttgttccaagggtatgcatatttggtgggaaagcttttgccacttacgtacaggcaaagaggattgtgaagtttatcacagatgtcgcagctactgtaaattatgaccctgatgttggagatttactgaaggttgtatttgtcccagattataatgttagcattgctgagaagctcattcctgccagtgaattgtcccagcatatcagtaccgctggaatggaagctagtggaacaagcaacatgaagtttgcaatgaatggttgtattcttattggaactttggatggtgcgaatgtggagatcagagaagaggttggagaggagaactttttcctctttggtgcacatgcacctgaaatcgctggtttgaggcaagagagagctgaaggaaagtttgtgcctgacccgagatttgaagaggttaaggaatacgtccgcagtggcatctttgggactagcaactatgatgaattgatgggttctttggaaggaaatgaaggttatggacgtgcagattattttcttgttggcaaggattttcccagttacattgaatgccagcaaaaagttgatgaagcataccgagatcagaagctatggacaagaatgtccatcctcaacacagttggttcccccaagttcagcagtgaccggacgatccacgagtatgccaaggacatatgggatatcagccccgtcatcatgccctga

SEQ ID NO: 39 - protein, 971 aa, Triticum turgidum
MATASPPLATAFRPLAAGGGAGGGGAHGVGAAGRVAPRRGRRGFVVRSVASDREVRGPASTEEELSAVLTSIDSSAIASNIQHHADFTPLFSPEHSSPLKAYHATAKSVFDSLIINWNATYDYYNKVNAKQAYYLSMEFLQGRALTNAIGNLELTGQYAEALKQLGQNLEDVASQEPDPALGNGGLGRLASCFLDSMATLNYPAWGYGLRYRYGLFKQIITKDGQEEVAENWLEMGNPWEIVRNDVSYPVKFYGKVVEGTDGRKHWIGGENIKAVAHDVPIPGYKTKTTNNLRLWSTTVPSQNFDLGAFNAGDHAKANEAHLNAEKICHVLYPGDESSEGKILRLKQQYTLCSASLQDIISRFESRAGDSLNWEDFPSKVAVQMNDTHPTLCIPELMRILMDIKGLSWNEAWSITERTVAYTNHTVLPEALEKWSLDIMQKLLPRHVEIIETIDEKLMNNIVSKYGTADISLLKQKLKDMRILDNVDLPASVAKLFIKPKEKKGKLLVESLESIAEGDEKTESQEEENILSETAEKKGGSDSEEAPDAEKEDTVYELDPFAKYDPQLPRVVRMANLCVVGGHSVNGVAEIHSEIVKQDVFNSFYEMWPTKFQNKTNGVTPRRWIRFCNPELSAIISKWIGSDDWILNTDKLAGLKKFADDEDLQSEWRTAKRNNKMKVVSLIRDKTGYVVSPDAMFDVQVKRIHEYKRQLLNILGIVYRYKKMKEMSAKDRIKSFVPRVCIFGGKAFATYVQAKRIVKFITDVAATVNYDPDVGDLLKVVFVPDYNVSIAEKLIPASELSQHISTAGMEASGTSNMKFAMNGCILIGTLDGANVEIREEVGEENFFLFGAHAPEIAGLRQERAEGKFVPDPRFEEVKEYVRSGIFGTSNYDELMGSLEGNEGYGRADYFLVGKDFPSYIECQQKVDEAYRDQKLWTRMSILNTVGSPKFSSDRTIHEYAKDIWDISPVIMP

SEQ ID NO: 40 - DNA, 2000 bp, Triticum turgidum
  source 1..2000: mol_type = genomic DNA
cccacccggtggacccccgggacccttccggtggttccagtacaataccggtgacccccgaaactttcccgatggccgaaacctgacttcctatatataattctttacctccaaaccattttggaactcctcgaacgtccgggatctcatccgggactccgaacaactttcggtttactgcatactcatatctctacaaccctagcgtcaccgaaccttaagtgtgtagaccctacggattcgggagacacgcagacatgaccgagacggctctccggtcaataaccaacagcgggatctagatacccatgttggctcccacatgctcctcgatgatcttcatcggatgaaccacgatgtcgaggattcaagcaaccccgtatacaattccctttgtcaatcgatacgttacttgcccgagattcgatcgtcggtatcccaatacctcgctcaatctcattaccggcaagtcactttactcgtaccgtaatgcatgatcccgtgaccaaacacttggtcactttgagctcattatgatgatgcattaccgagtgggcccagagatacctctccgtcatacggagtgacaaatcccagtctcgatccgtgtcaacccaacagacactttcggagatacccgtagtatacctttatagtcacccagttatgttgtgacgtttggtacacccaaagcactcctacggtgtccgggagttacacgatctcatggtctaaggaaaagatacttgacattggaaaagctctagcaaacgaactacacgatcttgtgctatgcttaggattgggtcttgtccatcacatcattctcctaatgatgtgatcccgttatcaacgacatccaatgtccatagtcaagaaaccatgactatcatttgatcaacgagctagtcaactagaggcttactagggacatgttgtggtctatgtattcacacatgtattacgatttccggataacacaattataacatgaataacagacgattatcatgaacaaggaaatataataataatcttttattattgcctctagggcatatttccaacaattgcggtgaggcgcgtggaggttggcgtgtccccaaatctagttatctgggtcaggttttttctttggtaggtttatctcagtggtagtgtcccgtgaagcggatgacacggctatgtgtcttggtcctccatgctctagttttgacatgtgaaggttggccaacttcggcgttgtcagggagcatatgagatttgtgttcctttacttcgcctggtcagacctacggtattatataagccctctcatgtgttttcttctcttgggcagcagtcagttacgcaaatcgtggtcgctgacatcttctagtccacatcggcgacttcccgaccgccgcttctacaagctccttatttttcaacaagttttctccaccgagatggaagtcaagaggcggcaacggctttgctcatggcgcactgcggatggatgcaggagaaggaagacattccacacccctcaaactatgaagatgtaatttttagtttttgtaagtatttttgtgaggatttgtgctacttttaatatatatggccttttgcctttttccaggaaaggaaaaactaaaccattacatgaggccattcccatccgtccaataaatacgacaaaacgctttacagcacgggggcaattcccagccgtcgaaatgcgctgcaacggaactagctctcccagacagacacagcggcacatgagccacgcctcccatcgcaaccgtgcaccggcggtgggcccaccggccagcgcccccacacgtgtaccggcgcccagcattgcacggcacggcgacggagagatatttttaacgccttccaattcagaagctcccccatcactcactcgggtacagagcgcagcgggccgcgtgactctcgcgtcgtcgcatcgcgcgcgctgcacaccaccgtcagcgcgaccactgccacca

SEQ ID NO: 41 - DNA, 8632 bp, Hordeum vulgare
  source 1..8632: mol_type = genomic DNA
aaaaaaagtgctaagacttcttaaagaaacagggcgtgagatggaagtgcagaggcggcagcgaattagctcacggggcactaccggtggatgtaggaaaaagaaggcatttgacacctctcaaaactatgaagatgtaaattttaatttctaagtgtttttgtgaggatttgtgctatttaatactccctcaattccaaaataaatgttttaattttgtattatctctaatacaaagttatactaactttaagatacctattttaaaacagaggaaatatatgcattttgcctttctcacaaaaaaaacccctaaaccatcacacgaggggaagagccattcccagccgtccaataaacacgacaaaaagctgtacagcacgggagcaattcccagccgtcgaaatgcgctgcaacggaagtagcgctccctgacacagcggcacatgagccacgcctcgcaccaccgcaccgtgcaccggcgcggggcccaccggccagcgtccccacacgcgtaccggcggccagcattgcgcggcacggcgacggagagatatttttaacgccttccagttcggaagcgccccccatcactcactcgggtacagggcgggccgcgtgagtctcgcgtcgtcgcatcgcgcgcgctgcacacccaccgtcgtcagcgcggaccaccggcgccccacccaatggcgaccgcctcgccgccgctcgccaccgccttccgcccgctcgccgccgccggaggcagcggggggctcgtcgtgggcgccggggccggggcggcgccgaggcgggggcggcgggggctcgtggtgcggagcgtggcgagcgatcgggaggtgcggggccccgcctccaccgaggaaggtaagcggccgcgcgctcccgttggcagcgacgacacggaatgccggtcggctgtaatgtggtttgggttcccgctggtgcggcccgtgaattcgtgatcctctgactgattgtgctcccgggtttcggtggcgcgggagctttgggtgggcgcgcgcattgctaatgtactgctctaggatttgttagggttcagaggaaacttaaattcgcgaataaacttcaacgaatgtattctgagtttctgaagctggcttagtttggcgcgtcaagttagaatgtgtatttatggtagcaaattctgagaaattcttctatgcgtggagagccacccccctgtggatgctgcagcaagcgctaatatactgtttgctagtatctgtagctgtaacaaaatccttcaaatctgaactagcgaaataaatatgagatttttgctgaagattatacagtgtcaatatttctgaaggtggttgggacagtatagcacttcaaaccagcaaccagcaggatcagtataccgactcatcttttttttctgaaggtggttggaataccggtatcgtttttataattattaaattaaggcgtaagatttggtagcaaaaactattttcaccagaaacgatgaaacatggcacaattcaacctgagtacctgactaccttttttgatgaaaaggctatgggagctccctttcatttattaattaatggataaaaagagaggtggagggttctagtagaatgcaaatggtgatacagtatattatctactattttgtacaagatttttcacacgaaaacaatatcccatgcagttttttgtttcgactcttgactgtttaatacctactctttataaggtttcggatcctaatattgctgttcttgcaactcagagctttcagccgtgctaacttccattgattcttccgccatcgcgtcaaatatccagcaccatgcagacttcacaccgttgttctcgccagagcactcttcacccttaaaggcttaccatgcaactgctaaaagtgtttttgattctctgataatgaattggaatgcaacatatgactattacaacaaagtgaatgcaaagcaagcttattacctgtccatggagtttttgcaggtctcgcaatatacttcctttagctttatctcataccttttggtaactgcgttacgatgttatatggttattttttgtgtactgcagggaagagctcttacaaatgctattggcaacctagaactaacaggacaatatgcagaagcattaaaacaacttggacataacctagaggatgttgctagtcaggtaagcgttttttgtcagagattggtttttagtccgtatataaattatcattgaaccattatctaaaaaacacattttattattaatcatcaagttccttttgtttaaaaactatctgaaaagaagaagtttttttcctgggggccaccatctgacttatattctttatgcgttttccagagtgagtgactataaaattacgccaaaacacgagcttattgatgcctagagtgccctgtagtttataaatcttaagattggatatgatacttttctattacaatgaaacaggaaccagatcctgcccttggcaatggtggtctaggccgtctagcgtcctgtttcctggattctttggcaaccctaaattatccagcatggggatatggacttcggtacagatatggcctctttaagcaaatcataacaaaggatggtcaggaggaggtagctgagaattggctagaggtagtgatatgtcttctacttgtttactgtttggaattgttgattatgttggtttatgttttaatgacctgcatgttagcttcttttgccattaactgatgctgtatctccagatgggaaatccatgggagattgtaagaaatgatgtctcttatcctgtgaaattctatggcaaagtggttgaaggcactgatgggagaaaacactggattggaggagagaatatcaaggctgtggcacatgacgttcctattcctggctacaagactaaaactactaacaatcttcgtctttggtcgacaacagtaccatcacaaaattttgatttgggagcttttaatgctggggaccatgccaaggccaatgaagcgcatctaaatgctgaaaaggtattctttcgtctacatttttttttgaatataaaccattaactctcaataactatcgattcaaaggctaaacagtgttttgctagtagttactgtgttttcattttagaatgtgtgcagtcaatcttttaataactttgattgtaataatatactgatatacacaaacctatttgaatttctcgtgtacaaatgtcgtcactagatttttcatgaaaatacttattgtattttcatgtaaatagaaaagctgatggttagatgttttgtgtcaagtgacaaatgaaaatgaacagtgtttgtacacccataacttcttcacatggtgtttgcaattctttcggttgtatttatgtgttttcttattctgagttaattaatctcagcatatgcatcaggctacattactcctgtgtgtcaagaaaaaataagtctgacgaggtgcagtccagtagtgtgtagatggcacattgtagaatagcttttctcatggaagcgttactttatgatttttgttataccttttgagtgtttgtgcattgtgcacatataccattaagtatgagtgttcaaaagtggttctctcaaactgtgcaaaacccgccactgtttgcgagtattcatttatctgaactagttaaacaacttgactggacaagatgcattgtactgacatctgaagctgccgggttggacagatttgtgctcacatctatttagcgtacatggattaaattgttcaattttcctcatttgtttagcttgaggagtactatctaattatatttatattcaaattttctttgtgtgttacgttataaacttctagtcatacttacatttgtggccattgtggacaaaatttatcatgtctaacttaagtttgtgattggtcttctcatcatgccagatatgccacgtattgtatccaggtgacgaatcatcagaggggaaaattcttcgcttgaaacaacaatatacattatgctcggcctccctgcaggacatcatttctcgttttgagtcgagggctggtgattctctcaactgggaggacttcccctctaaagttgcagtccagatgaatgacactcatccaacactgtgcattcccgagttaatgagaatactgatggatataaagggattgagctggaatgaggcttggagtatcacagaaaggtaccacagaagggtactgcagtttgatgtgttatatgctgttcctccagttctatttacctaattttatttcctcatctgtcttccattcaattatgtactgattttctgatatcatttgtagaaccgttgcatacactaaccacacggtattgcctgaagctctagagaagtggagcttggacattatgcagaaacttctacctcgacatgttgagatcatagaaacaatagatgaggaggtaacaaatgtgcaaataatattatacaattaatttcatgctctgtagttcaacttatggtttacttatcttaatcattgatttaaaaacagctgatgaacaacatcgtctcaaaatatggaacggcagatatttcactgttaaagcagaagcttaaagatatgaggatcttagacaacgttgatcttccagcctctgttgctaaactgtttattaaacctaaagagaaaaggggaaagttgcttgtcgaatctttggagtctattgctgaagctgacgagaaaactgagtcacaagaggtggaaaacattctatctgagacaacagagaaaaaggctgaatctgactctgaagaagctcctgatgcagaaaaggaggatcctgagtatgagttagatccatttgcaaaatacgatcctcagtttcctagagttgttcgaatggcaaacctctgtgttgttggtggacattcagttaatggtgtggctgaaattcacagtgaaattgttaagcaagatgtgttcaatagcttttatgaggtatttgcacgatgatttggaaactgtgatcttattttccaatggctgctataatcgtcaggtggtatggtattcaccatctgccattttgttgtgttctttgccagatgtggcctactaagttccaaaacaaaacaaatggagtgacccccaggcgttggatccgattttgtaatcctgaattaagtacaatcatttcaaaatggataggctctgatgactggatcctgaacactgataaacttgcaggactgaagaaggtacttccagtagttttattttccattaatcagtaatctaatgcttccttcacttctatgttacctgtgccttgttacattgatgtgtgttcatcctgtaacagtttgctgatgatgaggatctacaatcagaatggcgtactgctaaaaggaataacaagatgaaggtagtttcgctgataagagacaagactggatatattgtcagcccagatgcgatgtttgatgtgcaggtatattttatactagaaaacatgtgttttcttataaatatggtttagctcagctcttggtactggacacttaggtgaaaagaatacatgagtataagcggcagctgctaaatatccttggtatcgtttaccgctacaagaagatgaaagaaatgagtgcaaaagatagaagaaagagctttgttccaagggtatgcatatttggtgggaaagcttttgccacttacgtgcaggcaaagaggattgtgaagtttatcacagatgttgcagctactgtaaattatgatcctgatattggagatttactgaaggtaaatttgtgtctttgatgtcacttgtcgttcttaattctcatgttctgtcactggcaatttgactccgactagagttactgaaaagcagtgaatgttcagagtttcaaaagaaaacagagcagtgaatgttcgtaatgtcggtgcttgcaaaactgagaaactgaatagccacagatttttatagagaacaatatagccacaattgttttgacccttttattgctatcaagcatctgatgcttatattactgataactagatgaggtaaatacaaggagacacgctaagttctgaggatttttgtttgttatccaggttgtatttgtcccagattataatgttagcgttgctgagacgctcattcctgccagtgaattgtcccagcatatcaggtaatattctggctactctttttcatagcatgtgaatacatagagaaagaatcttagctggagttggctgatgtggaaattttcatgtgcctttttgtttgctctaatatatacctggagtcttaccaggatagtgccaccccagtttctatttctgtacaagacgtgcatttttaacactattactatccattcagtactgctggaatggaagctagtggaaccagcaacatgaagtttgcaatgaatggttgtcttcttattggaactttggatggtgcgaatgtggagatcagagaagaggttggagaggagaattttttcctctttggtgcacatgcacctgaaatcgctggtttgaggcaagagagagctgaaggaaaggtaggtaccacacttgtttatatagccgtgcccacgatcaaactagcaaagatctctcccaaatgtgtgccgcccaagtagtttatctgaaggaaaatcataactttaccatcttcctttgcacttgtagtttgtgcctgacctgagatttgaagaggttaaggaatacgtccgcagtggcgtctttgggactagcaactatgatgaattaatgggttctttggaaggaaatgaaggttatggacgtgcagattattttcttgttggcaaggattttcccagttacattgaatgccaggaaaaagttgatgaagcataccgagatcagaaggtaagttgtataactgacgccttttcttttatgactgaaatgcacacagtgattgtgtgacttcctggtatgtgatggctgacatcagtgtttcactatgaacgatttcactgatttgtaggaatatttcttaattcaaactgaagtatctattgatctattagtatgttggaataacatcataaacaatcagagaatacggttgctagatgtgatatatgagctttaaggaacctagctagacattcagggaagtgtttctatgcatttcaatccaaacataaactgacggaacaattaagtttaaactcaccgcggggctttgtgttattgggttttctgaaataaaatctggagcgaggagctcctcaattcgaaaagaaggccgaaacaattcagtgtaactggggacctggttccctctgttggagtctcccaagaattctgcagtcttgtttctgggagagtataaaatgcaccaccctgttcagattccccttcacccacttggttttcttgcttggcgacattttatgagacggcttagattatcatttgtacactatggatatttccactctgtttcaagtgtgattaacagtgttaatttgctactaaccagacatagactgatagatagttggagtgtttaatcctcctaatcacatgatatccgtctctacctgcagctatggacaagaatgtctatcctcaacacagcgggttcccccaagttcagcagtgaccggacgatccacgagtacgccaaggacatatgggatatcagccccgtcatcatgccctgagtaattcaagcagtttccaggaacctcgagtctgtgaggcgtcacagatttgcctcactcctcagtgagagggtcttgttgggcccagttttgtataggcatatgcaagctctgacatgaacctgagagctgagagcatgcaggatagttcatctttaacaaacttgcatgccggttagccagtgtcacccgggcttatagtgtgcgaatttcgccgaataatgttggagcagaatcatcaaacaaacaaactttggtgtatctgtgatgataaataaatggcaaatgattgaatgagaagtgtcactggcgcacacatgtagaagcatgtttcttcggagagggtgcaaagcggaaacaccccagctttgaagtgagaatgcagaaattactgccacgtttggtaccatgcatcatcagggcagtcaggcatactaaatgcagttttggcctgtttggttgaccgagttgcatcaaaatcctggtccgcatgaatattactccctccgttttctaaatataaatctttttaaagatttcactaagagactacatacatatgtatatggatatgctttagaaagtagattcattcattttactttgtatgtagtcctttaatcaaatctctaaaaagacttatatttaggaaccgagggagtaaaacacctctggtcaagagatgtttgaatcggccgttttttcacagccaggctcaagccatgcacctcgaggcatgtgggccatacctggccaaacgggccgggccgggccgggccgggccggcctggcccggccataaacaggccgacaagacccgcccagacccgaatattagccggaccgtgccgtgccggctcatgtacgcagcctttcggcccaggcacgacacggga

SEQ ID NO: 42 - DNA, 2907 bp, Hordeum vulgare
  source 1..2907: mol_type = genomic DNA
atggcgaccgcctcgccgccgctcgccaccgccttccgcccgctcgccgccgccggaggcagcggggggctcgtcgtgggcgccggggccggggcggcgccgaggcgggggcggcgggggctcgtggtgcggagcgtggcgagcgatcgggaggtgcggggccccgcctccaccgaggaagagctttcagccgtgctaacttccattgattcttccgccatcgcgtcaaatatccagcaccatgcagacttcacaccgttgttctcgccagagcactcttcacccttaaaggcttaccatgcaactgctaaaagtgtttttgattctctgataatgaattggaatgcaacatatgactattacaacaaagtgaatgcaaagcaagcttattacctgtccatggagtttttgcagggaagagctcttacaaatgctattggcaacctagaactaacaggacaatatgcagaagcattaaaacaacttggacataacctagaggatgttgctagtcaggaaccagatcctgcccttggcaatggtggtctaggccgtctagcgtcctgtttcctggattctttggcaaccctaaattatccagcatggggatatggacttcggtacagatatggcctctttaagcaaatcataacaaaggatggtcaggaggaggtagctgagaattggctagagatgggaaatccatgggagattgtaagaaatgatgtctcttatcctgtgaaattctatggcaaagtggttgaaggcactgatgggagaaaacactggattggaggagagaatatcaaggctgtggcacatgacgttcctattcctggctacaagactaaaactactaacaatcttcgtctttggtcgacaacagtaccatcacaaaattttgatttgggagcttttaatgctggggaccatgccaaggccaatgaagcgcatctaaatgctgaaaagatatgccacgtattgtatccaggtgacgaatcatcagaggggaaaattcttcgcttgaaacaacaatatacattatgctcggcctccctgcaggacatcatttctcgttttgagtcgagggctggtgattctctcaactgggaggacttcccctctaaagttgcagtccagatgaatgacactcatccaacactgtgcattcccgagttaatgagaatactgatggatataaagggattgagctggaatgaggcttggagtatcacagaaagaaccgttgcatacactaaccacacggtattgcctgaagctctagagaagtggagcttggacattatgcagaaacttctacctcgacatgttgagatcatagaaacaatagatgaggagctgatgaacaacatcgtctcaaaatatggaacggcagatatttcactgttaaagcagaagcttaaagatatgaggatcttagacaacgttgatcttccagcctctgttgctaaactgtttattaaacctaaagagaaaaggggaaagttgcttgtcgaatctttggagtctattgctgaagctgacgagaaaactgagtcacaagaggtggaaaacattctatctgagacaacagagaaaaaggctgaatctgactctgaagaagctcctgatgcagaaaaggaggatcctgagtatgagttagatccatttgcaaaatacgatcctcagtttcctagagttgttcgaatggcaaacctctgtgttgttggtggacattcagttaatggtgtggctgaaattcacagtgaaattgttaagcaagatgtgttcaatagcttttatgagatgtggcctactaagttccaaaacaaaacaaatggagtgacccccaggcgttggatccgattttgtaatcctgaattaagtacaatcatttcaaaatggataggctctgatgactggatcctgaacactgataaacttgcaggactgaagaagtttgctgatgatgaggatctacaatcagaatggcgtactgctaaaaggaataacaagatgaaggtagtttcgctgataagagacaagactggatatattgtcagcccagatgcgatgtttgatgtgcaggtgaaaagaatacatgagtataagcggcagctgctaaatatccttggtatcgtttaccgctacaagaagatgaaagaaatgagtgcaaaagatagaagaaagagctttgttccaagggtatgcatatttggtgggaaagcttttgccacttacgtgcaggcaaagaggattgtgaagtttatcacagatgttgcagctactgtaaattatgatcctgatattggagatttactgaaggttgtatttgtcccagattataatgttagcgttgctgagacgctcattcctgccagtgaattgtcccagcatatcagtactgctggaatggaagctagtggaaccagcaacatgaagtttgcaatgaatggttgtcttcttattggaactttggatggtgcgaatgtggagatcagagaagaggttggagaggagaattttttcctctttggtgcacatgcacctgaaatcgctggtttgaggcaagagagagctgaaggaaagtttgtgcctgacctgagatttgaagaggttaaggaatacgtccgcagtggcgtctttgggactagcaactatgatgaattaatgggttctttggaaggaaatgaaggttatggacgtgcagattattttcttgttggcaaggattttcccagttacattgaatgccaggaaaaagttgatgaagcataccgagatcagaagctatggacaagaatgtctatcctcaacacagcgggttcccccaagttcagcagtgaccggacgatccacgagtacgccaaggacatatgggatatcagccccgtcatcatgccctga

SEQ ID NO: 43 - protein, 968 aa, Hordeum vulgare
MATASPPLATAFRPLAAAGGSGGLVVGAGAGAAPRRGRRGLVVRSVASDREVRGPASTEEELSAVLTSIDSSAIASNIQHHADFTPLFSPEHSSPLKAYHATAKSVFDSLIMNWNATYDYYNKVNAKQAYYLSMEFLQGRALTNAIGNLELTGQYAEALKQLGHNLEDVASQEPDPALGNGGLGRLASCFLDSLATLNYPAWGYGLRYRYGLFKQIITKDGQEEVAENWLEMGNPWEIVRNDVSYPVKFYGKVVEGTDGRKHWIGGENIKAVAHDVPIPGYKTKTTNNLRLWSTTVPSQNFDLGAFNAGDHAKANEAHLNAEKICHVLYPGDESSEGKILRLKQQYTLCSASLQDIISRFESRAGDSLNWEDFPSKVAVQMNDTHPTLCIPELMRILMDIKGLSWNEAWSITERTVAYTNHTVLPEALEKWSLDIMQKLLPRHVEIIETIDEELMNNIVSKYGTADISLLKQKLKDMRILDNVDLPASVAKLFIKPKEKRGKLLVESLESIAEADEKTESQEVENILSETTEKKAESDSEEAPDAEKEDPEYELDPFAKYDPQFPRVVRMANLCVVGGHSVNGVAEIHSEIVKQDVFNSFYEMWPTKFQNKTNGVTPRRWIRFCNPELSTIISKWIGSDDWILNTDKLAGLKKFADDEDLQSEWRTAKRNNKMKVVSLIRDKTGYIVSPDAMFDVQVKRIHEYKRQLLNILGIVYRYKKMKEMSAKDRRKSFVPRVCIFGGKAFATYVQAKRIVKFITDVAATVNYDPDIGDLLKVVFVPDYNVSVAETLIPASELSQHISTAGMEASGTSNMKFAMNGCLLIGTLDGANVEIREEVGEENFFLFGAHAPEIAGLRQERAEGKFVPDLRFEEVKEYVRSGVFGTSNYDELMGSLEGNEGYGRADYFLVGKDFPSYIECQEKVDEAYRDQKLWTRMSILNTAGSPKFSSDRTIHEYAKDIWDISPVIMP

SEQ ID NO: 44 - DNA, 2000 bp, Hordeum vulgare
  source 1..2000: mol_type = genomic DNA
tcgagaactatgtagacatgacccgagagattcctcggtcaatatccaatagcgggacctggatgcccatattggatcctacgtattctacgaagatcttatcgtttgaacctcagtgccaaggattcatataatcccgtatgtcattccctttgtccttcggtatgttacttgcccgagattcgatcgtcagtatccgtatacgtatttcaatctcgtttaccggcaagtctctttactcgttccgtaatacaagatcccgcaacttacactaagtcacattgcttgcaaggcttgtgtgtgatgttgtattaccgagtgggccccgagatacctctccgtcacacggagtgacaaatcccagtctcgatccatactaactcaatgaacaccttcggagatacctgtagagcatctttatagtcacccagttacgttgcgacgtttgatacacacaaagtattcctccggtgtcagtgagttatatgatctcatggtcataggaacaaatacttgacacgcagaaaactgtagcaacaaaatgacacgatcaacatgctacgtctattagtttgggtctagtccatcacgtgattctcctaatgacgtgatccagttatcaagcaacaacaccttgtttataatcagaagacactgactatctttgatcaactggctagccaactagaggcttgctagggacagtgttttgtctatgtatccacacatgtatataagtcttcattcaatacaattatagcatggataataaacgattatcttgatacaggaattataataataactatattattattgcctctaggacataattccaacaatatatgccatcttccgtgttttattcttctgggcagcggtctgttatgcaaaacgtggtcgccgatatcttctagtctatatcgatgacttttcgacaactgcatctgcaagctccttagtttcaacaagtttattccactgagggggtgtttctttacagggactttttgctgtagggactaaaaaacgtcccttttagtcccatgtgaaagaaacaggagagacttttagggactaaaagggggtatttggaactaaaggaagaagtccctatgggagggactttttgagacttttttgacactttttacaacagtgcccctacttttatcatgtcatttaataacttctagtacattactaggggtaacatggtctttttacatgtcatttaatgacttccaatccctatttagtccctggaaagaaacgggtagggactagagactttttagttggtattaaaaaaaaagtgctaagacttcttaaagaaacagggcgtgagatggaagtgcagaggcggcagcgaattagctcacggggcactaccggtggatgtaggaaaaagaaggcatttgacacctctcaaaactatgaagatgtaaattttaatttctaagtgtttttgtgaggatttgtgctatttaatactccctcaattccaaaataaatgttttaattttgtattatctctaatacaaagttatactaactttaagatacctattttaaaacagaggaaatatatgcattttgcctttctcacaaaaaaaacccctaaaccatcacacgaggggaagagccattcccagccgtccaataaacacgacaaaaagctgtacagcacgggagcaattcccagccgtcgaaatgcgctgcaacggaagtagcgctccctgacacagcggcacatgagccacgcctcgcaccaccgcaccgtgcaccggcgcggggcccaccggccagcgtccccacacgcgtaccggcggccagcattgcgcggcacggcgacggagagatatttttaacgccttccagttcggaagcgccccccatcactcactcgggtacagggcgggccgcgtgagtctcgcgtcgtcgcatcgcgcgcgctgcacacccaccgtcgtcagcgcggaccaccggcgccccaccca

SEQ ID NO: 45 - DNA, 8331 bp, Secale cereale
  source 1..8331: mol_type = genomic DNA
catcgatgacttccagaccgccgcttctacaagctccttattttcaagtcgcttctacaagctccatattttcaacaagtttccttggcaacggctttgctcatggcgcactgcccgtggttgcaagagaaagaatacattcgacacccctaaaaccatgcagatgtaatttctttttgtaaatgtttttgtgaccttttgcctttttccaaaaaaacaaacaaactaaaccatcacacgaggagaagagccattcccagccgtccaataaacacgacaaaacgctttacagcacgggggcaactcccagccgtcgaaaatgcattgcaacggaactactagctctccctgacacagcggcacatgagccacgcctcgcaccgcaaccgtgcaccggcggtgggcccaccggccagtgtcccccacacgtgtaccggcggccagcattgcacggcacggcgacggagagatattttaacgccttccagttcagaagcaccccccatcactcactcgggtacagagcggggccgcgtgactctcgcgtcgtcgcatcgcgcgcgctgcacaccaccgtcgtcagtgcgaccactggcaccaatggcgaccgcctcgccgccgctcgccaccgccttccgcccgctcgccgccggcagcggcggcacccggggaggaggagaaggagcgcacgccgcgggccgggtcgcgccgaggcgggggcgccgggggttcgtggtgcggagcgtggcgagcgatcgagaggtgcgggggcccgcctcgaccgaggaaggtaagcggccgcgcgctcccgttggcttcgacaacccggatgctggccggctgtaatgtggtttggatttccgctggtgcgacccgtgaatttatgatcctctgactgatggtctctgtgttcccgggtttcgggctattggttttgtgcgcgcgcattgctaatctactgctctaggatttgttagggttcagaggaatcttaattcgcgaataaactttcgaggaatgtgttctgaatttactgaaactggtttagtttggtgcgtcaagttagaatgatgtatttatggtacagtagcaaactctgagtaattctactgtgcgtggacagccacccctggtggatgatgcagcaagggtcaacatactgtcgctactatctgtagctgcaacaaaatacttggaatctgaactagtgaaataaatcatttaagatttttgctgaagattacacagtatcaatgtttctgaaggtgattggaacagtatagcacttcaaaatggcaaccaagcaggatcagtatactgacacatcttttttctaaaggtggttcgaataccggtatgaagtttataattatttaattaaggcgtaagatttggtagcaaaaactatttttacgggaaatgatgaaacatggcacaaaaactctaggagctgcctttcatttattaatcgataaaaagagaggtgcagggttctagtagaatgcaaatggtgatacagtatattatctaccattttgtgcaagcattttcacacgaaaacaatatcccatgcagtttttagttttgactctcgactgtttaatacctactctttgtaagatttcagatcctaatattgctgttcttgcaactcagagctttcagccgtgctaacttccattgattcttccgccatcgcgtcaaatatccagcaccatgcagacttcacaccgttgttctcgccagagcactcttcacccctaaaggcttaccatgcaactgctaaaagtgtttttgattctctgatcatgaattggaatgcaacgtatgactattacaacaaagtgaatgcaaagcaagcttattacctgtccatggagtttttgcaggtctcgcaacatacttcctttagccttatctcatacgttttggtaactgcgttatgatgttatatgtttttttttttttttgtactgcagggaagagctcttacaaatgctattggcaacctagagctaacaggacaatatgcagaagcgttaaaacaacttggacagaatctagaggatgttgctagtcaggtaagcgatttctgtcaaagaatagtttttagtccatatcaaaattattgccgagccatttttttttgaaaaagtaacattttattattattcatcaacttccattttattcaaaactatctgcaaaaggagaaaattttctcctgggggttccaccatctgtcttatatttgttatgcagtttttagagtgattgactatgaaattacgccaaaacatgagctttgcctagagtgccctgtagtttagaaatcttaagattggatatgttacttttctattacaatgaaacaggaaccagatcctgcccttggcaatggtggtctaggccgtctagcgtcctgttttctggattctatggctaccctaaattatccagcatggggatatggacttcggtacagatatggcctttttaagcaaatcataacgaaggatggtcaggaggaggtagctgagaattggctagaggtagtgatatgttttctacttgtttactgtttggaagtgttgattatgttggtttcagttttaatgacctgcacgttagcttcttttgccatttataactgttgctgtatctccagatgggaaatccatgggagattgtaagaaatgatgtctcttatcctgtgaaattctatggcaaagtggttgaaggcactgatgggagaaaacactggattggaggagagaatatcaaggctgtggcacatgatgttcctattcctggctacaagactaaaactactaataatcttcgcctttggtcaacaacagtaccatcacaaaactttgatttgggagcttttaatgctggggatcatgccaaggccaatgaagcacacctaaatgctgaaaaggtattcttttgtctgcatttattttgaatataaatcattaaatctcaataactattgatttgaatggtaaacactaaacaatgtctagctagtagtcaccacggtgtcccttgatgtcattttagaatgtgcgcagtcaaatgtttgataactttgattgtaataatatacaaatattcagaaactttgattgacactagatttttcataaacatacttattgtatttccatgtaaatagaaaagctgatgttcagaggtttgtgtgtcaaaatgacaagtaaatatgaacagagtttgtgcacacataacctcttcatctggtgtttgcagttctctaggttgtgtttgtgttttctcctgctctaccttaactaatctcagcatatgcatcaggtacattgctcctctgtgtctataaaaaataagtctgatgagatatggttgcagtccagtagttcgtagttggcacagcatggaagaacttttcacatggaagccttacccttatgattttaattataccttcggagtgtttgtgcatcgtgtacatagaccattaagtatgggtgttcaaaaatagttttcccaaactgtgtaaaagccaccactgtttgtgtattcattttttcgaactagctaaacaacttgaccggagaagatacatacaattgtactcacatctgaagctgctcagttggacagatttgttccaatcccgtgcatgggttaaattgtcgacgtttcctcacttgttcagtagtaggagtactatctattatatttatactaaagttttctttttgtgatatggtacaaacttttaataatacttacatttgcaaacactgtggacaaaatttattatgtctaacttaactttatgactggtcttcgcatcatgccagatatgccacgtattgtatccaggggacgaatcgtcagaggggaaaattcttcgcctgaaacaacaatatacactatgctcggcctccctgcaggacattatttctcgttttgagtcgagggctggtgattctctcaactggcaggacttcccctctaaagttgcagttcagatgaatgacacccatccaacactgtgcattcccgagttaatgagaatactgatggatgttaagggattgagctggactgagtcttggagtatcacggaaaggtatcacagaagggtactgcagtttgatgtgttatgctgttcctcctgttctaactaattttatttcctcatcagtgttccattcaatcatgtactgattttctgacacccacatcatttgtagaaccgttgcatacactaaccacacagtacttcctgaagctctggagaagtggagcttggacattatgcagaaacttctacctcgacatgttgagattatagaaagaatagatgaggaggtatcaaatgtgcagataatattatacgattaatttcattctctgtatttcaacttatggtttacttgtcttcatcgttgattttaaacagctaatgaacaccatcatctcaaaatatggaacggcagatatttcactgttaaaacagaagcttaaagatatgaggatcttagacaacgttgatcttccagcctctgttgctaaactgtttattaaacctaaagagaaaaaggggaagttgcttgttgaatctttggagtctattgctgaagctgacgagaaaagtgagtcacaagaggaggaaaacattcaatctgagacaacagagaaaaagggtgaatctgactctgaagaagccgctgatgcagaaaaggaggatcctgagtatgagttagatccatttgcaaaatacgatcctcagttacctagagttgttcgtatggcaaacctctgtgttgttggtggacattcagttaatggtgttgccgagattcacagtgaaattgttaagcaagatgtgttcaatagcttttatgaggtatttgcacaatgatttggaaaccgtgatcttatttttcaatgactgctataatcgtcaggtgatatgctattcaccatctgccattttcttgtgttctttgccagatgtggcctgctaagttccaaaacaaaacaaatggagtgacccccaggcgttggatccggttttgtaatcctgaattaagtacaatcatttcaaaatggataggctccgatgactggattctgaacactgataaacttgcaggactgaagaaggtacttccagtagctttattccccaataatcagtaatctaatgcttccttcacttcaatgttaactgtaccttgtttttaaattgatgtgttttcatcctgtaacagtttgctgatgatgaggatctgcaatcagaatggcgtactgctaaaaggaataacaagatgaaggtagtttcgctgataagagacaagactggatatgttgtcagcccagatgcaatgtttgatgtgcaggtatattttgtactagaaaacatgcgtctttttataaatatggtttgactcagctcctggtactggacacttaggtgaaaagaatacatgaatataaacggcagctgctaaatatccttggtatcgtttaccgctacaagaagatgaaagaaatgagtgcaaaagatagaataaagagctttgttccaagggtatgcatatttggcgggaaagcttttgccacttacgtacaggcaaagaggattgtgaagtttatcacagatgtcgcagctactgtaaattatgatcctgatattggagatttactgaaggtaaatttgtgtctttgatgccacttatcgattttaattttcatgttctgtcattggcaagttgactcagactgaaaagcagtgaatgttcagagtttcaaaagaaaacagagcagtgaatgttcttaatgccggtgcttgtacaactgagaaactgaataaccgcagttcttttggagaacaatatagccacaattgttttgacccttttattgctatcaagcatctgatgctgatattattgataactagatgaggtaaatacaaggcgacaggttaagtctgaggatttttgtttgttatccaggttgtatttgtcccagattataatgttagcgttgctgagacgctcattcctgccagtgaattgtcccagcatatcaggtaatattctgcctattcttatatagtactccctctgtaaacaaatataagagcatttagatcactattttagtgatctaaacgctcttatgtttgtttacacagggagtatgtgagtacatcaaaaaagaatattaccttttcgtttgctataatatatacctagagtcttactgggatagtgccaccccagtttctatttctgtacaagatgtgcatttttaacagttactatccattcagtaccgctggaatggaagctagtggaaccagcaacatgaagtttgcaatgaatggttgtattcttattggaactttggatggtgcgaatgtggagatcagagaagaggttggagaggagaactttttcctctttggtgcacatgcacctgaaatcgctggtttgcggcaagagagggctgaaggaaaggtaccacacttgtttatatagctgtgcctactatcaaactagtaaagatatctcccatatgtgtgctgtccaaatagtttagctgagggaaaatcataactttaccattttcctttgcacttgtagtttgtgcctgacccgagatttgaagaggttaaggaatacgtccgcagtggcgtctttgggactagtaactatgatgaattgatgggttctttggaaggaaatgaaggttatggacgtgcagattattttcttgttggcaaggattttcccagttacattgaatgccaggaaaaagttgatgaagcataccgagatcagaaggtaagttgtataactgacgctgtttgttttctggctgaaatgcacatggtgattatgcaaactttctgatatgtgatggctgacatcagtgtttcgctgtgaacgacttcactgattcgtaggaaggaatatttcttagttcaaagtttcaaaccggagtatctatcgatgtgttggaacttggaagatcataaacaatcagagaatatagttgctagatgtgctatactactcatctgttggctgttgagtgttgatacatgagctttgatgaacctagctagaccttcatgaaaatgcttctatgcattccaataaaaaaaataactttataacttaacaaaacaattaggttgaacctcgccagggggccttttgtaatctgttttctgaaatgaaaatcagaagcagggagctcctcgattctaaaatgaaaaataaaatcagggataactgaggatctggttttcctctgttggagccccaaagaattctgtggtcttattattgtaagagtataagatgcactgctccgataagtttctccttcatccatttgtttttattgctttgtgacatttcgtgtgacagctcagatcaccatgcatcccagttgtacacattattttgctatcgtggatatttccactctgtttcatgtgcgacatttttaatttgttacattgttactaactacacatactctgatagattttcagagttttggtcctcctaatcacataatatctgtctctacctgcagctatggacaagaatgtctatcctcaacacagctggttcccccaagttcagcagtgaccggacgatccacgagtacgccaaggacatatgggacatcagccccgtcatcatgccctgaataattcaagcagcttcctggaaccttgagagtctatgaggcgcgattggtcgcagatttgcctccattcagcgagaatgtattgcgccccccagttttgtataggcatatgcaagttctgacatggacctgagtgctgagagcatgcagggtagttcatccttagcaatattgcatgtcagttagccagtaagtattacccaggcttatggtgtgtgtatttcgccgaataaagttggagcagaatcatcaaacaaacatactttggtgtatctgtaatgataaataaatggtaaatgactgaacaaaaagtgtcgttggcgcacacatgtagtagaagcatgtttcttccggagagggtgcaaagctgaaagcaccccagttttgaagtgagaatgcagaagttacaaaaacatttgtttacatgcaaagtgcaacgggattcgtcaagagttcagggctcctttgattcaaaggatttgcataggaattttggaggattagagtccttaggaattttttctacgttgaggcctcatttgatttataggatagaaatatcataggaataggaaaatcatagtaagtgagatgatatgc

SEQ ID NO: 46 - DNA, 2916 bp, Secale cereale
  source 1..2916: mol_type = genomic DNA
atggcgaccgcctcgccgccgctcgccaccgccttccgcccgctcgccgccggcagcggcggcacccggggaggaggagaaggagcgcacgccgcgggccgggtcgcgccgaggcgggggcgccgggggttcgtggtgcggagcgtggcgagcgatcgagaggtgcgggggcccgcctcgaccgaggaagagctttcagccgtgctaacttccattgattcttccgccatcgcgtcaaatatccagcaccatgcagacttcacaccgttgttctcgccagagcactcttcacccctaaaggcttaccatgcaactgctaaaagtgtttttgattctctgatcatgaattggaatgcaacgtatgactattacaacaaagtgaatgcaaagcaagcttattacctgtccatggagtttttgcagggaagagctcttacaaatgctattggcaacctagagctaacaggacaatatgcagaagcgttaaaacaacttggacagaatctagaggatgttgctagtcaggaaccagatcctgcccttggcaatggtggtctaggccgtctagcgtcctgttttctggattctatggctaccctaaattatccagcatggggatatggacttcggtacagatatggcctttttaagcaaatcataacgaaggatggtcaggaggaggtagctgagaattggctagagatgggaaatccatgggagattgtaagaaatgatgtctcttatcctgtgaaattctatggcaaagtggttgaaggcactgatgggagaaaacactggattggaggagagaatatcaaggctgtggcacatgatgttcctattcctggctacaagactaaaactactaataatcttcgcctttggtcaacaacagtaccatcacaaaactttgatttgggagcttttaatgctggggatcatgccaaggccaatgaagcacacctaaatgctgaaaagatatgccacgtattgtatccaggggacgaatcgtcagaggggaaaattcttcgcctgaaacaacaatatacactatgctcggcctccctgcaggacattatttctcgttttgagtcgagggctggtgattctctcaactggcaggacttcccctctaaagttgcagttcagatgaatgacacccatccaacactgtgcattcccgagttaatgagaatactgatggatgttaagggattgagctggactgagtcttggagtatcacggaaagaaccgttgcatacactaaccacacagtacttcctgaagctctggagaagtggagcttggacattatgcagaaacttctacctcgacatgttgagattatagaaagaatagatgaggagctaatgaacaccatcatctcaaaatatggaacggcagatatttcactgttaaaacagaagcttaaagatatgaggatcttagacaacgttgatcttccagcctctgttgctaaactgtttattaaacctaaagagaaaaaggggaagttgcttgttgaatctttggagtctattgctgaagctgacgagaaaagtgagtcacaagaggaggaaaacattcaatctgagacaacagagaaaaagggtgaatctgactctgaagaagccgctgatgcagaaaaggaggatcctgagtatgagttagatccatttgcaaaatacgatcctcagttacctagagttgttcgtatggcaaacctctgtgttgttggtggacattcagttaatggtgttgccgagattcacagtgaaattgttaagcaagatgtgttcaatagcttttatgagatgtggcctgctaagttccaaaacaaaacaaatggagtgacccccaggcgttggatccggttttgtaatcctgaattaagtacaatcatttcaaaatggataggctccgatgactggattctgaacactgataaacttgcaggactgaagaagtttgctgatgatgaggatctgcaatcagaatggcgtactgctaaaaggaataacaagatgaaggtagtttcgctgataagagacaagactggatatgttgtcagcccagatgcaatgtttgatgtgcaggtgaaaagaatacatgaatataaacggcagctgctaaatatccttggtatcgtttaccgctacaagaagatgaaagaaatgagtgcaaaagatagaataaagagctttgttccaagggtatgcatatttggcgggaaagcttttgccacttacgtacaggcaaagaggattgtgaagtttatcacagatgtcgcagctactgtaaattatgatcctgatattggagatttactgaaggttgtatttgtcccagattataatgttagcgttgctgagacgctcattcctgccagtgaattgtcccagcatatcagtaccgctggaatggaagctagtggaaccagcaacatgaagtttgcaatgaatggttgtattcttattggaactttggatggtgcgaatgtggagatcagagaagaggttggagaggagaactttttcctctttggtgcacatgcacctgaaatcgctggtttgcggcaagagagggctgaaggaaagtttgtgcctgacccgagatttgaagaggttaaggaatacgtccgcagtggcgtctttgggactagtaactatgatgaattgatgggttctttggaaggaaatgaaggttatggacgtgcagattattttcttgttggcaaggattttcccagttacattgaatgccaggaaaaagttgatgaagcataccgagatcagaagctatggacaagaatgtctatcctcaacacagctggttcccccaagttcagcagtgaccggacgatccacgagtacgccaaggacatatgggacatcagccccgtcatcatgccctga

SEQ ID NO: 47 - protein, 971 aa, Secale cereale
MATASPPLATAFRPLAAGSGGTRGGGEGAHAAGRVAPRRGRRGFVVRSVASDREVRGPASTEEELSAVLTSIDSSAIASNIQHHADFTPLFSPEHSSPLKAYHATAKSVFDSLIMNWNATYDYYNKVNAKQAYYLSMEFLQGRALTNAIGNLELTGQYAEALKQLGQNLEDVASQEPDPALGNGGLGRLASCFLDSMATLNYPAWGYGLRYRYGLFKQIITKDGQEEVAENWLEMGNPWEIVRNDVSYPVKFYGKVVEGTDGRKHWIGGENIKAVAHDVPIPGYKTKTTNNLRLWSTTVPSQNFDLGAFNAGDHAKANEAHLNAEKICHVLYPGDESSEGKILRLKQQYTLCSASLQDIISRFESRAGDSLNWQDFPSKVAVQMNDTHPTLCIPELMRILMDVKGLSWTESWSITERTVAYTNHTVLPEALEKWSLDIMQKLLPRHVEIIERIDEELMNTIISKYGTADISLLKQKLKDMRILDNVDLPASVAKLFIKPKEKKGKLLVESLESIAEADEKSESQEEENIQSETTEKKGESDSEEAADAEKEDPEYELDPFAKYDPQLPRVVRMANLCVVGGHSVNGVAEIHSEIVKQDVFNSFYEMWPAKFQNKTNGVTPRRWIRFCNPELSTIISKWIGSDDWILNTDKLAGLKKFADDEDLQSEWRTAKRNNKMKVVSLIRDKTGYVVSPDAMFDVQVKRIHEYKRQLLNILGIVYRYKKMKEMSAKDRIKSFVPRVCIFGGKAFATYVQAKRIVKFITDVAATVNYDPDIGDLLKVVFVPDYNVSVAETLIPASELSQHISTAGMEASGTSNMKFAMNGCILIGTLDGANVEIREEVGEENFFLFGAHAPEIAGLRQERAEGKFVPDPRFEEVKEYVRSGVFGTSNYDELMGSLEGNEGYGRADYFLVGKDFPSYIECQEKVDEAYRDQKLWTRMSILNTAGSPKFSSDRTIHEYAKDIWDISPVIMP

SEQ ID NO: 48 - DNA, 2000 bp, Secale cereale
  source 1..2000: mol_type = genomic DNA
ttgctcgatcgatagaggggcgcgctatactcgggacggtcttcacggggcctgcggaggcgcgtgcaaccgacgggccaggccggtcgcaccgcgtagatgaggcagatcgcggcgacgggcgggtgaccaattcgatcgcgcgcgaggtcggggacactgctcggtggaggtggggtggcggcggagtccgagatgaagccggcgatgacaaacgtcgtgggacatcgtgcggacgagcgcgtcagcaccggcgcccgggcaacgaggcccgagcgaccaaggtagcagcggcgcccgagcccgacgcaaccgacgagcctgacgcagccggcccgacacagccgaggacgaggccgacgaggtaacacgcagggtcgccgtgtagacgcgacgaagacggatggcgtgaatcgattgacgtgccggcacgcaagcgacggctatgattggccgccgcatggcgcgaggccgccggctgggggcgatgcaggtgccccggtcggagcagggcggtgacggccggcgcggggcgacgggcgtgccggcgcgcgaacgacggccttgaggggccgcctgtcgtgcgaggccgccgggttggtgaagtagccggccagtcgcgtcggtgttagagtagaggcgcgcggatgtcaacggcggcgggtggacgacgcaaaccgatcttagatcggaaaaccaaaaagtaaacgccgatcaaagcgaccggtgagagagagaaaaaactggagcaaaggctcgggaaaagactctctagggaagtcggtcaacacaactgacggacgaaccctaggtatgggtgacgcggcccccagcggcggtcatggaggccgaccgctccgggggcggcgcgcgggtgcggaagccgcggcggtggctagggtttaggatcgattaggctgataccatgtcagaagggagatagaggtttggcggaatagttgttgtattgcttgagcctcgtgggcatatatatatagaagtacatggtcatcttgaagtacaaggcaaggtaaaataatatcctatgctatcctagctttcctaataatcacgatactcaacattgtcattgtagtgaggcatgtgaagggtggtttgtcccaaatctagttagatgggttggggtttttctttggcaggtttatctcagtggtagtgtcccgtgaagcggatgacacgactaggtgtcttggtccttcacgctctagttttggcaggtgaaggttggtcagcttcgcctcgtcggggagcttatgagatttgtgtatctttacttcgtccggtcaaacctgtggcattatataagcactctcatgtgttttcttcttttggacagcagtctgttgtgtaaatcgtagtcgtcgacatcttctagtccacatcgatgacttccagaccgccgcttctacaagctccttattttcaagtcgcttctacaagctccatattttcaacaagtttccttggcaacggctttgctcatggcgcactgcccgtggttgcaagagaaagaatacattcgacacccctaaaaccatgcagatgtaatttctttttgtaaatgtttttgtgaccttttgcctttttccaaaaaaacaaacaaactaaaccatcacacgaggagaagagccattcccagccgtccaataaacacgacaaaacgctttacagcacgggggcaactcccagccgtcgaaaatgcattgcaacggaactactagctctccctgacacagcggcacatgagccacgcctcgcaccgcaaccgtgcaccggcggtgggcccaccggccagtgtcccccacacgtgtaccggcggccagcattgcacggcacggcgacggagagatattttaacgccttccagttcagaagcaccccccatcactcactcgggtacagagcggggccgcgtgactctcgcgtcgtcgcatcgcgcgcgctgcacaccaccgtcgtcagtgcgaccactggcacca

SEQ ID NO: 49 - DNA, 23 bp, synthetic construct
  source 1..23: mol_type = other DNA
accctaaattatccagcatgggg

SEQ ID NO: 50 - DNA, 23 bp, synthetic construct
  source 1..23: mol_type = other DNA
caggaaccagatcctgcccttgg

SEQ ID NO: 51 - DNA, 22 bp, synthetic construct
  source 1..22: mol_type = other DNA
gttgcatggtaagcctttaggg

SEQ ID NO: 52 - DNA, 23 bp, synthetic construct
  source 1..23: mol_type = other DNA
caggggacgaatcatcagagggg

SEQ ID NO: 53 - DNA, 20 bp, synthetic construct
  source 1..20: mol_type = other DNA
accctaaattatccagcatg

SEQ ID NO: 54 - DNA, 20 bp, synthetic construct
  source 1..20: mol_type = other DNA
caggaaccagatcctgccct

SEQ ID NO: 55 - DNA, 19 bp, synthetic construct
  source 1..19: mol_type = other DNA
gttgcatggtaagccttta

SEQ ID NO: 56 - DNA, 20 bp, synthetic construct
  source 1..20: mol_type = other DNA
caggggacgaatcatcagag

SEQ ID NO: 57 - RNA, 83 nt, synthetic construct
  source 1..83: mol_type = other RNA
gttttagagctagaaatagcaagttaaaataaggctagtccgttatcaacttgaaaaagtggcaccgagtcggtgcttttttt

SEQ ID NO: 58 - DNA, 103 bp, synthetic construct
  source 1..103: mol_type = other DNA
accctaaattatccagcatggttttagagctagaaatagcaagttaaaataaggctagtccgttatcaacttgaaaaagtggcaccgagtcggtgcttttttt

SEQ ID NO: 59 - DNA, 103 bp, synthetic construct
  source 1..103: mol_type = other DNA
caggaaccagatcctgccctgttttagagctagaaatagcaagttaaaataaggctagtccgttatcaacttgaaaaagtggcaccgagtcggtgcttttttt

SEQ ID NO: 60 - DNA, 102 bp, synthetic construct
  source 1..102: mol_type = other DNA
gttgcatggtaagcctttagttttagagctagaaatagcaagttaaaataaggctagtccgttatcaacttgaaaaagtggcaccgagtcggtgcttttttt

SEQ ID NO: 61 - DNA, 103 bp, synthetic construct
  source 1..103: mol_type = other DNA
caggggacgaatcatcagaggttttagagctagaaatagcaagttaaaataaggctagtccgttatcaacttgaaaaagtggcaccgagtcggtgcttttttt

SEQ ID NO: 62 - RNA, 103 nt, synthetic construct
  source 1..103: mol_type = other RNA
accctaaattatccagcatggttttagagctagaaatagcaagttaaaataaggctagtccgttatcaacttgaaaaagtggcaccgagtcggtgcttttttt

SEQ ID NO: 63 - RNA, 103 nt, synthetic construct
  source 1..103: mol_type = other RNA
caggaaccagatcctgccctgttttagagctagaaatagcaagttaaaataaggctagtccgttatcaacttgaaaaagtggcaccgagtcggtgcttttttt

SEQ ID NO: 64 - RNA, 102 nt, synthetic construct
  source 1..102: mol_type = other RNA
gttgcatggtaagcctttagttttagagctagaaatagcaagttaaaataaggctagtccgttatcaacttgaaaaagtggcaccgagtcggtgcttttttt

SEQ ID NO: 65 - RNA, 103 nt, synthetic construct
  source 1..103: mol_type = other RNA
caggggacgaatcatcagaggttttagagctagaaatagcaagttaaaataaggctagtccgttatcaacttgaaaaagtggcaccgagtcggtgcttttttt

SEQ ID NO: 66 - DNA, 4203 bp, Streptococcus pyogenes
  source 1..4203: mol_type = genomic DNA
atggctcctaagaagaagcggaaggttggtattcacggggtgcctgcggctgacaagaagtactccatcggcctcgacatcggcaccaacagcgtcggctgggcggtgatcaccgacgagtacaaggtcccgtccaagaagttcaaggtcctgggcaacaccgaccgccactccatcaagaagaacctcatcggcgccctcctcttcgactccggcgagacggcggaggcgacccgcctcaagcgcaccgcccgccgccgctacacccgccgcaagaaccgcatctgctacctccaggagatcttctccaacgagatggcgaaggtcgacgactccttcttccaccgcctcgaggagtccttcctcgtggaggaggacaagaagcacgagcgccaccccatcttcggcaacatcgtcgacgaggtcgcctaccacgagaagtaccccactatctaccaccttcgtaagaagcttgttgactctactgataaggctgatcttcgtctcatctaccttgctctcgctcacatgatcaagttccgtggtcacttccttatcgagggtgaccttaaccctgataactccgacgtggacaagctcttcatccagctcgtccagacctacaaccagctcttcgaggagaaccctatcaacgcttccggtgtcgacgctaaggcgatcctttccgctaggctctccaagtccaggcgtctcgagaacctcatcgcccagctccctggtgagaagaagaacggtcttttcggtaacctcatcgctctctccctcggtctgacccctaacttcaagtccaacttcgacctcgctgaggacgctaagcttcagctctccaaggatacctacgacgatgatctcgacaacctcctcgctcagattggagatcagtacgctgatctcttccttgctgctaagaacctctccgatgctatcctcctttcggatatccttagggttaacactgagatcactaaggctcctctttctgcttccatgatcaagcgctacgacgagcaccaccaggacctcaccctcctcaaggctcttgttcgtcagcagctccccgagaagtacaaggagatcttcttcgaccagtccaagaacggctacgccggttacattgacggtggagctagccaggaggagttctacaagttcatcaagccaatccttgagaagatggatggtactgaggagcttctcgttaagcttaaccgtgaggacctccttaggaagcagaggactttcgataacggctctatccctcaccagatccaccttggtgagcttcacgccatccttcgtaggcaggaggacttctaccctttcctcaaggacaaccgtgagaagatcgagaagatccttactttccgtattccttactacgttggtcctcttgctcgtggtaactcccgtttcgcttggatgactaggaagtccgaggagactatcaccccttggaacttcgaggaggttgttgacaagggtgcttccgcccagtccttcatcgagcgcatgaccaacttcgacaagaacctccccaacgagaaggtcctccccaagcactccctcctctacgagtacttcacggtctacaacgagctcaccaaggtcaagtacgtcaccgagggtatgcgcaagcctgccttcctctccggcgagcagaagaaggctatcgttgacctcctcttcaagaccaaccgcaaggtcaccgtcaagcagctcaaggaggactacttcaagaagatcgagtgcttcgactccgtcgagatcagcggcgttgaggaccgtttcaacgcttctctcggtacctaccacgatctcctcaagatcatcaaggacaaggacttcctcgacaacgaggagaacgaggacatcctcgaggacatcgtcctcactcttactctcttcgaggatagggagatgatcgaggagaggctcaagacttacgctcatctcttcgatgacaaggttatgaagcagctcaagcgtcgccgttacaccggttggggtaggctctcccgcaagctcatcaacggtatcagggataagcagagcggcaagactatcctcgacttcctcaagtctgatggtttcgctaacaggaacttcatgcagctcatccacgatgactctcttaccttcaaggaggatattcagaaggctcaggtgtccggtcagggcgactctctccacgagcacattgctaaccttgctggttcccctgctatcaagaagggcatccttcagactgttaaggttgtcgatgagcttgtcaaggttatgggtcgtcacaagcctgagaacatcgtcatcgagatggctcgtgagaaccagactacccagaagggtcagaagaactcgagggagcgcatgaagaggattgaggagggtatcaaggagcttggttctcagatccttaaggagcaccctgtcgagaacacccagctccagaacgagaagctctacctctactacctccagaacggtagggatatgtacgttgaccaggagctcgacatcaacaggctttctgactacgacgtcgaccacattgttcctcagtctttccttaaggatgactccatcgacaacaaggtcctcacgaggtccgacaagaacaggggtaagtcggacaacgtcccttccgaggaggttgtcaagaagatgaagaactactggaggcagcttctcaacgctaagctcattacccagaggaagttcgacaacctcacgaaggctgagaggggtggcctttccgagcttgacaaggctggtttcatcaagaggcagcttgttgagacgaggcagattaccaagcacgttgctcagatcctcgattctaggatgaacaccaagtacgacgagaacgacaagctcatccgcgaggtcaaggtgatcaccctcaagtccaagctcgtctccgacttccgcaaggacttccagttctacaaggtccgcgagatcaacaactaccaccacgctcacgatgcttaccttaacgctgtcgttggtaccgctcttatcaagaagtaccctaagcttgagtccgagttcgtctacggtgactacaaggtctacgacgttcgtaagatgatcgccaagtccgagcaggagatcggcaaggccaccgccaagtacttcttctactccaacatcatgaacttcttcaagaccgagatcaccctcgccaacggcgagatccgcaagcgccctcttatcgagacgaacggtgagactggtgagatcgtttgggacaagggtcgcgacttcgctactgttcgcaaggtcctttctatgcctcaggttaacatcgtcaagaagaccgaggtccagaccggtggcttctccaaggagtctatccttccaaagagaaactcggacaagctcatcgctaggaagaaggattgggaccctaagaagtacggtggtttcgactcccctactgtcgcctactccgtcctcgtggtcgccaaggtggagaagggtaagtcgaagaagctcaagtccgtcaaggagctcctcggcatcaccatcatggagcgctcctccttcgagaagaacccgatcgacttcctcgaggccaagggctacaaggaggtcaagaaggacctcatcatcaagctccccaagtactctcttttcgagctcgagaacggtcgtaagaggatgctggcttccgctggtgagctccagaagggtaacgagcttgctcttccttccaagtacgtgaacttcctctacctcgcctcccactacgagaagctcaagggttcccctgaggataacgagcagaagcagctcttcgtggagcagcacaagcactacctcgacgagatcatcgagcagatctccgagttctccaagcgcgtcatcctcgctgacgctaacctcgacaaggtcctctccgcctacaacaagcaccgcgacaagcccatccgcgagcaggccgagaacatcatccacctcttcacgctcacgaacctcggcgcccctgctgctttcaagtacttcgacaccaccatcgacaggaagcgttacacgtccaccaaggaggttctcgacgctactctcatccaccagtccatcaccggtctttacgagactcgtatcgacctttcccagcttggtggtgataagcgtcctgctgccaccaaaaaggccggacaggctaagaaaaagaagtag

SEQ ID NO: 67 - DNA, 561 bp, Pseudomonas aeruginosa
  source 1..561: mol_type = genomic DNA
atggaccactacctcgacatcaggctcaggccagacccagagttcccaccagcccagctcatgtccgtcctcttcggcaagctccaccaggccctcgtggcccagggcggcgacaggatcggcgtgtccttcccagacctcgacgagtccaggtccaggctcggcgagaggctccgcatccacgcctccgccgacgacctcagggccctcctcgccaggccgtggctggagggcctcagggaccacctccagttcggcgagccagccgtggtgccacacccaaccccatacaggcaagtgtccagggtgcaagccaagtccaacccagagaggctcaggaggaggctcatgaggaggcacgacctctccgaggaagaggccaggaagcgcatcccagacaccgtggccagggccctcgacctcccattcgtgaccctcaggtcccagtccaccggccagcacttccgcctcttcatcaggcacggcccactccaggtgaccgccgaggagggcggctttacctgctacggcctctccaagggcggcttcgtgccgtggttc

SEQ ID NO: 68 - DNA, 363 bp, Triticum aestivum
  source 1..363: mol_type = genomic DNA
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttg

SEQ ID NO: 69 - DNA, 982 bp, Zea mays
  source 1..982: mol_type = genomic DNA
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaag

SEQ ID NO: 70 - DNA, 25 bp, synthetic construct
  source 1..25: mol_type = other DNA
tttttgattctctgatattgaattg

SEQ ID NO: 71 - DNA, 20 bp, synthetic construct
  source 1..20: mol_type = other DNA
atatccgtttcgacctgcag

SEQ ID NO: 72 - DNA, 25 bp, synthetic construct
  source 1..25: mol_type = other DNA
agagacatcatttcttacgatctcc

SEQ ID NO: 73 - DNA, 24 bp, synthetic construct
  source 1..24: mol_type = other DNA
aaaataaagtagacgaaagaatac

SEQ ID NO: 74 - DNA, 25 bp, synthetic construct
  source 1..25: mol_type = other DNA
tttttgattctctgatattgaatta

SEQ ID NO: 75 - DNA, 20 bp, synthetic construct
  source 1..20: mol_type = other DNA
atatccgtttcgacctgcaa

SEQ ID NO: 76 - DNA, 25 bp, synthetic construct
  source 1..25: mol_type = other DNA
agagacatcatttcttacgatctct

SEQ ID NO: 77 - DNA, 24 bp, synthetic construct
  source 1..24: mol_type = other DNA
aaaataaagtagacgaaagaatat

SEQ ID NO: 78 - DNA, 23 bp, synthetic construct
  source 1..23: mol_type = other DNA
aaggtatgagataaggctgaaga

SEQ ID NO: 79 - DNA, 22 bp, synthetic construct
  source 1..22: mol_type = other DNA
ccatatgtccttggcatactcg

SEQ ID NO: 80 - DNA, 23 bp, synthetic construct
  source 1..23: mol_type = other DNA
acctgcatgttagcttcttttct

SEQ ID NO: 81 - DNA, 22 bp, synthetic construct
  source 1..22: mol_type = other DNA
acaaaattttgatttgggtgcc

SEQ ID NO: 82 - DNA, 21 bp, synthetic construct
  source 1..21: mol_type = other DNA
gaaggtcggagtcaacggatt

SEQ ID NO: 83 - DNA, 21 bp, synthetic construct
  source 1..21: mol_type = other DNA
gaaggtgaccaagttcatgct

SEQ ID NO: 84 - DNA, 23 bp, synthetic construct
  source 1..23: mol_type = other DNA
cggaaggcggtggcgagcggcgg

SEQ ID NO: 85 - DNA, 23 bp, synthetic construct
  source 1..23: mol_type = other DNA
gagcgtggcgagcgatcgggagg

SEQ ID NO: 86 - DNA, 20 bp, synthetic construct
  source 1..20: mol_type = other DNA
cggaaggcggtggcgagcgg

SEQ ID NO: 87 - DNA, 20 bp, synthetic construct
  source 1..20: mol_type = other DNA
gagcgtggcgagcgatcggg

SEQ ID NO: 88 - DNA, 103 bp, synthetic construct
  source 1..103: mol_type = other DNA
cggaaggcggtggcgagcgggttttagagctagaaatagcaagttaaaataaggctagtccgttatcaacttgaaaaagtggcaccgagtcggtgcttttttt

SEQ ID NO: 89 - DNA, 103 bp, synthetic construct
  source 1..103: mol_type = other DNA
gagcgtggcgagcgatcggggttttagagctagaaatagcaagttaaaataaggctagtccgttatcaacttgaaaaagtggcaccgagtcggtgcttttttt

(19) United States
(12) Patent Application Publication
(10) Pub. No.: US 2026/0043039 A1
(43) Pub. Date: Feb. 12, 2026

(54) METHODS OF ALTERING THE STARCH GRANULE PROFILE IN PLANTS

(71) Applicant: Plant Bioscience Limited, Norwich (GB)

(72) Inventors: David SEUNG, Norwich (GB); Nitin Uttam Kamble, Norwich (GB)

(21) Appl. No.: 19/100,249

(22) PCT Filed: May 30, 2023
(86) PCT No.: PCT/EP2023/064465

§ 371 Date: Jan. 31, 2025

(30) Foreign Application Priority Data
Aug. 1, 2022 (LU) LU502613

(51) Int. Cl. C12N 15/82; C12N 15/113

(52) U.S. Cl. CPC C12N 15/8245; C12N 15/1137; C12Y 204/01001; C12N 2310/14; C12N 2310/20

(57) ABSTRACT

The invention relates to methods for altering the starch granule composition in starch storage organs. Also described are genetically altered plants characterised by the above phenotype as well as methods of producing such plants.

### FIELD OF THE INVENTION

The invention relates to methods for altering the starch granule composition in starch storage organs. Also described are genetically altered plants characterised by the above phenotype as well as methods of producing such plants.

### BACKGROUND OF THE INVENTION

Starch is a vital source of calories in human diets and an important industrial raw material for the manufacture of many food and non-food products (such as biofuels, paper, pharmaceuticals and textiles) (Jobling, 2004; Smith, 2008; Santelia and Zeeman, 2010). Most plants store a portion of carbon fixed through photosynthesis during the day as starch in leaf chloroplasts. This starch is then degraded to provide energy for growth and metabolism at night. Some plants also accumulate high levels of starch in amyloplasts (i.e. non-photosynthetic chloroplasts for starch storage) of seeds and storage organs. Starch is the major carbohydrate component of many of our staple crops, including cereal grains (wheat, barley, maize, rye, oat), tubers and storage roots (potato, cassava, yam, sweet potato), and banana fruits.

Native starch exists as insoluble, semi-crystalline granules that are composed of two distinct glucose polymers, amylopectin and amylose (Zeeman et al., 2010; Pfister and Zeeman, 2016; Goren et al., 2018). Amylopectin is the major component of the starch granule and is a highly branched polymer consisting of α-1,4-linked glucan chains with α-1,6-linked branch points. The structure of amylopectin facilitates the formation of double helices between adjacent branches, which form the crystalline regions of the starch granule. Amylose consists of long linear α-1,4-linked chains with very few branches, and is thought to reside in the more amorphous regions of the starch granule. The ratio of amylopectin to amylose, as well as the size and shape of starch granules vary greatly depending on botanical source and organ. In Arabidopsis leaves, starch granules are approximately 1 μm in diameter, and contain about 8-10% amylose (Zeeman et al., 2002; Seung et al., 2015). Cereal starches typically contain approximately 15-30% amylose, but the morphology of the granules vary greatly between species (Jane, 1994; Jane et al., 1999). In rice and maize, starch granules have a size distribution between 3-8 μm and 5-20 μm respectively (Jane, 1994; Lindeboom et al., 2004). Cereal crops of the Triticeae (wheat, rye and barley) have a bimodal distribution of granule size, with larger A-type granules and smaller B-type granules. In wheat, A-type granules are 20-30 μm in diameter, while B-type granules (which initiate about 10-15 days after the initiation of A-type granules) are 2-7 μm in diameter (Bechtel et al., 1990; Howard et al., 2011). A-type granules account for more than 70% of wheat endosperm starch by weight, but less than 10% of the granules by number (Lindeboom et al., 2004).

Studying granule initiation not only has potential to reveal mechanisms underpinning this unique spatiotemporal pattern of A- and B-type granule formation, but can also lead to new approaches to modify starch granule size distributions. There is commercial interest in reducing the number of B-type granules in wheat and barley, and/or increasing their size. In brewing, B-type granules can escape filtration due to their small size. These granules gelatinise during wort boiling, and produce an undesirable “starch haze” in the final beer (Stark and Lynn 1992). B-type granules also ineffectively sediment in a liquid suspension, for example during the industrial preparation of starch and gluten from flour—where B-type granules are lost in the waste water during starch purification and hinders the effective separation of starch from gluten (Stoddard and Sarker, 2000). In wheat, B-type granules have also been proposed to have negative effects on both flour processing and bread-making quality (Park et al., 2009). The lack of B-type granules can also confer unique functional properties to wheat starch and flour and increase water absorption, creating further variation in starch properties that can be exploited to produce a range of food and non-food products (Saccomanno et al. 2022). Accordingly, there is significant value in reducing the number of B-type starch granules.

The initiation of proper A-type granules in wheat requires both STARCH SYNTHASE 4 (SS4) and B-GRANULE CONTENT1 (BGC1). Loss of either protein results in supernumerary granule initiations in most amyloplasts at early grain development, which later fuse to form ‘compound’ starch granules (Chia et al., 2020; Hawkins et al., 2021). To date, there have been no proteins identified as being specifically involved in B-type granule formation.

There therefore exists a need to alter the starch granule composition of plants, and in particular to inhibit B-type granule formation or otherwise reduce the levels of B-type granules in plants. The present invention addresses this need.

### SUMMARY OF THE INVENTION

We demonstrate that PHS1 is required for normal starch granule number and size distribution in the wheat endosperm, due to a specific role of the enzyme in B-type granule formation. We presented three lines of evidence to support this role: Firstly, mutants of durum wheat defective in phs1 had fewer but larger B-type granules, with no differences in the number or size of A-type granules (FIGS. 4 and 7). Secondly, granule size distributions in the mutants were identical to those of the wild type during the early stages of grain development, but deviated from the wild type at 18 dpa—the timepoint after B-type granules started forming in the wild type (FIGS. 7-8). Finally, the bgc1-1 phs1-1 double mutant had similar granule size distributions to the bgc1-1 mutant, suggesting that the further loss of PHS1 in a background that only has A-type granules has no effect on granule size distribution (FIG. 9). Thus, PHS1 appears to be only required for normal B-type granule formation in the wheat endosperm, and not for A-type granule formation.

Based on our findings, we present a model where A- and B-type granule initiation occur via distinct mechanisms involving different enzymes (FIG. 12). B-type granule initiation is fundamentally different to A-type granule initiation in that B-type granules initiate within amyloplasts that already contain an A-type granule. They can therefore be considered “secondary” granule initiations. The A-type granule can act as a source of substrates that can prime B-type granule formation, particularly as MOS are released through the process of amylopectin trimming. By contrast, A-type granules are the first granules to form within each amyloplast during early grain development, and are more likely to require de novo primer formation. We propose that PHS1 may only be important in the secondary B-type granule initiations, perhaps as it processes existing MOS substrates in the plastid in a manner that allows B-type granule initiation.

PHS1 catalyses a reversible reaction, whereby α-1,4-linked glucan chains can be degraded via a phosphorolysis reaction that releases glucose-1-phosphate (G1P), or the glucan chain can be extended using G1P as a substrate. In addition to plastidial PHS1, most plants have a cytosolic isoform, PHS2, which is important for maltooligosaccharide (MOS) metabolism during nighttime starch mobilisation. Compared to PHS2 and other non-plant phosphorylases, PHS1 has a unique 78-amino-acid (L78) insertion that may hinder binding to larger branched substrates, increasing its relative affinity for linear MOS.

It was not expected that PHS1 would be involved in the initiation of B-type granules, as previous reports suggested that in plants without a bimodal distribution of granules, PHS1 was largely redundant or had a negative effect on total starch content of the grain and thus yield (Zeeman et al. 2004; Satoh et al. 2008). Furthermore, in plants with a bimodal distribution of granules, RNAi silencing of PHS1 was previously reported to have no effect on starch granule size distributions (Higgins et al 2013). In contrast to previous reports, we show here that severely reducing or abolishing the expression or activity of PHS1 significantly inhibits the number and decreases the size of B-type granules without affecting starch content. This result could not have been predicted.

Since PHS1 mutations also did not affect plant growth or grain yield (FIG. 3), PHS1 represents another gene target to reduce the content of B-type granule content in plants with a bimodal distribution of starch granules, such as wheat. Varieties with low B-type granules are desirable for bread making. Recently work with BGC1 mutants also demonstrated that starch without B-type granules had higher water absorption, reduced grain hardness and higher protein content (Saccomanno et al., 2022). As described above, B-type granules also cause processing problems due to their small size (Stoddard and Sarker, 2000; Park et al., 2009). Since reduced B-type granules can only be achieved through BGC1 by reducing gene dosage rather than through knockouts, the phenotype is easier to achieve in a polyploid species like wheat than in a diploid species like barley. Thus, PHS1 is a more suitable gene target for reducing B-type granules than BGC1 as the phenotype can be achieved through a homozygous knockout mutation.

In one aspect of the invention, there is provided a genetically altered plant, part thereof or plant cell comprising reduced or abolished expression or activity of at least one PHS1 (plastidial a-glucan phosphorylase) gene.

Preferably, the plant comprises a mutation in at least one gene encoding PHS1 and/or the PHS1 promoter. More preferably, the mutation is a loss of function or partial loss of function mutation, wherein preferably the mutation reduces or abolishes the phosphorylase activity of PHS1.

In an alternative embodiment, the plant comprises an RNAi interference construct that reduces or abolishes the expression of at least one PHS1 gene.

In one embodiment, the PHS1 gene encodes a PHS1 polypeptide comprising SEQ ID NO: 3, 7, 11, 15, 19, 23, 27, 31, 35 or 39 or a functional variant or homologue thereof. Preferably, the functional variant or homologue has at least 60% overall sequence identity to SEQ ID NO: 3, 7, 11, 15, 19, 23, 27, 31, 35, 39, 43 or 47. In another embodiment, the homologue is selected from SEQ ID NO: 43 and 47.

Preferably, the plant has a bimodal starch granule composition comprising a first population of starch granules of a first granule size range and at least a second population of starch granules of a second, smaller size range, and wherein the method comprises reducing the number of granules in the second population and/or increasing the size of starch granules in the second population.

In one embodiment the plant is selected from the Triticeae tribe. In another embodiment, the plant is selected from wheat, barley, rye, maize, potato and sorghum. Preferably, the plant is a plant with a bimodal distribution of starch granules. In one embodiment, the plant is selected from wheat or barley or rye.

In another aspect of the invention, there is provided a method of altering the starch granule characteristics of a plant, the method comprising reducing the expression or activity of at least one PHS1 (plastidial a-glucan phosphorylase) gene.

In a further aspect of the invention, there is provided a method of producing a genetically altered plant with an altered starch granule characteristic, the method comprising reducing the expression or activity of at least one PHS1 (plastidial a-glucan phosphorylase) gene.

Preferably, the plant has a bimodal starch granule composition comprising a first population of starch granules of a first granule size range and at least a second population of starch granules of a second, smaller size range, and wherein the method comprises reducing the number of granules in the second population and/or increasing the size of starch granules in the second population.

In one embodiment the plant is selected from the Triticeae tribe. In another embodiment, the plant is selected from wheat, barley, rye, maize, potato and sorghum. Preferably, the plant is a plant with a bimodal distribution of starch granules. In one embodiment, the plant is selected from wheat or barley or rye.

In one embodiment, the plant comprises A-type and B-type starch granules, and wherein the method comprises reducing the number and/or increasing the size of B-type starch granules.

In one embodiment, the method comprises introducing at least one mutation into at least one gene encoding PHS1 and/or the PHS1 promoter. Preferably, the mutation is a loss of function or partial loss of function mutation, wherein preferably the mutation reduces or abolishes the phosphorylase activity of PHS1.

In another embodiment, the method comprises introducing and expressing in the plant an RNAi interference construct that reduces or abolishes the expression of at least one PHS1 gene.

In one embodiment, the PHS1 gene encodes a PHS1 polypeptide comprising SEQ ID NO: 3, 7, 11, 15, 19, 23, 27, 31, 35 or 39 or a functional variant or homologue thereof. Preferably, the functional variant or homologue has at least 60% overall sequence identity to SEQ ID NO: 3, 7, 11, 15, 19, 23, 27, 31, 35, 39, 43 or 47. In another embodiment, the homologue is selected from SEQ ID NO: 43 and 47.

In one embodiment the soluble sugar contents of the grains of said plant is not affected.

In a preferred embodiment A-type starch granule initiation is not affected.

In another aspect there is provided a plant, plant part or plant cell obtained or obtainable by the method of the invention.

In another aspect of the invention, there is provided a grain derived from the genetically altered plant of the invention, wherein the grain is characterised by reduced or abolished expression of a PHS1 gene and/or reduced or abolished activity of a PHS1 polypeptide. Preferably, the grain comprises at least one mutation in a PHS1 gene and/or promoter.

In another aspect of the invention there is provided starch obtained or obtainable from at least one plant cell of the genetically altered plant of the invention or the grain of the invention.

In another aspect, there is provided a food or feed composition prepared from the grain of the invention or the starch of the invention.

In another aspect, there is provided the use of the grain of the invention or the starch of the invention as a food or feedstuff.

In another aspect, there is provided the use of the grain of the invention or the starch of the invention in any pharmaceutical or industrial application.

### DESCRIPTION OF THE FIGURES

The invention is further described in the following non-limiting figures:

FIG. 1. Identification of wheat PHS1 loci and generation of loss-of-function mutants. A) Gene models of PHS1 homeologs in Chinese Spring. Exons are depicted in dark blue and the 5′ and 3′ UTRs are depicted in light blue. The position of the start (ATG) and stop (TGA) codons are indicated with green arrows. The position of the mutations in the TILLING mutants are indicated with red arrows. B) Native PAGE of phosphorylase activity. Crude extracts of leaves from 4-week-old seedlings of the wild type (WT), phs1-1 single (aa BB and AA bb) and double (aa bb) mutants, and wild-type segregant controls (AA BB) were separated on 7.5% polyacrylamide gels containing 0.3% glycogen (180 μg protein per well). Activity bands were visualised by staining in Lugol's iodine solution. C) As for B), but with phs1-6.

FIG. 2. Plant and grain phenotypes of Taphs1 mutants. The wild-type sibling control (AA BB), single mutants (aa BB and AA bb) and the double mutant (aa bb) from phs1-1 and phs1-6 lines were compared with the Kronos wild type (WT). A) Photograph of plants at maturity. Bar=5 cm. B) Grain yield per plant. C) Photographs of grains showing both dorsal and ventral sides, as well as a cut section through the middle of the grain. D) Thousand grain weight. E) Grain size, indicated as area. For panels D and E, data represent the mean±SEM from n=15-16 plants. Values with different letters are significantly different under a one-way ANOVA and Tukey's posthoc test at p<0.05.

FIG. 3. The Taphs1 mutants have normal leaf starch content. The wild-type sibling control (AA BB), single mutants (aa BB and AA bb) and double mutants (aa bb—for both Taphs1-1 and Taphs1-6) were compared with the Kronos wild type (WT). Seedlings were grown for two weeks under 16 h day/8 h night, and harvested at the End of Day (ED) and End of Night (EN). Values are the mean±SEM from n=5 plants, and those with different letters are significantly different under a one-way ANOVA and Tukey's posthoc test at p<0.05.

FIG. 4. Endosperm starch from the Taphs1-1 mutant has fewer B-type granules. A) Scanning electron micrographs of purified endosperm starch. Bar=10 μm. B) Granule size distributions were determined using a Coulter counter, and the data were expressed as relative % volume (of total starch) vs. granule diameter plots. C and D) B-type granule volume (% of total starch) and the average diameter of B-type granules were extracted from the relative volume vs. diameter plots by fitting a mixed normal distribution. E) Same as B, but expressed as relative starch granule number (% of total granule number) vs. diameter plots. F) The percentage of small granules by number (smaller than 10 μm) were calculated from the coulter counter data. G) The average diameter of A-type granules was calculated from the relative volume vs. diameter plots, as for panels C and D. For panels B-G, plots show the mean from the analysis of n=4-6 replicate starch extractions, each from grains from a separate plant. The shading (on panels B and E) and error bars (on panels C, D, F, G) represent the SEM. Values with different letters are significantly different under a one-way ANOVA with Tukey's post-hoc test (p<0.05).

FIG. 5. Interaction between wheat PHS1 and BGC1 in Nicotiana benthamiana. A) Plastidial localisation of YFP-tagged PHS1 and BGC1 transiently expressed in Nicotiana leaves. Bar=5 μm. B) Pairwise immunoprecipitation (IP) of PHS1: YFP and BGC1: RFP co-expressed in Nicotiana leaves, using anti-YFP beads. Input and IP samples were blotted with YFP (top panels) and RFP antibodies (bottom panels). Chloroplast-targeted YFP (cTP:YFP) and RFP (cTP:RFP) were used as controls to exclude unspecific binding to the fluorescent protein tags.

FIG. 6. Expression of wheat PHS1 loci. A) Expression levels of PHS1 homeologs in the endosperm of durum wheat across different stages of grain development. Values are in transcripts per million (TPM) and are means±SEM from n=3 replicates per time point. B) Native PAGE of phosphorylase activity. Crude extracts were produced from dissected endosperms across different stages of grain development and were separated on 7.5% polyacrylamide gels containing 0.3% glycogen (180 μg protein per well). Activity bands were visualised after incubation with G1P by staining in Lugol's iodine solution.

FIG. 7. Loss of PHS1 affects granule number but not total starch content. The endosperm was dissected from developing grains of WT, phs1-1 and phs1-6 double mutants and corresponding wild-type controls, harvested at 8, 14, 18 and 22 dpa, with n=3 individual plants for each genotype per time point. A) Starch content of the endosperm. Values are expressed relative to the fresh weight of the dissected endosperm. B) Starch granule number in the endosperm. Starch was purified from dissected endosperm and the number of granules was determined using a Coulter counter running in volumetric mode (analysing 2 mL of the suspension). Values are expressed relative to the fresh weight of the dissected endosperm. Values with different letters are significantly different under a one-way ANOVA with Tukey's post-hoc test (p<0.05).

FIG. 8. PHS1 affects granule size distributions during mid-grain development. The endosperm was dissected from developing grains of WT, phs1-1 and phs1-6 double mutants and corresponding wild-type controls, harvested at A-E) 8 dpa, B-F) 14 dpa, C-G) 18 dpa and D-H) 22 dpa, with n=3 individual plants for each genotype per time point. A-D) Granule size distributions were analysed on the Coulter counter and the data were expressed as relative % volume (of total starch) vs. granule diameter plots. The shading represents the ±SEM. E-H) Starch granule morphology observed using scanning electron microscopy. Bars=15 μm.

FIG. 9. Loss of PHS1 does not affect granule size distribution in the bgc1-1 mutant. A) Scanning electron micrographs of purified endosperm starch. Bar=15 μm. B) Granule size distributions were determined using a Coulter counter, and the data were expressed as relative % volume (of total starch) vs. granule diameter plots. C) A-type granule diameter extracted from the relative volume vs. diameter plots by fitting a bimodal mixed normal distribution, except for genotypes with bgc1-1, where a unimodal distribution was fitted. D) The percentage of small granules by number (smaller than 10 μm) were calculated from the Coulter counter data. For panels B-D, plots show the mean from the analysis of n=3 replicate starch extractions, each from grains from a separate plant. The shading (in panel B) and error bars (in panels C and D) represent the ±SEM. Values with different letters are significantly different under a one-way ANOVA with Tukey's post-hoc test (p<0.05).

FIG. 10. Soluble glucan quantification in the developing endosperm tissue. A) Total soluble glucans, B) methanol precipitable fraction of soluble glucans (phytoglycogen and long MOS), and C) the non-precipitable fraction (short MOS) in perchloric acid extracts of grain of WT, phs1-1 and phs1-6 double mutants and corresponding wild-type controls, harvested at 8, 14, 18 and 22 dpa, with n=3 individual plants for each genotype per time point. Values are expressed relative to the fresh weight of the dissected endosperm. Values with different letters are significantly different under a one-way ANOVA with Tukey's post-hoc test (p<0.05).

FIG. 11. Soluble sugar quantification in the developing endosperm tissue. A) Representative HPAEC-PAD chromatograms, used for the quantification of B) glucose, C) fructose, D) sucrose and E) maltose. Perchloric acid extracts of dissected endosperm harvested at 8, 14, 18 and 22 dpa were analysed, with n=3 individual plants for each genotype per time point. Values are expressed relative to the fresh weight of the dissected endosperm. Values with different letters are significantly different under a one-way ANOVA with Tukey's post-hoc test (p<0.05).

FIG. 12. Model of PHS1 action in B-type starch granules initiation in wheat endosperm. During wheat grain development, a single A-type granule initiates in each amyloplast during early grain development and grows around 6-8 days post anthesis (dpa). B-type granules initiate later in grain development, at least partially in stromules, around 15-20 days post anthesis. We propose PHS1 acts on maltooligosaccharides (MOS), likely released from A-type granules by isoamylases (ISA), to initiate B-type granules. This process is disrupted in mutants lacking a functional PHS1 protein, resulting in fewer initiations of B-type starch granules. The fewer B-type granules means each one has a greater share of substrates for granule growth, leading to larger B-type granules in the mutant at grain maturity.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

The term “granule” or “starch granule” as used herein refers to a semi-crystalline granule that is composed of two glucose polymers of glucans-amylopectin and amylose. The size, shape, amylose to amylopectin ratio, any crystalline to amorphous material ratio, starch supramolecular architecture, and amylose-lipid complexes may vary from plant to plant. Starch granules are synthesised and stored in plastids-particularly chloroplasts of leaves and non-photosynthetic plastids specialised for starch storage (amyloplasts) in seeds and vegetative storage organs.

The aspects of the invention involve recombinant DNA technology and exclude embodiments that are solely based on generating plants by traditional breeding methods.

In an aspect of the invention, there is provided a method for altering a starch granule characteristic in a plant by reducing or abolishing the expression of at least one plastidial a-glucan phosphorylase (referred to herein as “PHS1” or “PHO1” (phosphate 1)) nucleic acid and/or reducing or abolishing the activity of a PHS1 polypeptide.

As used here, altering a starch granule characteristic in a plant may mean altering the characteristic in all plastids or plastid-types (e.g. photosynthetic plastids (in leaves)) and non-photosynthetic plastids (e.g. amyloplasts). For example therefore, the starch granule characteristic may be altered in leaves or seeds or vegetative storage organs, or all of leaves, seeds and vegetative storage organs. Most preferably the starch granule characteristic is altered in seeds and/or vegetative storage organs.

The plant may comprise at least two populations of starch granules of different sizes—e.g. a first and second size. In one embodiment, the plant comprises a first population of starch granules that have a mean diameter larger than the mean diameter of the second population of starch granules. The first population may be referred to “A-type granules”. The second population may be referred to as “B-type granules”. Granules with a mean diameter of between 10 and 30 μm, preferably between 16 and 23 μm, more preferably above 10 μm and even more preferably above 15 μm may be referred to as A-type granules. Granules with a mean diameter of between 1 and 10 μm, preferably below 10 μm, and even more preferably between 4 and 7 μm may be referred to as B-type granules. The shape of the different granule populations may also differ. For example, A-type granules may be discoid or lenticular, and B-type granules may be spherical or polygonal. In wheat, for example, A-type granules typically make up more than 70% of the total weight of starch, whereas B-type granules comprise up to 90% of the granules in number.

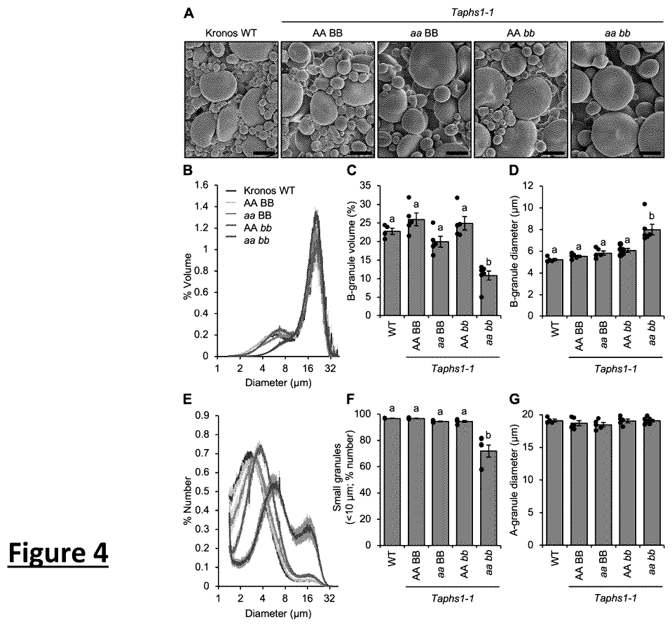

In one embodiment, there is provided a method of reducing the number of granules in one population of starch granules or abolishing one population of starch granules. Preferably the method comprises reducing the number of B-type granules or abolishing B-type granules. By “reducing” is meant a reduction in the number of B-type starch granules in the plant by at least 10%, 20%, 30%, 40%, 50%, 60%, 70%, 80%, 90% 95% or more, more preferably a reduction of between 10 and 40%, more preferably between 25 and 30% in the number of B-type starch granules (as shown in FIG. 4F) compared to the number of B-type starch granules present in a plant where the expression and/or activity of PHS1 is not altered. By “abolishing” is meant that no B-type granules are produced or can be detected. The percentage of small starch granules (including B-type granules and small A-type granules) (for example, below 10 μm, relative to the total number of starch granules) may be measured, as shown for example in FIG. 4E. As shown in FIG. 4F. there is a 25-30% reduction in the relative number of B-type granules in the PHS1 mutant.

Alternatively, the volume of B-type granules may be considered as a percentage of the total volume of granules (i.e. A and B-type granules combined). This may also be referred to herein as the granule volume. Typically, for example in wheat, the volume of B-type granules is approx. 20-30%. As shown in FIG. 4C, the methods of the invention can be used to reduce the volume percent of B-type granules to around 10 and 15%. Thus, the methods of the invention can be used to reduce the B-granule volume (compared to wild-type) by at least 10%, 20%, 30%, 40%, 50%, 60%, 70%, 80%, 90% 95% or more, more preferably between 30 and 70% and even more preferably between 50 and 60%. This results of the method of the invention can also be considered as causing an increase in the mean granule size (or diameter) of the total granule population.

In an alternative or additional embodiment, the method may also comprise increasing the size (for example, the average granule diameter) of starch granules, and in particular where there exists two or more populations of starch granules, as described above, the method may comprise increasing the size of one population of starch granules. Accordingly, the method may comprise increasing the size, such as the average granule diameter, of B-type granules. This increase in size may be at least 10%, 20%, 30%, 40%, 50%, 60%, 70%, 80%, 90% 95% or more, preferably between 30 and 70% and even more preferably between 45 and 55% compared to the size of B-type starch granules present in a plant where the expression and/or activity of PHS1 is not reduced/abolished. As shown in FIG. 4, the methods of the invention can be used to increase the size of B-type granules by about 60% (e.g. from about 5 to 8 μm).

Reducing the amount of B-type granules, as well as increasing the size of B-type granules, can bring several advantages by reducing the heterogeneity in starch granule size. For example in brewing, B-type granules can escape filtration due to their small size, gelatinise during wort boiling, and produce an undesirable “starch haze” in the final beer (Stark and Lynn 1992). Reducing the total amount of B-type granules would help alleviate this problem, and increasing the size of any remaining B-type granules would reduce the chance that they escape filtration.

In the industrial preparation of starch and gluten from flour, B-type granules are lost in the waste during starch purification because they do not effectively sediment, and they hinder the effective separation of starch from gluten (Stoddard and Sarker, 2000). The amount of non-sedimenting granules can be reduced by both reducing total B-type granule volume, and increasing the volume of any remaining B-type granules so that they have increased sedimentation efficiency.

In wheat, B-type granules have been proposed to have negative effects on both flour processing and bread-making quality (Park et al., 2009). The lack of B-type granules can also confer unique functional properties to wheat starch and flour, including higher water absorption (Saccomanno et al. 2022). Thus, starches with altered B-type granule volume and size may have novel physical and chemical properties, which can be exploited in making various food and non-food products.

Thus, there are significant advantages to increasing both the number and the size of the B-type granules.

In summary, the method of the invention may reduce the number but increase the size of B-type granules in a plant.

Granule size may be measured by a number of techniques that would be known to the skilled person. In one embodiment, granule size can be measured using a particle size analyser, which uses laser scattering to measure the total volume of particles of a given size, expressed as a percentage of the total volume of all particles. In one example, purified starch can be suspended in water and measured on a particle size analyser, such as a Beckman-Coulter Multisizer 4e Coulter counter, or the Coulter LS-230 laser-scattering instrument (Beckman Coulter). In another embodiment, granule size can be measured using light microscopy, where starch granule area in the images are measured using the Particle Analysis plugin of ImageJ software (v.2.0.0; https://imagej.net/). The area can be used to calculate diameter, assuming the granules were perfect circles. Unlike the first method with the particle size analyser, this method calculates the percentage of granules with a given size relative to the total number of granules (rather than as volumes as measured on a Coulter counter), and is a direct measure of size (rather than inferred from laser scattering).

The methods described herein may also comprise the step of measuring an altered starch characteristic, for example, a decrease in the number of B-type granules and/or an increase in the size of B-type granules using the methods described above.

As used herein, the terms “reducing” means a decrease in the levels of PHS1 expression and/or activity by up to or more than 10%, 20%, 30%, 40%, 50%, 60%, 70%, 80% or 90% when compared to the level in a wild-type or control plant. In one embodiment, reducing means a decrease in at least 50% compared to the level in a wild-type or control plant. Reducing may or may not encompass changes in the absolute PHS1 transcript level, preferably it does not. Reducing also may or may not encompass abolishing expression. The term “abolish” expression means that no expression of PHS1 is detectable (no transcript) or that no functional PHS1 polypeptide is produced. Methods for determining the level of PHS1 expression and/or activity would be well known to the skilled person. These reductions can be measured by any standard technique known to the skilled person. For example, a reduction in the expression and/or content levels of at least PHS1 expression may be a measure of protein and/or nucleic acid levels and can be measured by any technique known to the skilled person, such as, but not limited to, any form of quantitative PCR, gel electrophoresis and immunoblotting or chromatography (e.g. HPLC).

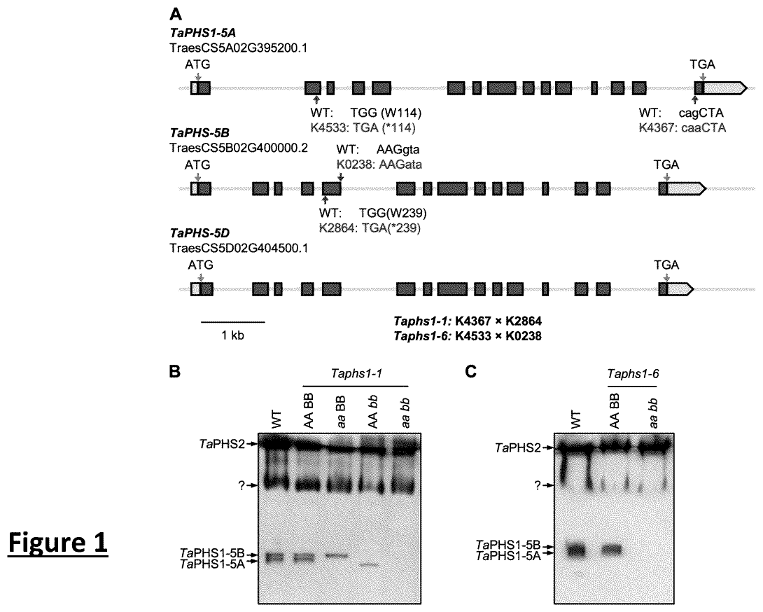

A reduction in activity may also refer to a complete or partial loss-of-function. Accordingly, the method may result in complete or partial loss of phosphorylase activity. By complete, may mean that no phosphorylase activity can be detected. The phosphorylase activity can be measured using any known technique in the art, for example, using native PAGE with phosphorylase activity staining (as in FIG. 1), or using in vitro assays for phosphorylase that measure Glc-1-P incorporation into any glucan acceptor substrate. The incorporation may be quantified by measuring Glc-1-P depletion or release of inorganic phosphate.

In a particular embodiment, the method comprises reducing or abolishing the expression of at least one PHS1 nucleic acid and/or reducing or abolishing the activity of a PHS1 polypeptide in a starch storage organ, such as a tuber or grain. Alternatively, or additionally, the method comprises reducing or abolishing the expression of at least one PHS1 nucleic acid and/or reducing or abolishing the activity of a PHS1 polypeptide in an amyloplast, preferably in the endosperm of the plant. Alternatively, or additionally, the method comprises reducing or abolishing the expression of at least one PHS1 nucleic acid and/or reducing or abolishing the activity of a PHS1 polypeptide in the leaves of the plant. As a result, the characteristics of starch granules is altered in at least one plastid or plastid-type in a plant.

In one embodiment, the method comprises introducing at least one mutation into the, preferably endogenous, gene encoding PHS1 and/or the PHS1 promoter. The mutation may be any mutation that reduces or abolishes the phosphorylase activity of PHS1. As such, the mutation may be considered to be a partial or loss-of-function mutation.

The method may not comprise introducing a mutation into any other gene. In other words, only the PHS1 gene and/or promoter is mutated.

In another aspect of the invention there is provided a genetically altered plant, part thereof or plant cell characterised in that the expression of PHS1 is reduced or abolished or the plant does not express a functional or fully functional PHS1 protein. In one embodiment, the plant is a reduction (knock down) or loss of function (knock out) mutant wherein the function of the PHS1 nucleic acid sequence is reduced or lost compared to a wild type or control plant. Preferably, the plant is a knock out and not a knock down, meaning that PHS1 expression is abolished or significantly abolished (e.g. by at least 80%, 90% or 95% or more) or that the plant expresses a PHS1 protein with no detectable function and/or activity (e.g. phosphorylase function). To this end, a mutation is introduced into either the PHS1 gene sequence or the corresponding promoter sequence which disrupts the transcription of the gene or the function of the protein. The mutation may be any mutation that reduces or abolishes the phosphorylase activity of PHS1.

In one embodiment, the plant has a bimodal size distribution of starch granules (such as, but not limited to) wheat, rye and barley, and preferably said bimodal distribution comprises a first population of starch granules of a first size or size range and a second population of starch granules of a second smaller size or size range. For example, the plant may comprise A-type and B-type granules, as described in detail above. Preferably the plant is characterised by a reduced number of starch granules in the second population and/or an increase in size of starch granules in the second population. As such, the plant, plant part or plant cell may be characterised by fewer (e.g. a decrease in number compared to a wild-type or control plant) B-type granules and/or larger (e.g. an increase in granule diameter compared to a wild-type or control plant) B-type granules).

The plant may also be characterised by an increase in the average granule size.

The plant may comprise at least one mutation in the promoter and/or at least one mutation in at least one gene encoding PHS1. In one embodiment the plant may comprise a mutation in both the promoter and the at least one gene for PHS1. Preferably said mutation is introduced into a least one plant cell and a plant regenerated from the at least one mutated plant cell.

The plant may not comprise a mutation introduced into any other gene. In other words, only the PHS1 gene and/or promoter is mutated.

The mutation may be introduced into the coding region of the PHS1 gene. Alternatively, the mutation may be in an intronic sequence, a splice site or the 5′UTR or 3′UTR. In a further alternative, at least one mutation or structural alteration may be introduced into the PHS1 promoter such that the PHS1 gene is either not expressed (i.e. expression is abolished) or expression is reduced, as defined herein. In an alternative embodiment, at least one mutation may be introduced into the PHS1 gene such that the altered gene does not express a full-length (i.e. expresses a truncated) PHS1 protein or does not express a fully functional PHS1 protein. In this manner, the (phosphorylase) activity of the PHS1 polypeptide can be considered to be reduced or abolished as described herein.

In any case, the mutation may result in the expression of PHS1 with no, significantly reduced or altered biological activity in vivo.

By “at least one mutation” is meant that where the PHS1 gene is present as more than one copy or homeologue (with the same or slightly different sequence) there is at least one mutation in at least one gene. In one embodiment, all genes are mutated. In another embodiment, where the plant is a tetraploid, for example tetraploid wheat, the PHS1 gene is mutated on the A genome only or the A and B genome. In another embodiment, where the plant is a hexaploid, for example hexaploid wheat, the PHS1 gene is mutated on the A and/or B and/or D genome or more preferably, the A and B and D genomes

In one embodiment, the sequence of the PHS1 gene comprises or consists of a nucleic acid sequence selected from SEQ ID NO: 1, 5, 9, 13, 17, 21, 25, 29, 33 and 37 (genomic) or 2, 6, 10, 14, 18, 22, 26, 30, 34 and 38 (CDS) or a functional variant or homologue thereof and encodes a polypeptide as defined in one of SEQ ID NO: 3, 7, 11, 15, 19, 23, 27, 31, 35 and 39 respectively or a functional variant or homologue thereof. The genomic DNA sequences of the 6A homeolog is shown in SEQ ID NO: 1, 13, 25 and 33 respectively the 6B homelog is shown in SEQ ID NO: 17, 29 and 37, and the 6D (homeolog for e.g. Cadenza) is shown in SEQ ID NO: 21, and functional variants thereof. The cDNA sequences are shown in SEQ ID NOs 14, 26 and 35 (6A), 18, 30 and 38 (6B) and 22 (6D-Cadenza).

As used throughout, by “PHS1 promoter” is meant a region extending at least or approx. 2 kbp upstream of the ATG codon of the PHS1 ORF. In one embodiment, the sequence of the PHS1 promoter comprises or consists of a nucleic acid sequence as defined in any one of SEQ ID NO: 4, 8, 12, 16, 20, 24, 28, 32, 36 and 40 or a functional variant or homologue thereof. In one embodiment, the PHS1 promoter may also include 5′ UTR sequences.

In the above embodiments an ‘endogenous’ nucleic acid may refer to the native or natural sequence in the plant genome. Also included in the scope of this invention are functional variants (as defined herein) and homologs of the above identified sequences. Examples of homologs are shown in SEQ ID NOs: 41 to 48. Accordingly, in one embodiment, the homolog is barley and encodes a polypeptide selected from SEQ ID NOs: 43; or the homolog comprises or consists of a nucleic acid sequence selected from SEQ ID NOs: 41 or 42. In another embodiment, the PHS1 promoter homolog comprises or consists of a nucleic acid sequence as defined in SEQ ID NO: 44. In another embodiment, the homolog is rye and encodes a polypeptide selected from SEQ ID NOs: 43; or the homolog comprises or consists of a nucleic acid sequence selected from SEQ ID NOs: 41 or 42. In another embodiment, the PHS1 promoter homolog comprises or consists of a nucleic acid sequence as defined in SEQ ID NO: 44.

The term “functional variant” (or “variant”) as used herein with reference to any of the sequences described herein refers to a variant sequence or part of the sequence which retains the biological function of the full non-variant sequence. Accordingly, in the context of PHS1, a functional variant also has phosphorylase activity. As used herein, a homolog may also be referred to as functional. That is, the homolog also has phosphorylase activity.

The functional variant may comprise variant of the PHS1 gene, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active (e.g. has phosphorylase activity). Alterations in a nucleic acid sequence which result in the production of a different amino acid at a given site that do not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

In one embodiment, a functional variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

The term homolog, as used herein, also designates a PHS1 promoter or PHS1 gene orthologue from other plant species. A homolog may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by any of SEQ ID NO: 3, 7, 11, 15, 19, 23, 27, 31, 35, 39, 43 and 47 or to the nucleic acid sequences as shown by SEQ ID NOs: 1, 2, 5, 6, 9, 10, 13, 14, 17, 18, 21, 22, 25, 26, 29, 30, 33, 34, 37, 38, 41, 42, 45 and 46. A PHS1 promoter orthologue may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the nucleic acid sequences as shown in SEQ ID NOs 4, 8, 12, 16, 20, 24, 28, 32, 36, 40, 44 and 48. In one embodiment, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or at least 99%. Functional variants of PHS1 homologs as defined above are also within the scope of the invention.

PHS1 features a highly conserved Glycosyltransferase-35 (GT-35) domain typically found in starch and glycogen phosphorylases, and which constitutes most of the amino acid sequence (amino acids 77-962 of wheat PHS1-5A and PHS1-5D, and 80-965 of PHS1-5B of SEQ ID NO: 3, 11, 15, 23, 27 or 35, or homologous positions in for example barley or rye). All plastidial phosphorylase isoforms also have an “L78 insertion” that is proposed to be involved in substrate selection (amino acids 483-559 of wheat PHS1-5A and PHS1-5D, and 486-562 of PHS1-5B of SEQ ID NO: 3, 11, 15, 23, 27 or 35 or homologous positions in for example barley or rye). Accordingly, in one embodiment the PHS1 protein comprises at least one conserved GT-35 domain and/or L78 insertion domain and one mutation is introduced into at least GT-35 domain and/or L78 insertion domain to affect protein function. The mutation may encode an amino acid substitution that disrupts the function of the GT-35 domain and/or the L78 insertion domain.

Accordingly, in a further embodiment, the PHS1 nucleic acid (coding) sequence encodes a PHS1 protein with at least one conserved domain, wherein the conserved domain is a GT-35 domain and/or L78 insertion domain, as described above.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms. The overall sequence identity of a variant can be determined using any number of sequence alignment programs known in the art. As an example, Emboss Stretcher from the EMBL-EBI may be used:

$$https \text{://} www . ebi . ac . uk / Tools / psa / emboss stretcher / ( using default parameters : pair output format , Matrix = BLOSUM62 , Gap open = 1 , Gap extend = 1 for proteins ; pair output format , Matrix = DNAfull , Gap open = 16 , Gap extend = 4 for nucleotides ) .$$

The skilled person would understand that suitable homologues and the homologous positions in these sequences can be identified by sequence comparisons (e.g. BLAST, alignments) and identifications of conserved domains. Phylogenetic tree analysis using nucleotide or amino acid sequences can be used to establish orthology to PHS1. There are predictors in the art that can be used to identify such sequences. The function of the homologue can be identified as described herein and a skilled person would thus be able to confirm the function, for example using a phosphorylase assay. Homologous positions or as used herein “corresponding positions in homologous sequences” can thus be determined by performing sequence alignments once the homologous sequence has been identified. For example, homologues can be identified using a BLAST search of the plant genome of interest using the wheat PHS1 as a query (i.e. one of the sequences defined in SEQ ID NOs: 1 to 40).

Thus, the nucleotide sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domains structure can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than 24 hours, usually about 4 to 12. Stringent conditions may also be achieved with the addition of destabilizing agents such as formamide.

In a further embodiment, a variant as used herein can comprise a nucleic acid sequence encoding a PHS1 polypeptide as defined herein that is capable of hybridising under stringent conditions as defined herein to a nucleic acid sequence as defined herein, such as in any of SEQ ID NOs: 1, 5, 9, 13, 17, 21, 25, 29, 33, 37, 41 and 45 (genomic) or 2, 6, 10, 14, 18, 22, 26, 30, 34 and 38, 42 and 46 (CDS).

In one embodiment, the method comprises reducing or abolishing the expression of at least one nucleic acid encoding a PHS1 polypeptide or reducing or abolishing the activity of an PHS1 polypeptide, as described herein, wherein the method comprises introducing at least one mutation into at least one PHS1 gene and/or promoter, wherein the PHS1 gene comprises or consists of
a. a nucleic acid sequence encoding a polypeptide as defined in one of SEQ ID NOs: SEQ ID NO: 3, 7, 11, 15, 19, 23, 27, 31, 35, 39, 43 and 47; or
b. a nucleic acid sequence as defined in one of SEQ ID NOs: 1, 5, 9, 13, 17, 21, 25, 29, 33, 37, 41 and 45 (genomic) or 2, 6, 10, 14, 18, 22, 26, 30, 34 and 38, 42 and 46 (CDS) or
c. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (a) or (b); or
d. a nucleic acid sequence encoding a PHS1 polypeptide as defined herein that is capable of hybridising under stringent conditions as defined herein to the nucleic acid sequence of any of (a) to (c);
   and wherein the PHS1 promoter comprises or consists of
e. a nucleic acid sequence as defined in one of SEQ ID NOs: 4, 8, 12, 16, 20, 24, 28, 32, 36, 40, 44 and 48;
f. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to (e); or
g. a nucleic acid sequence capable of hybridising under stringent conditions as defined herein to the nucleic acid sequence of any of (e) to (f).

In another embodiment, the genetically altered plant, plant part thereof or plant cell reduced or abolished expression of at least one nucleic acid encoding a PHS1 polypeptide or reduced or abolished activity of an PHS1 polypeptide, as described herein, wherein the genetically altered plant, part thereof or plant cell comprises at least one mutation in at least one PHS1 gene and/or promoter, wherein the PHS1 gene comprises or consists of
a. a nucleic acid sequence encoding a polypeptide as defined in one of SEQ ID NOs: SEQ ID NO: 3, 7, 11, 15, 19, 23, 27, 31, 35, 39, 43 and 47; or
b. a nucleic acid sequence as defined in one of SEQ ID NOs: 1, 5, 9, 13, 17, 21, 25, 29, 33, 37, 41 and 45 (genomic) or 2, 6, 10, 14, 18, 22, 26, 30, 34 and 38, 42 and 46 (CDS) or
c. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (a) or (b); or
d. a nucleic acid sequence encoding a PHS1 polypeptide as defined herein that is capable of hybridising under stringent conditions as defined herein to the nucleic acid sequence of any of (a) to (c);
   and wherein the PHS1 promoter comprises or consists of
e. a nucleic acid sequence as defined in one of SEQ ID NOs: 4, 8, 12, 16, 20, 24, 28, 32, 36, 40, 44 and 48;
f. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to (e); or
g. a nucleic acid sequence capable of hybridising under stringent conditions as defined herein to the nucleic acid sequence of any of (e) to (f).

In one embodiment, a mutation is introduced into two PHS1 genes, wherein the first PHS1 gene encodes a protein selected from SEQ ID NO: 3, 7, 11, 15, 19, 23, 27, 31, 35 and 39 (preferably the PHS1 gene comprises or consists of a nucleic acid sequence selected from SEQ ID NO: 1, 2, 5, 6, 9, 10, 13, 14, 17, 18, 21, 22, 25, 26, 29, 30, 33, 34, 37 and 38) and wherein the second PHS1 gene encodes a protein selected from (different from the first) SEQ ID NO: 3, 7, 11, 15, 19, 23, 27, 31, 35 and 39 (preferably the PHS1 gene comprises or consists of a nucleic acid sequence selected, different from the first, from SEQ ID NO: 1, 2, 5, 6, 9, 10, 13, 14, 17, 18, 21, 22, 25, 26, 29, 30, 33, 34, 37 and 38).

In a further embodiment, a mutation is introduced into three PHS1 genes, wherein the first PHS1 gene encodes a protein as defined in SEQ ID NO: SEQ ID NO: 3, 7, 11, 15, 19, 23, 27, 31, 35 and 39 (preferably the PHS1 gene comprises or consists of a nucleic acid sequence selected from SEQ ID NO: 1, 2, 5, 6, 9, 10, 13, 14, 17, 18, 21, 22, 25, 26, 29, 30, 33, 34, 37 and 38); and wherein the second PHS1 gene encodes a protein selected from (different from the first) SEQ ID NO: 3, 7, 11, 15, 19, 23, 27, 31, 35 and 39 (preferably the PHS1 gene comprises or consists of a nucleic acid sequence selected, different from the first, from SEQ ID NO: 1, 2, 5, 6, 9, 10, 13, 14, 17, 18, 21, 22, 25, 26, 29, 30, 33, 34, 37 and 38); and wherein the third PHS1 gene encodes a protein selected from (different from the first and second) SEQ ID NO: 3, 7, 11, 15, 19, 23, 27, 31, 35 and 39 (preferably the PHS1 gene comprises or consists of a nucleic acid sequence selected, different from the first and second, from SEQ ID NO: 1, 2, 5, 6, 9, 10, 13, 14, 17, 18, 21, 22, 25, 26, 29, 30, 33, 34, 37 and 38.

In a preferred embodiment, the mutation that is introduced into the endogenous PHS1 gene or promoter thereof to alter the biological activity and/or expression levels of the PHS1 gene or protein can be selected from the following mutation types:
1. a “missense mutation”, which is a change in the nucleic acid sequence that results in the substitution of one amino acid for another amino acid;
2. a “nonsense mutation” or “STOP codon mutation”, which is a change in the nucleic acid sequence that results in the introduction of a premature STOP codon and, thus, the termination of translation (resulting in a truncated protein); in plants, the translation stop codons may be selected from “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.
3. an “insertion mutation” of one or more nucleotides or one or more amino acids, due to one or more codons having been added in the coding sequence of the nucleic acid;
4. a “deletion mutation” of one or more nucleotides or of one or more amino acids, due to one or more codons having been deleted in the coding sequence of the nucleic acid;
5. a “frameshift mutation”, resulting in the nucleic acid sequence being translated in a different frame downstream of the mutation. A frameshift mutation can have various causes, such as the insertion, deletion or duplication of one or more nucleotides.
6. a “splice site” mutation, which is a mutation that results in the insertion, deletion or substitution of a nucleotide at the site of splicing (i.e. either a splice acceptor or splice donor mutation)
   where any one or more of the above mutations leads to a loss or partial loss of function in PHS1 (e.g. a complete or partial loss of phosphorylase activity).

In one embodiment, the mutation is a STOP codon mutation. For example, the mutation may be a G to A mutation at position 1755 of SEQ ID NO: 33 or a corresponding position in a homologous sequence (this may be referred to as the K4533 mutation); or a G to A mutation at position 2025 of SEQ ID NO: 37 or a corresponding position in a homologous sequence (this may be referred to as the K2864 mutation).

In another embodiment, the mutation is a splice acceptor mutation. For example, the mutation may be a G to A mutation at position 4790 of SEQ ID NO: 33 or a corresponding position in a homologous sequence. This may be referred to as the K4367 mutation.

In another embodiment, the mutation is a splice donor mutation. For example, the mutation may be a G to A mutation at position 2287 of SEQ ID NO: 37 or a corresponding position in a homologous sequence. This may be referred to as the K0238 mutation.

In one embodiment, the method may comprise introducing one or more of the K4533, K4367, K0238 and K2864 (or homologous mutations) as described herein into a PHS1 nucleic acid. In a further embodiment, the method may comprise introducing the following mutations.
a K4367 and a K2864 mutation (or homologous mutations) into a PHS1-1 nucleic acid. A plant comprising both mutations is described herein as PHS1-1; or
a K4533 and a K0238 mutation (or homologous mutations) into a PHS1 nucleic acid. A plant comprising both mutations is described herein as PHS1-6.

In general, the skilled person will understand that at least one mutation as defined above and which leads to the insertion, deletion or substitution of at least one nucleic acid or amino acid compared to the wild-type PHS1 promoter or PHS1 nucleic acid or protein sequence can affect the biological activity of the PHS1 protein.

In one embodiment a mutation may be introduced into the PHS1 promoter and at least one mutation is introduced into the PHS1 gene.

In one embodiment, the mutation is introduced using mutagenesis or targeted genome editing. That is, in one embodiment, the invention relates to a method and plant that has been generated by genetic engineering methods as described above, and does not encompass naturally occurring varieties.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of FokI to direct nucleolytic activity toward specific genomic loci.

Upon delivery into host cells via the bacterial type III secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD that determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the FokI nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Cermak T et al. describes a set of customized plasmids that can be used with the Golden Gate cloning method to assemble multiple DNA fragments. As described therein, the Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct. Accordingly, using techniques known in the art it is possible to design a TAL effector that targets a PHS1 gene or promoter sequence as described herein.

Another genome editing method that can be used according to the various aspects of the invention is CRISPR. The use of this technology in genome editing is well described in the art, for example in U.S. Pat. No. 8,697,359 and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Accordingly, using techniques known in the art it is possible to design sgRNA molecules that targets a PHS1 gene or promoter sequence as described herein. In one embodiment, the method comprises using any of the nucleic acid constructs or sgRNA molecules described herein.

Cas9 expression plasmids for use in the methods of the invention can be constructed as described in the art.

In one embodiment, the method uses a sgRNA to introduce a targeted SNP or mutation, in particular one of the substitutions described herein, into a PHS1 gene. As explained herein, the introduction of a template DNA strand, following a sgRNA-mediated snip in the double-stranded DNA, can be used to produce a specific targeted mutation (i.e. a SNP) in the gene using homology directed repair. In an alternative embodiment, at least one mutation may be introduced into the PHS1 gene and/or promoter, particularly at the positions described above, using any CRISPR technique known to the skilled person. In another example, sgRNA (for example, as described herein) can be used with a modified Cas9 protein, such as nickase Cas9 or nCas9 or a “dead” Cas9 (dCas9) fused to a “Base Editor”-such as an enzyme, for example a deaminase such as cytidine deaminase, or TadA (tRNA adenosine deaminase) or ADAR or APOBEC. These enzymes are able to substitute one base for another. As a result no DNA is deleted, but a single substitution is made.

Alternatively, more conventional mutagenesis methods can be used to introduce at least one mutation into a PHS1 gene or PHS1 promoter sequence. These methods include both physical and chemical mutagenesis. A skilled person will know further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

In one embodiment, insertional mutagenesis is used, for example using T-DNA mutagenesis (which inserts pieces of the T-DNA from the Agrobacterium tumefaciens Ti-Plasmid into DNA causing r loss of gene function mutations); site-directed nucleases (SDNs) or transposons as a mutagen. Insertional mutagenesis is an alternative means of disrupting gene function and is based on the insertion of foreign DNA into the gene of interest (see Krysan et al, The Plant Cell, Vol. 11, 2283-2290, December 1999). Accordingly, in one embodiment, T-DNA is used as an insertional mutagen to disrupt PHS1 gene or PHS1 promoter expression. T-DNA not only disrupts the expression of the gene into which it is inserted, but also acts as a marker for subsequent identification of the mutation. Since the sequence of the inserted element is known, the gene in which the insertion has occurred can be recovered, using various cloning or PCR-based strategies. The insertion of a piece of T-DNA in the order of 5 to 25 kb in length generally produces a disruption of gene function. If a large enough population of T-DNA transformed lines is generated, there are reasonably good chances of finding a transgenic plant carrying a T-DNA insert within any gene of interest. Transformation of spores with T-DNA is achieved by an Agrobacterium-mediated method which involves exposing plant cells and tissues to a suspension of Agrobacterium cells.

The details of this method are well known to a skilled person. In short, plant transformation by Agrobacterium results in the integration into the nuclear genome of a sequence called T-DNA, which is carried on a bacterial plasmid. The use of T-DNA transformation leads to stable single insertions. Further mutant analysis of the resultant transformed lines is straightforward and each individual insertion line can be rapidly characterized by direct sequencing and analysis of DNA flanking the insertion. Gene expression in the mutant is compared to expression of the PHS1 nucleic acid sequence in a wild type plant and phenotypic analysis is also carried out.

In another embodiment, mutagenesis is physical mutagenesis, such as application of ultraviolet radiation, X-rays, gamma rays, fast or thermal neutrons or protons. The targeted population can then be screened to identify an PHS1 mutant with reduced expression or activity.

In another embodiment of the various aspects of the invention, the method comprises mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz (a) anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. Again, the targeted population can then be screened to identify a PHS1 gene or promoter mutant.

In another embodiment, the method used to create and analyse mutations is targeting induced local lesions in genomes (TILLING), reviewed in Henikoff et al, 2004. In this method, seeds are mutagenised with a chemical mutagen, for example EMS. The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the PHS1 target gene or promoter using any method that identifies heteroduplexes between wild type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild type and mutant sequences. Cleavage products are electrophoresed using an automated sequencing gel apparatus, and gel images are analyzed with the aid of a standard commercial image-processing program. Any primer specific to the PHS1 nucleic acid sequence may be utilized to amplify the PHS1 nucleic acid sequence within the pooled DNA sample. Preferably, the primer is designed to amplify the regions of the PHS1 gene where useful mutations are most likely to arise, specifically in the areas of the PHS1 gene that are highly conserved and/or confer activity as explained elsewhere. To facilitate detection of PCR products on a gel, the PCR primer may be labelled using any conventional labelling method. In an alternative embodiment, the method used to create and analyse mutations is EcoTILLING. EcoTILLING is molecular technique that is similar to TILLING, except that its objective is to uncover natural variation in a given population as opposed to induced mutations. The first publication of the EcoTILLING method was described in Comai et al. 2004.

Rapid high-throughput screening procedures thus allow the analysis of amplification products for identifying a mutation conferring the reduction or inactivation of the expression of the PHS1 gene as compared to a corresponding non-mutagenised wild type plant. Once a mutation is identified in a gene of interest, the seeds of the M2 plant carrying that mutation are grown into adult M3 plants and screened for the phenotypic characteristics associated with the target gene PHS1. Loss of and reduced function mutants with an altered starch granule size and/or number compared to a control can thus be identified.

In an alternative embodiment, the expression of the PHS1 gene may be reduced at either the level of transcription or translation. For example, expression of a PHS1 nucleic acid or PHS1 promoter sequence, as defined herein, can be reduced or silenced using a number of gene silencing methods known to the skilled person, such as, but not limited to, the use of small interfering nucleic acids (siNA) against PHS1. “Gene silencing” is a term generally used to refer to suppression of expression of a gene via sequence-specific interactions that are mediated by RNA molecules. The degree of reduction may be so as to totally abolish production of the encoded gene product, but more usually the abolition of expression is partial, with some degree of expression remaining. The term should not therefore be taken to require complete “silencing” of expression.

In one embodiment, the siNA may include, short interfering RNA (siRNA), double-stranded RNA (dsRNA), micro-RNA (miRNA), antagomirs and short hairpin RNA (shRNA) capable of mediating RNA interference. The inhibition of expression and/or activity can be measured by determining the presence and/or amount of PHS1 transcript using techniques well known to the skilled person (such as Northern Blotting, RT-PCR and so on).

Thus, according to the various aspects of the invention a plant may be transformed to introduce a RNAi, shRNA, snRNA, dsRNA, siRNA, miRNA, ta-siRNA, amiRNA or co-suppression molecule that has been designed to target the expression of an PHS1 nucleic acid sequence and selectively decreases or inhibit the expression of the gene or stability of its transcript. Preferably, the RNAi, snRNA, dsRNA, shRNA siRNA, miRNA, amiRNA, ta-siRNA or cosuppression molecule used according to the various aspects of the invention comprises a fragment of at least 17 nt, preferably 22 to 26 nt and can be designed on the basis of the information shown in any of SEQ ID NOs: 1, 2, 5, 6, 9, 10, 13, 14, 17, 18, 21, 22, 25, 26, 29, 30, 33, 34, 37, 38, 41, 42, 45 or 46. Guidelines for designing effective siRNAs are known to the skilled person.

Plants obtained or obtainable by such method which carry a mutation in the endogenous PHS1 gene or promoter locus are also within the scope of the invention, where preferably the mutation is a loss or partial loss of function mutation as described above.

Accordingly, the genetically altered plant or plant cell may alternatively comprise a nucleic acid construct expressing an RNAi molecule targeting the PHS1 gene as described herein. In one embodiment, said construct is stably incorporated into the plant genome. These techniques also include gene targeting using vectors that target the PHS1 gene and which allows for integration of a transgene at a specific site. The targeting construct is engineered to recombine with the target gene (PHS1), which is accomplished by incorporating sequences from the gene itself into the construct.

Recombination then occurs in the region of that sequence within the gene, resulting in the insertion of a foreign sequence to disrupt the gene. With its sequence interrupted, the altered gene will be translated into a nonfunctional protein, if it is translated at all.

In a further aspect of the invention, there is provided a method of altering a physiochemical property of starch, the method comprising reducing the expression of at least one PHS1 nucleic acid and/or reducing the activity of a PHS1 polypeptide as described above. The physiochemical property may be selected from gelatinisation temperature, swelling power and viscosity. An increase as used herein may be at least 1%, 2%, 3%, 4%, 5%, 6%, 7%, 8%, 9%, 10% 11%, 12%, 13%, 14%, 15%, 16%, 17%, 18%, 19%, 20%, 30%, 40% or 50% in comparison to a control plant.

In another aspect of the invention, there is provided a method of shifting a plant with a bimodal size distribution of starch granules towards a unimodal distribution of starch granules, the method comprising reducing or abolishing the expression of at least one PHS1 nucleic acid and/or reducing or abolishing the activity of a PHS1 polypeptide as described above.

In a further aspect of the invention, there is provided a method of altering the initiation of B-type granule formation in a starch storage organ of a plant, the method comprising reducing or abolishing the expression of at least one PHS1 nucleic acid and/or reducing or abolishing the activity of at least one PHS1 polypeptide as described above. Preferably, said method does not affect initiation of A-type granule formation.

As described above, preferably the plant has a bimodal size distribution of starch granules, such as wheat, rye and barley, wherein preferably said bimodal distribution comprises a first population of starch granules of a first size or size range and a second population of starch granules of a second size or size range. In one embodiment the plant is selected from the Triticeae tribe.

In one embodiment, the method has no (obvious detectable) effect on total starch content and/or plant growth. By “plant growth” here is meant for example, but not limited to, grain yield, thousand grain weight and grain size.

In an embodiment, the method has no (detectable) effect on the soluble sugar content of grains. Examples of soluble sugars include but are not limited to maltooligosacharides, glucose, fructose and sucrose.

In an embodiment, the method has no (detectable) effect on A-type granule initiation.

In an embodiment, the method disrupts or stops the interaction between PHS1 and BGC1, altering the initiation of B-type granule formation, resulting in fewer and/or larger B-type granules. Preferably, A-type granule initiation is not (detectably) affected.

In another aspect of the invention there is provided a method for producing a genetically altered plant as described herein. In one embodiment, the method comprises introducing at least one mutation into the PHS1 gene and/or PHS1 promoter of preferably at least one plant cell using any mutagenesis technique described herein. Preferably said method further comprising regenerating a plant from the mutated plant cell.

The method may further comprise selecting one or more mutated plants, preferably for further propagation. Preferably said selected plants comprise at least one mutation in the PHS1 gene and/or promoter sequence. Preferably said plants are characterised by reduced or abolished PHS1 expression and/or a reduced or abolished levels of PHS1 polypeptide activity. Expression and/or activity levels (e.g. phosphorylase activity) of PHS1 can be measured by any standard technique known to the skilled person, including those described herein. A reduction is as described herein.

The selected plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further aspect of the invention there is provided a plant obtained or obtainable by the above described methods.

For the purposes of the invention, a “genetically altered plant” or “mutant plant” is a plant that has been genetically altered compared to the naturally occurring wild type (WT) plant. In one embodiment, a mutant plant is a plant that has been altered compared to the naturally occurring wild type (WT) plant using a mutagenesis method, such as any of the mutagenesis methods described herein. In one embodiment, the mutagenesis method is targeted genome modification or genome editing. In one embodiment, the plant genome has been altered compared to wild type sequences using a mutagenesis method. Such plants have an altered phenotype as described herein, such as an altered starch granule characteristic. Therefore, in this example, an altered starch granule characteristic is conferred by the presence of an altered plant genome, for example, a mutated endogenous PHS1 gene or PHS1 promoter sequence. In one embodiment, the endogenous promoter or gene sequence is specifically targeted using targeted genome modification and the presence of a mutated gene or promoter sequence is not conferred by the presence of transgenes expressed in the plant. In other words, the genetically altered plant can be described as transgene-free.

A plant according to the various aspects of the invention, including the transgenic plants, methods and uses described herein may be a monocot or a dicot plant. Preferably, the plant is a crop plant or a biofuel plant. By crop plant is meant any plant which is grown on a commercial scale for human or animal consumption or use. In a preferred embodiment, the plant is a cereal. In one embodiment the plant is not Arabidopsis. In another embodiment, the plant is not rice.

In a most preferred embodiment, the plant is a plant that has a bimodal distribution of starch granules. For example, in one embodiment the plant is selected from the Triticeae tribe, including but not limited to wheat, barley and rye. In a further embodiment, the plant is wheat. In a further embodiment, the plant is barley.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein. The term “plant” also encompasses plant cells, suspension cultures, callus tissue, embryos, meristematic regions, gametophytes, sporophytes, pollen and microspores, again wherein each of the aforementioned comprises the nucleic acid construct as described herein.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. Another product that may derived from the harvestable parts of the plant of the invention is biodiesel. The invention also relates to food products and food supplements comprising the plant of the invention or parts thereof. In one embodiment, the food products may be animal feed. In another aspect of the invention, there is provided a product derived from a plant as described herein or from a part thereof.

The terms “seed” and “grain” as used herein can be used interchangeably. The terms “increase”, “improve” or “enhance” as used herein are also interchangeable. Similarly, the terms starch “grain” or “granule” are also interchangeable.

In a most preferred embodiment, the plant part or harvestable product is a seed or grain. Therefore, in a further aspect of the invention, there is provided a seed or grain produced from a genetically altered plant as described herein.

In another embodiment of the invention, the plant part is a starch storage organ comprising starch granules which have a mean granule size which is increased by 3% or more, 5% or more, 10% or more, 15% or more, 20% or more, 25% or more, 30% or more, 35% or more, 40% or more or 45% or more compared to the mean granule size in a control or wild-type plant.

In an alternative embodiment, the plant part is pollen, a propagule or progeny of the genetically altered plant described herein. Accordingly, in a further aspect of the invention there is provided pollen, a propagule or progeny of the genetically altered plant as described herein.

In another aspect of the invention, there is provided starch or a starch component obtained or obtainable from at least one plant cell of the genetically altered plant described herein or the grain of a genetically altered plant described herein.

There is also provided a food or feed composition prepared from the grain or starch or starch composition described herein.

Finally, there is also provided the use of the grain or starch described herein as a food or feedstuff, in biofuel (bioethanol) production or in any pharmaceutical, cosmetic or industrial application. Examples of industrial applications include the brewing, papermaking and plastic industries. Examples of food include bread, biscuits, baked goods based on wheat flour and pasta.

A control plant as used herein according to all of the aspects of the invention is a plant which has not been modified according to the methods of the invention. Accordingly, in one embodiment, the control plant does not have altered expression of a PHS1 nucleic acid and/or altered activity of a PHS1 polypeptide, as described herein. In an alternative embodiment, the plant been genetically modified, as described above. In one embodiment, the control plant is a wild type plant. The control plant is typically of the same plant species, preferably having the same genetic background as the modified plant.

Genome Editing Constructs for Use with the Methods for Targeted Genome Modification Described Herein

By “crRNA” or CRISPR RNA is meant the sequence of RNA that contains the protospacer element and additional nucleotides that are complementary to the tracrRNA.

By “tracrRNA” (transactivating RNA) is meant the sequence of RNA that hybridises to the crRNA and binds a CRISPR enzyme, such as Cas9 thereby activating the nuclease complex to introduce double-stranded breaks at specific sites within the genomic sequence of at least one PHS1 nucleic acid or promoter sequence.

By “protospacer element” is meant the portion of crRNA (or sgRNA) that is complementary to the genomic DNA target sequence, usually around 20 nucleotides in length. This may also be known as a spacer or targeting sequence.

By “sgRNA” (single-guide RNA) is meant the combination of tracrRNA and crRNA in a single RNA molecule, preferably also including a linker loop (that links the tracrRNA and crRNA into a single molecule). “sgRNA” may also be referred to as “gRNA” and in the present context, the terms are interchangeable. The sgRNA or gRNA provide both targeting specificity and scaffolding/binding ability for a Cas nuclease. A gRNA may refer to a dual RNA molecule comprising a crRNA molecule and a tracrRNA molecule.

By “TAL effector” (transcription activator-like (TAL) effector) or TALE is meant a protein sequence that can bind the genomic DNA target sequence (a sequence within the PHS1 gene or promoter sequence) and that can be fused to the cleavage domain of an endonuclease such as FokI to create TAL effector nucleases or TALENS or meganucleases to create megaTALs. A TALE protein is composed of a central domain that is responsible for DNA binding, a nuclear-localisation signal and a domain that activates target gene transcription. The DNA-binding domain consists of monomers and each monomer can bind one nucleotide in the target nucleotide sequence. Monomers are tandem repeats of 33-35 amino acids, of which the two amino acids located at positions 12 and 13 are highly variable (repeat variable diresidue, RVD). It is the RVDs that are responsible for the recognition of a single specific nucleotide. HD targets cytosine; NI targets adenine, NG targets thymine and NN targets guanine (although NN can also bind to adenine with lower specificity).

In another aspect of the invention there is provided a nucleic acid construct wherein the nucleic acid construct comprises a nucleic acid sequence that encodes at least one DNA-binding domain. In one embodiment, the DNA-binding domain can bind to a sequence in the PHS1 gene and/or promoter. Preferably said sequence is selected from one of SEQ ID NO: 49 to 52, 84 and 85 and are target sequences in a PHS1 gene. In one embodiment, the nucleic acid construct comprises one or more DNA-binding domains, such that the construct can bind to one or more, preferably at least two or three sequences in the PHS1 gene.

In a further embodiment, said construct further comprises a nucleic acid encoding at least one sequence specific nuclease (SSN) such as FokI or a Cas protein.

In one embodiment, the nucleic acid construct encodes at least one protospacer element wherein the sequence of the protospacer element is selected from SEQ ID NO: 53 to 56, 86 and 87 or a variant thereof. In one example, the nucleic acid construct may comprise one, two or three protospacer sequences, wherein the sequence of the protospacer sequences is selected from SEQ ID NO:53, 54, 55, 56, 86 and 87.

In a further embodiment, the nucleic acid construct comprises a crRNA-encoding sequence. As defined above, a crRNA sequence may comprise the protospacer elements as defined above and preferably additional nucleotides that are complementary to the tracrRNA. An appropriate sequence for the additional nucleotides will be known to the skilled person as these are defined by the choice of Cas protein.

In another embodiment, the nucleic acid construct further comprises a tracrRNA sequence. Again, an appropriate tracrRNA sequence would be known to the skilled person as this sequence is defined by the choice of Cas protein. Nonetheless, in one embodiment said sequence comprises or consists of a sequence as defined in SEQ ID NO: 57 or a variant thereof.

In a further embodiment, the nucleic acid construct comprises at least one nucleic acid sequence that encodes a sgRNA (or gRNA). Again, as already discussed, sgRNA typically comprises a crRNA sequence or protospacer sequence and a tracrRNA sequence and preferably a sequence for a linker loop. In a preferred embodiment, the nucleic acid construct comprises at least one nucleic acid sequence that encodes a sgRNA sequence as defined in any of SEQ ID NO: 62 to 65 or variant thereof. More preferably the nucleic acid sequence that encodes a sgRNA comprises or consists of a sequence selected from SEQ ID NO: 58 to 61, 88 and 89 or a variant thereof.

In a further embodiment, the nucleic acid construct may further comprise at least one nucleic acid sequence encoding an endoribonuclease cleavage site. Preferably the endoribonuclease is Csy4 (also known as Cas6f). Where the nucleic acid construct comprises multiple sgRNA nucleic acid sequences the construct may comprise the same number of endoribonuclease cleavage sites. In another embodiment, the cleavage site is 5′ of the sgRNA nucleic acid sequence. Accordingly, each sgRNA nucleic acid sequence is flanked by a endoribonuclease cleavage site.

The term ‘variant’ refers to a nucleotide sequence where the nucleotides are substantially identical to one of the above sequences. The variant may be achieved by modifications such as insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to any one of the above described sequences. In one embodiment, sequence identity is at least 90%. In another embodiment, sequence identity is 100%. Sequence identity can be determined by any one known sequence alignment program in the art.

The invention also relates to a nucleic acid construct comprising a nucleic acid sequence operably linked to a suitable plant promoter. A suitable plant promoter may be a constitutive or strong promoter or may be a tissues-specific promoter. In one embodiment, suitable plant promoters are selected from, but not limited to, cestrum yellow leaf curling virus (CmYLCV) promoter or switchgrass ubiquitin 1 promoter (PvUbi1) wheat U6 RNA polymerase III (TaU6) CaMV35S, wheat U6 or maize ubiquitin (e.g. Ubi1) promoters. Alternatively, expression can be specifically directed to particular tissues of wheat seeds through gene expression-regulating sequences. In one embodiment, the promoter is selected from the U6 promoter (for example as defined in SEQ ID NO:68) and the ubiquitin 1 promoter (for example as defined in SEQ ID NO:69). In a preferred embodiment the sequences are codon-optimised for the plant in question.

The nucleic acid construct of the present invention may also further comprise a nucleic acid sequence that encodes a CRISPR enzyme. By “CRISPR enzyme” is meant an RNA-guided DNA endonuclease that can associate with the CRISPR system. Specifically, such an enzyme binds to the tracrRNA sequence. In one embodiment, the CRIPSR enzyme is a Cas protein (“CRISPR associated protein), preferably Cas 9 or Cpf1, more preferably Cas9. In a specific embodiment Cas9 is codon-optimised Cas9 (optimised for the plant in which it is expressed). In one example, Cas9 has the sequence described in SEQ ID NO: 66 or a functional variant or homolog thereof. In another embodiment, the CRISPR enzyme is a protein from the family of Class 2 candidate×proteins, such as C2c1, C2C2 and/or C2c3. In one embodiment, the Cas protein is from Streptococcus pyogenes. In an alternative embodiment, the Cas protein may be from any one of Staphylococcus aureus, Neisseria meningitides, Streptococcus thermophiles or Treponema denticola.

The term “functional variant” as used herein with reference to Cas9 refers to a variant Cas9 gene sequence or part of the gene sequence which retains the biological function of the full non-variant sequence, for example, acts as a DNA endonuclease, or recognition and/or binding to DNA. A functional variant also comprises a variant of the gene of interest which has sequence alterations that do not affect function, for example non-conserved residues. Also encompassed is a variant that is substantially identical, i.e. has only some sequence variations, for example in non-conserved residues, compared to the wild type sequences as shown herein and is biologically active. In one embodiment, a functional variant of SEQ ID NO: 66 has at least 90%, 91%, 92%, 93%, 94%, 95%, 96%, 97%, 98%, or 99% overall sequence identity to the nucleic acid represented by SEQ ID NO:66. In a further embodiment, the Cas9 protein has been modified to improve activity.

Suitable homologs or orthologs can be identified by sequence comparisons and identifications of conserved domains. The function of the homolog or ortholog can be identified as described herein and a skilled person would thus be able to confirm the function when expressed in a plant.

In a further embodiment, the Cas9 protein has been modified to improve activity. For example, in one embodiment, the Cas9 protein may comprise the D10A amino acid substitution, this nickase cleaves only the DNA strand that is complementary to and recognized by the gRNA. In an alternative embodiment, the Cas9 protein may alternatively or additionally comprise the H840A amino acid substitution, this nickase cleaves only the DNA strand that does not interact with the sRNA. In this embodiment, Cas9 may be used with a pair (i.e. two) sgRNA molecules (or a construct expressing such a pair) and as a result can cleave the target region on the opposite DNA strand, with the possibility of improving specificity by 100-1500 fold. In a further embodiment, the Cas9 protein may comprise a D1135E substitution. The Cas 9 protein may also be the VQR variant. Alternatively, the Cas protein may be comprise a mutation in both nuclease domains, HNH and RuvC0 like and therefore is catalytically inactive. Rather than cleaving the target strand, this catalytically inactive Cas protein can be used to prevent the transcription elongation process, leading to a loss of function of incompletely translated proteins when co-expressed with a sgRNA molecule. An example of a catalytically inactive protein is dead Cas9 (dCas9) caused by a point mutation in RuvC and/or the HNH nuclease domains (Komor et al., 2016 and Nishida et al., 2016).

In a further embodiment, a Cas protein, such as Cas9 may be further fused with a repression effector, such as a histone-modifying/DNA methylation enzyme or a Cytidine deaminase (Komor et al. 2016) to effect site-directed mutagenesis. In the latter, the cytidine deaminase enzyme does not induce dsDNA breaks, but mediates the conversion of cytidine to uridine, thereby effecting a C to T (or G to A) substitution.

In a further embodiment, the nucleic acid construct comprises an endoribonuclease. Preferably the endoribonuclease is Csy4 (also known as Cas6f) and more preferably a codon optimised csy4, for example as defined in SEQ ID NO: 67. In one embodiment, where the nucleic acid construct comprises a cas protein, the nucleic acid construct may comprise sequences for the expression of an endoribonuclease, such as Csy4 expressed as a 5′ terminal P2A fusion (used as a self-cleaving peptide) to a cas protein, such as Cas9.

In one embodiment, the cas protein, the endoribonuclease and/or the endoribonuclease-cas fusion sequence may be operably linked to a suitable plant promoter. Suitable plant promoters are already described above, but in one embodiment, may be the Zea Mays Ubiquitin 1 promoter or U6 promoter.

Suitable methods for producing the CRISPR nucleic acids and vectors system are known, and for example are published in Molecular Plant (Ma et al., 2015, Molecular Plant, DOI: 10.1016/j.molp.2015.04.007), which is incorporated herein by reference.

In an alternative aspect of the invention, the nucleic acid construct comprises at least one nucleic acid sequence that encodes a TAL effector, wherein said effector targets a PHS1 gene and/or promoter sequence, preferably selected from SEQ ID NO 49, 50, 51, 52, 84 and 85. Methods for designing a TAL effector would be well known to the skilled person, given the target sequence. Examples of suitable methods are given in Sanjana et al., and Cermak T et al, both incorporated herein by reference. Preferably, said nucleic acid construct comprises two nucleic acid sequences encoding a TAL effector, to produce a TALEN pair. In a further embodiment, the nucleic acid construct further comprises a sequence-specific nuclease (SSN). Preferably such SSN is a endonuclease such as FokI. In a further embodiment, the TALENs are assembled by the Golden Gate cloning method in a single plasmid or nucleic acid construct.

In another aspect of the invention, there is provided a sgRNA molecule, wherein the sgRNA molecule comprises a crRNA sequence and a tracrRNA sequence and wherein the crRNA sequence can bind to at least one sequence selected from SEQ ID NOs 49 to 52, 84 and 85 or a variant thereof. In one embodiment, the nucleic sequence of the sgRNA molecule is defined in any of SEQ ID NO: 58 to 61, 88 or 89 or variant thereof. In other words, the RNA sequence of the sgRNA is encoded by a nucleic acid sequence selected from SEQ ID NO: 58 to 61, 88 or 89. In one example only, the RNA sequence of one sgRNA of the invention is defined in SEQ ID NO: 62 to 65 or a variant thereof. A “variant” is as defined herein. In one embodiment, the sgRNA molecule may comprise at least one chemical modification, for example that enhances its stability and/or binding affinity to the target sequence or the crRNA sequence to the tracrRNA sequence. Such modifications would be well known to the skilled person, and include for example, but not limited to, the modifications described in Rahdar et al., 2015, incorporated herein by reference. In this example the crRNA may comprise a phosphorothioate backbone modification, such as 2′-fluoro (2′-F), 2′-O-methyl (2′-O-Me) and S-constrained ethyl (CET) substitutions.

In another aspect of the invention, there is provided an isolated nucleic acid sequence that encodes for a protospacer element (as defined in any of SEQ ID NOs: 43 to 56, 86 or 87), or a sgRNA (as described in any of SEQ ID NO: 58 to 61, 88 or 89). There is also provided an isolated sgRNA molecule as defined in any of SEQ ID NO: 62 to 65.

In another aspect of the invention, there is provided a plant or part thereof or at least one isolated plant cell transfected with at least one nucleic acid construct as described herein. Cas9 and sgRNA may be combined or in separate expression vectors (or nucleic acid constructs, such terms are used interchangeably). In other words, in one embodiment, an isolated plant cell is transfected with a single nucleic acid construct comprising both sgRNA and Cas9 as described in detail above. In an alternative embodiment, an isolated plant cell is transfected with two nucleic acid constructs, a first nucleic acid construct comprising at least one sgRNA as defined above and a second nucleic acid construct comprising Cas9 or a functional variant or homolog thereof. The second nucleic acid construct may be transfected below, after or concurrently with the first nucleic acid construct. The advantage of a separate, second construct comprising a cas protein is that the nucleic acid construct encoding at least one sgRNA can be paired with any type of cas protein, as described herein, and therefore are not limited to a single cas function (as would be the case when both cas and sgRNA are encoded on the same nucleic acid construct).

In one embodiment, the nucleic acid construct comprising a cas protein is transfected first and is stably incorporated into the genome, before the second transfection with a nucleic acid construct comprising at least one sgRNA nucleic acid. In an alternative embodiment, a plant or part thereof or at least one isolated plant cell is transfected with mRNA encoding a cas protein and co-transfected with at least one nucleic acid construct as defined herein. Alternatively, as described in Example 2, the nucleic acid constructs can be transiently expressed in the target plant cell.

Cas9 expression vectors for use in the present invention can be constructed as described in the art. Examples of suitable promoters include the Actin, CaMV35S, wheat U6 or maize ubiquitin (e.g. Ubi1) promoter, as described above.

In an alternative aspect of the present invention, there is provided an isolated plant cell (transiently or stably) transfected with at least one nucleic acid construct or sgRNA molecule as described herein.

In a further aspect of the invention, there is provided a genetically modified or edited plant comprising the transfected cell described herein. In one embodiment, the nucleic acid construct or constructs may be integrated in a stable form. In an alternative embodiment, the nucleic acid construct or constructs are not integrated (i.e. are transiently expressed). Accordingly, in a preferred embodiment, the genetically modified plant is free of any sgRNA and/or Cas protein nucleic acid. In other words, the plant is transgene free.

The term “introduction”, “transfection” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art.

The transfer of foreign genes into the genome of a plant is called transformation. Transformation of plants is now a routine technique in many species. Any of several transformation methods known to the skilled person may be used to introduce the nucleic acid construct or sgRNA molecule of interest into a suitable ancestor cell. The methods described for the transformation and regeneration of plants from plant tissues or plant cells may be utilized for transient or for stable transformation.

Transformation methods include the use of liposomes, electroporation, chemicals that increase free DNA uptake, injection of the DNA directly into the plant (microinjection), gene guns (or biolistic particle delivery systems (biolistics)) as described in the examples, lipofection, transformation using viruses or pollen and microprojection. Methods may be selected from the calcium/polyethylene glycol method for protoplasts, ultrasound-mediated gene transfection, optical or laser transfection, transfection using silicon carbide fibers, electroporation of protoplasts, microinjection into plant material, DNA or RNA-coated particle bombardment, infection with (non-integrative) viruses and the like. Transgenic plants, can also be produced via Agrobacterium tumefaciens mediated transformation, including but not limited to using the floral dip/Agrobacterium vacuum infiltration method as described in Clough & Bent (1998) and incorporated herein by reference.

Accordingly, in one embodiment, at least one nucleic acid construct or sgRNA molecule as described herein can be introduced to at least one plant cell using any of the above described methods. In an alternative embodiment, any of the nucleic acid constructs described herein may be first transcribed to form a preassembled Cas9-sgRNA ribonucleoprotein and then delivered to at least one plant cell using any of the above described methods, such as lipofection, electroporation or microinjection.

Optionally, to select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. As described in the examples, a suitable marker can be bar-phosphinothricin or PPT. Alternatively, the transformed plants are screened for the presence of a selectable marker, such as, but not limited to, GFP, GUS (β-glucuronidase). Other examples would be readily known to the skilled person. Alternatively, no selection is performed, and the seeds obtained in the above-described manner are planted and grown and PHS1 expression or protein levels measured at an appropriate time using standard techniques in the art. This alternative, which avoids the introduction of transgenes, is preferable to produce transgene-free plants.

Following DNA transfer and regeneration, putatively transformed plants may also be evaluated, for instance using PCR to detect the presence of the gene of interest, copy number and/or genomic organisation. Alternatively or additionally, integration and expression levels of the newly introduced DNA may be monitored using Southern, Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

In a further related aspect of the invention, there is also provided, a method of obtaining a genetically modified plant as described herein, the method comprising
a. selecting a part of the plant;
b. transfecting at least one cell of the part of the plant of paragraph (a) with at least one nucleic acid construct as described herein or at least one sgRNA molecule as described herein, using the transfection or transformation techniques described above;
c. regenerating at least one plant derived from the transfected cell or cells;
d. selecting one or more plants obtained according to paragraph (c) that show altered expression or activity of PHS1.

In a further embodiment, the method also comprises the step of screening the genetically modified plant for SSN (preferably CRISPR)-induced mutations in the PHS1 gene or promoter sequence. In one embodiment, the method comprises obtaining a DNA sample from a transformed plant and carrying out DNA amplification to detect a mutation in at least one PHS1 gene or promoter sequence.

In a further embodiment, the methods comprise generating stable T2 plants preferably homozygous for the mutation (that is a mutation in in at least one PHS1 gene or promoter sequence).

Plants that have a mutation in at least one PHS1 gene or promoter sequence can also be crossed with another plant also containing at least one mutation in at least one PHS1 gene or promoter sequence to obtain plants with additional mutations in the PHS1 gene or promoter sequence. The combinations will be apparent to the skilled person. Accordingly, this method can be used to generate a T2 plants with mutations on all or an increased number of homoelogs, when compared to the number of homoeolog mutations in a single T1 plant transformed as described above.

A plant obtained or obtainable by the methods described above is also within the scope of the invention.

A genetically altered plant of the present invention may also be obtained by transference of any of the sequences of the invention by crossing, e.g., using pollen of the genetically altered plant described herein to pollinate a wild-type or control plant, or pollinating the gynoecia of plants described herein with other pollen that does not contain a mutation in at least one of the PHS1 gene or promoter sequence. The methods for obtaining the plant of the invention are not exclusively limited to those described in this paragraph; for example, genetic transformation of germ cells from the ear of wheat could be carried out as mentioned, but without having to regenerate a plant afterward.

In a further final aspect of the invention, there is provided a method of screening a population of plants and identifying and/or selecting a plant that will have altered expression and/or activity of PHS1 and therefore an alteration in granule size distribution in a plant, as described herein, compared to a control or wild-type plant, the method comprising detecting at least one polymorphism or mutation in the PHS1 gene and/or promoter, wherein said mutation or polymorphism leads to an alteration in the level of expression and/or activity of the PHS1 protein compared to the level in a plant not carrying said mutation or polymorphism (e.g. a control or wild-type plant). Said mutation or polymorphism may comprise at least one insertion and/or at least one deletion and/or substitution.

Suitable tests for assessing the presence of a polymorphism would be well known to the skilled person, and include but are not limited to, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs). In one embodiment, Kompetitive Allele Specific PCR (KASP) genotyping is used.

The method may also comprise the step of assessing whether the polymorphism has an effect on a starch granule characteristic as described herein. Methods to screen for an effect on starch granule characteristic would be well known to the skilled person, but could be carried out using a coulter counter, a microscope, a flow cytometer or any other particle sizing instrument, again as described above.

In one embodiment, the method comprises
a) obtaining a nucleic acid sample from a plant and
b) carrying out nucleic acid amplification of one or more PHS1 gene and/or promoter alleles using one or more primer pairs.

In a further embodiment, the method may further comprise introgressing the chromosomal region comprising at least one of said low-PHS1-expressing/activity polymorphisms into a second plant or plant germplasm to produce an introgressed plant or plant germplasm. Preferably the expression or activity of PHS1 in said second plant will be altered (compared to a control or wild-type plant), and more preferably said second plant will display an alteration in at least one of starch B-type granule number and/or size, as described above.

In a further aspect of the invention there is provided a method of altering a starch granule characteristic, as described above, in a plant, the method comprising
a. screening a population of plants for at least one plant with at least one of the above described polymorphisms or mutations; and
b. further altering (i.e. reducing/abolishing or increasing) the expression of at least one PHS1 nucleic acid and/or altering (i.e. reducing/abolishing or increasing) the activity of a PHS1 polypeptide in said plant by introducing at least one mutation into the nucleic acid sequence encoding PHS1 or at least one mutation into the promoter of PHS1 as described herein or using RNA interference as described herein.

By “further altering” is meant reducing or increasing the level of PHS1 expression to a level lower than that in the plant with the at least one of the above-described PHS1 polymorphisms. The terms “reducing” or “increasing” means a decrease in the levels of PHS1 expression and/or activity by up to 10%, 20%, 30%, 40%, 50%, 60%, 70%, 80% or 90% when compared to the level in a control plant or the level in the initially screened plants.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting examples.

### Example 1: The Phs1 Mutant of Wheat has No Obvious Effects on Vegetative Growth

To study the role of PHS1 in wheat, we first identified the gene models corresponding to PHS1. BLAST searches against the wheat RefSeq 1.1 genome of cultivar Chinese Spring (Appels et al., 2018) on Ensembl plants (Kersey et al., 2018) revealed three homeologs of PHS1 encoded on group 5 chromosomes. These were PHS1-5A (TraesCS5A02G395200.1), PHS-5B (TraesCS5B02G400000.2) and PHS-5D (TraesCS5D02G404500.1) (FIG. 1A). In the durum wheat reference genome, these corresponded to PHS1-5A (TRITD5Av1G205670) and PHS1-5B (TRITD5Bv1G201740). All homeologs had 15 exons and 14 introns.

We examined gene expression of the different PHS1 homeologs in Chinese Spring using the wheat expression browser (Borrill et al., 2016). All homeologs showed similar levels of expression across all examined tissues. PHS1 transcripts were detectable in leaves/shoots, roots, spike and grains. However, consistent with previous studies (Schupp and Ziegler, 2004), the highest level of expression in the endosperm was during early grain development (10 dpa), although h robust levels of transcript were detectable at later timepoints (20-30 dpa).

We generated two mutant wheat lines: phs1-1 and phs1-6 with mutations in both the A and B homeologs. The AA BB (wild-type sibling control), aa BB and AA bb (single mutants), and aa bb (double mutant) genotypes were selected in the F2 generation using KASP genotyping.

To verify the effect of the mutations on PHS1 activity, we separated leaf protein extracts on native PAGE gels and stained for phosphorylase activity. Wheat protein extracts are reported to produce three distinct bands using this technique, of which the plastidial phosphorylase has the highest mobility (Schupp and Ziegler, 2004). We observed four distinct activity bands, with two closely migrating bands in the region expected for plastidial phosphorylase (FIG. 1B). Both bands were absent in the phs1-1 and phs1-6 aa bb double mutants, and the two aa BB and AA bb single mutants of phs1-1 were missing either one of the two bands. We concluded that the two bands represent PHS1-5A and PHS1-5B activities, and the selected mutations for phs1-1 and phs1-6 mutant lines eliminate all detectable PHS1 activity.

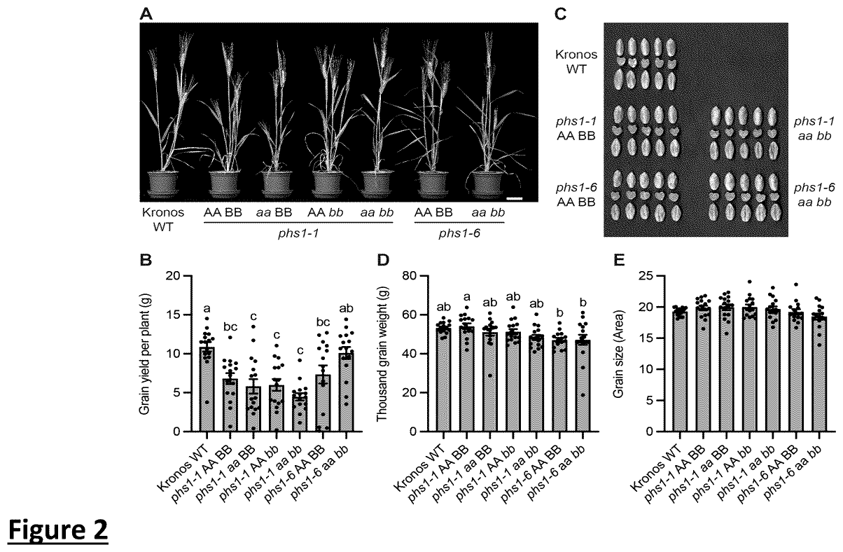

Overall, we observed no detectable effect of the phs1 mutants on plant growth in glasshouses (FIG. 2A). However, in some experiments, plants from all phs1-1 lines (including the mutants and the wild-type siblings) were slightly shorter than wild-type plants. This is likely due to background mutations rather than the loss of PHS1 activity, since it was also observed in the wild-type sibling from the phs1-1 cross, and was not observed in any of the phs1-6 lines. Similarly, there was no effect of phs1 mutations on the grain yield per plant, but a decrease was seen in all phs1-1 lines (FIG. 2B) (on this point, it is important to note that the TILLING mutants are full of other mutations and therefore here, the comparison is not with wild-type but with the plants not carrying a mutation in PHS1 (e.g. AA BB)). Again, there was no significant difference in grain yield per plant or seed number between the phs1-1 double mutant and its wild-type sibling control, or between the phs1-6 double mutant and its wild-type sibling control. All lines produced seeds that appeared normal (FIG. 2C), and we did not observe any significant effects of phs1 mutations on grain size or thousand grain weight (FIG. 2D, E). We therefore conclude that loss of PHS1 activity does not affect vegetative growth or seed yield.

### Example 2: Granule Initiation in Leaves

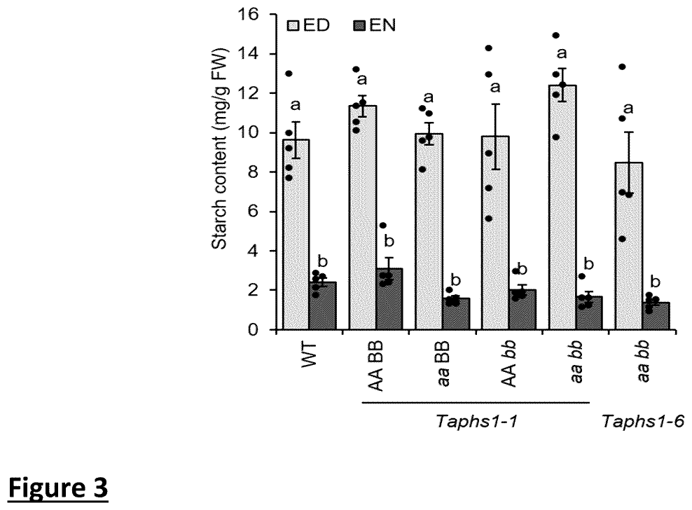

To test whether the loss of PHS1 affected leaf starch metabolism, we first quantified starch content in leaves of seedlings. We did not see a significant difference in starch content between the wild type and mutants, at either the end of day or end of night, and all lines turned over starch during the night (FIG. 3).

### Example 3: PHS1 is Required for Normal B-Type Granule Size and Number

We investigated the effect of phs1 mutations on starch synthesis in the endosperm. First, we measured starch content. The phs1-1 double mutant had identical starch content to the wild-type sibling control and the wild type (Table 1). Interestingly, the double mutant had a small but significant reduction in amylose content.

We then purified starch granules from mature grains and examined their morphology using Scanning Electron Microscopy (SEM). Interestingly, starch from the phs1 double mutants had visibly fewer B-type granules than the wild type (FIG. 4A). No differences were observed between the single mutants and the control lines. Quantification of granule size distribution on the Coulter counter confirmed that the double mutant had fewer, larger B-type granules. When the relative volume of starch was plotted against granule diameter, the B-type granule peak was much less prominent in the double mutant than in the wild type, and appeared to be shifted towards the larger size range (FIG. 4B). By fitting a mixed normal distribution to the diameter-volume plots, we calculated that the double mutant had less than half of the B-type granule content (by volume) compared to the wild-type and single mutants, despite having significantly larger B-type granules (FIG. 4C, D). As the large size of the B-type granules in the double mutant affects the area of the B-type granule peak when plotted on a volume basis, we also plotted the size distributions on a relative number basis (FIG. 4E), which showed a large shift in the proportion of A-type vs. B-type granules in the double mutant. As curves cannot be reliably fitted to the number-diameter plots due to the small size of the A-type granule peak, we set a numerical size cutoff for small granules (<10 μm), which includes B-type granules and some smaller A-type granules. This showed a significant 26% decrease in the relative number of small granules in the double mutant compared to the wild type (FIG. 4F). By contrast, the size of A-type granules was unaffected by the Taphs1 mutations (FIG. 4G).

Taken together, these data suggest that loss of PHS1 results in the synthesis of fewer, but larger, B-type granules, suggesting it has a role in the initiation of B-type granules. Thus, mutations in PHS1 can be exploited to reduce the heterogeneity in starch granule size in wheat and other related Triticeae crops, creating higher quality grains for brewing and the purification of starch and gluten. Starch with fewer, larger B-type granules may also have novel physical and chemical properties that can be exploited in manufacturing both food and non-food products.

TABLE 1
Starch content and composition of phs1-1 mutants

| | Starch content (g/g flour) | Amylose content (%) |
|---|---|---|
| Kronos WT | 47 ± 2 ^{ab} | 28.6 ± 2.4 ^{a} |
| Taphs1-1 AA BB | 44 ± 2 ^{ab} | 28.8 ± 0.8 ^{a} |
| Taphs1-1 aa BB | 39 ± 3 ^{b} | 26.7 ± 0.6 ^{a} |
| Taphs1-1 AA bb | 40 ± 1 ^{b} | 26.6 ± 1.3 ^{a} |
| Taphs1-1 aa bb | 49 ± 2 ^{a} | 21.1 ± 0.6 ^{b} |

Starch content was quantified relative to the dry weight of whole flour. Values are the mean ± SEM on grains from n = 4 different plants. Amylose content was measured on purified starch. Values are the mean ± SEM on starch extracted from n = 4-6 different plants.

### Example 4: BGC1 Interacts with Proteins Involved in Starch Synthesis and Other Cellular Processes

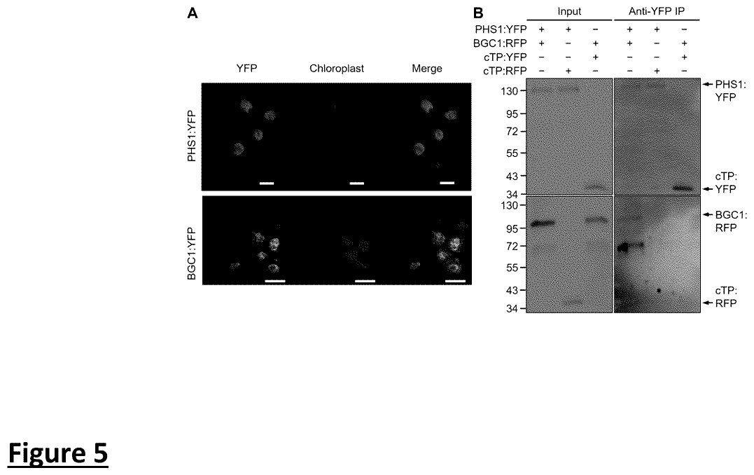

We confirmed that BGC1 interacts with PHS1, in a series of heterologous assays carried out using transiently expressed proteins in Nicotiana benthamiana leaves. We confirmed that both PHS1 and BGC1 located to the chloroplast in this heterologous system (FIG. 5A). PHS1 was located in the stroma, whereas BGC1 was located around starch granules, and in punctate structures. We then used pairwise immunoprecipitations with YFP/RFP-tagged proteins in Nicotiana (FIG. 5B). BGC1: RFP co-purified with PHS1: YFP in the IP with anti-YFP beads when the two fusion proteins were co-expressed (FIG. 5B). BGC1: RFP did not purify in control reactions when co-expressed with a chloroplast-targeted YFP, nor did a chloroplast-targeted RFP co-purify with PHS1: YFP.

### Example 5: PHS1 Expression

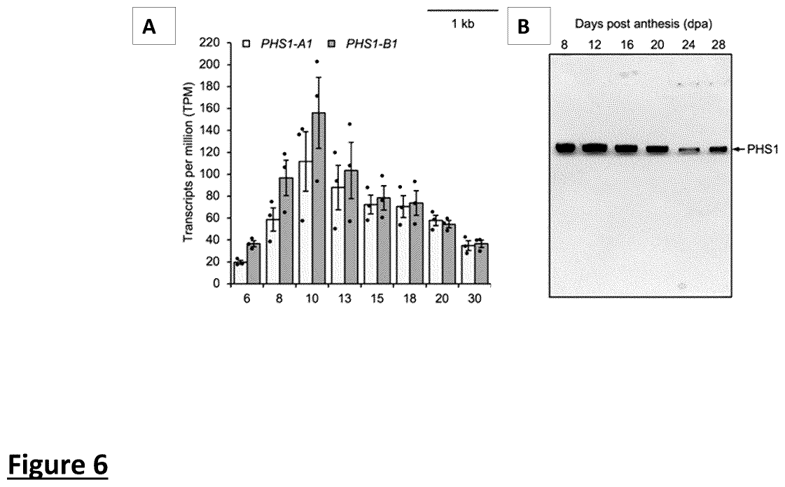

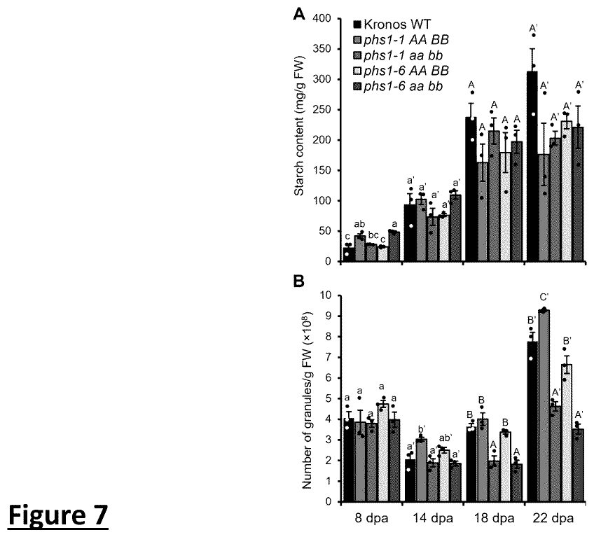

The expression patterns of PHS1 and BGC1 were compared during grain development. We used our previously generated RNAseq dataset for endosperm development in durum wheat cultivar, Kronos (Chen et al., 2022). PHS1 expression was detectable at all stages of grain development tested between 6-30 days post anthesis (dpa) but was particularly strong between 8-13 dpa (FIG. 6A). No differences in expression were observed between the A- and B-homeologs. PHS1 activity levels in endosperm extracts were visualised on native PAGE gels. Consistent with the transcript levels, robust PHS1 activity was detected at all timepoints tested between 8-28 dpa, but was stronger at the earlier than later timepoints, especially at 12 dpa (FIG. 6B). To assess PHS1 expression in different tissues, we used public data for bread wheat cultivar Chinese Spring in the wheat expression browser (Borrill et al., 2016). PHS1 transcripts were detectable in leaves/shoots, roots, spike, and grains, and all homeologs showed similar levels of expression across all examined tissues.

### Example 6: PHS1 Acts During B-Type Granule Initiation in Grain Development

To examine if the loss of PHS1 specifically affected B-type granule formation, we measured starch content, granule number and granule size distributions throughout grain development. We harvested developing grains from the phs1-1 and phs1-6 double mutants, as well as their corresponding wild-type controls, and carried out all starch analyses on dissected endosperms. Similar to in mature grains, we did not observe significant differences in total starch content between the mutants and wild-type controls at any time point during grain development (FIG. 7A). We then used the Coulter counter to quantify the number of starch granules per mg of endosperm, as well as to assess granule size distributions. The number of starch granules did not differ between the mutants and the wild type at 8 or 14 dpa. However, at 18 and 22 dpa, both the phs1 mutants had significantly fewer starch granules than the wild-type controls (FIG. 7B).

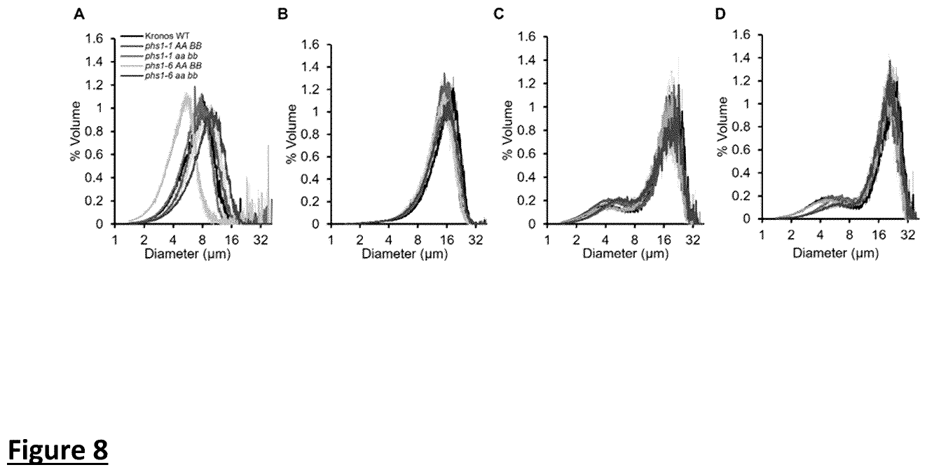

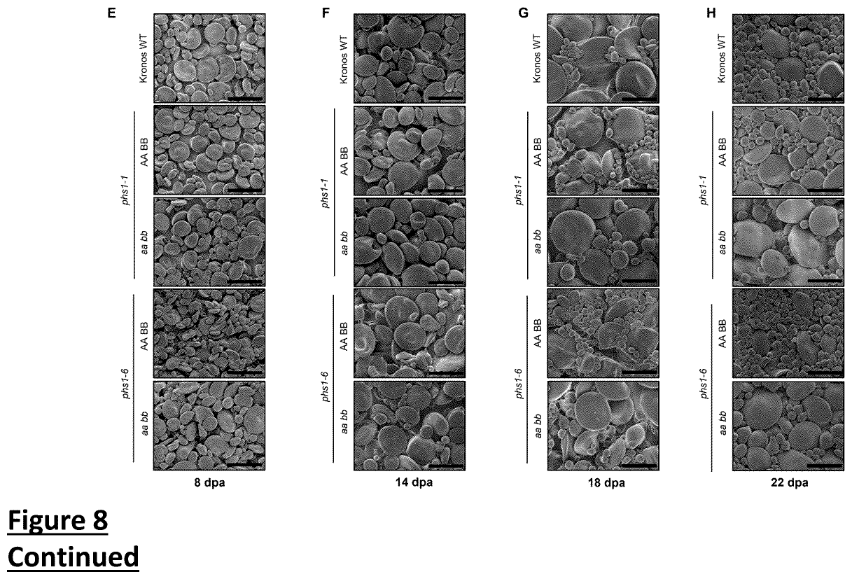

Since B-type granules initiate typically between 15-20 dpa, and only A-type granules are present before this timepoint, phs1 mutants contain normal numbers of A-type granules (FIG. 8A, B), but have fewer B-type granules than the wild type (FIG. 8C, D). This was also strongly supported by the granule size distributions from the Coulter counter and in SEM (FIG. 8A-H). The distributions were unimodal in the mutants and the wild-type controls at the 8 and 14 dpa timepoints (FIG. 8A, B, E, F). However, at the 18 and 22 dpa timepoints, the distributions turned bimodal-suggesting all genotypes could initiate B-type granules (FIG. 8C, D, G, H). However, the B-type granule peaks were smaller and shifted towards the larger sizes in the phs1 mutants (FIG. 8C, D). Larger, and fewer B-type granules were observed in the SEM images of starch from these timepoints (FIG. 8G, H). There were no defects in A-type granule morphology at any timepoint. Together, these data suggest that PHS1 is required for normal B-type granule initiation, but not A-type granule initiation (FIG. 8).

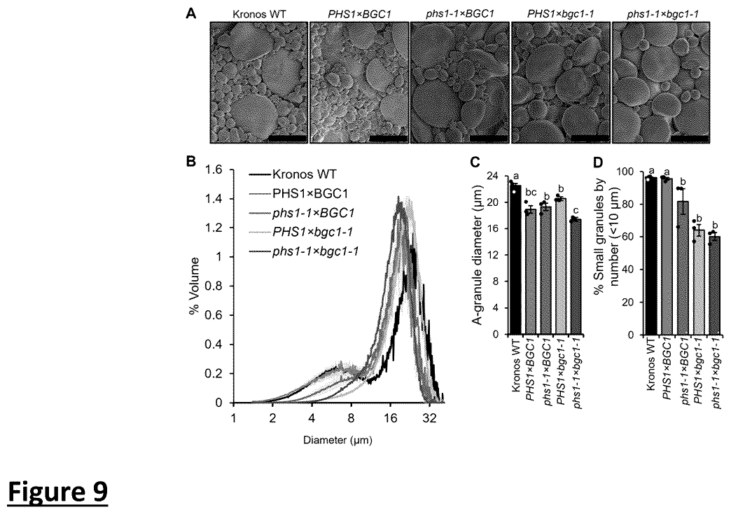

Our finding that PHS1 is required solely for B-type granule initiation was further supported in a genetic approach. Reduced gene dosage of BGC1 affects the number of B-type granules—such as in the bgc1-1 mutant of durum wheat, Kronos, which almost has no B-type granules, due to a loss-of-function mutation in the BGC1-A1 homeolog and a missense mutation in the BGC1-B1 homeolog. We crossed this bgc1-1 line to the phs1-1 line, and isolated a bgc1-1 phs1-1 quadruple mutant in the F2 generation. We also isolated from the cross a wild-type sibling control, and bgc1-1 and phs1-1 mutant siblings as controls. We purified starch granules from mature grains of these lines and examined their morphology using Scanning Electron Microscopy (SEM). Starch from the bgc1-1 and bgc1-1 phs1-1 mutants had visibly fewer B-type granules than the wild type, wild-type sibling control, and phs1-1 mutant siblings (FIG. 9A). Coulter counter analysis of granule size distributions showed that the reduction of B-type granule number was stronger in bgc1-1 than in phs1-1 (FIG. 9B), since bgc1-1 had almost no detectable B-type granule peak (when plotted by volume), whereas phs1-1 had a smaller B-type granule peak that was between that of the wild-type control and bgc1-1. Importantly, the granule size distribution of bgc1-1 phs1-1 quadruple mutants was almost identical to bgc1-1. This shows that the loss of PHS1 in the bgc1-1 background (which only has A-type granules), has no further effect on starch granule size distributions. Using curve-fitting on these plots, we calculated that the size of A-type granules was unaffected by the phs1 mutations in the bgc1-1 background (which only has A-type granules) (FIG. 9C). Next, we calculated the percentage of starch granules that were smaller than <10 μm. There was a significant decrease in the relative number of small granules in the bgc1-1 phs1-1 quadruple mutant and bgc1-1 and phs1-1 mutant siblings compared to the wild type and wild-type sibling control (FIG. 9D). Interestingly, like the phs1 double mutants, we observed that the bgc1-1 also has a small reduction in amylose content. Taken together, our results show that PHS1 has an exclusive role in B-type granule initiation, and does not appear to be required for A-type granule initiation.

### Example 7: Loss of PHS1 in Wheat does not Affect Total Maltooligosaccharides (MOS) Levels in the Endosperm

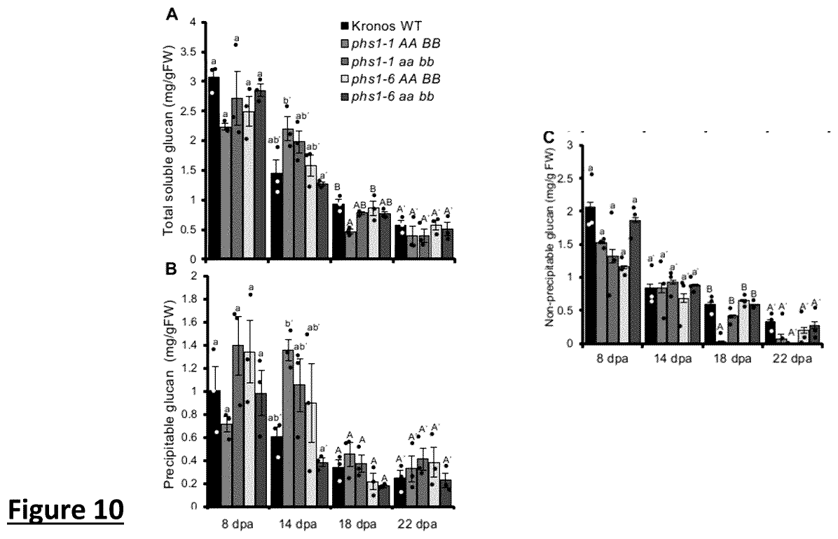

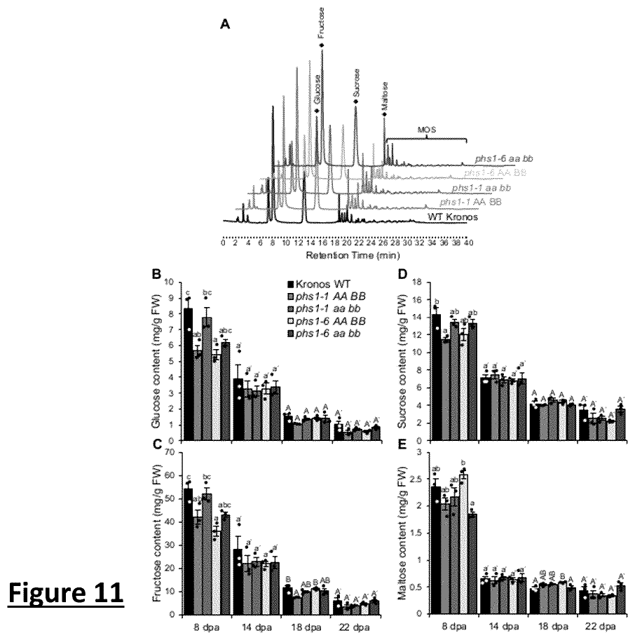

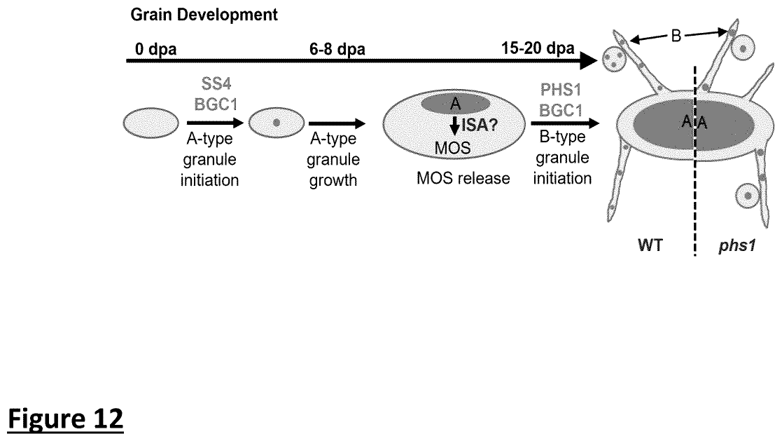

We investigated whether there was MOS accumulation in the developing endosperm of wheat phs1 mutants. However, levels of MOS or other soluble sugars were unaltered in the phs1 mutants. We quantified the total soluble glucans in perchloric acid extracts. For all genotypes, soluble glucans were highest at 8 DPA, and decreased in abundance during grain development, but were always relatively low compared to starch content (approx. 10% of starch content at 8 dpa, and <0.001% at 22 dpa) (FIG. 10). There were no consistent differences between the phs1 mutants and their controls. We then quantified the methanol precipitable fraction of soluble glucans (phytoglycogen and long MOS), and calculated the non-precipitable fraction (short MOS) as the difference between the total soluble glucan and precipitable glucans. Like the total glucans, these decreased in abundance as the grain developed, and there were no consistent differences between the phs1 mutants and controls. Additional High Performance Anion Exchange Chromatography with Pulsed Amperometric Detection (HPAEC-PAD) analyses confirmed this result, since maltose and other detectable MOS did not change in abundance or chain length distribution pattern in the phs1 mutants (FIG. 11). In addition, glucose, fructose and sucrose levels remained unchanged in the mutants at all timepoints (FIG. 11).

### Materials and Methods

### Plant Materials and Growth

Wheat plants were grown in climate-controlled glasshouses for all grain analyses, and in controlled environment rooms (CERs) for the analysis of starch in leaves. The glasshouses were set to provide a minimum 16 h of light at 20° C., and 16° C. during the dark. The CERs were set to provide 16 h light at 20° C. (light intensity was 300 μmol photons m⁻² s⁻¹) and 8 h dark at 16° C. The glasshouses and CERs were set to 60% relative humidity.

Mutant lines of durum wheat (Triticum turgidum cv. Kronos) were obtained from the wheat in silico TILLING resource (http://www.wheat-tilling.com) (Krasileva et al., 2017). Lines Kronos4533 (K4533) and Kronos4367 (K4367) were obtained for PHS1-5A, and lines Kronos2864 (K2864) and Kronos0238 (K0238) were obtained for PHS1-5B (FIG. 1). Plants were crossed to combine mutations in the A- and B-homeologs. The wild-type segregant (AA BB), single homeolog mutants (aa BB, AA bb) and the double homeolog mutant (aa bb) were selected in the F2 generation using KASP V4.0 genotyping (LGC, Teddington) with the primers in Table S1.

### Native Gel Analysis of PHS Activity

The visualisation of a-glucan phosphorylase activity by native PAGE was carried out using the method of (Zeeman et al., 2004) with minor modifications. Leaf samples were homogenised in extraction medium [100 mM MOPS (3-(N-morpholino) propanesulfonic acid), pH 7.2, 1 mM DTT (dithiothreitol), 1 mM EDTA, and 10% (v/v) ethanediol, Complete protease inhibitor cocktail (Roche)], then spun at 20,000 g for 10 min. Soluble proteins were collected in the supernatant, and the protein concentration of the extracts was determined using the Bradford method. Equal amounts of protein (180 μg) were loaded per lane on native PAGE gels (7.5% acrylamide and 0.3% oyster glycogen in resolving gel; 3.75% acrylamide in stacking gel) and were electrophoresed at 200V for 2 h at 4° C. Gels were washed twice in 100 mM Tris-HCl, PH 7.0, 1 mM DTT, then incubated in 100 mM Tris-HCl, PH 7.0, 1 mM DTT, 50 mM Glc-1-P overnight at 20° C. Activity bands were stained with Lugol's iodine solution (L6146, Sigma, St. Louis).

### Grain Morphometrics

Grain yield per plant was quantified as the total weight of grains harvested from each plant. The thousand grain weight and grain size were quantified using the MARVIN seed analyser (Marvitech GmbH, Wittenburg).

### Starch and Amylose Content

Starch content of leaves and grains (in glucose equivalents) were quantified as described in Hawkins et al. (2021). Briefly, for leaves, two-week-old seedlings were harvested at the base of the lowest leaf and flash frozen in liquid N₂, then homogenised in 0.7 M perchloric acid. Insoluble material was collected by centrifugation, washed three times in 80% ethanol, then resuspended in water. Starch was digested using a-amylase/amyloglucosidase (Roche, Basel), and the released glucose was assayed using the hexokinase/glucose-6-phosphate dehydrogenase assay (Roche). For grains, flour (5-10 mg) was dispersed in 100 mM sodium acetate buffer, pH 5, and the starch was digested with thermostable a-amylase at 99° C. for 7 min. Amyloglucosidase was added to the digestion, and was further incubated at 50° C. for 35 min. Both the thermostable a-amylase and amyloglucosidase were from the Total Starch Assay kit (K-TSTA, Megazyme, Bray). The digested sample was centrifuged to remove insoluble material, and glucose was measured in the supernatant as for the leaf starch quantification.

### Starch Purification—Granule Morphology, Size Distribution and Amylose Content

For starch purification from mature grains, grains (3-5 grains per extraction) were soaked overnight in ddH2O at 4° C., then homogenised in a mortar and pestle with excess ddH2O. The homogenates were filtered through a 70 μm nylon mesh, then centrifuged at 3000 g, 5 min, before resuspending the starch pellet in water. The starch suspension was centrifuged at 2500 g, 5 min on a cushion of 90% (v/v) Percoll, 50 mM Tris-HCl, pH 8. The pellet was washed twice in 50 mM Tris-HCl, pH 6.8, 10 mM EDTA, 4% SDS (v/v), 10 mM DTT, then twice in ddH2O, before finally resuspending in ddH2O.

The morphology of purified granules was examined using a Nova NanoSEM 450 (FEI, Hillsboro) scanning electron microscope (SEM). To quantify starch granule size distributions, the purified starch was suspended in Isoton II electrolyte solution (Beckman Coulter, Indianapolis), and particle sizes were measured using a Multisizer 4e Coulter counter (Beckman Coulter) fitted with a 70 μm aperture tube. A minimum of 100,000 particles was measured per sample. These data were used to produce relative volume vs. diameter and relative number vs. diameter plots. A mixed bimodal (two log-normal distributions) were fitted to the relative volume vs. diameter plots to calculate mean diameters of A- and B-type granules, and the B-type granule volume percentage (defined as volume occupied by B-type granules as a percentage of the total volume of starch). Amylose content of the purified starch granules was estimated using an iodine colourimetry method (Washington et al., 2000). Briefly, starch (1 mg) was dispersed overnight at room temperature in 1 M NaOH. The solution was neutralised to pH 7 using 1 M HCl, and 5 μL of this solution was diluted in 220 μL of water and 25 μL Lugol's iodine solution (Sigma). The reaction was incubated at room temperature for 10 minutes prior to absorbance measurements at 535 nm and 620 nm. Apparent amylose content was estimated using the formula:

$$Apparent amylose content = 1.4935 * {exp}^{[ 2.7029 * ( Absorbance 620 / Absorbance 535 ) ]}$$

TABLE 2
Primers used for KASP genotyping

| Allele | WT | mutant | common |
|---|---|---|---|
| K4533 | Tttttgattctctgatattgaattg | Tttttgattctctgatattgaatta | Aaggtatgagataaggctgaaga |
| | (SEQ ID NO: 70) | (SEQ ID NO: 74) | (SEQ ID NO: 78) |
| K4367 | Atatccgtttcgacctgcag | Atatccgtttcgacctgcaa | Ccatatgtccttggcatactcg |
| | (SEQ ID NO: 71) | (SEQ ID NO: 75) | (SEQ ID NO: 79) |
| K2864 | Agagacatcatttcttacgatctcc | Agagacatcatttcttacgatctct | Acctgcatgttagcttcttttct |
| | (SEQ ID NO: 72) | (SEQ ID NO: 76) | (SEQ ID NO: 80) |
| K0238 | Aaaataaagtagacgaaagaatac | Aaaataaagtagacgaaagaatat | Acaaaattttgatttgggtgcc |
| | (SEQ ID NO: 73) | (SEQ ID NO: 77) | (SEQ ID NO: 81) |

All primer sequences are provided in 5′ to. 3′ orientation. For KASP genotyping, the VIC/HEX tail (gaaggtcggagtcaacggatt) (SEQ ID NO: 82) was added to the 5′ end of the WT allele primers, and the FAM tail (gaaggtgaccaagttcatgct) (SEQ ID NO: 83) was added to the 5′ end of the mutant allele primers.

### Isolation, Cloning, Plasmid Construction for PHS1 and BGC1

Total RNA was extracted from leaves of two-week-old wheat seedlings using RNease kit with on-column DNase I digestion (Qiagen). cDNA was synthesized (using 2 μg RNA) using the Go Script™ Reverse Transcriptase Kit (Promega) following the manufacturer's instruction. The full-length wheat PHS1 cDNA sequence was amplified using gene specific primers listed in Supplemental Table S3 and inserted into Gateway entry vector, pENTR, using the pENTR/D-TOPO kit (Invitrogen). To produce constructs with a C-terminal YFP or RFP tag, we recombined the coding sequences from PHS1:pENTR and BGC1:pDONR221 (Hawkins et al., 2020) into the plant expression vectors pB7YWG2 (35S promoter; C-terminal YFP) or pB7RWG2 (35S promoter; C-terminal RFP) using Gateway LR clonase (Invitrogen). To generate the chloroplast-targeted YFP and RFP proteins, the rubisco small subunit (RbcS) transit peptide (Kim et al., 2010) was cloned into pDONR221, and recombined into the pB7YWG2 and pB7RWG2.

Transient Expression of PHS1 and BGC1 in N. benthamiana and Protein Localisation

Constructs for YFP-tagged PHS1 and BGC1 expression were transformed into Agrobacterium tumefaciens (strain GV3101). Agrobacterium cells were grown at 28° C. in Luria-Bertani medium with the appropriate antibiotics. Cells were pelleted, washed, and resuspended in the infiltration medium [10 mM 2-(N-morpholino) ethanesulfonic acid, 10 mM MgCl2, and 0.15 mM acetosyringone, pH 5.6]. The cell suspension was then infiltrated into the intercellular spaces between abaxial epidermal cells of intact Nicotiana benthamiana leaves using a 1 mL plastic syringe. Infiltrated plants were incubated overnight in dark followed by 2 days in a 16 h/8 h light-dark cycle. YFP fluorescence in leaf samples was observed using a laser scanning confocal microscope (SP8; Leica) with a 63× water-immersion lens. Images were processed using LAS X software.

### Pulldown Assay, Immunoprecipitations and Mass Spectrometry

For the pulldown assay to identify proteins associating with BGC1, the bait protein (recombinant His-tagged BGC1) was expressed in E. coli as described in Hawkins et al. (2020) and purified in its native state using Ni-NTA agarose beads (Qiagen) as previously described (Seung et al., 2013). To produce endosperm extract, endosperms were dissected from developing grains (collected at 18 dpa) of wild-type Kronos plants and homogenised in ice-cold extraction medium [50 mM Tris-HCl, pH 8, 1 mM DTT, 1% (v/v) Triton X-100, 150 mM NaCl, and Roche Complete Protease Inhibitor cocktail] at a rate of 1 mL buffer per 100 mg tissue. Insoluble material was removed by centrifugation at full speed for 5 min at 4° C., and proteins were collected in the supernatant. Recombinant BGC1-His protein (2.5 μg) was added to the supernatant (1 mL) and was incubated for 1 h at 4° C. μMACS magnetic beads conjugated to anti-His (Miltenyi Biotec) were added and incubated for 1 h at 4° C. to retrieve the bait protein together with interacting proteins. The beads were captured with a μColumn on a magnetic stand (Miltenyi Biotec), washed three times with wash medium [50 mM Tris-HCl, pH 8, 1 mM DTT, 1% (v/v) Triton X-100, 300 mM NaCl, and Roche Complete Protease Inhibitor cocktail], then three times with wash medium without Triton X-100, before eluting the bound proteins with elution medium [50 mM Tris-HCl, pH 6.8, and 2% (w/v) SDS].

The eluted proteins were precipitated with chloroform/methanol according to (Pankow et al., 2016). Protein pellets were resuspended in 50 μl of 2.5% sodium deoxycholate (SDC; Merck) in 0.2 M EPPS-buffer (Merck), pH 8.5 and reduced, alkylated, and digested with trypsin in the SDC buffer according to standard procedures. After the digest, the SDC was precipitated by adjusting to 0.2% trifluoroacetic acid (TFA), and the clear supernatant subjected to C18 SPE. Samples were dried in a SpeedVac concentrator (Thermo Fisher Scientific, #SPD120) and the peptides dissolved in 0.1% TFA/3% acetonitrile.

Peptides were analysed by nanoLC-MS/MS on an Orbitrap Eclipse™ Tribrid™ mass spectrometer with a FAIMS Pro Duo source, coupled to an UltiMate® 3000 RSLCnano LC system (Thermo Fisher Scientific, Hemel Hempstead, UK). The samples were loaded and trapped using a trap cartridge (Pepmap Neo C18, 5 μm, 300 μm×5 mm, Thermo) with 0.1% TFA at 15 μl min⁻¹ for 3 min. The trap was then switched in-line with the analytical column (nanoEase M/Z column, HSS C18 T3, 100 Å, 1.8 μm; Waters, Wilmslow, UK) for separation using the following gradient of solvents A (water, 0.1% formic acid) and B (80% acetonitrile, 0.1% formic acid) at a flow rate of 0.2 μl min⁻¹:0-3 min 3% B (during trapping); 3-10 min linear increase B to 7%; 10-100 min increase B to 32%; 100-148 min increase B to 50%; followed by a ramp to 99% B and re-equilibration to 3% B, for a total running time of 180 minutes. Mass spectrometry data were acquired with the FAIMS device set to three compensation voltages (−35V, −50V, −65V) at standard resolution for 1 s each with the following MS settings in positive ion mode: MS1/OT: resolution 120K, profile mode, mass range m/z 300-1800, spray voltage 2800 V, AGC 4e⁵, maximum injection time 50 ms; MS2/IT: data dependent analysis was performed using HCD fragmentation with the following parameters: cycle time of 1 s in IT turbo for each FAIMS CV, centroid mode, isolation window 1.0 Da, charge states 2-5, threshold 1.0e⁴, CE=30, normalised AGC target 100%, max. inject time set to Auto, dynamic exclusion 1 count, 15 s exclusion, exclusion mass window±10 ppm.

The acquired raw data were processed and quantified in Proteome Discoverer 3.0 (Thermo) using the incorporated search engine CHIMERYS® (MSAID® Munich, Germany). The processing workflow included recalibration of MS1 spectra (RC) and the Minora Feature Detector for quantification with min. trace length=7 and S/N threshold=3. The Top N Peak Filter (10 per 100 Da) was applied and the CHIMERYS® search was performed with the prediction model inferys_2.1_fragmentation, enzyme trypsin with 2 missed cleavages, peptide length 7-25, fragment tolerance 0.5 Da, variable modification oxidation (M), fixed modifications carbamidomethyl (C). Percolator was used for validation using q-value and FDR 0.01 (strict) and 0.05 (relaxed).

In the consensus workflow quantification was performed with a maximum RT Shift of 3 min and a mass tolerance of 4 ppm between runs. Protein quantification was based on the top 3 most abundant unique peptides per protein group. Missing values were replaced by low abundance resampling. Protein abundance ratios were calculated from the 3 replicates per sample. The hypothesis test was performed by a background-based t-test and the p-values adjusted according to BH.

For pairwise co-immunoprecipitations, proteins were transiently expressed in N. benthamiana leaves as described above, and proteins were extracted as described for the pulldown assay. The supernatant was incubated for 1 h at 4° C. with μMACS magnetic beads conjugated to anti-YFP. After incubation, the beads were recovered using a μColumn (Miltenyi Biotec) on a magnetic stand. The beads were washed five times with wash medium [50 mM Tris-HCl, pH 8, 1 mM DTT, 1% (v/v) Triton X-100, 300 mM NaCl, and Roche Complete Protease Inhibitor cocktail] before eluting the bound proteins with SDS-PAGE loading buffer [50 mM Tris-HCl, pH 6.8, 2% (w/v) SDS, 100 mM DTT, 3% (v/v) glycerol, 0.005% (w/v) bromophenol blue]. The eluates were analysed using SDS-PAGE and immunoblotting, using anti-YFP (Torrey Pines; TP401-1:5000) and anti-RFP (Abcam plc; ab34771-1:2000) primary antibodies. Proteins were detected using chemiluminescence from horseradish peroxidase-coupled secondary antibodies Anti-rabbit HRP (Sigma; A0545), 1:15,000.

### Quantification of Starch, Sugars, MOS, Granule Number and Size in Developing Grains

Endosperms were dissected from developing grains collected at 8, 14, 18 and 22 dpa. In each extraction, three to five individual endosperms of known fresh weight were used. After homogenisation in 0.7 M perchloric acid, homogenates were spun at 10,000 g for 5 mins, and the supernatant was immediately neutralized using neutralisation buffer (2 M KOH, 400 mM MES). This neutralised soluble fraction was used for sugar and MOS quantification (see below).

The pellet was resuspended in ddH2O and equally divided into two fractions. One fraction was used to quantify starch content following the method described above for leaves. The other was used for starch purifications (as described above for mature grains), and all granules purified within this fraction was resuspended in known volumes of Isoton II electrolyte solution (Beckman Coulter, Indianapolis). The suspension was analysed in a Multisizer 4e Coulter counter (Beckman Coulter) fitted with a 70 μm aperture tube and running in volumetric mode (analysing 2 mL of the suspension). This gave the number of granules in the suspension, which could be used to calculate the number of granules per starting fresh weight of endosperm; as well as granule size distribution plots.

For the quantification of soluble glucans: Total soluble glucans (neutralised soluble fraction without precipitation) and methanol-precipitable soluble glucans (after precipitation) were quantified. For methanol precipitation, one volume of neutralised soluble fraction was mixed with 4 volumes of pure methanol, mixed, and incubated overnight at −20° C. Precipitated glucans were collected by centrifugation at 10,000 g, 5 min, then washed with 75% methanol, and dried. Dry pellets were suspended in one volume of ddH2O. To quantify glucans, the neutralised soluble fraction and resuspended precipitated glucans were digested using a-amylase/amyloglucosidase (Roche, Basel), and released glucose was assayed using the hexokinase/glucose-6-phosphate dehydrogenase assay (Roche).

HPAEC-PAD was used to quantify glucose, fructose, sucrose, and maltose, and to visualise MOS accumulation patterns. The neutralised soluble fraction was purified on sequential columns of Dowex 50W and Dowex 1 (Sigma) as described previously Seung et al. (2013). Purified samples were separated on an ICS-5000 HPLC fitted with a CarboPac™ PA20 column (3×250 mm, CV=1.06 ml; Dionex). The mobile phase consisted of eluate A (100 mM NaOH) and eluate B (150 mM NaOH, 500 mM sodium acetate), following a gradient program of: 0-7 min, 0% B; 7.0-26.5 min, a concave gradient to 80% B; 26.5-32.0 min, 80% B; 32.0-32.1 min, linear gradient to 0% B, 32.1-40.0 min, 0% B with flow rate of 0.25 ml/min.

### Statistical Analysis

Statistical analyses were carried out using the SPSS program (SPSS Statistics, IBM).

### Accession Numbers

The accession numbers corresponding to the genes investigated in this study are:
TraesCS5A02G395200 (PHS1-A1), TraesCS5B02G400000 (PHS1-B1), TRITD5Av1G205670 (PHS1-A1), TRITD5Bv1G201740 (PHS1-B1), TraesCS4A02G284000 (BGC1-A1), TraesCS4B02G029700 (BGC1-B1), TRITD4Av1G198830 (BGC1-A1), TRITD0Uv1G034540 (BGC1-B1).

### REFERENCES

Hawkins E, Chen J, Watson-Lazowski A, Ahn-Jarvis J, Barclay J E, Fahy B, Hartley M, Warren F J, Seung D. STARCH SYNTHASE 4 is required for normal starch granule initiation in amyloplasts of wheat endosperm. New Phytol. 2021 June; 230 (6): 2371-2386.
Park, S. H. et al. “Starch granule size distribution of hard red winter and hard red spring wheat: Its effects on mixing and breadmaking quality.” Journal of Cereal Science 49 (2009): 98-105.
Saccomanno B, Berbezy P, Findlay K, Shoesmith J, Uauy C, Viallis B, Trafford K. Characterization of wheat lacking B-type starch granules. J Cereal Sci. 2022 March; 104:103398
Stark J R, Lynn A. Starch granules large and small. Biochem Soc Trans. 1992 February; 20 (1): 7-12.
Stoddard, F. L. and Sarker, R. (2000), Characterization of Starch in Aegilops Species. Cereal Chemistry, 77:445-447.
Washington, J. M., et al. (2000). “Developing Waxy Barley Cultivars for Food, Feed and Malt.” Barley Genetics VIII: 303-306.
Zeeman S C, Thorneycroft D, Schupp N, Chapple A, Week M, Dunstan H, Haldimann P, Bechtold N, Smith A M, Smith S M. Plastidial alpha-glucan phosphorylase is not required for starch degradation in Arabidopsis leaves but has a role in the tolerance of abiotic stress. Plant Physiol. 2004 June; 135 (2): 849-58.

SEQUENCE LISTING

SEQ ID NO: 1 >Wheat (hexaploid) PHS1 5A (Chinese Spring reference genome) genomic DNA
CATCACTCACTCGGGTACAGAGCGCAGCGGGCCGCGTGACTCTCGCGTCGTCGCATCGC
GCGCGCTGCACACCACCGTCAGCGCGACCACTGCCGCCAATGGCGACCGCCTCGCCGCC
GCTCGCCACCCCCTTCCGCCCGCTCGCCGTCGCCGGAGGTAGCGGCGGACTCGTCGTGG
GCGCCAGGGCCGTGGTACCGCCGAGGCGGGGGGGGCGGGGGTTCGTGGTGCGGAGCG
TGGCGAGCGATCGGGAGGTGCGGGGGCCCGCCCCGACCGAGGAAGGTAAGCGGCCGCG
CGCTCCCATTGGCTGCCACGACCTGGAATGCTGGTCGGCTGTAATGTGGTTCGGATTTTC
GCTGGTGCGACCTGAATTTGTGATCCTCTGACTGACGGCCTATGTGTTCCCCGGGTTTCG
GGCTGTTGATTTTGTGCGCGCGCATTGCTAATCTACTTCTCTAGGATTTGTTAGGGCTCAG
AGCAATCTTAAATTCGCGAATACACTTCGACGAATGTAGTCTGAATTTACTGAAACTAGCTT
AGTTTGGCGCGTCAAGATAGACTGATGTATTTATGGTAGCAAATTCTGAAAACTTCTTCTGT
GCGTGGACAGCCACCCCTCGTGGATGATGCAGCAAGTGCTAACACAGTGTTTGCTAGTAC
TTGCTAGTATCTGTAGCTGTAACAAAATCCTTCGAATCTGAACTAGTGAGATAAATCGTTTG
AGATTTTTGCTGAAGATTACACAGTATCAATATTTCTGAAGGTGATTGGAACAGTATAGCAT
TTCAAACTGGCAACCAAGCAGGATCAGTATACTGACGCAATTTTTTTCTGAAGGTGGTTGG
AATACCGGTATGAATTTTATAATCATTGAATTAAGGCGTAAGATTTGGTAACAAAAACTGCTT
TTCACGGGAAACGATGAAACATGGCACAATTCAACCTGACTTTCTTTTATGAAAAGGCTCTC
AAGCTGCCTTGTACTCCCTCCGTCCCATAATATAAGAACGTTTTTGACATTAGTGTAGTACC
AAAAACGTTCTTATATTATGGGACAGAGGGAGTATTTATTAATGGATAAAAAGAGAGGTACA
GGGTTCTAGTAGTAACATATGCAAATGGTGATACAGTATATTATCTACGATTTTGTGCAAGA
ATTTTCACACGAAAACAATATTCCATGCAGTTTTTAGTTTTGACTTTTTTTTTTGNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNCGAACCCAACATACAG
TACAATAGTACAAATGCCGGGGCGGCAGCAGAGAGAGAGGCAGGGGGCGACCCCCCCAT
GCTATGTTACATGCAGCTTTGTTCTACCCACGCAGAGCCTGTAGCCAGTTTTGACTCTTTTT
GAATTGTTTGGATTGCTTACCACATGTGTCACGTGGTTTTAGTTTTGACTCTTGACTGTTTAA
TACCTACTCTTTTTAAGATTTTGGATTCTAATGTCATCTGTCTTGCAACTCAGAGCTTTCAGC
CGTGCTAACTTCCATTGATTCATCCGCCATCGCATCAAACATCCAGCACCATGCAGACTTC
ACACCGTTATTCTCGCCAGAGCACTCTTCACCCCTAAAGGCTTACCATGCAACTGCCAAAA
GTGTTTTTGATTCTCTGATATTGAATTGGAATGCAACATATGACTATTACAACAAAGTGAATG
CAAAGCAAGCTTATTACCTATCCATGGAATTTTTGCAGGTCTCGCAATATACTTTCTTCAGC
CTTATCTCATACCTTTTGGTAACTGCGTTATGATGTTATATGTTTTTTTTTGTACTGCAGGGA
AGAGCTCTTACAAATGCTATTGGCAACCTAGAGCTAACAGGACAATATGCAGAAGCGTTAA
AACAACTTGGACAGAACCTAGAGGATGTTGCTAGTCAGGTAAGCCATTTTTGTCAGAGAAT
GGTTTTTAGTCCATATCAAAATTATTGCCGAGCCATTTTTCTAAATAACATTTTATTATTATTC
ATGAACTTCCATATTTTTTTCAAATCTGCAAAAGGAGAAGTTTTTTTCCTGGGGGTTCCACC
ATCTGACTTATATTTGTTATGCAGTTTTCAGAGTGATTGACTATGAAATTACGCCAAAACAC
GAGCATTGCCTAGAGTGCCCTGTAGTTTTAGAAATGTTAAGATTGGATATGTTACTTTTCTA
TTACAATGAAACAGGAACCAGATCCTGCCCTTGGCAATGGTGGACTAGGCCGTCTAGCGT
CCTGTTTTCTGGATTCTATGGCAACCCTAAATTATCCAGCATGGGGATATGGACTTCGGTA
CAGATATGGCCTCTTTAAGCAAATCATAACAAAGGATGGTCAGGAGGAGGTAGCTGAGAAT
TGGCTAGAGGTAGTGATATGTCTTCTACTTGTTTACTGTTTGGAAGCGTTGATTATGTTGGT
TTCTGTTTTAATGATCTGCATGTTAGCTTCTTTTGCCATTTATAACTGATGCTGTATCTCCAG
ATGGGAAATCCATGGGAGATTGTAAGAAATGATGTCTCTTATCCTCTGAAATTCTATGGCAA
AGTGGTTGAAGGCACTGATGGGAGAAAACACTGGATTGGAGGAGAGAATATCAAGGCTGT
GGCACATGATGTTCCTATTCCTGGCTACAAGACTAAAACTACTAATAATCTTCGTCTTTGGT
CAACAACAGTACCATCACAAAACTTTGATTTGGGAGCTTTTAATGCTGGGGATCATGTCAAG
GCCAACGAAGCACATCTAAATGCTGAAAAGGTATTCCTTTGTCTGCATTTATTTTGAATATA
AATCATTAAATCTCAATAACTATTGATTTAAATGGTAAACACTAAACATTGTCTAGCTAGTAG
TCACTGTGGTGTCCCTCAATGTCATTTTAGAATGTGCACAGTCAAATGTTTGATAACTTTGA
TTGTAATAATATGATAATATACAGAAACTTTGATTGACACTAGATTTTTCATAAACATACTTAT
TGTATTTCCATGTAAATAGAAAAGCTGATGTTCAGAGGTTTGTGTGTCAAAATGAACAGAGT
TTGTGCACACATAATATCTTCATCTGGTGTTTGCAGTTCTCTAGGTTGTGTTTGCGTTTTCTC
CTGCTCTGCCTTAACTAATCTCAGCATATGCATCAGGCTACATTGCTCCTCTGTGTCTATAA
AAAATAAGTCTGATGATATATGGTTGCAGTCCAGTAGTTCGTAGATGGCACAGCATGGAAG
AACTTTTCACATGGAAGCCTTACCCTTATAATTTTAATTATACCTTTTGAGTGTTTGTGCATT
GTGTACATACACCATTAAGTATGGGTGCTCAAAAATGGTTTATCCCAAACTGTGCAAAAGCC
GCCACTGTTTGTGTATTAATTTTTTTGAACTAGCTAAACAACTTGACCGAACAAGATACATAC
AATTGCACTCACTTCTGAAGCAGCTCAGTTGGACGGATTTGTTCCAATCCCGTGCATGGGT
TAAATTGTCGAGTTGTCCTCGCTTGTTCAGTAGTAGGAGTACTATCTATTATATTTATACTAA
AGTTTTCTTTTTGTGATACAGTACAAACTTTTAGTAATACTTACATTTGCGACCACTGTGGAC
AAAATTTATTATGTCTAACTTAACTTTATCACTGGTCTTCTCATCATGCCAGATATGCCACGT
ATTGTATCCAGGGGACGAATCATCAGAGGGGAAAATTCTTCGCCTGAAACAACAATATACA
CTATGCTCGGCCTCCCTGCAGGACATTATTTCTCGTTTTGAGTCGAGGGCTGGTGATTCTC
TCAACTGGGAGGACTTCCCCTCTAAAGTTGCAGTTCAGATGAATGACACCCATCCAACACT
GTGCATTCCCGAGTTAATGAGAATACTGATGGATGTTAAGGGATTGAGCTGGAATGAGTCT
TGGAGTATCACGGAAAGGTATCACAGAAGGGTACTGCAGTTTGATGTGTTATGCTGTTCCT
CCAGTTCCAGTTAACTAATTTTATTTCCTCATCTGTCTTCCATTCAATCATGTACTGATTTTCT
GATATCCACATCATTTGTAGAACCGTTGCATACACTAACCACACAGTACTTCCTGAAGCTCT
GGAGAAGTGGAGCTTGGACATTATGCAGAAACTTCTACCTCGACATGTTGAGATTATAGAA
AGAATAGATGAGGAGGTATCAAATGTGCAGATAATATTATACAATTAATTTCATGCTCTGTA
GTTCAACTTATGGTTTACTTGTCTTCATCGTTGATTTTAAACAGCTGATGAACACCATCGTCT
CAAAATATGGAACGGCAGATATTTCACTGTTAAAACAGAAGCTTAAAGATATGAGGATCTTA
GACAACGTTGATCTTCCAGCCTCTGTTGCTAAACTGTTTATTAAACCAAAAGAGAAAAAGGG
GAAGTTGCTTGTTGAATCTTTGGAGTCTATTGCTGAAGCTGACGAGAAAACTGAGTCAGAA
GAGGAGGAAAACATTCTATCCGAGACAACAGAGAAAAAGGGCGAATCTGACTCTGAAGAA
GCTCCTGATGCAGAAAAGGAGGATCCTGAGTATGAGTTAGATCCATTTACAAAATACGATC
CTCAGTTACCTAGAGTTGTTCGAATGGCGAACCTCTGCGTTGTTGGTGGACATTCAGTTAA
TGGTGTGGCTGAGATTCACAGTGAAATTGTTAAGCAAGATGTGTTCAATAGCTTTTATGAGG
TATTTGCATGATGATTTGGAAACCGTGATCTTATTTTCCAATGGCTGCTATAATCGTCAGGT
GATATGGTATTCACCATCTGCCATTTTCTTGTGTTCTTTGCCAGATGTGGCCTACTAAGTTC
CAAAACAAAACAAATGGAGTGACCCCCAGGCGTTGGATCCGGTTTTGTAATCCTGAATTAA
GTACAATAATTTCAAAATGGATAGGCTCTGATGACTGGATTCTGAACACTGATAAACTTGCA
GGACTGAAGAAGGTACTTCCAGTAGTTTTATTCCCCAATAATCAGTAATCTAATGCTTCCTT
CACTTCAATGTTAACTGTGCATTGTTTTTACATTGATGTGTGTTCATCCTGTAACAGTTTGCT
GATGATGAGGATCTGCAATCAGAATGGCGTACTGCTAAAAGGAATAACAAGATGAAGGTAG
TTTCGCTGATAAGAGACAAGACTGGATATGTTGTCAGCCCAGATGCAATGTTTGATGTGCA
GGTATATTTTGTACTAGAAAACATGTGTCTTCTTATAAATATGGTTTAACTCAGCTCTTGGTA
CTGGACACTTAGGTGAAAAGAATACATGAATATAAGCGGCAGCTTCTAAATATCCTTGGTAT
CATTTACCGCTACAAGAAGATGAAAGAAATGAGTGCAAAAGATAGAATAAAGAGCTTTGTTC
CAAGGGTATGCATATTTGGCGGGAAAGCTTTTGCCACTTACGTACAGGCAAAGAGGATTGT
GAAGTTTATCACAGATGTCGCAGCTACTGTAAATTATGATCCTGATATTGGAGATCTACTGA
AGGTAAATTTGTGTCTTTGATGCCACTTATCAGTCTTAATTTTCATGTTCTGTCACTGGCAAG
TTGACTCCGACTGAAAAGCATTGAATGTTCAGAGTTTCAAAGGAAAACAGAGCAGTGAATG
TTCTTAATGTCGGTGCTTGTAAAACTGAGCAACTGAATAGCCACAGGTTTTTTAGAGAACAA
TATAGCCACAAATTTTTTTGACACTTTTATTGCTATCAGCATCTGATGCTGATATTACTGATA
ACTAGATGAGGTAAATATAAGGTGACAGGTTAAGTCTGAGGATTTTTGTTTGTTATCCAGGT
TGTATTTGTCCCAGATTATAATGTTAGCGTTGCTGAGACGCTCATTCCTGCCAGTGAATTGT
CCCAGCATATCAGGTAATATTCTGCCTACTCTTATATAGTATGTGAATACATAAAAAAAGAAT
ATTACCTGGAGTTGGCTGACATAGAATTTTTCATGTGCCTTTTTGTTTGCTATAATATGTACC
TGGAGTCTTACTGGGATAGTGCCATCCAGAAGTGCCACCCCAGTTTCTATTTCTGTATAAG
ACGTGCATTTTTAACAGTATTACTATCCATTCAGTACCGCTGGAATGGAAGCTAGTGGAACC
AGCAACATGAAGTTTGCAATGAATGGCTGTATTCTTATTGGAACTTTGGATGGTGCGAATGT
GGAGATCAGAGAAGAGGTTGGAGAGGAGAACTTTTTCCTCTTTGGTGCACATGCACCTGAA
ATCGCTGGTTTGAGGCAAGAGAGAGCTGAAGGGAAGGTACCACACTTGTTTATATAGCTGT
GCCTACTATCAAACTAGTAAAGATCTCTCCCATATGTGCTGTCCAAATAGTTTAGCTGAAGG
AAAATCATAACTTTACAAGTTTCCTTTGCACTTGTAGTTTGTGCCTGACCCGAGATTTGAAG
AGGTTAAGGAATACGTCCGCAGTGGCATCTTTGGGACTGGCAACTATGATGAATTGATGGG
TTCTTTGGAAGGAAATGAAGGTTATGGACGTGCAGATTATTTTCTTGTTGGCAAGGATTTTC
CCAGTTACATTGAATGCCAGGAAAAAGTTGATGAAGCATACCGAGATCAGAAGGTAAGTTG
TATAACTGACGCTGTTTGTTTTCTGGCTGAAATGCTCACAGTGATTATGCAAACTTTCTGAT
ATGTGATGGCTGACATCAGTGTTTCACTGTGAACGACTTCATTGATTCGTAGGAAGGAATAT
TTCTTAGTTTAAAGTTTCAAACTGAAGTATCTATCGGTGTGTTGGAAGATCATAAACAATCA
GAGAATATAGTTGCTAGATGTGCTATATTACTCATCTGTTGGCTGTTGAGTGTTGATACATG
AGCTTTGATGTAGCTAGACCTTCAGGAAAGTGCTTCTGTGCATTCCAATAAAAAAATAACTT
ATAACTTAACAAAACAAAACAATTAGGTTGAACCTCACCAGGGGGCCTTTTGTTATCTGTTT
TCTGAAATGAAAATCAGAAGTGGGGAGCTCCTCAATTCGAAAAAGAAAAAGAAAATCAGGG
GGTAACTGAGGGTCTGGTTTCCCTCTGTTGGAGCCCCCAAGAATTCTGTGGTCTTATTTTT
GTAAGAGTATAAGATGCACTGCTCCGTTAAGTTTCTCCTTCACCCATTTGTTTTCATTGCTT
CGTGACATTTCGTGTGACAGCTCAGATCACCATATGCATCCCATTTGTACACATTATTTTGC
TACCGTGGATATTTCCACTCTGTTTCATGTGTGACATTTTTAATTCGTTACATTGTTACTCTA
AACATTAGTCTAATAGATTTTCAGAGTTTTGACCCTCCTAATCACATAATATCCGTTTCGACC
TGCAGCTATGGACAAGAATGTCTATCCTCAACACAGCGGGTTCCCCCAAGTTCAGCAGTGA
CCGGACGATCCACGAGTATGCCAAGGACATATGGGGTATCAGCCCCGTCATCATGCCCTG
AATAATTCAAGCAGCTTCCAGGAACCTTGAGAGTCTATGAGGCGCGATTGGTCGCTGATTT
GCCTTGATTCAGCGAGAATGTATTGCTCCCCCCAGTTTTGTATAGGCATATGCAAGTTCTG
ACATGGACCTGAGTGCTGAGAGCATGCAGGGTAGTTCATCCTTAGCAATATTGCATGTCAG
TTAGCCAGTATTACCCAGGCTTATGGTGTGTGTATTTCGCTGAATAAAGTTGGAGCAGAGT
CATCAAACAAACATACTTTGGTGTATCTGTAATGATAAATAAATGGTACATGTGACTGAACA
AAAAGTGTCGTTGGCGTGCACATGTAGTAGAAGCATGTTTCTTCAGAGAGGGTGCAAAGCT
GAAAGCACCCCAGCTTTGGAGTGAGAATGCAGAAGTTACGAAAACATTTGTTTACATCCAA
AGTGCAACGGGATTCGTCAAGAGTTCAGAGTCTTCCATCACGGTCATAGAGCATGTCCAAT
AGCAGCCCTATAAACCTATAACAGAGCAGTAGCAGTGGTCCCTCACATTTTGAACCAAAAA
TGCAGCTCCAACGGCTGCCCTATTTGTAAAATTTACTTCAAAATTTGTTGGACAGCCCTAAA
ATGCAACACCAACTGATGTAAATGTACAACTATTCGGCTGCTGCCCTAAAATTTGCTCCAAG
TGGCGAACCACTCAGGACCAC
SEQ ID NO: 2 >Wheat (hexaploid) PHS1 5A (Chinese Spring reference genome) CDS
ATGGCGACCGCCTCGCCGCCGCTCGCCACCCCCTTCCGCCCGCTCGCCGTCGCCGGAG
GTAGCGGCGGACTCGTCGTGGGCGCCAGGGCCGTGGTACCGCCGAGGCGGGGGGGGC
GGGGGTTCGTGGTGCGGAGCGTGGCGAGCGATCGGGAGGTGCGGGGGCCCGCCCCGA
CCGAGGAAGAGCTTTCAGCCGTGCTAACTTCCATTGATTCATCCGCCATCGCATCAAACAT
CCAGCACCATGCAGACTTCACACCGTTATTCTCGCCAGAGCACTCTTCACCCCTAAAGGCT
TACCATGCAACTGCCAAAAGTGTTTTTGATTCTCTGATATTGAATTGGAATGCAACATATGA
CTATTACAACAAAGTGAATGCAAAGCAAGCTTATTACCTATCCATGGAATTTTTGCAGGGAA
GAGCTCTTACAAATGCTATTGGCAACCTAGAGCTAACAGGACAATATGCAGAAGCGTTAAA
ACAACTTGGACAGAACCTAGAGGATGTTGCTAGTCAGGAACCAGATCCTGCCCTTGGCAAT
GGTGGACTAGGCCGTCTAGCGTCCTGTTTTCTGGATTCTATGGCAACCCTAAATTATCCAG
CATGGGGATATGGACTTCGGTACAGATATGGCCTCTTTAAGCAAATCATAACAAAGGATGG
TCAGGAGGAGGTAGCTGAGAATTGGCTAGAGATGGGAAATCCATGGGAGATTGTAAGAAA
TGATGTCTCTTATCCTCTGAAATTCTATGGCAAAGTGGTTGAAGGCACTGATGGGAGAAAA
CACTGGATTGGAGGAGAGAATATCAAGGCTGTGGCACATGATGTTCCTATTCCTGGCTACA
AGACTAAAACTACTAATAATCTTCGTCTTTGGTCAACAACAGTACCATCACAAAACTTTGATT
TGGGAGCTTTTAATGCTGGGGATCATGTCAAGGCCAACGAAGCACATCTAAATGCTGAAAA
GATATGCCACGTATTGTATCCAGGGGACGAATCATCAGAGGGGAAAATTCTTCGCCTGAAA
CAACAATATACACTATGCTCGGCCTCCCTGCAGGACATTATTTCTCGTTTTGAGTCGAGGG
CTGGTGATTCTCTCAACTGGGAGGACTTCCCCTCTAAAGTTGCAGTTCAGATGAATGACAC
CCATCCAACACTGTGCATTCCCGAGTTAATGAGAATACTGATGGATGTTAAGGGATTGAGC
TGGAATGAGTCTTGGAGTATCACGGAAAGAACCGTTGCATACACTAACCACACAGTACTTC
CTGAAGCTCTGGAGAAGTGGAGCTTGGACATTATGCAGAAACTTCTACCTCGACATGTTGA
GATTATAGAAAGAATAGATGAGGAGCTGATGAACACCATCGTCTCAAAATATGGAACGGCA
GATATTTCACTGTTAAAACAGAAGCTTAAAGATATGAGGATCTTAGACAACGTTGATCTTCC
AGCCTCTGTTGCTAAACTGTTTATTAAACCAAAAGAGAAAAAGGGGAAGTTGCTTGTTGAAT
CTTTGGAGTCTATTGCTGAAGCTGACGAGAAAACTGAGTCAGAAGAGGAGGAAAACATTCT
ATCCGAGACAACAGAGAAAAAGGGCGAATCTGACTCTGAAGAAGCTCCTGATGCAGAAAA
GGAGGATCCTGAGTATGAGTTAGATCCATTTACAAAATACGATCCTCAGTTACCTAGAGTTG
TTCGAATGGCGAACCTCTGCGTTGTTGGTGGACATTCAGTTAATGGTGTGGCTGAGATTCA
CAGTGAAATTGTTAAGCAAGATGTGTTCAATAGCTTTTATGAGATGTGGCCTACTAAGTTCC
AAAACAAAACAAATGGAGTGACCCCCAGGCGTTGGATCCGGTTTTGTAATCCTGAATTAAG
TACAATAATTTCAAAATGGATAGGCTCTGATGACTGGATTCTGAACACTGATAAACTTGCAG
GACTGAAGAAGTTTGCTGATGATGAGGATCTGCAATCAGAATGGCGTACTGCTAAAAGGAA
TAACAAGATGAAGGTAGTTTCGCTGATAAGAGACAAGACTGGATATGTTGTCAGCCCAGAT
GCAATGTTTGATGTGCAGGTGAAAAGAATACATGAATATAAGCGGCAGCTTCTAAATATCCT
TGGTATCATTTACCGCTACAAGAAGATGAAAGAAATGAGTGCAAAAGATAGAATAAAGAGC
TTTGTTCCAAGGGTATGCATATTTGGCGGGAAAGCTTTTGCCACTTACGTACAGGCAAAGA
GGATTGTGAAGTTTATCACAGATGTCGCAGCTACTGTAAATTATGATCCTGATATTGGAGAT
CTACTGAAGGTTGTATTTGTCCCAGATTATAATGTTAGCGTTGCTGAGACGCTCATTCCTGC
CAGTGAATTGTCCCAGCATATCAGTACCGCTGGAATGGAAGCTAGTGGAACCAGCAACAT
GAAGTTTGCAATGAATGGCTGTATTCTTATTGGAACTTTGGATGGTGCGAATGTGGAGATC
AGAGAAGAGGTTGGAGAGGAGAACTTTTTCCTCTTTGGTGCACATGCACCTGAAATCGCTG
GTTTGAGGCAAGAGAGAGCTGAAGGGAAGTTTGTGCCTGACCCGAGATTTGAAGAGGTTA
AGGAATACGTCCGCAGTGGCATCTTTGGGACTGGCAACTATGATGAATTGATGGGTTCTTT
GGAAGGAAATGAAGGTTATGGACGTGCAGATTATTTTCTTGTTGGCAAGGATTTTCCCAGT
TACATTGAATGCCAGGAAAAAGTTGATGAAGCATACCGAGATCAGAAGCTATGGACAAGAA
TGTCTATCCTCAACACAGCGGGTTCCCCCAAGTTCAGCAGTGACCGGACGATCCACGAGT
ATGCCAAGGACATATGGGGTATCAGCCCCGTCATCATGCCCTGA
SEQ ID NO: 3 >Wheat (hexaploid) PHS1 5A (Chinese Spring reference genome) protein
MATASPPLATPFRPLAVAGGSGGLVVGARAVVPPRRGRRGFVVRSVASDREVRGPAPTEEEL
SAVLTSIDSSAIASNIQHHADFTPLFSPEHSSPLKAYHATAKSVFDSLILNWNATYDYYNKVNAK
QAYYLSMEFLQGRALTNAIGNLELTGQYAEALKQLGQNLEDVASQEPDPALGNGGLGRLASCF
LDSMATLNYPAWGYGLRYRYGLFKQIITKDGQEEVAENWLEMGNPWEIVRNDVSYPLKFYGK
VVEGTDGRKHWIGGENIKAVAHDVPIPGYKTKTTNNLRLWSTTVPSQNFDLGAFNAGDHVKAN
EAHLNAEKICHVLYPGDESSEGKILRLKQQYTLCSASLQDIISRFESRAGDSLNWEDFPSKVAV
QMNDTHPTLCIPELMRILMDVKGLSWNESWSITERTVAYTNHTVLPEALEKWSLDIMQKLLPRH
VEIIERIDEELMNTIVSKYGTADISLLKQKLKDMRILDNVDLPASVAKLFIKPKEKKGKLLVESLESI
AEADEKTESEEEENILSETTEKKGESDSEEAPDAEKEDPEYELDPFTKYDPQLPRVVRMANLC
VVGGHSVNGVAEIHSEIVKQDVFNSFYEMWPTKFQNKTNGVTPRRWIRFCNPELSTIISKWIGS
DDWILNTDKLAGLKKFADDEDLQSEWRTAKRNNKMKVVSLIRDKTGYVVSPDAMFDVQVKRIH
EYKRQLLNILGIIYRYKKMKEMSAKDRIKSFVPRVCIFGGKAFATYVQAKRIVKFITDVAATVNYD
PDIGDLLKVVFVPDYNVSVAETLIPASELSQHISTAGMEASGTSNMKFAMNGCILIGTLDGANVEI
REEVGEENFFLFGAHAPEIAGLRQERAEGKFVPDPRFEEVKEYVRSGIFGTGNYDELMGSLEG
NEGYGRADYFLVGKDFPSYIECQEKVDEAYRDQKLWTRMSILNTAGSPKFSSDRTIHEYAKDI
WGISPVIMP
SEQ ID NO: 4 >Wheat (hexaploid) PHS1 5A (Chinese Spring reference genome) promoter
(−2 kb upstream of ATG)
CACCACGCAGCCTAGGGGCACCGTCTGGCCATCTGGAGCCCCGGTGGCTCCCTTGGACC
CCATCTTCTCGCTAGTGGGGCCTTGGTCCAAAATAATCCATGTAATTTTTCATGTTTTCCGG
CTTCGTATTTTTTTGGTAAAAACCCAAAAACAATAGAAAAGAAGAATTGGCCTCTGACACTA
GATTGATAGGTTAGTCCAAATCAATATTATCAAAAATTGTGCCCAAACCATATACAAATGATA
TAAATATAACTAGAATACTTTATAAATTTAGATACGTTTGATAAAGTGCCAAGAATGGAAGAG
AAGTGATCATATGCCGCCCTGTCCATATCGGGCTCATCAGAATGTTCCCACCATCAGACTC
CAGCCCCAGGATTATTAACTTATGTCGGCGGTGCGCTGAATGGATCTTGAAGAAGGCGGA
ATTTGTGTCGCCCTCACGTAACCAGGAGAACTTGGCAGGTTGACGCGCAATCGACCTCTC
CAGCAAAGCAAGTCCTAGGTAGGCGCGCTTCAGCTCGCAGAGTAACCGTACTTCTGGAGG
AGCAAGCGGGTGGTGATCTTGAGCCGCCGAGACGAGCAATCAATTACCGAGCGAGAAGC
ATCTGAGTGGAGATGTTACCGAGCGTTCTGGAACTCCAACTCTGCCGTCAGTGCATTGTCA
GCTTGTGCTTTGCGTAGATTCGCCGGAAAGGATCGGGGTCATGCACGCCAGAATTCCAGG
TGTCAGAGACCACCTAATGAAAACCATCGAGCTTTGATCAAAATAGCTCAAGATGGAATCT
CCGATCACCTTGGGAGCGAAAATCTTACTAGTTTCGTTCAAGGCCAAGGTAGTTATATATTT
GTGTGTGCAATTACTAATCATTGGCCGCTTAGCAAACTCTTATTGGTTCGATAAACGTTAAA
GTCATCCACTTTGAGAAAAAATGCTACTTGCTACAAACCCTTTCACTTTGGGAGGAGGGGG
GGTGGGTGGGGCAACATGTATCAGGATGATGGTGCCACCCTTGGCAAGGAATAAATCCTT
GCCGCCTCGTCCTTGTTTGGATGCTGCTTCTTGTCATCTCAAGGAGGCGTGTGGAGGGTG
GTGTGTCCCCGGATCTTGTTCTCTCGGTTGGGTTTTTTCTATGGCAGGTTTATCTCAGTGGT
AGTGTCACGTGAAGCAGATGACGCGACTATGCGTCTTGGTCCTCTGCGCTCTAATTCCGG
CAGATGAAGGTTGACCAGCTTCAGCCTCGTCGGGGAGCGTATGAGATTTGTGTTTTTTGAC
TCCATCTGGTCGGACCTGTGGCATTATGTAGACCCTCTCATGTATTTTTTTCTCCTGGACAG
TAGTCTGTTACACAAATCGTGGTCATCGGATCTTCTAGTCCACATCGGCGACTTTTTCGACC
GCCACTTCTACAAACTCCTTATTTTCAACAAGTTTCCTTCATTGAGATGGAAGTCCAGGCAA
TGGCTTTGCTCACGGCACACTGCCGGCGGATGCAAGAGAAAGAAGACATTCGACACCCCT
AAAACTATGAAGATGTAATTTTTAATCTTTGTAAGTGTTTTTGTGAGGATTTGTGCTACTTTT
AATATATTGCCTTTTGCCTTTTGCAAGGGAAAAAAACTAAACCATTACACGAAGAGAAGAGC
CATTCCCAGCCGTCCAATAAACACGACAAAACGCTTTACAGCACGGGGGCAATTCCCAGC
CGTCGAAATGCGCTGCAACGGAACCAGCGCTCCCCGACACGGCGGCACATGAGCCACAA
CTCGTACCATGCACCGTGCACCGGCGGTGGGCCCACTGGCCAGCGTCCCCACACGTGTA
CCGGCGGCCACAATTACACGGCACGGCACGGCGACGGAGAGATATTTTAACGCCTTCCAA
TTCAGAAGCTCCCCCATCACTCACTCGGGTACAGAGCGCAGCGGGCCGCGTGACTCTCGC
GTCGTCGCATCGCGCGCGCTGCACACCACCGTCAGCGCGACCACTGCCGCCA
SEQ ID NO: 5 >Wheat (hexaploid) PHS1 5B (Chinese Spring reference genome) genomic DNA
TTGCACGGCACGGCGACGGAGAGATATTTTTAACGCCTTCCAATTCAGAAGCTCCCCATCA
CTCGCTCGGGTATCAGAGCGGGCCGCGTGACTCTCGCGTCGTCGCATCGCGCGCGCTGC
ACACCACCGTCAGCGCGACCACTGGCGCCAATGGCGACCGCCTCGCCGCCGCTCGCCAC
CGCCTTCCGCCCGCTCGCCGCCGCCGGCGGCGCCGGAGGAGGAGGAGCGCACGCCGTG
GGCGCCGCGGGCCGGGTCGCGCCGAGGCGGGGGCGCCGGGGCTTCGTGGTGCGGAGC
GTGGCGAGCGATCGGGAGGTGCGGGGGCCCGCCTCGACCGAGGAAGGTAAGCGGCCGC
GCGCTCCCGTTGGCTACGACGACCCGGAATGCTGGTCGGCTGTAATGTGGTTCGGATTTT
CGCTGGTGCGACCTGAATTTGTGATCCTCTGACTGATGGTCTATGTGTTCCCGGGTTTCGG
GCTGTTGATTTTGTGCGCGCGCATTGCTAATCTACTGCTCTAGGATCTGCTAGGGTTCAGA
GGAATCTTAAATTCGTGAATAAGCTTCGACGAATGTATTCTGAATTTACTGAAACTGGCTTA
GTTTGGGGCGTCAAGTTAGAATGATGTATTTATGGCACAGTAGCAAATTCTGACAAATTCTT
CTGTGCGTGGACAGCCACCCCTTGTGGATGATGCAGCAAGTGTCAACATACTGTCGCTAC
TATCTGTAGCTGTAACAAAATCTTACGCCTTGTTTTCTGGTGGTACAGTATATTATCATTTTA
CAAAGAGTAGCTGTAACAAAATCTTACGCCTTGTTTTCTGGTGGGTTGCCATTGAGGAGTA
AGTAGAATGCAAATGGTGATACAGTATATTATCTACCATTTTGTGCAAGAATTTTCACACGG
AAACAATATCCCATGCAATTTTTAGTTTTGACTCTCGACTGTTTATACCTACTCTTTGTAAAA
TTTTGGATCCTAATATTGTTGTTCTTGCAACTCAGAGCTTTCAGCCGTGCTAACTTCCATTG
ATTCTTCCGCCATCGCGTCAAATATCCAGCACCATGCAGACTTCACACCGTTGTTCTCACC
AGAGCACTCTTCACCCCTAAAGGCTTACCATGCAACTGCTAAAAGTGTTTTTGATTCTCTGA
TCATCAATTGGAATGCAACATATGACTATTACAACAAAGTGAATGCAAAGCAAGCTTATTAC
CTGTCCATGGAGTTTTTGCAGGTCTCACAATATACTTCCTTTAGCCTTATCTCAATACCTTTT
GGTAACTGCGTTATGATGTTATATGGTTGTTATCTGTGTACTGCAGGGAAGAGCTCTCACA
AATGCTATTGGCAATCTAGAGCTAACAGGACAATATGCAGAAGCGTTAAAACAACTTGGAC
AGAACCTAGAGGATGTTGCTAGTCAGGTAAGTGATTTTTGTCGGAGATTGGTTTTTAGTCC
GTATCAAAATTATCGCAGGGCCATTGTTTTTATTTTGAAAACGCATTTTATTATTAATCATCA
AGTTCCCTTTGTTTAAAAACTATCTGAAAAGAAGAAGTTTTTTTCCTGAGGGTTCCACCGTC
TGACTTATGATCTTTATGCAGTTTTCAGAGTGACTATGAAATTACGCCAAAACACGATCTTAT
TGATGCCTAGAGTGCCCTGTAGTTTAGAAATCTAAGATTGGATATGTTACTTTTCTATTACAA
TGAAACAGGAACCAGATCCTGCCCTTGGCAATGGTGGTCTAGGCCGTCTAGCGTCCTGTT
TTCTGGATTCTATGGCAACCCTAAATTATCCAGCATGGGGATATGGACTTCGGTACAGATAT
GGCCTCTTTAAGCAAATCATAGCAAAGGATGGTCAGGAGGAGGTAGCTGAGAATTGGCTA
GAGGTAGTGATATGTCTTCTACTTGTTTACTGTTTGGAAGTGTTGATTATGTTGGTTTATTTA
TGTTCTAATGACCTGCATGTTAGCTTCTTTTCTATTTATAACTGATGCTGTATCTCCAGATGG
GAAATCCATGGGAGATCGTAAGAAATGATGTCTCTTATCCTGTGAAATTCTATGGCAAAGTG
GTTGAAGGCACTGATGGGAGAAAGCACTGGATTGGAGGAGAGAATATCAAGGCTGTGGCA
CATGATGTTCCTATTCCTGGCTACAAGACTAAAACTACTAATAATCTTCGTCTTTGGTCAAC
AACAGTACCATCACAAAATTTTGATTTGGGTGCCTTTAATGCTGGGGATCATGCCAAGGCC
AATGAAGCACATCTAAATGCTGAAAAGGTATTCTTTCGTCTACTTTATTTTGAATATAAACCA
TTAACTCTCAACAACTACTGATTCAAAGGCTAAACAGTGCTTTGCTAGTAGTTACTGTGTTTT
CTCTTAGATGTCATTTTATAATGTGTGCAGTCAATCGTTTGATATACACAAACCTATTTGGAT
TTCTCCTGTAAAAATGTTGTCACTAGATTTTTCATGAAAAAACTTATTGTATATGTAAATAGA
AAAGCTGAGGCTCAGGTGCTTTGTGTTCAAATGACAAGTAAAAATGAACAGAGTTTGTGCA
CCCATAACTTCTTCACCTGGTGTGTGCAATTCTTTCGGTTGTATTTATGCGTGTTCTTATTCT
GCCTGAATGATTCTCAGCATATGCATCAGGCTACATTTCTCCTCTGTGTCAAGAAAAAATAA
GTCTGACCAGATGCAGTCCAGTAGTCTTTAGATCTTTTCACATGGAAGCGTTACTCTATGAT
TTTTATTATACCTTTAGAGTGTTTGTGCATTGTGCACATATACCATGAAGTATGGGTGTTCAA
ATGTGGTTCTCTCAAACTGTGCTAAATCCACCACTGTTTGCCAGTATTCATTTTTCTGAACTA
GCAACAACTTGACTGGACAAGATACATTGTACTCACATCTGAAGCTGCTGGGTTGGACAGA
TTTGTCCGACCCGTCTTTGAACATCTCTACTATTTAGTGTGCATGGGTTAAATTGTTGACTTT
TCCTCGTTTGTTCAGTAGTAGGAGCACTATCTAATTATATTTATATTCAAGTTTTCTCTGTGT
GTTACGTTATAAACTTTTAGTCATACTTACATTTGTGGACAAAAGTTATCATGTCTAGCTTAA
GTTTGTGACTGATCTTCTCATCATGCCAGATATGCCACGTATTGTATCCAGGGGACGAATC
ATCAGAGGGGAAAATTCTTCGCTTGAAACAACAATATACATTATGCTCGGCCTCCCTGCAG
GACATTATTTCTCGTTTTGAGTCGAGGGCTGGTGATTCTCTCAACTGGGAGGACTTCCCCT
CTAAAGTTGCAGTTCAGATGAATGACACTCATCCAACACTGTGCATTCCGGAGTTAATGAG
AATACTGATGGATATAAAGGGATTGAGCTGGAATGAGGCTTGGAGTATCACAGAAAGGTAC
CACAGAAGGGTACTGCAGTTTGATGTGTTATGCTGTTCCTCCAGCTCCAATTAACTAATTGT
ATTGCCTCATCTGTCTTCCATTCAATTATGTACTGATTCTCTGATATCATTTGTAGAACCGTT
GCATACACTAACCACACGGTACTTCCTGAAGCTCTGGAGAAGTGGAGCTTGGACATTATGC
AGAAACTTCTACCCCGACATGTTGAGATCATAGAAACAATAGATGAGAAGGTAACAAATGT
GCAGATAATATTATGCAATAAATTTCATTCTCTGTAGTTCAACTTATGGTTTACTTATCAACA
TCGTTGATTTTAAAACAGCTGATGAACAACATCGTCTCAAAATATGGAACGGCAGATATTTC
ACTGTTAAAACAGAAGCTTAAAGATATGAGGATCTTAGACAACGTTGATCTTCCAGCCTCTG
TTGCTAAACTGTTTATTAAACCTAAAGAGAAAACGGGGAAGTTGCTTGTTCAATCTTTGGAG
TCTATTGCTGAAGGTGACGAGAAAACTGAGTCACAAGAGGAGGAAAACATTCTATCCGAGA
CAGCAGAGAAAAAGGGCGGATCTGACTCTGAAGAAGCTCCTGATGCAGAAAAGGAGGATC
CTGTGTATGAGTTAGATCCATTTGCAAAATACGATCCTCAGTTACCTAGAGTTGTTCGAATG
GCAAATCTCTGTGTTGTTGGTGGACATTCAGTTAATGGTGTGGCTGAGATTCACAGTGAAA
TTGTTAAGCAAGATGTGTTCAATAGCTTTTATGAGGTATTTGCACGATGATTTGGAAACCAT
GATCTTATTTTCTAACGGCTGCTATAATCGCCAGGTAATATGGTATCACCATCTGCCATTTT
CTTATGTTATTTGCCAGATGTGGCCTACTAAGTTCCAAAACAAAACAAACGGAGTAACTCCC
AGGCGTTGGATCCGGTTTTGTAATCCTGAATTAAGTGCAATCATTTCAAAATGGATAGGCTC
TGATGACTGGATTCTGAACACTGATAAACTTGCAGGACTGAAGAAGGTACTTCCAGTAATTT
TAATTTCCAAATATCAGTAATCTAATGCTTCCTTCACTTCCATGTTACCTGTGCCTTGTTTTT
ACATTGATGTGTGTTCATCCCGTAATAGTTTGCTGATGATGAGGATCTGCAATCAGAATGG
CGTACTGCTAAAAGGAATAACAAGATGAAGGTAGTTTCGCTGATAAGAGACAAGACTGGAT
ATGTTGTCAGCCCAGATGCGATGTTTGATGTGCAGGTATATTTTGGACTAGAAACATGTGT
CTCCTGTTAAATATGGTTTAACTCGGATCTTGGTACTTGACACTTAGGTGAAAAGAATACAT
GAATATAAGCGGCAGCTGCTAAATATCCTTGGTATCGTTTACCGATACAAGAAGATGAAAG
AAATGAGTGCCAAAGATAGAATAAAGAGCTTTGTTCCAAGGGTATGCATATTTGGTGGGAA
AGCTTTTGCCACTTACGTACAGGCAAAGAGGATTGTGAAGTTTATCACAGATGTCGCAGCT
ACTGTAAATTATGACCCTGATGTTGGAGATTTACTGAAGGTAAATTTGTGTCTTCAATGGCA
CTTATCATTCTAAATTTTCATGATCTGTCACTAGCAATTTGACTCCGACTAGGGGTTACTGA
AAAGCACTGAATGTTCCGAGTTTCAAAAGAAAACAGAGCAGTGAATGTTCTTAATGTCGGT
GCTTGTAGAACTGAGAAACTGAATAGCCACAGTTTTTTTTTAGAGAACAATAGCCACAATTG
TTCTGATGCTGATTTTGCTGATAACTAGATGAGGTAAATACAAGGTGACAGGTTAAGTTCTG
AGGATTTTTGTTTGTTATCCAGGTTGTATTTGTCCCAGATTATAATGTTAGCGTTGCTGAGA
AGCTCATTCCTGCCAGTGAATTGTCCCAGCATATCAGGTAATATTCTGGCTACTCTTATATA
GTGCTCCCTCCGTCCCAAAATTCTTGTCTTAGATTTGTCCAAATACGGATGTATCTAGTTAC
ATTTTAGTGTTAGATACATCCGTATCTAGACAAATGTAAGAGAAGAATTTTGGGACGGAGG
GAGTATGTGAATACAAAGAAAAGGAGTCTTATCTGGAGGTGACTGATGTGGAAATTTTCAT
GTGCCTTTTTGTTTGCTACTCCCTCCGTCCCATAATATAAGAGCGTTTTTGACAGTACACTA
TTTTCAAAAATGCTCTTATAATATGGGACGGAGGGAGTATAATATATACCTGGAGTCTTGCT
GGGATAGTGACATCCAGAAGTGCCACGCCAGCTTCTATTTCTGTACAAGACGTGCATTTTT
AACACTATTACTATCCATTCAGTACCGCTGGAATGGAAGCTAGTGGAACCAGCAACATGAA
GTTTGCAATGAATGGTTGTATTCTTATTGGAACTTTGGATGGTGCGAATGTGGAGATCAGA
GAAGAGGTTGGAGAGGAGAACTTTTTCCTCTTTGGTGCACATGCACCTGAAATCGCTGGTT
TGAGGCAAGAGAGGGCTGAAGGAAAGGTACCACACTTGTTTATATAGCTGTGCCTACTATC
AAACTAGTAAAGATATCTCCCATATGTGTGCTGTCCAAATAGTTTAGCTGAAGGAAAATCAT
AACTTTATCATTTTCCTTTGCACTTGTAGTTTGTGCCTGACCCGAGATTTGAAGAGGTTAAG
GAATACGTCCGCAGTGGCGTCTTTGGGACTAGCAACTATGATGAATTGATGGGTTCTTTGG
AAGGAAATGAAGGTTATGGACGTGCAGATTATTTTCTTGTTGGCAAGGATTTTCCCAGTTAC
ATTGAATGCCAGCAAAAAGTTGATGAAGCATACCGAGATCAGAAGGTAAGTTGTATAACTG
ACGCTGTTTGTTTTCTGGCTGAAATGCATACGGTGATTATGCAAACTTTCTGATATGTGATG
GCTGACATCAGTGTTTCACTGTTAATGACTTTACTGATTCGTAGGAAGGAATATTTCTTAATT
CAAAGTTTCAAACCGGAGTATCTATCGATGTGTTGGAACTTGGAAGATCATAAACAATCAGA
GAATATAGTTGCTAGATGTGCTATACTACTCATCTGTTGGCTGTTGAGTGTTGATACATGAG
CTTTGATAAACCTAGCTAGACCTTCAGGAAAGTGCTTCTATGCATTCAATAAAAAAATAACTT
TATAACTTAACAAAATAATTAGGTTGAACCTCGCCAGGGGGGCCCTTTTGTAATCTGTTTTC
TGAAATGAAAATCAGAAGCAGGGAGCTCCTCGATTCTAAAATGAAAAATAAAATCAGGGAT
AACTGAGGGTCTGGTTTCCCTCTGTTGGAGCCCCAGAGAATTCTGCGGTCTTATTTTTGTA
AGAGTATAAGATGCACTGCTCCGTTAAGTTTCTCCTTCATCCATTTGTTTTCTTTGCTTCGTG
ACATTTGGTGTAACAGAGCATCCCATTTGTACACATTATTTTGCTATCGTGGATATTTCCACT
CTGTTTCATGTGCGACATTTTAAATTCGTTACATCGTTACTAACTACACTTAGTCTGATAGAT
TTTCAGAGTTTTGGTCCTCCTAATAACATAATATCCGTTTCTACCTGCAGCTATGGACAAGA
ATGTCTATCCTCAACACAGCGGGTTCCCCCAAGTTCAGCAGTGACCGGACGATCCATGAG
TACGCCAAGGACATATGGGATATCAGCCCCGTCATCATGCCCTGAATAATTCAAGCAGCTT
CCAGGAACCTTGAGAGTCTATGAGGCGCGATTGGTCGCATATTTGCCTCGATTCAGCGAG
AATGCATTGCTCCCCCCCAGTTTTGTATAGGCATATGCAAGTTCTGACATGGACCTGAGTG
CTGAGAGCATGCAGGGTAGTTAATCCTTAGCAATCTTGCGTGTCAGTTGGCCAGTATTACC
CAGGCTTATGGTGTGTGTATTTCGCCGAATAAAGTTGGAGCAGAATCATCAAACAAACATA
CTTTGGTGTATCTGTAATGATAAATAAATGGTAAATGACTGAACAAAAAGTGTCGTTGGCGC
ACACATGTAGTAGAAGCATGTTTCTTCAGAGAGGGTGCAAAGCTGAAAGCACCCCAACTTT
GAAGTGAGAATGCAGAAGTCACGAAAACATTTGTTTACATCCAAAGTGCAATGGGATTCGT
CAAGAGTTCAGAATCTTCCCTATAATAGAGTAGTAGCAGTGGTCCCTCACATTTTGA
SEQ ID NO: 6 >Wheat (hexaploid) PHS1 5B (Chinese Spring reference genome) CDS
ATGGCGACCGCCTCGCCGCCGCTCGCCACCGCCTTCCGCCCGCTCGCCGCCGCCGGCG
GCGCCGGAGGAGGAGGAGCGCACGCCGTGGGCGCCGCGGGCCGGGTCGCGCCGAGGC
GGGGGCGCCGGGGCTTCGTGGTGCGGAGCGTGGCGAGCGATCGGGAGGTGCGGGGGC
CCGCCTCGACCGAGGAAGAGCTTTCAGCCGTGCTAACTTCCATTGATTCTTCCGCCATCGC
GTCAAATATCCAGCACCATGCAGACTTCACACCGTTGTTCTCACCAGAGCACTCTTCACCC
CTAAAGGCTTACCATGCAACTGCTAAAAGTGTTTTTGATTCTCTGATCATCAATTGGAATGC
AACATATGACTATTACAACAAAGTGAATGCAAAGCAAGCTTATTACCTGTCCATGGAGTTTT
TGCAGGGAAGAGCTCTCACAAATGCTATTGGCAATCTAGAGCTAACAGGACAATATGCAGA
AGCGTTAAAACAACTTGGACAGAACCTAGAGGATGTTGCTAGTCAGGAACCAGATCCTGCC
CTTGGCAATGGTGGTCTAGGCCGTCTAGCGTCCTGTTTTCTGGATTCTATGGCAACCCTAA
ATTATCCAGCATGGGGATATGGACTTCGGTACAGATATGGCCTCTTTAAGCAAATCATAGC
AAAGGATGGTCAGGAGGAGGTAGCTGAGAATTGGCTAGAGATGGGAAATCCATGGGAGAT
CGTAAGAAATGATGTCTCTTATCCTGTGAAATTCTATGGCAAAGTGGTTGAAGGCACTGAT
GGGAGAAAGCACTGGATTGGAGGAGAGAATATCAAGGCTGTGGCACATGATGTTCCTATT
CCTGGCTACAAGACTAAAACTACTAATAATCTTCGTCTTTGGTCAACAACAGTACCATCACA
AAATTTTGATTTGGGTGCCTTTAATGCTGGGGATCATGCCAAGGCCAATGAAGCACATCTA
AATGCTGAAAAGATATGCCACGTATTGTATCCAGGGGACGAATCATCAGAGGGGAAAATTC
TTCGCTTGAAACAACAATATACATTATGCTCGGCCTCCCTGCAGGACATTATTTCTCGTTTT
GAGTCGAGGGCTGGTGATTCTCTCAACTGGGAGGACTTCCCCTCTAAAGTTGCAGTTCAG
ATGAATGACACTCATCCAACACTGTGCATTCCGGAGTTAATGAGAATACTGATGGATATAAA
GGGATTGAGCTGGAATGAGGCTTGGAGTATCACAGAAAGAACCGTTGCATACACTAACCA
CACGGTACTTCCTGAAGCTCTGGAGAAGTGGAGCTTGGACATTATGCAGAAACTTCTACCC
CGACATGTTGAGATCATAGAAACAATAGATGAGAAGCTGATGAACAACATCGTCTCAAAATA
TGGAACGGCAGATATTTCACTGTTAAAACAGAAGCTTAAAGATATGAGGATCTTAGACAAC
GTTGATCTTCCAGCCTCTGTTGCTAAACTGTTTATTAAACCTAAAGAGAAAACGGGGAAGTT
GCTTGTTCAATCTTTGGAGTCTATTGCTGAAGGTGACGAGAAAACTGAGTCACAAGAGGAG
GAAAACATTCTATCCGAGACAGCAGAGAAAAAGGGCGGATCTGACTCTGAAGAAGCTCCT
GATGCAGAAAAGGAGGATCCTGTGTATGAGTTAGATCCATTTGCAAAATACGATCCTCAGT
TACCTAGAGTTGTTCGAATGGCAAATCTCTGTGTTGTTGGTGGACATTCAGTTAATGGTGTG
GCTGAGATTCACAGTGAAATTGTTAAGCAAGATGTGTTCAATAGCTTTTATGAGATGTGGCC
TACTAAGTTCCAAAACAAAACAAACGGAGTAACTCCCAGGCGTTGGATCCGGTTTTGTAAT
CCTGAATTAAGTGCAATCATTTCAAAATGGATAGGCTCTGATGACTGGATTCTGAACACTGA
TAAACTTGCAGGACTGAAGAAGTTTGCTGATGATGAGGATCTGCAATCAGAATGGCGTACT
GCTAAAAGGAATAACAAGATGAAGGTAGTTTCGCTGATAAGAGACAAGACTGGATATGTTG
TCAGCCCAGATGCGATGTTTGATGTGCAGGTGAAAAGAATACATGAATATAAGCGGCAGCT
GCTAAATATCCTTGGTATCGTTTACCGATACAAGAAGATGAAAGAAATGAGTGCCAAAGATA
GAATAAAGAGCTTTGTTCCAAGGGTATGCATATTTGGTGGGAAAGCTTTTGCCACTTACGTA
CAGGCAAAGAGGATTGTGAAGTTTATCACAGATGTCGCAGCTACTGTAAATTATGACCCTG
ATGTTGGAGATTTACTGAAGGTTGTATTTGTCCCAGATTATAATGTTAGCGTTGCTGAGAAG
CTCATTCCTGCCAGTGAATTGTCCCAGCATATCAGTACCGCTGGAATGGAAGCTAGTGGAA
CCAGCAACATGAAGTTTGCAATGAATGGTTGTATTCTTATTGGAACTTTGGATGGTGCGAAT
GTGGAGATCAGAGAAGAGGTTGGAGAGGAGAACTTTTTCCTCTTTGGTGCACATGCACCT
GAAATCGCTGGTTTGAGGCAAGAGAGGGCTGAAGGAAAGTTTGTGCCTGACCCGAGATTT
GAAGAGGTTAAGGAATACGTCCGCAGTGGCGTCTTTGGGACTAGCAACTATGATGAATTGA
TGGGTTCTTTGGAAGGAAATGAAGGTTATGGACGTGCAGATTATTTTCTTGTTGGCAAGGA
TTTTCCCAGTTACATTGAATGCCAGCAAAAAGTTGATGAAGCATACCGAGATCAGAAGCTAT
GGACAAGAATGTCTATCCTCAACACAGCGGGTTCCCCCAAGTTCAGCAGTGACCGGACGA
TCCATGAGTACGCCAAGGACATATGGGATATCAGCCCCGTCATCATGCCCTGA
SEQ ID NO: 7 >Wheat (hexaploid) PHS1 5B (Chinese Spring reference genome) protein
MATASPPLATAFRPLAAAGGAGGGGAHAVGAAGRVAPRRGRRGFVVRSVASDREVRGPAST
EEELSAVLTSIDSSAIASNIQHHADFTPLFSPEHSSPLKAYHATAKSVFDSLIINWNATYDYYNKV
NAKQAYYLSMEFLQGRALTNAIGNLELTGQYAEALKQLGQNLEDVASQEPDPALGNGGLGRLA
SCFLDSMATLNYPAWGYGLRYRYGLFKQIIAKDGQEEVAENWLEMGNPWEIVRNDVSYPVKF
YGKVVEGTDGRKHWIGGENIKAVAHDVPIPGYKTKTTNNLRLWSTTVPSQNFDLGAFNAGDHA
KANEAHLNAEKICHVLYPGDESSEGKILRLKQQYTLCSASLQDIISRFESRAGDSLNWEDFPSK
VAVQMNDTHPTLCIPELMRILMDIKGLSWNEAWSITERTVAYTNHTVLPEALEKWSLDIMQKLLP
RHVEIIETIDEKLMNNIVSKYGTADISLLKQKLKDMRILDNVDLPASVAKLFIKPKEKTGKLLVQSL
ESIAEGDEKTESQEEENILSETAEKKGGSDSEEAPDAEKEDPVYELDPFAKYDPQLPRVVRMA
NLCVVGGHSVNGVAEIHSEIVKQDVFNSFYEMWPTKFQNKTNGVTPRRWIRFCNPELSAIISK
WIGSDDWILNTDKLAGLKKFADDEDLQSEWRTAKRNNKMKVVSLIRDKTGYVVSPDAMFDVQ
VKRIHEYKRQLLNILGIVYRYKKMKEMSAKDRIKSFVPRVCIFGGKAFATYVQAKRIVKFITDVAA
TVNYDPDVGDLLKVVFVPDYNVSVAEKLIPASELSQHISTAGMEASGTSNMKFAMNGCILIGTL
DGANVEIREEVGEENFFLFGAHAPEIAGLRQERAEGKFVPDPRFEEVKEYVRSGVFGTSNYDE
LMGSLEGNEGYGRADYFLVGKDFPSYIECQQKVDEAYRDQKLWTRMSILNTAGSPKFSSDRTI
HEYAKDIWDISPVIMP
SEQ ID NO: 8 >Wheat (hexaploid) PHS1 5B (Chinese Spring reference genome) promoter
(−2 kb upstream of ATG)
TCATCCTCGGTGAAGGGCTGCTCAAGTGCAGGGAGGTCAAAGTGATGTGGGTCAATGCTA
TGGGGATCAAGGGAGAGGGTCGGTGCTCAAAAGTGCCAAGAATGGAAGAGAAGTGATCAT
ATACCGCCTTGGCCATATTGGGCTCGCTGGAATGTGCCCACCATCAGGCTGCAGCCCTAG
AATCATTAACTTCTGTCGCTAGTGCGTGGAATGGATCTTGAAGAAGGCGGAATTTGTGTCG
CCCTCACGTAGCCAGGAGAACTTGACAGGGACCTCTCCAGTGAAGCAAGTCCTAGGTAGG
CGCGCTTCAGCTCGCGGAGTAACTAGACTTCTGGAGGAGCTACCAGGTGGTGATCTTGAG
CCGCATCGAGACGAGCGGTCTACTCCCGAGCGAGAAGCATCTGAGTGGACATGTTACTGA
GCGTTCTGGGGATCCAACTCTATAGTCAGCGCATTGTCAGCTTGAGCTTTGCGTAGATCCA
TCGGAAAGGATCGGGGTCATGCACGCTAGAATTCCAGGTGTCAGAGACCACCTGACTAAA
ACCATCGAGCTTTCGCCAAAATAGCTGAAGATGGAATCTTTGATCACCTTGGGAGCAAAAA
TTTCATTGGAGGCCAAGTTAGTTCTATATTTGTGTCTGCAATTACTGATCATTGGCCGCTTA
GCAAACTCCTATTGGTTCGGTAAAGCTTAAGGTCATCCACTTTGGAAAAATGCTACTTGCTA
CAAACCCTCTCANNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNTGGCTTTTCTAGTTGAGAGGAAGCAAAGACGTATCAAGACGACAATATCACCCTC
GGCAAGGAATAAGTCCTTCCCGCCTCGTCCTCGTTTCGATGCTGCTTCTTGTCATTGCGGT
GAGGCGCGTGGAGGTTGGCGTGTCCCCAAATCTAGTTATCTGGGTCGGGGTTTTTCTTTG
GCAGGTTTATCTCAGTGGTAGTGTCCCGTGAAGGGGATGACACGACTATGTGTCTTGGTC
CTCCATGCTCTAGTTTTGACATGTGAAGGTTGGCCAACTTCAGCGTTGTCGGGGAGCATAT
GAGATTTGTGTTCCTTTACTTCGCCTGGTCAGACCTGCGGTATTATATAAGCCCTCTCATGT
GTTTTCTTCTCTTGGGCAGCAGTCAGTTACGCAAATCGTGGTCGCTGACATCTTCTAGTCC
ACATTGGCGACTTCCCGACCGCCGCTTCTACAAGCTCCTTATTTTTCAACAAGTTTTCTCCA
CCGAGATGGAAATCAAGAGGCGGCAACGGCTTTGCTCATGGCGCACTGCCCATGGATGCA
GGAGAAGGAAGACATTCCACACCCCTCAAACTATGAAGATGTAATTTTTAGTTTTTGTAAGT
ATTTTTGTGAGGATTTGTGCTACTTTTAATATATATGGCCTTTTGCCTTTTTCCAGGAAAGGA
AAAACTAAACCATTACATGAGGCCATTCCCATCCGTCCAATAAATACGACAAAACGCTTTAC
AGCACGGGGGCAATTCCCAGCCGTCGAAATGCGCTGCAACGGAACTAGCTCTCCCAGACA
GACACAGCGGCACATGAGCCACGCCTCCCATCGCAACCGTGCACCGGCGGTGGGCCCAC
CGGCCAGCGCCCCCACACGTGTACCGGCGGCCAGCATTGCACGGCACGGCGACGGAGA
GATATTTTTAACGCCTTCCAATTCAGAAGCTCCCCATCACTCGCTCGGGTATCAGAGCGGG
CCGCGTGACTCTCGCGTCGTCGCATCGCGCGCGCTGCACACCACCGTCAGCGCGACCAC
TGGCGCCA
SEQ ID NO: 9 >Wheat (hexaploid) PHS1 5D (Chinese Spring reference genome) genomic DNA
AGTGGTAGTGTCTCGTGAAAGCGGATGACGCGACTATGTGTCTTGGTCCTCCACGCTCTA
GTTTCGACAGGTGAAGGTTGGCCAGCTTCGGCCTCGTCGGGGAGCGTATGAGATTTGTGT
TCCTTTACTTCGTCTGGCCAGACTTCCCGACCGCCGCTTCTACAAGCTCCTTATTTCAAGC
AAGTTTTCTTCACCGAGATGGAAGTCCAGAGGCGGCAACAGCTTTGCTCACGGCGCAGTG
GCGGTGGATGCAGGAGAAAGAAGACATTCAACACCCCTAAACTATGAAGATGTAATTTTTT
ATTTTTGTAAGTGCTTTTGTGAGGACTTGTGCTACTTTTAATATGTGGCCTTTTGCCTTTTTC
CAGAAAAAAAAAACTAAACCATTACACGAGGAGAAGAGCCATTCCCAGCCGTCCAATAAAC
ACGACAAAACGCTTTACAGCACGGGGGCAGTTCCCAGCCGTCGAAATGCGCTGCAACGGA
ACTAGTAGCTCTCCCTGACACGGCGGCACATGAGCCACGCCTTGCGCCGTGCACCGGCG
GTGGGCCCACCGGCCAGTGCCCCCACACGTGTACCGGCGGCCAGCATTGCACGGCACG
GCGACGGAGAGATATTTTTAACGCCTTCCAATTCAGAAGCTCCCCCATCACTCACTCGGGT
ACAGAGCGGGCCGCGTGACTCTCGCGTCGTCGCATCGCGCGCGCTGCACACCACCGTCA
GCGCGACCACTGCCACCAATGGCGACCGCCTCGCCGCCGCTCGCCACCGCCTTCCGCCC
GCTCGCCGCCGGCGGCGGCACCGGAGGAGGAGGAGCGCACGCCGCGGGCTGGGTCGC
GCCGAGGCGGGGGCGCCGGGGGTTCGTGGTGCGGAGCGTGGCGAGCGATCGGGAGGT
GCGGGGGCCGGCCTCGACCGAGGAAGGTAAGCGCCGCGCGCTCCCGTTGGCTACGACG
ACCCGGAATGCTGGTCGGCTGTAATGTGGTTCGGATTTTCGCTGGTGCGACCTGAATTTGT
GATCCTCTGACTGACGGTCTCTGTGTTCCCCGGTTTCGGGCTATTGATTTTGTGCGCGCGC
ATTGCTAATCTACTGCTCTAGGATTTGTTAGGGTTCAGAGGAATCTTAAATTCGTGCATAAA
CTTCGACGAATGTATTCTGAATTTACTGAAACTGGCTTAGTTTGGCGCATCAAGTTAGAATG
ATGTATTTATGGTACAGTAGCAAATTCTGAGAAATTCTTCTGTGCCTGCACAGCCACCCCTT
GTGGATGATGCAGCAAGTGTCAACATACTGTCGCTACTATCTGTAGCTGTAACAAAATCTTA
CGCCTTGTTTTCTGGTGGTACAGTAGTATTATCATTTTACAAAGAGTAGCTGTAACAAAATC
TTACGCCTTGTTTTCTGGTGGCTTGCCATTGGGGAGTAGAATGCAAATGGTGATACAGTAT
ATTATCTACGATTTTGTGCAAGAATTTTCACACGAAAACAATGTCCCATGCAGTTTTTAGTTT
TGACTCTTGACTGTTTAATACCTACTCTTTGTAAGATTTCGGATCCTAATATTGTTGTTCTTG
CAACTCAGAGCTTTCAGCCGTGCTAACTTCCATTGATTCTTCCGCCATCGCGTCAAATATCC
AGCACCATGCAGACTTCACACCGTTGTTCTCGCCAGAGCACTCTTCACCCCTAAAGGCTTA
CCATGCAACTGCTAAAAGTGTTTTTGATTCTCTGATCATCAATTGGAATGCAACATATGACT
ATTACAACAAAGTGAATGCAAAGCAAGCTTATTACCTGTCCATGGAGTTTTTGCAGGTCTCA
CAATATACTTCCTTTAGCCTTATCTCAATACTTTTTGGTAACTGCGTTATGATGTTATATGGT
TATTATCTGTGTACTGCAGGGAAGAGCTCTCACAAATGCTATTGGCAATCTAGAGCTAACA
GGACAATATGCAGAAGCGTTAAAACAACTTGGACATAACCTAGAGGATGTTGCTAGTCAGG
TAAGTGATTTTTGTCAGAGATTGGTTTTTAGTCTGTATCAAAATTATCGCAGAGCCATTTTTT
TTTTCTAAAAAACACATTTTATTATTAATCATCAAGTTCCTTTTGTTTAAAAACTATCTGAAAA
GAAGAAGTATTTTCCCCTGGGGGTTCCAGCATCTGACTTATATTCTTTATGCAGTTTTCAGA
GTGAGTGACTATGAAATTACGCCAAAACACGAGCTTATTGATGCCTAGAGTGCCCTGTAGT
TTAGAAATCTATTATTGGATATGTTACTTTTCTATTACAATGAAACAGGAACCAGATCCTGCC
CTTGGCAATGGTGGTCTAGGCCGTCTAGCGTCCTGTTTTCTGGATTCTATGGCAACCCTAA
ATTATCCAGCATGGGGATATGGACTTCGGTACAGATATGGCCTCTTTAAGCAAATCATAACA
AAGGATGGTCAGGAGGAGGTAGCTGAGAATTGGCTAGAGGTAGTGATATGTCTTCTACTTG
TTTACTGTTTCGAAGTGTTGACTATGTTGGTTTATGTTTTAATGACCTGCATGTTAGCTTCTT
TTGCTGTTATAACTGATGCTGTATCTCCAGATGGGAAATCCATGGGAGATTGTAAGAAATGA
TGTCTCTTATCCTGTGAAATTCTACGGCAAAGTGGTTGAAGGGACTGATGGGAGAAAACAC
TGGATTGGAGGAGAGAATATTAAGGCTGTGGCACATGATGTTCCTATTCCTGGCTACAAGA
CTAAAACTACCAATAATCTTCGTCTTTGGTCAACAACAGTACCATCACAAAATTTTGATCTG
GGAGCTTTCAATGCTGGGGATCATGCCAAGGCCAATGAAGCACATCTAAATGCTGAAAAG
GTATTCTTCCGTCTACATTTATTTTGAACGTAAACCATTAACTCTCAACAACTATTGATTCAA
AGGCTAAACACTGTCTTGCTAGTAGTTACTGTGTTTTCTCTTAGATGTCATTTTAGAATGTG
CGCGGTCAATCGTTTGATAACTTTGATAGTAATAATATACTGGTATACACAAACCTATTTGG
GTTTCTCATGTAAAAATGTCGTCACCAAATTTTTCATGAAAATACTTATTGTATTTTCATGTAA
ATAGAAAAGCTGATGGTCAGGTGTTTTGTGTGTCAAATGACAAGTAAAAATGAACAGAGTTT
GTGCACCAATAACTTCTTCACCTGGTGTTTGTAATTCTTTCGGTTGTATTTATGTGTTTTCTT
ATTCTGCCTTAATTAACTCAGCATATGCATCAGGTTACATTACTCCTCTGTGTCAAGAAAAA
AGAAGTCTGATGAGATGCAGTCCAGTAGTCTGTAGATGGCACATTGTAGAAGAACTTTTCA
CATGGAAACGTTACTCTGTGATTTTTGTTATGCCTTTTGAGTGTTTGTGCATTGTGCACATAT
ACCATTAAGTATAAGTGTTCAAAAGTGGTTCTCTCATACTGTGCAAACTAGCTAAACAACTT
GACTGGACAAGATAAATTGTACTCACATCTGAAGCTGCCGGGTTGGACAGATTTGTCCGAA
CCATCTTTGAACATCCCTACTAGTGTGCATGGGTTAAATTGTTGACTTTTCCTCGTTTGTTC
AGTAGTAGGACTATCTAAATATATTTATATTCAAGTTTTCTTTGTGTGTTACGTTATAAACTTT
TAGTCATAATTACATTTGTGACCATTGTGGACAAAATTTATCATGTCTAACTTAAGTTTGTGA
CTGGTCTTCTCATCATGCCAGATATGCCACGTATTGTATCCAGGGGACGAATCATCAGAGG
GGAAAATTCTTCGCTTGAAACAACAATATACATTATGCTCGGCCTCCCTGCAGGACATTATT
TCTCGTTTTGAGTCGAGGGCTGGTGATTCTCTCAACTGGGAGGACTTCCCCTCTAAAGTTG
CAGTTCAGATGAATGACACTCATCCAACACTGTGCATTCCCGAGTTAATGAGAATACTGAT
GGATATAAAGGGATTGAGCTGGAATGAGGCTTGGAGTATCACAGAAAGGTACCACAGAAG
GGTACTGCAGTTTGATGTGTTCTGCTGTTCCTCCAGTTTCAATTAACTAATTTTATCCCTCAT
ATGTCTTCCATTCAATAATAAACTGATTGTCTGATATCATTTGTAGAACCGTTGCATACACTA
ACCACACGGTACTTCCTGAAGCCCTGGAGAAGTGGAGCTTGGACATTATGCAGAAACTTCT
ACCTCGACATGTTGAGATCATAGAAACAATAGATGAGGAGGTAACAAATGTGCAGATAATA
TTATACAATTAATTTCATGCTCTGTAGGTCAACTTATGGTTTACTTATCTTCATCGTTGATTTT
AAAACAGCTGATGAACAACATCGTCTCAAAATATGGAACGGCAGATATTTCACTGTTAAAAC
AGAAGCTTAAAGATATGAGGATCTTAGACAACGTTGATCTTCCAGCCTCTGTGGCTAAACT
GTTTGTTAAAACAAAAATGAAAAAGGGGAAGTTGCTTGTTGAATCTTTGGAGTCTATTGCTG
AAGCTGACGAGAAAACTGAGCCAGAAGAAGAGGAAAACATTCTATCCGAGACAACAGAGA
AGAAGGGCGAGTCTGACTCTGAAGAAGCTCCTGATGCAGAAAAGGAGGATCCTGAGTATG
AGTTAGATCCATTTGCAAAATACGATCCTCAGTTACCTAGAGTTGTTCGAATGGCAAACCTC
TGTGTTGTTGGTGGACATTCAGTTAATGGTGTGGCTGAGATTCACAGTGAAATTGTTAAGC
AAGATGTGTTCAATAGCTTTTATGAGGTATTTGCACGATGATTTGGAAACCATGATCTTATC
TTCTAACGGCTGCTATAATCGCCAGGTAATATGGTATTCACCATCTGCCATTTTCTTGTGTT
CTTTGCCAGATGTGGCCTACTAAGTTCCAAAACAAAACAAACGGAGTAACTCCCAGGCGTT
GGATCCGGTTTTGTAATCCTGAATTAAGTGCAATCATTTCAAAATGGATAGGCTCTGATGAC
TGGATTCTGAACACTGATAAACTTGCAGGACTGAAGAAGGTACTTCCAGTAATTTTAATTTC
CAATAATCAGTAATCTAATGCTTCCTTCACTTCCATGTTACCCGTGCCTTGTTTTTACATTGA
TATGTGTTCATCCCGTAATAGTTTGCTGATGATGAGGATCTGCAATCAGAATGGCGTACTG
CTAAAAGGAATAACAAGATGAAAGTAGTTTCACTGATAAGAGATAAGACTGGATATGTTGTC
AGCCCAGATGCGATGTTTGATGTGCAGGTATATTTTGGACAAGAAACATCTGTCTTCTGTTA
AATATGGTTTAACTCGGATCTTGGTACTTGACACTTAGGTGAAAAGAATACATGAATATAAG
CGGCAGCTGCTAAATATCCTTGGTATCGTTTACCGGTACAAGAAGATGAAAGAAATGAGTG
CAAAAGATAGAATAAAGAGCTTTGTTCCAAGGGTATGCATATTTGGTGGGAAAGCTTTTGC
CACTTATGTACAGGCAAAGAGGATTGTGAAGTTTATCACAGATGTCGCAGCTACTGTAAATT
ATGATCCTGATATTGGAGATTTACTGAAGGTAAATTTGTGTCTTCAATGGCACTTATTATTCT
TAATTTTCATGTTCTGTCACTGGGAATTTGACTCCGACTAGGGGTTACTGAAAAGCAGTGAA
TGTTCCGAGTTTCAAAAGAAAACAGAGCAGTGAATGTTCTTAATGTCGGTGCTTGTAGAAC
CGAGAAACTGAATAGCCACAGTATTTTTTAGAGAACAATAGCCACAATTGTTTTGACCCTTC
TATTGCTATCAAGCATCTGATGCCGATTTTACTGATAACTAGATGAGGTAAATACAAGGTGA
CAGGTTAAGTTCTGAGGATTTTTGTTTGTTATACAGGTTGTATTTGTCCCAGATTATAATGTT
AGCGTTGCTGAGAAGCTCATTCCTGCCAGTGAATTGTCTCAGCATATCAGGTAATATTCTG
GCTACTCTTATATAGTATGTGAATACATAGAAAAGGAGTCTTACCTGGAGTTGGCTGATGTG
GAAATTTTCATGTGCCGTTTTGTTTGCTATAATATATACCTGGAGTCTTGCTGGGATAGTGC
CATCCAGAAGTGCCACGCCAGCTTCTATTTCTGTACAGGATGTGCATTTTTAACACTATTAC
TATCCATTCAGTACCGCTGGAATGGAGGCTAGTGGAACCAGCAACATGAAGTTTGCAATGA
ATGGTTGTATTCTTATTGGAACTTTGGATGGTGCGAATGTGGAGATCAGAGAAGAGGTTGG
AGAGGAGAACTTTTTCCTCTTTGGTGCACATGCACCTGAAATCGCTGGTTTGAGGCAAGAG
AGGGCTGAAGGAAAGGTACCATCACTTTGCAAACATCTCCAAATTCGTTCCTCCAGTATGT
GAAACAAATATCCACTTGTTTATATAGCTGTGCCTACTATCAAACTACTCCCTCCGTCCATA
ATATAAGAGTGTTTTTTACACTACACTAGTGTCAAAAACGCTCTTATATTATGGGACGGAGG
GAGTAGTAAAGATCTCTCCCAAATGTGTGCTGTCCAAATAGTTTAGCTGAAGGAAAATTATA
AATCATTTTCCTTTGCACTTGTAGTTTGTGCCTGACCCGAGATTTGAAGAGGTTAAGGAATA
CGTCCGCAGTGGCGTCTTTGGGACTAGCAACTATGATGAATTGATGGGTTCTTTGGAAGGA
AATGAAGGTTATGGACGTGCAGATTATTTTCTTGTTGGCAAGGATTTTCCCAGTTACATTGA
ATGCCAGGAAAAAGTTGATGAAGCATACCGAGATCAGAAGGTAAGTTGTATAACTGACTCT
GTTTGTTTTCTGGCTGAAATGCACACAGTGATTATGCAAACTTTCTGATATGTGATGGCTGA
CATCAGTGTTTCACTGTGAACGACTTCACTGATTCGTAGGAAGGAATATTTCTTAGTTCAAA
GTTTCAAACTGAAGTATCTATCGGTGTGTTGGAAGATCATAAACAATCAGAGAATATAGTTG
CTCGATGTGCTATATTACTCATCTGTTGGCTGTTGAGTGTTGATACATGAGCTTTGATGAAC
CTAGCTAGACCTTCGGGAAAGTGCTTCTATGCATTCCAATAAAAAAAACTTATATCTTAACA
AAACAATTAGGTTGAACCTCGCCAGGGGGCTTTTTGTTATCTGTTTCCTGAAATGAAAATCA
GAAGCGGGGAGCTCCTCAATTTGAAAAAAAAATCAAGGGTAACTGAGGGTCTGGTTTCCCT
CTGTTGGAGCCCCCAAGAATTCTGTGGTCTTATTTTTGTAATAGTATAAGATGCACTGCTCC
GTTAAGTTTCTCCTTCATCCATTTGCTTTTATTGCTTCGTGACATTTCGTGTAACAGCTCAGA
TTACCATGCATCCCATTACACATTATTTTGCTACTGTGGATATTTCCACTCTGTTTCATGCTT
GACATTTTTAATTCGTTACATCGTGACCAACTAAACATAGTCTGATAGATTTTCAGAGTTTTG
ATCCTCCTAATCACATAATATCCGTTTCTACCTGCAGCTATGGACAAGAATGTCTATCCTCA
ACACAGCGGGTTCCCCCAAGTTCAGCAGTGACCGGACGATCCACGAGTACGCCAAGGACA
TATGGGATATCAGCCCTGTCATCATGCCCTGAATAATTCAAGCAGCTTCCAGGAACCTTGA
GAGTCTATGAGGCGCGATTGGTCGCAGATTTGCCTCGATTCAGCGAGAATGTATTGCTCCC
CCCAGTTTTGTATAGGCATATGCAAGTTCTGGCATGGACCTGAGTGCTGAGAGCATGCAG
GGTAGTTCATCCTTAGCAATCTTGCATGTCAGTTGGCCAGTATACCCAGGCTTATGGTGTG
TATTTCGCCGAATAAAGTTGGAGCAGAATCATCAAACAAACATACTTTGGTGTATCTGTAAT
GATAAATAAAGGGTAAATGACTGAACAAAAAGTGTCGTTGGCGCACACATGTAGTAGAAGC
ATGTTTCTTCAGAGAGGGTGCAAAGCTGAAAGCACCCCCAGCTTTGAAATGAGAATGCAGA
AGTTGGGAAAACATTTGTTTACATCCAAAGTGCAACGGGATTCGTCAAGAGTTCAGAGTCT
TCCATCACGGCCATAGAGCATGTCCAATAGCAGTCCTATAATAGAGCAGTAGCAGTGGTCC
CTCATATTTTGAACCAAAAACGCAGCTCCAACGGCTGCCCTATTTGTAAAATTTACTTCAAA
ATTTGTTGGACAGCCCGAAAATGCAACACCAGCTGATGTAAATGTACAACACTTCGGCTGC
TGCCCTAAAATTTGCTTCAAGTGGCGAACCACTCAAGACCACAAGAAAACAGTTGTTTTAC
GTTACTTTGTGCCAAAAACGTTGCTCCAACAACTGCGCTATAATAGGCCACCCTAATGTATT
TTAGGGCTGTTGTTGGAGATACATATTTTCGGGTGCCTAAAAATAAAATCATTTTTTGGTGC
CTTACGTTTTACGCCAACCACTCTTTTGTGCCCTATTATAGGGCTGCTATTGGAGATACTCT
AACAGTTCCATTTTTCCATAAAAGTTTCTGATTTGGAGGGAGTTGAGCAAAAAACAAAGTTT
TTGTAAACGTGAATTTTTAGTGGCCTTTATAGTTAGCCCAACTCCCCCTCCACCTAA
SEQ ID NO: 10 >Wheat (hexaploid) PHS1 5D (Chinese Spring reference genome) CDS
ATGGCGACCGCCTCGCCGCCGCTCGCCACCGCCTTCCGCCCGCTCGCCGCCGGCGGCG
GCACCGGAGGAGGAGGAGCGCACGCCGCGGGCTGGGTCGCGCCGAGGCGGGGGCGCC
GGGGGTTCGTGGTGCGGAGCGTGGCGAGCGATCGGGAGGTGCGGGGGCCGGCCTCGAC
CGAGGAAGAGCTTTCAGCCGTGCTAACTTCCATTGATTCTTCCGCCATCGCGTCAAATATC
CAGCACCATGCAGACTTCACACCGTTGTTCTCGCCAGAGCACTCTTCACCCCTAAAGGCTT
ACCATGCAACTGCTAAAAGTGTTTTTGATTCTCTGATCATCAATTGGAATGCAACATATGAC
TATTACAACAAAGTGAATGCAAAGCAAGCTTATTACCTGTCCATGGAGTTTTTGCAGGGAAG
AGCTCTCACAAATGCTATTGGCAATCTAGAGCTAACAGGACAATATGCAGAAGCGTTAAAA
CAACTTGGACATAACCTAGAGGATGTTGCTAGTCAGGAACCAGATCCTGCCCTTGGCAATG
GTGGTCTAGGCCGTCTAGCGTCCTGTTTTCTGGATTCTATGGCAACCCTAAATTATCCAGC
ATGGGGATATGGACTTCGGTACAGATATGGCCTCTTTAAGCAAATCATAACAAAGGATGGT
CAGGAGGAGGTAGCTGAGAATTGGCTAGAGATGGGAAATCCATGGGAGATTGTAAGAAAT
GATGTCTCTTATCCTGTGAAATTCTACGGCAAAGTGGTTGAAGGGACTGATGGGAGAAAAC
ACTGGATTGGAGGAGAGAATATTAAGGCTGTGGCACATGATGTTCCTATTCCTGGCTACAA
GACTAAAACTACCAATAATCTTCGTCTTTGGTCAACAACAGTACCATCACAAAATTTTGATCT
GGGAGCTTTCAATGCTGGGGATCATGCCAAGGCCAATGAAGCACATCTAAATGCTGAAAA
GATATGCCACGTATTGTATCCAGGGGACGAATCATCAGAGGGGAAAATTCTTCGCTTGAAA
CAACAATATACATTATGCTCGGCCTCCCTGCAGGACATTATTTCTCGTTTTGAGTCGAGGG
CTGGTGATTCTCTCAACTGGGAGGACTTCCCCTCTAAAGTTGCAGTTCAGATGAATGACAC
TCATCCAACACTGTGCATTCCCGAGTTAATGAGAATACTGATGGATATAAAGGGATTGAGC
TGGAATGAGGCTTGGAGTATCACAGAAAGAACCGTTGCATACACTAACCACACGGTACTTC
CTGAAGCCCTGGAGAAGTGGAGCTTGGACATTATGCAGAAACTTCTACCTCGACATGTTGA
GATCATAGAAACAATAGATGAGGAGCTGATGAACAACATCGTCTCAAAATATGGAACGGCA
GATATTTCACTGTTAAAACAGAAGCTTAAAGATATGAGGATCTTAGACAACGTTGATCTTCC
AGCCTCTGTGGCTAAACTGTTTGTTAAAACAAAAATGAAAAAGGGGAAGTTGCTTGTTGAAT
CTTTGGAGTCTATTGCTGAAGCTGACGAGAAAACTGAGCCAGAAGAAGAGGAAAACATTCT
ATCCGAGACAACAGAGAAGAAGGGCGAGTCTGACTCTGAAGAAGCTCCTGATGCAGAAAA
GGAGGATCCTGAGTATGAGTTAGATCCATTTGCAAAATACGATCCTCAGTTACCTAGAGTT
GTTCGAATGGCAAACCTCTGTGTTGTTGGTGGACATTCAGTTAATGGTGTGGCTGAGATTC
ACAGTGAAATTGTTAAGCAAGATGTGTTCAATAGCTTTTATGAGATGTGGCCTACTAAGTTC
CAAAACAAAACAAACGGAGTAACTCCCAGGCGTTGGATCCGGTTTTGTAATCCTGAATTAA
GTGCAATCATTTCAAAATGGATAGGCTCTGATGACTGGATTCTGAACACTGATAAACTTGCA
GGACTGAAGAAGTTTGCTGATGATGAGGATCTGCAATCAGAATGGCGTACTGCTAAAAGGA
ATAACAAGATGAAAGTAGTTTCACTGATAAGAGATAAGACTGGATATGTTGTCAGCCCAGAT
GCGATGTTTGATGTGCAGGTGAAAAGAATACATGAATATAAGCGGCAGCTGCTAAATATCC
TTGGTATCGTTTACCGGTACAAGAAGATGAAAGAAATGAGTGCAAAAGATAGAATAAAGAG
CTTTGTTCCAAGGGTATGCATATTTGGTGGGAAAGCTTTTGCCACTTATGTACAGGCAAAG
AGGATTGTGAAGTTTATCACAGATGTCGCAGCTACTGTAAATTATGATCCTGATATTGGAGA
TTTACTGAAGGTTGTATTTGTCCCAGATTATAATGTTAGCGTTGCTGAGAAGCTCATTCCTG
CCAGTGAATTGTCTCAGCATATCAGTACCGCTGGAATGGAGGCTAGTGGAACCAGCAACAT
GAAGTTTGCAATGAATGGTTGTATTCTTATTGGAACTTTGGATGGTGCGAATGTGGAGATCA
GAGAAGAGGTTGGAGAGGAGAACTTTTTCCTCTTTGGTGCACATGCACCTGAAATCGCTGG
TTTGAGGCAAGAGAGGGCTGAAGGAAAGTTTGTGCCTGACCCGAGATTTGAAGAGGTTAA
GGAATACGTCCGCAGTGGCGTCTTTGGGACTAGCAACTATGATGAATTGATGGGTTCTTTG
GAAGGAAATGAAGGTTATGGACGTGCAGATTATTTTCTTGTTGGCAAGGATTTTCCCAGTTA
CATTGAATGCCAGGAAAAAGTTGATGAAGCATACCGAGATCAGAAGCTATGGACAAGAATG
TCTATCCTCAACACAGCGGGTTCCCCCAAGTTCAGCAGTGACCGGACGATCCACGAGTAC
GCCAAGGACATATGGGATATCAGCCCTGTCATCATGCCCTGA
SEQ ID NO: 11 >Wheat (hexaploid) PHS1 5D (Chinese Spring reference genome) protein
MATASPPLATAFRPLAAGGGTGGGGAHAAGWVAPRRGRRGFVVRSVASDREVRGPASTEEE
LSAVLTSIDSSAIASNIQHHADFTPLFSPEHSSPLKAYHATAKSVFDSLIINWNATYDYYNKVNAK
QAYYLSMEFLQGRALTNAIGNLELTGQYAEALKQLGHNLEDVASQEPDPALGNGGLGRLASCF
LDSMATLNYPAWGYGLRYRYGLFKQIITKDGQEEVAENWLEMGNPWEIVRNDVSYPVKFYGK
VVEGTDGRKHWIGGENIKAVAHDVPIPGYKTKTTNNLRLWSTTVPSQNFDLGAFNAGDHAKAN
EAHLNAEKICHVLYPGDESSEGKILRLKQQYTLCSASLQDIISRFESRAGDSLNWEDFPSKVAV
QMNDTHPTLCIPELMRILMDIKGLSWNEAWSITERTVAYTNHTVLPEALEKWSLDIMQKLLPRH
VEIIETIDEELMNNIVSKYGTADISLLKQKLKDMRILDNVDLPASVAKLFVKTKMKKGKLLVESLES
IAEADEKTEPEEEENILSETTEKKGESDSEEAPDAEKEDPEYELDPFAKYDPQLPRVVRMANLC
VVGGHSVNGVAEIHSEIVKQDVFNSFYEMWPTKFQNKTNGVTPRRWIRFCNPELSAIISKWIGS
DDWILNTDKLAGLKKFADDEDLQSEWRTAKRNNKMKVVSLIRDKTGYVVSPDAMFDVQVKRIH
EYKRQLLNILGIVYRYKKMKEMSAKDRIKSFVPRVCIFGGKAFATYVQAKRIVKFITDVAATVNYD
PDIGDLLKVVFVPDYNVSVAEKLIPASELSQHISTAGMEASGTSNMKFAMNGCILIGTLDGANVE
IREEVGEENFFLFGAHAPEIAGLRQERAEGKFVPDPRFEEVKEYVRSGVFGTSNYDELMGSLE
GNEGYGRADYFLVGKDFPSYIECQEKVDEAYRDQKLWTRMSILNTAGSPKFSSDRTIHEYAKDI
WDISPVIMP
SEQ ID NO: 12 >Wheat (hexaploid) PHS1 5D (Chinese Spring reference genome) promoter
(−2 kb upstream of ATG)
TATGTTAGTCCAAATAAATATCATCTAAAATTGTGCCTAAACCATATACAAATGATGTAAATG
TGGACATAATACTTCATAAAGTATAGATATGTTTGAGACGTATCAGGAGCGTCGACCTTTTT
GGGAAGCCAAGTGAGCAAAGCCTCATCAAGTCTTGGGAATCCACGGTCATTGAGGGAGAA
AAATTTATCAAAGGTCTCACACATACCCCCTTTGATGATGGACCAGCAGGACCGCAGTAAT
TTGGCCGTGAAGCCATCTAGCCCCGTCAACTTGCCGGAAGGGAACCTCTTGACCGCCTCC
TAAATTTCATCCTCAGTGAAGGGTTGCTCAAGTGCAGGGAGGTCAAAGTGATGCGGGTCG
GTACTATGGAGATCAAGGGAGAGAGTCAGTGCTCAAAAGTGTCAAGAATGGAAGAGAAGT
GATCATATGCCGCCCTGGCCATATCGGGCTCGCCAGAATGTGCCCACCATCAGACTGCAC
CCCTAGGATTATTAACTTCTGTCGTCGGTGCACAGAATGGATCTTGAAGAAGGTGGAATTT
GTGTCGCCCTCACGTAGCCAGGAGAACCTGGCAGGCAGATGTGCAATCGACCTCTCCACC
GAAGCAAACCCTAGGTAGGCGCGCTTCAGCTCGCGGAGTAACCATACTTCTGGAGGAGCA
AGCAGGCGGTGATCTTGAGCCGCATCGAGATGAGCGGTCAACTCCCGAGCGAGAAGCAT
CTGAGTGGAGATGTTACCAAGCGTTCTAGACCTCCAACTCTACAGTCAGCGCATTGTCAGC
TTGAGCTTGGCGTAGATCCCCTGAAAATGATTGGGGTCATGCACGCCAGAATTCCAGGTGT
CAGAGACCACCTAATGAAAACAATCGAGCTTTGGCCAAAATAGCTCAAAATGGAATCTCCG
ATCACCTTGGGAGTGAAAATGTTACTAGTTTCGTTGGAGGCCAAGGTAGTTATGTATTTGTG
TGTGCAATTACTAATCATTGGCCCTTAGCAAACTCCTATTGGTTCAAACCGTTTCACTTTGA
AAAAAATGCTACTTGCTACAAACCGTTTCACTTGGAGGGGCGTCTTTTCTAGTTGAGAGGG
AGCAAAAGGCGTATCAAGACGACAACGCCACCCTCGGCAATGTACAAGTCCTTCCCCGTC
CTCGTTTCGATGCTGCTTTTTGTCATCGCAGCGAGGCGTGTAGAGGGTGGTGTGTCCCCA
GATCTAGTTATCTGGGTTGGGGTTTTTCTTTGGCAGGTTTATCTCAGTGGTAGTGTCTCGTG
AAAGCGGATGACGCGACTATGTGTCTTGGTCCTCCACGCTCTAGTTTCGACAGGTGAAGG
TTGGCCAGCTTCGGCCTCGTCGGGGAGCGTATGAGATTTGTGTTCCTTTACTTCGTCTGGC
CAGACTTCCCGACCGCCGCTTCTACAAGCTCCTTATTTCAAGCAAGTTTTCTTCACCGAGAT
GGAAGTCCAGAGGCGGCAACAGCTTTGCTCACGGCGCAGTGGCGGTGGATGCAGGAGAA
AGAAGACATTCAACACCCCTAAACTATGAAGATGTAATTTTTTATTTTTGTAAGTGCTTTTGT
GAGGACTTGTGCTACTTTTAATATGTGGCCTTTTGCCTTTTTCCAGAAAAAAAAAACTAAAC
CATTACACGAGGAGAAGAGCCATTCCCAGCCGTCCAATAAACACGACAAAACGCTTTACAG
CACGGGGGCAGTTCCCAGCCGTCGAAATGCGCTGCAACGGAACTAGTAGCTCTCCCTGAC
ACGGCGGCACATGAGCCACGCCTTGCGCCGTGCACCGGCGGTGGGCCCACCGGCCAGT
GCCCCCACACGTGTACCGGCGGCCAGCATTGCACGGCACGGCGACGGAGAGATATTTTTA
ACGCCTTCCAATTCAGAAGCTCCCCCATCACTCACTCGGGTACAGAGCGGGCCGCGTGAC
TCTCGCGTCGTCGCATCGCGCGCGCTGCACACCACCGTCAGCGCGACCACTGCCACCA
SEQ ID NO: 13 >Wheat (hexaploid) PHS1 5A (Cadenza) genomic DNA
GGCACGGCGACGGAGAGATATTTTAACGCCTTCCAATTCAGAAGCTCCCCCATCACTCACT
CGGGTACAGAGCGCAGCGGGCCGCGTGACTCTCGCGTCGTCGCATCGCGCGCGCTGCA
CACCACCGTCAGCGCGACCACTGCCGCCAATGGCGACCGCCTCGCCGCCGCTCGCCACC
CCCTTCCGCCCGCTCGCCGTCGCCGGAGGTAGCGGCGGACTCGTCGTGGGCGCCAGGG
CCGTGGTACCGCCGAGGCGGGGGGGGCGGGGGTTCGTGGTGCGGAGCGTGGCGAGCG
ATCGGGAGGTGCGGGGGCCCGCCCCGACCGAGGAAGGTAAGCGGCCGCGCGCTCCCAT
TGGCTGCCACGACCTGGAATGCTGGTCGGCTGTAATGTGGTTCGGATTTTCGCTGGTGCG
ACCTGAATTTGTGATCCTCTGACTGACGGCCTATGTGTTCCCCGGGTTTCGGGCTGTTGAT
TTTGTGCGCGCGCATTGCTAATCTACTTCTCTAGGATTTGTTAGGGCTCAGAGCAATCTTAA
ATTCGCGAATACACTTCGACGAATGTAGTCTGAATTTACTGAAACTAGCTTAGTTTGGCGCG
TCAAGATAGACTGATGTATTTATGGTAGCAAATTCTGAAAACTTCTTCTGTGCGTGGACAGC
CACCCCTCGTGGATGATGCAGCAAGTGCTAACACAGTGTTTGCTAGTACTTGCTAGTATCT
GTAGCTGTAACAAAATCCTTCGAATCTGAACTAGTGAGATAAATCGTTTGAGATTTTTGCTG
AAGATTACACAGTATCAATATTTCTGAAGGTGATTGGAACAGTATAGCATTTCAAACTGGCA
ACCAAGCAGGATCAGTATACTGACGCAATTTTTTTCTGAAGGTGGTTGGAATACCGGTATG
AATTTTATAATCATTGAATTAAGGCGTAAGATTTGGTAACAAAAACTGCTTTTCACGGGAAA
CGATGAAACATGGCACAATTCAACCTGACTTTCTTTTATGAAAAGGCTCTCAAGCTGCCTTG
TACTCCCTCCGTCCCATAATATAAGAACGTTTTTGACATTAGTGTAGTACCAAAAACGTTCT
TATATTATGGGACAGAGGGAGTATTTATTAATGGATAAAAAGAGAGGTACAGGGTTCTAGTA
GTAACATATGCAAATGGTGATACAGTATATTATCTACGATTTTGTGCAAGAATTTTCACACG
AAAACAATATTCCATGCAGTTTTTAGTTTTGACTTTTTTTTTTGAATTTTCACGCGCGCGGGG
GGGGGGGGGGGGGGGGGGGGNNNNNNNNNNNNNNNNNNNNNNNNNNNNNNNNNNNNN
NNNNNNNNNNNNNNNCAGAGAGAGAGGCAGGGGGCGACCCCCCCATGCTATGTTACATG
CAGCTTTGTTCTACCCACGCAGAGCCTGTAGCCAGTTTTGACTCTTTTTGAATTGTTTGGAT
TGCTTACCACATGTGTCACGTGGTTTTAGTTTTGACTCTTGACTGTTTAATACCTACTCTTTT
TAAGATTTTGGATTCTAATGTCATCTGTCTTGCAACTCAGAGCTTTCAGCCGTGCTAACTTC
CATTGATTCATCCGCCATCGCATCAAACATCCAGCACCATGCAGACTTCACACCGTTATTCT
CGCCAGAGCACTCTTCACCCCTAAAGGCTTACCATGCAACTGCCAAAAGTGTTTTTGATTC
TCTGATATTGAATTGGAATGCAACATATGACTATTACAACAAAGTGAATGCAAAGCAAGCTT
ATTACCTATCCATGGAATTTTTGCAGGTCTCGCAATATACTTTCTTCAGCCTTATCTCATACC
TTTTGGTAACTGCGTTATGATGTTATATGTTTTTTTTTGTACTGCAGGGAAGAGCTCTTACAA
ATGCTATTGGCAACCTAGAGCTAACAGGACAATATGCAGAAGCGTTAAAACAACTTGGACA
GAACCTAGAGGATGTTGCTAGTCAGGTAAGCCATTTTTGTCAGAGAATGGTTTTTAGTCCAT
ATCAAAATTATTGCCGAGCCATTTTTCTAAATAACATTTTATTATTATTCATGAACTTCCATAT
TTTTTTCAAATCTGCAAAAGGAGAAGTTTTTTTCCTGGGGGTTCCACCATCTGACTTATATTT
GTTATGCAGTTTTCAGAGTGATTGACTATGAAATTACGCCAAAACACGAGCATTGCCTAGA
GTGCCCTGTAGTTTTAGAAATGTTAAGATTGGATATGTTACTTTTCTATTACAATGAAACAGG
AACCAGATCCTGCCCTTGGCAATGGTGGACTAGGCCGTCTAGCGTCCTGTTTTCTGGATTC
TATGGCAACCCTAAATTATCCAGCATGGGGATATGGACTTCGGTACAGATATGGCCTCTTT
AAGCAAATCATAACAAAGGATGGTCAGGAGGAGGTAGCTGAGAATTGGCTAGAGGTAGTG
ATATGTCTTCTACTTGTTTACTGTTTGGAAGCGTTGATTATGTTGGTTTCTGTTTTAATGATC
TGCATGTTAGCTTCTTTTGCCATTTATAACTGATGCTGTATCTCCAGATGGGAAATCCATGG
GAGATTGTAAGAAATGATGTCTCTTATCCTCTGAAATTCTATGGCAAAGTGGTTGAAGGCAC
TGATGGGAGAAAACACTGGATTGGAGGAGAGAATATCAAGGCTGTGGCACATGATGTTCC
TATTCCTGGCTACAAGACTAAAACTACTAATAATCTTCGTCTTTGGTCAACAACAGTACCAT
CACAAAACTTTGATTTGGGAGCTTTTAATGCTGGGGATCATGTCAAGGCCAACGAAGCACA
TCTAAATGCTGAAAAGGTATTCCTTTGTCTGCATTTATTTTGAATATAAATCATTAAATCTCAA
TAACTATTGATTTAAATGGTAAACACTAAACATTGTCTAGCTAGTAGTCACTGTGGTGTCCC
TCAATGTCATTTTAGAATGTGCACAGTCAAATGTTTGATAACTTTGATTGTAATAATATGATA
ATATACAGAAACTTTGATTGACACTAGATTTTTCATAAACATACTTATTGTATTTCCATGTAAA
TAGAAAAGCTGATGTTCAGAGGTTTGTGTGTCAAAATGAACAGAGTTTGTGCACACATAATA
TCTTCATCTGGTGTTTGCAGTTCTCTAGGTTGTGTTTGCGTTTTCTCCTGCTCTGCCTTAAC
TAATCTCAGCATATGCATCAGGCTACATTGCTCCTCTGTGTCTATAAAAAATAAGTCTGATG
ATATATGGTTGCAGTCCAGTAGTTCGTAGATGGCACAGCATGGAAGAACTTTTCACATGGA
AGCCTTACCCTTATAATTTTAATTATACCTTTTGAGTGTTTGTGCATTGTGTACATACACCAT
TAAGTATGGGTGCTCAAAAATGGTTTATCCCAAACTGTGCAAAAGCCGCCACTGTTTGTGT
ATTAATTTTTTTGAACTAGCTAAACAACTTGACCGAACAAGATACATACAATTGCACTCACTT
CTGAAGCAGCTCAGTTGGACGGATTTGTTCCAATCCCGTGCATGGGTTAAATTGTCGAGTT
GTCCTCGCTTGTTCAGTAGTAGGAGTACTATCTATTATATTTATACTAAAGTTTTCTTTTTGT
GATACAGTACAAACTTTTAGTAATACTTACATTTGCGACCACTGTGGACAAAATTTATTATGT
CTAACTTAACTTTATCACTGGTCTTCTCATCATGCCAGATATGCCACGTATTGTATCCAGGG
GACGAATCATCAGAGGGGAAAATTCTTCGCCTGAAACAACAATATACACTATGCTCGGCCT
CCCTGCAGGACATTATTTCTCGTTTTGAGTCGAGGGCTGGTGATTCTCTCAACTGGGAGGA
CTTCCCCTCTAAAGTTGCAGTTCAGATGAATGACACCCATCCAACACTGTGCATTCCCGAG
TTAATGAGAATACTGATGGATGTTAAGGGATTGAGCTGGAATGAGTCTTGGAGTATCACGG
AAAGGTATCACAGAAGGGTACTGCAGTTTGATGTGTTATGCTGTTCCTCCAGTTCCAGTTAA
CTAATTTTATTTCCTCATCTGTCTTCCATTCAATCATGTACTGATTTTCTGATATCCACATCAT
TTGTAGAACCGTTGCATACACTAACCACACAGTACTTCCTGAAGCTCTGGAGAAGTGGAGC
TTGGACATTATGCAGAAACTTCTACCTCGACATGTTGAGATTATAGAAAGAATAGATGAGGA
GGTATCAAATGTGCAGATAATATTATACAATTAATTTCATGCTCTGTAGTTCAACTTATGGTT
TACTTGTCTTCATCGTTGATTTTAAACAGCTGATGAACACCATCGTCTCAAAATATGGAACG
GCAGATATTTCACTGTTAAAACAGAAGCTTAAAGATATGAGGATCTTAGACAACGTTGATCT
TCCAGCCTCTGTTGCTAAACTGTTTATTAAACCAAAAGAGAAAAAGGGGAAGTTGCTTGTTG
AATCTTTGGAGTCTATTGCTGAAGCTGACGAGAAAACTGAGTCAGAAGAGGAGGAAAACAT
TCTATCCGAGACAACAGAGAAAAAGGGCGAATCTGACTCTGAAGAAGCTCCTGATGCAGAA
AAGGAGGATCCTGAGTATGAGTTAGATCCATTTACAAAATACGATCCTCAGTTACCTAGAGT
TGTTCGAATGGCGAACCTCTGCGTTGTTGGTGGACATTCAGTTAATGGTGTGGCTGAGATT
CACAGTGAAATTGTTAAGCAAGATGTGTTCAATAGCTTTTATGAGGTATTTGCATGATGATT
TGGAAACCGTGATCTTATTTTCCAATGGCTGCTATAATCGTCAGGTGATATGGTATTCACCA
TCTGCCATTTTCTTGTGTTCTTTGCCAGATGTGGCCTACTAAGTTCCAAAACAAAACAAATG
GAGTGACCCCCAGGCGTTGGATCCGGTTTTGTAATCCTGAATTAAGTACAATAATTTCAAAA
TGGATAGGCTCTGATGACTGGATTCTGAACACTGATAAACTTGCAGGACTGAAGAAGGTAC
TTCCAGTAGTTTTATTCCCCAATAATCAGTAATCTAATGCTTCCTTCACTTCAATGTTAACTG
TGCATTGTTTTTACATTGATGTGTGTTCATCCTGTAACAGTTTGCTGATGATGAGGATCTGC
AATCAGAATGGCGTACTGCTAAAAGGAATAACAAGATGAAGGTAGTTTCGCTGATAAGAGA
CAAGACTGGATATGTTGTCAGCCCAGATGCAATGTTTGATGTGCAGGTATATTTTGTACTAG
AAAACATGTGTCTTCTTATAAATATGGTTTAACTCAGCTCTTGGTACTGGACACTTAGGTGA
AAAGAATACATGAATATAAGCGGCAGCTTCTAAATATCCTTGGTATCATTTACCGCTACAAG
AAGATGAAAGAAATGAGTGCAAAAGATAGAATAAAGAGCTTTGTTCCAAGGGTATGCATATT
TGGCGGGAAAGCTTTTGCCACTTACGTACAGGCAAAGAGGATTGTGAAGTTTATCACAGAT
GTCGCAGCTACTGTAAATTATGATCCTGATATTGGAGATCTACTGAAGGTAAATTTGTGTCT
TTGATGCCACTTATCAGTCTTAATTTTCATGTTCTGTCACTGGCAAGTTGACTCCGACTGAA
AAGCATTGAATGTTCAGAGTTTCAAAGGAAAACAGAGCAGTGAATGTTCTTAATGTCGGTG
CTTGTAAAACTGAGCAACTGAATAGCCACAGGTTTTTTAGAGAACAATATAGCCACAAATTT
TTTTGACACTTTTATTGCTATCAGCATCTGATGCTGATATTACTGATAACTAGATGAGGTAAA
TATAAGGTGACAGGTTAAGTCTGAGGATTTTTGTTTGTTATCCAGGTTGTATTTGTCCCAGA
TTATAATGTTAGCGTTGCTGAGACGCTCATTCCTGCCAGTGAATTGTCCCAGCATATCAGG
TAATATTCTGCCTACTCTTATATAGTATGTGAATACATAAAAAAAGAATATTACCTGGAGTTG
GCTGACATAGAATTTTTCATGTGCCTTTTTGTTTGCTATAATATGTACCTGGAGTCTTACTGG
GATAGTGCCATCCAGAAGTGCCACCCCAGTTTCTATTTCTGTATAAGACGTGCATTTTTAAC
AGTATTACTATCCATTCAGTACCGCTGGAATGGAAGCTAGTGGAACCAGCAACATGAAGTT
TGCAATGAATGGCTGTATTCTTATTGGAACTTTGGATGGTGCGAATGTGGAGATCAGAGAA
GAGGTTGGAGAGGAGAACTTTTTCCTCTTTGGTGCACATGCACCTGAAATCGCTGGTTTGA
GGCAAGAGAGAGCTGAAGGGAAGGTACCACACTTGTTTATATAGCTGTGCCTACTATCAAA
CTAGTAAAGATCTCTCCCATATGTGCTGTCCAAATAGTTTAGCTGAAGGAAAATCATAACTT
TACAAGTTTCCTTTGCACTTGTAGTTTGTGCCTGACCCGAGATTTGAAGAGGTTAAGGAATA
CGTCCGCAGTGGCATCTTTGGGACTGGCAACTATGATGAATTGATGGGTTCTTTGGAAGGA
AATGAAGGTTATGGACGTGCAGATTATTTTCTTGTTGGCAAGGATTTTCCCAGTTACATTGA
ATGCCAGGAAAAAGTTGATGAAGCATACCGAGATCAGAAGGTAAGTTGTATAACTGACGCT
GTTTGTTTTCTGGCTGAAATGCTCACAGTGATTATGCAAACTTTCTGATATGTGATGGCTGA
CATCAGTGTTTCACTGTGAACGACTTCATTGATTCGTAGGAAGGAATATTTCTTAGTTTAAA
GTTTCAAACTGAAGTATCTATCGGTGTGTTGGAAGATCATAAACAATCAGAGAATATAGTTG
CTAGATGTGCTATATTACTCATCTGTTGGCTGTTGAGTGTTGATACATGAGCTTTGATGTAG
CTAGACCTTCAGGAAAGTGCTTCTGTGCATTCCAATAAAAAAATAACTTATAACTTAACAAAA
CAAAACAATTAGGTTGAACCTCACCAGGGGGCCTTTTGTTATCTGTTTTCTGAAATGAAAAT
CAGAAGTGGGGAGCTCCTCAATTCGAAAAAGAAAAAGAAAATCAGGGGGTAACTGAGGGT
CTGGTTTCCCTCTGTTGGAGCCCCCAAGAATTCTGTGGTCTTATTTTTGTAAGAGTATAAGA
TGCACTGCTCCGTTAAGTTTCTCCTTCACCCATTTGTTTTCATTGCTTCGTGACATTTCGTGT
GACAGCTCAGATCACCATATGCATCCCATTTGTACACATTATTTTGCTACCGTGGATATTTC
CACTCTGTTTCATGTGTGACATTTTTAATTCGTTACATTGTTACTCTAAACATTAGTCTAATA
GATTTTCAGAGTTTTGACCCTCCTAATCACATAATATCCGTTTCGACCTGCAGCTATGGACA
AGAATGTCTATCCTCAACACAGCGGGTTCCCCCAAGTTCAGCAGTGACCGGACGATCCAC
GAGTATGCCAAGGACATATGGGGTATCAGCCCCGTCATCATGCCCTGAATAATTCAAGCAG
CTTCCAGGAACCTTGAGAGTCTATGAGGCGCGATTGGTCGCTGATTTGCCTTGATTCAGCG
AGAATGTATTGCTCCCCCCAGTTTTGTATAGGCATATGCAAGTTCTGACATGGACCTGAGT
GCTGAGAGCATGCAGGGTAGTTCATCCTTAGCAATATTGCATGTCAGTTAGCCAGTATTAC
CCAGGCTTATGGTGTGTGTATTTCGCTGAATAAAGTTGGAGCAGAGTCATCAAACAAACAT
ACTTTGGTGTATCTGTAATGATAAATAAATGGTACATGTGACTGAACAAAAAGTGTCGTTGG
CGTGCACATGTAGTAGAAGCATGTTTCTTCAGAGAGGGTGCAAAGCTGAAAGCACCCCAG
CTTTGGAGTGAGAATGCAGAAGTTACGAAAACATTTGTTTACATCCAAAGTGCAACGGGAT
TCGTCAAGAGTTCAGAGTCTTCCATCACGGTCATAGAGCATGTCCAATAGCAGCCCTATAA
ACCTATAACAGAGCAGTAGCAGTGGTCCCTCACATTTTGAACCAAAAATGCAGCTCCAACG
GCTGCCCTATTTGTAAAATTTACTTCAAAATTTGTTGGACAGCCCTAAAATGCAACACCAAC
TGATGTAAATGTACAACTATTCGGCTGCTGCCCTAAAATTTGCTCCAAGTGGCGAACCACT
CAGGACCACAAGAAAACAGTTGTTTTACGTTGTGCCAAAAATGTTGCTTCAACTACTGC
SEQ ID NO: 14 >Wheat (hexaploid) PHS1 5A (Cadenza) CDS
ATGGCGACCGCCTCGCCGCCGCTCGCCACCCCCTTCCGCCCGCTCGCCGTCGCCGGAG
GTAGCGGCGGACTCGTCGTGGGCGCCAGGGCCGTGGTACCGCCGAGGCGGGGGGGGC
GGGGGTTCGTGGTGCGGAGCGTGGCGAGCGATCGGGAGGTGCGGGGGCCCGCCCCGA
CCGAGGAAGAGCTTTCAGCCGTGCTAACTTCCATTGATTCATCCGCCATCGCATCAAACAT
CCAGCACCATGCAGACTTCACACCGTTATTCTCGCCAGAGCACTCTTCACCCCTAAAGGCT
TACCATGCAACTGCCAAAAGTGTTTTTGATTCTCTGATATTGAATTGGAATGCAACATATGA
CTATTACAACAAAGTGAATGCAAAGCAAGCTTATTACCTATCCATGGAATTTTTGCAGGGAA
GAGCTCTTACAAATGCTATTGGCAACCTAGAGCTAACAGGACAATATGCAGAAGCGTTAAA
ACAACTTGGACAGAACCTAGAGGATGTTGCTAGTCAGGAACCAGATCCTGCCCTTGGCAAT
GGTGGACTAGGCCGTCTAGCGTCCTGTTTTCTGGATTCTATGGCAACCCTAAATTATCCAG
CATGGGGATATGGACTTCGGTACAGATATGGCCTCTTTAAGCAAATCATAACAAAGGATGG
TCAGGAGGAGGTAGCTGAGAATTGGCTAGAGATGGGAAATCCATGGGAGATTGTAAGAAA
TGATGTCTCTTATCCTCTGAAATTCTATGGCAAAGTGGTTGAAGGCACTGATGGGAGAAAA
CACTGGATTGGAGGAGAGAATATCAAGGCTGTGGCACATGATGTTCCTATTCCTGGCTACA
AGACTAAAACTACTAATAATCTTCGTCTTTGGTCAACAACAGTACCATCACAAAACTTTGATT
TGGGAGCTTTTAATGCTGGGGATCATGTCAAGGCCAACGAAGCACATCTAAATGCTGAAAA
GATATGCCACGTATTGTATCCAGGGGACGAATCATCAGAGGGGAAAATTCTTCGCCTGAAA
CAACAATATACACTATGCTCGGCCTCCCTGCAGGACATTATTTCTCGTTTTGAGTCGAGGG
CTGGTGATTCTCTCAACTGGGAGGACTTCCCCTCTAAAGTTGCAGTTCAGATGAATGACAC
CCATCCAACACTGTGCATTCCCGAGTTAATGAGAATACTGATGGATGTTAAGGGATTGAGC
TGGAATGAGTCTTGGAGTATCACGGAAAGAACCGTTGCATACACTAACCACACAGTACTTC
CTGAAGCTCTGGAGAAGTGGAGCTTGGACATTATGCAGAAACTTCTACCTCGACATGTTGA
GATTATAGAAAGAATAGATGAGGAGCTGATGAACACCATCGTCTCAAAATATGGAACGGCA
GATATTTCACTGTTAAAACAGAAGCTTAAAGATATGAGGATCTTAGACAACGTTGATCTTCC
AGCCTCTGTTGCTAAACTGTTTATTAAACCAAAAGAGAAAAAGGGGAAGTTGCTTGTTGAAT
CTTTGGAGTCTATTGCTGAAGCTGACGAGAAAACTGAGTCAGAAGAGGAGGAAAACATTCT
ATCCGAGACAACAGAGAAAAAGGGCGAATCTGACTCTGAAGAAGCTCCTGATGCAGAAAA
GGAGGATCCTGAGTATGAGTTAGATCCATTTACAAAATACGATCCTCAGTTACCTAGAGTTG
TTCGAATGGCGAACCTCTGCGTTGTTGGTGGACATTCAGTTAATGGTGTGGCTGAGATTCA
CAGTGAAATTGTTAAGCAAGATGTGTTCAATAGCTTTTATGAGATGTGGCCTACTAAGTTCC
AAAACAAAACAAATGGAGTGACCCCCAGGCGTTGGATCCGGTTTTGTAATCCTGAATTAAG
TACAATAATTTCAAAATGGATAGGCTCTGATGACTGGATTCTGAACACTGATAAACTTGCAG
GACTGAAGAAGTTTGCTGATGATGAGGATCTGCAATCAGAATGGCGTACTGCTAAAAGGAA
TAACAAGATGAAGGTAGTTTCGCTGATAAGAGACAAGACTGGATATGTTGTCAGCCCAGAT
GCAATGTTTGATGTGCAGGTGAAAAGAATACATGAATATAAGCGGCAGCTTCTAAATATCCT
TGGTATCATTTACCGCTACAAGAAGATGAAAGAAATGAGTGCAAAAGATAGAATAAAGAGC
TTTGTTCCAAGGGTATGCATATTTGGCGGGAAAGCTTTTGCCACTTACGTACAGGCAAAGA
GGATTGTGAAGTTTATCACAGATGTCGCAGCTACTGTAAATTATGATCCTGATATTGGAGAT
CTACTGAAGGTTGTATTTGTCCCAGATTATAATGTTAGCGTTGCTGAGACGCTCATTCCTGC
CAGTGAATTGTCCCAGCATATCAGTACCGCTGGAATGGAAGCTAGTGGAACCAGCAACAT
GAAGTTTGCAATGAATGGCTGTATTCTTATTGGAACTTTGGATGGTGCGAATGTGGAGATC
AGAGAAGAGGTTGGAGAGGAGAACTTTTTCCTCTTTGGTGCACATGCACCTGAAATCGCTG
GTTTGAGGCAAGAGAGAGCTGAAGGGAAGTTTGTGCCTGACCCGAGATTTGAAGAGGTTA
AGGAATACGTCCGCAGTGGCATCTTTGGGACTGGCAACTATGATGAATTGATGGGTTCTTT
GGAAGGAAATGAAGGTTATGGACGTGCAGATTATTTTCTTGTTGGCAAGGATTTTCCCAGT
TACATTGAATGCCAGGAAAAAGTTGATGAAGCATACCGAGATCAGAAGCTATGGACAAGAA
TGTCTATCCTCAACACAGCGGGTTCCCCCAAGTTCAGCAGTGACCGGACGATCCACGAGT
ATGCCAAGGACATATGGGGTATCAGCCCCGTCATCATGCCCTGA
SEQ ID NO: 15 >Wheat (hexaploid) PHS1 5A (Cadenza) protein
MATASPPLATPFRPLAVAGGSGGLVVGARAVVPPRRGRRGFVVRSVASDREVRGPAPTEEEL
SAVLTSIDSSAIASNIQHHADFTPLFSPEHSSPLKAYHATAKSVFDSLILNWNATYDYYNKVNAK
QAYYLSMEFLQGRALTNAIGNLELTGQYAEALKQLGQNLEDVASQEPDPALGNGGLGRLASCF
LDSMATLNYPAWGYGLRYRYGLFKQIITKDGQEEVAENWLEMGNPWEIVRNDVSYPLKFYGK
VVEGTDGRKHWIGGENIKAVAHDVPIPGYKTKTTNNLRLWSTTVPSQNFDLGAFNAGDHVKAN
EAHLNAEKICHVLYPGDESSEGKILRLKQQYTLCSASLQDIISRFESRAGDSLNWEDFPSKVAV
QMNDTHPTLCIPELMRILMDVKGLSWNESWSITERTVAYTNHTVLPEALEKWSLDIMQKLLPRH
VEIIERIDEELMNTIVSKYGTADISLLKQKLKDMRILDNVDLPASVAKLFIKPKEKKGKLLVESLESI
AEADEKTESEEEENILSETTEKKGESDSEEAPDAEKEDPEYELDPFTKYDPQLPRVVRMANLC
VVGGHSVNGVAEIHSEIVKQDVFNSFYEMWPTKFQNKTNGVTPRRWIRFCNPELSTIISKWIGS
DDWILNTDKLAGLKKFADDEDLQSEWRTAKRNNKMKVVSLIRDKTGYVVSPDAMFDVQVKRIH
EYKRQLLNILGIIYRYKKMKEMSAKDRIKSFVPRVCIFGGKAFATYVQAKRIVKFITDVAATVNYD
PDIGDLLKVVFVPDYNVSVAETLIPASELSQHISTAGMEASGTSNMKFAMNGCILIGTLDGANVEI
REEVGEENFFLFGAHAPEIAGLRQERAEGKFVPDPRFEEVKEYVRSGIFGTGNYDELMGSLEG
NEGYGRADYFLVGKDFPSYIECQEKVDEAYRDQKLWTRMSILNTAGSPKFSSDRTIHEYAKDI
WGISPVIMP*
SEQ ID NO: 16 >Wheat (hexaploid) PHS1 5A (Cadenza) promoter (−2 kb upstream of ATG)
CACCACGCAGCCTAGGGGCACCGTCTGGCCATCTGGAGCCCCGGTGGCTCCCTTGGACC
CCATCTTCTCGCTAGTGGGGCCTTGGTCCAAAATAATCCATGTAATTTTTCATGTTTTCCGG
CTTCGTATTTTTTTGGTAAAAACCCAAAAACAATAGAAAAGAAGAATTGGCCTCTGACACTA
GATTGATAGGTTAGTCCAAATCAATATTATCAAAAATTGTGCCCAAACCATATACAAATGATA
TAAATATAACTAGAATACTTTATAAATTTAGATACGTTTGATAAAGTGCCAAGAATGGAAGAG
AAGTGATCATATGCCGCCCTGTCCATATCGGGCTCATCAGAATGTTCCCACCATCAGACTC
CAGCCCCAGGATTATTAACTTATGTCGGCGGTGCGCTGAATGGATCTTGAAGAAGGCGGA
ATTTGTGTCGCCCTCACGTAACCAGGAGAACTTGGCAGGTTGACGCGCAATCGACCTCTC
CAGCAAAGCAAGTCCTAGGTAGGCGCGCTTCAGCTCGCAGAGTAACCGTACTTCTGGAGG
AGCAAGCGGGTGGTGATCTTGAGCCGCCGAGACGAGCAATCAATTACCGAGCGAGAAGC
ATCTGAGTGGAGATGTTACCGAGCGTTCTGGAACTCCAACTCTGCCGTCAGTGCATTGTCA
GCTTGTGCTTTGCGTAGATTCGCCGGAAAGGATCGGGGTCATGCACGCCAGAATTCCAGG
TGTCAGAGACCACCTAATGAAAACCATCGAGCTTTGATCAAAATAGCTCAAGATGGAATCT
CCGATCACCTTGGGAGCGAAAATCTTACTAGTTTCGTTCAAGGCCAAGGTAGTTATATATTT
GTGTGTGCAATTACTAATCATTGGCCGCTTAGCAAACTCTTATTGGTTCGATAAACGTTAAA
GTCATCCACTTTGAGAAAAAATGCTACTTGCTACAAACCCTTTCACTTTGGGAGGAGGGGG
GGTGGGTGGGGCAACATGTATCAGGATGATGGTGCCACCCTTGGCAAGGAATAAATCCTT
GCCGCCTCGTCCTTGTTTGGATGCTGCTTCTTGTCATCTCAAGGAGGCGTGTGGAGGGTG
GTGTGTCCCCGGATCTTGTTCTCTCGGTTGGGTTTTTTCTATGGCAGGTTTATCTCAGTGGT
AGTGTCACGTGAAGCAGATGACGCGACTATGCGTCTTGGTCCTCTGCGCTCTAATTCCGG
CAGATGAAGGTTGACCAGCTTCAGCCTCGTCGGGGAGCGTATGAGATTTGTGTTTTTTGAC
TCCATCTGGTCGGACCTGTGGCATTATGTAGACCCTCTCATGTATTTTTTTCTCCTGGACAG
TAGTCTGTTACACAAATCGTGGTCATCGGATCTTCTAGTCCACATCGGCGACTTTTTCGACC
GCCACTTCTACAAACTCCTTATTTTCAACAAGTTTCCTTCATTGAGATGGAAGTCCAGGCAA
TGGCTTTGCTCACGGCACACTGCCGGCGGATGCAAGAGAAAGAAGACATTCGACACCCCT
AAAACTATGAAGATGTAATTTTTAATCTTTGTAAGTGTTTTTGTGAGGATTTGTGCTACTTTT
AATATATTGCCTTTTGCCTTTTGCAAGGGAAAAAAACTAAACCATTACACGAAGAGAAGAGC
CATTCCCAGCCGTCCAATAAACACGACAAAACGCTTTACAGCACGGGGGCAATTCCCAGC
CGTCGAAATGCGCTGCAACGGAACCAGCGCTCCCCGACACGGCGGCACATGAGCCACAA
CTCGTACCATGCACCGTGCACCGGCGGTGGGCCCACTGGCCAGCGTCCCCACACGTGTA
CCGGCGGCCACAATTACACGGCACGGCACGGCGACGGAGAGATATTTTAACGCCTTCCAA
TTCAGAAGCTCCCCCATCACTCACTCGGGTACAGAGCGCAGCGGGCCGCGTGACTCTCGC
GTCGTCGCATCGCGCGCGCTGCACACCACCGTCAGCGCGACCACTGCCGCCA
SEQ ID NO: 17 >Wheat (hexaploid) PHS1 5B (Cadenza) genomic DNA
TTGCACGGCACGGCGACGGAGAGATATTTTTAACGCCTTCCAATTCAGAAGCTCCCCATCA
CTCGCTCGGGTATCAGAGCGGGCCGCGTGACTCTCGCGTCGTCGCATCGCGCGCGCTGC
ACACCACCGTCAGCGCGACCACTGGCGCCAATGGCGACCGCCTCGCCGCCGCTCGCCAC
CGCCTTCCGCCCGCTCGCCGCCGCCGGCGGCGCCGGAGGAGGAGGAGCGCACGCCGTG
GGCGCCGCGGGCCGGGTCGCGCCGAGGCGGGGGCGCCGGGGCTTCGTGGTGCGGAGC
GTGGCGAGCGATCGGGAGGTGCGGGGGCCCGCCTCGACCGAGGAAGGTAAGCGGCCGC
GCGCTCCCGTTGGCTACGACGACCCGGAATGCTGGTCGGCTGTAATGTGGTTCGGATTTT
CGCTGGTGCGACCTGAATTTGTGATCCTCTGACTGATGGTCTATGTGTTCCCGGGTTTCGG
GCTGTTGATTTTGTGCGCGCGCATTGCTAATCTACTGCTCTAGGATCTGCTAGGGTTCAGA
GGAATCTTAAATTCGTGAATAAGCTTCGACGAATGTATTCTGAATTTACTGAAACTGGCTTA
GTTTGGGGCGTCAAGTTAGAATGATGTATTTATGGCACAGTAGCAAATTCTGACAAATTCTT
CTGTGCGTGGACAGCCACCCCTTGTGGATGATGCAGCAAGTGTCAACATACTGTCGCTAC
TATCTGTAGCTGTAACAAAATCTTACGCCTTGTTTTCTGGTGGTACAGTATATTATCATTTTA
CAAAGAGTAGCTGTAACAAAATCTTACGCCTTGTTTTCTGGTGGGTTGCCATTGAGGAGTA
AGTAGAATGCAAATGGTGATACAGTATATTATCTACCATTTTGTGCAAGAATTTTCACACGG
AAACAATATCCCATGCAATTTTTAGTTTTGACTCTCGACTGTTTATACCTACTCTTTGTAAAA
TTTTGGATCCTAATATTGTTGTTCTTGCAACTCAGAGCTTTCAGCCGTGCTAACTTCCATTG
ATTCTTCCGCCATCGCGTCAAATATCCAGCACCATGCAGACTTCACACCGTTGTTCTCACC
AGAGCACTCTTCACCCCTAAAGGCTTACCATGCAACTGCTAAAAGTGTTTTTGATTCTCTGA
TCATCAATTGGAATGCAACATATGACTATTACAACAAAGTGAATGCAAAGCAAGCTTATTAC
CTGTCCATGGAGTTTTTGCAGGTCTCACAATATACTTCCTTTAGCCTTATCTCAATACCTTTT
GGTAACTGCGTTATGATGTTATATGGTTGTTATCTGTGTACTGCAGGGAAGAGCTCTCACA
AATGCTATTGGCAATCTAGAGCTAACAGGACAATATGCAGAAGCGTTAAAACAACTTGGAC
AGAACCTAGAGGATGTTGCTAGTCAGGTAAGTGATTTTTGTCGGAGATTGGTTTTTAGTCC
GTATCAAAATTATCGCAGGGCCATTGTTTTTATTTTGAAAACGCATTTTATTATTAATCATCA
AGTTCCCTTTGTTTAAAAACTATCTGAAAAGAAGAAGTTTTTTTCCTGAGGGTTCCACCGTC
TGACTTATGATCTTTATGCAGTTTTCAGAGTGACTATGAAATTACGCCAAAACACGATCTTAT
TGATGCCTAGAGTGCCCTGTAGTTTAGAAATCTAAGATTGGATATGTTACTTTTCTATTACAA
TGAAACAGGAACCAGATCCTGCCCTTGGCAATGGTGGTCTAGGCCGTCTAGCGTCCTGTT
TTCTGGATTCTATGGCAACCCTAAATTATCCAGCATGGGGATATGGACTTCGGTACAGATAT
GGCCTCTTTAAGCAAATCATAGCAAAGGATGGTCAGGAGGAGGTAGCTGAGAATTGGCTA
GAGGTAGTGATATGTCTTCTACTTGTTTACTGTTTGGAAGTGTTGATTATGTTGGTTTATTTA
TGTTCTAATGACCTGCATGTTAGCTTCTTTTCTATTTATAACTGATGCTGTATCTCCAGATGG
GAAATCCATGGGAGATCGTAAGAAATGATGTCTCTTATCCTGTGAAATTCTATGGCAAAGTG
GTTGAAGGCACTGATGGGAGAAAGCACTGGATTGGAGGAGAGAATATCAAGGCTGTGGCA
CATGATGTTCCTATTCCTGGCTACAAGACTAAAACTACTAATAATCTTCGTCTTTGGTCAAC
AACAGTACCATCACAAAATTTTGATTTGGGTGCCTTTAATGCTGGGGATCATGCCAAGGCC
AATGAAGCACATCTAAATGCTGAAAAGGTATTCTTTCGTCTACTTTATTTTGAATATAAACCA
TTAACTCTCAACAACTACTGATTCAAAGGCTAAACAGTGCTTTGCTAGTAGTTACTGTGTTTT
CTCTTAGATGTCATTTTATAATGTGTGCAGTCAATCGTTTGATATACACAAACCTATTTGGAT
TTCTCCTGTAAAAATGTTGTCACTAGATTTTTCATGAAAAAACTTATTGTATATGTAAATAGA
AAAGCTGAGGCTCAGGTGCTTTGTGTTCAAATGACAAGTAAAAATGAACAGAGTTTGTGCA
CCCATAACTTCTTCACCTGGTGTGTGCAATTCTTTCGGTTGTATTTATGCGTGTTCTTATTCT
GCCTGAATGATTCTCAGCATATGCATCAGGCTACATTTCTCCTCTGTGTCAAGAAAAAATAA
GTCTGACCAGATGCAGTCCAGTAGTCTTTAGATCTTTTCACATGGAAGCGTTACTCTATGAT
TTTTATTATACCTTTAGAGTGTTTGTGCATTGTGCACATATACCATGAAGTATGGGTGTTCAA
ATGTGGTTCTCTCAAACTGTGCTAAATCCACCACTGTTTGCCAGTATTCATTTTTCTGAACTA
GCAACAACTTGACTGGACAAGATACATTGTACTCACATCTGAAGCTGCTGGGTTGGACAGA
TTTGTCCGACCCGTCTTTGAACATCTCTACTATTTAGTGTGCATGGGTTAAATTGTTGACTTT
TCCTCGTTTGTTCAGTAGTAGGAGCACTATCTAATTATATTTATATTCAAGTTTTCTCTGTGT
GTTACGTTATAAACTTTTAGTCATACTTACATTTGTGGACAAAAGTTATCATGTCTAGCTTAA
GTTTGTGACTGATCTTCTCATCATGCCAGATATGCCACGTATTGTATCCAGGGGACGAATC
ATCAGAGGGGAAAATTCTTCGCTTGAAACAACAATATACATTATGCTCGGCCTCCCTGCAG
GACATTATTTCTCGTTTTGAGTCGAGGGCTGGTGATTCTCTCAACTGGGAGGACTTCCCCT
CTAAAGTTGCAGTTCAGATGAATGACACTCATCCAACACTGTGCATTCCGGAGTTAATGAG
AATACTGATGGATATAAAGGGATTGAGCTGGAATGAGGCTTGGAGTATCACAGAAAGGTAC
CACAGAAGGGTACTGCAGTTTGATGTGTTATGCTGTTCCTCCAGCTCCAATTAACTAATTGT
ATTGCCTCATCTGTCTTCCATTCAATTATGTACTGATTCTCTGATATCATTTGTAGAACCGTT
GCATACACTAACCACACGGTACTTCCTGAAGCTCTGGAGAAGTGGAGCTTGGACATTATGC
AGAAACTTCTACCCCGACATGTTGAGATCATAGAAACAATAGATGAGAAGGTAACAAATGT
GCAGATAATATTATGCAATAAATTTCATTCTCTGTAGTTCAACTTATGGTTTACTTATCAACA
TCGTTGATTTTAAAACAGCTGATGAACAACATCGTCTCAAAATATGGAACGGCAGATATTTC
ACTGTTAAAACAGAAGCTTAAAGATATGAGGATCTTAGACAACGTTGATCTTCCAGCCTCTG
TTGCTAAACTGTTTATTAAACCTAAAGAGAAAACGGGGAAGTTGCTTGTTCAATCTTTGGAG
TCTATTGCTGAAGGTGACGAGAAAACTGAGTCACAAGAGGAGGAAAACATTCTATCCGAGA
CAGCAGAGAAAAAGGGCGGATCTGACTCTGAAGAAGCTCCTGATGCAGAAAAGGAGGATC
CTGTGTATGAGTTAGATCCATTTGCAAAATACGATCCTCAGTTACCTAGAGTTGTTCGAATG
GCAAATCTCTGTGTTGTTGGTGGACATTCAGTTAATGGTGTGGCTGAGATTCACAGTGAAA
TTGTTAAGCAAGATGTGTTCAATAGCTTTTATGAGGTATTTGCACGATGATTTGGAAACCAT
GATCTTATTTTCTAACGGCTGCTATAATCGCCAGGTAATATGGTATCACCATCTGCCATTTT
CTTATGTTATTTGCCAGATGTGGCCTACTAAGTTCCAAAACAAAACAAACGGAGTAACTCCC
AGGCGTTGGATCCGGTTTTGTAATCCTGAATTAAGTGCAATCATTTCAAAATGGATAGGCTC
TGATGACTGGATTCTGAACACTGATAAACTTGCAGGACTGAAGAAGGTACTTCCAGTAATTT
TAATTTCCAAATATCAGTAATCTAATGCTTCCTTCACTTCCATGTTACCTGTGCCTTGTTTTT
ACATTGATGTGTGTTCATCCCGTAATAGTTTGCTGATGATGAGGATCTGCAATCAGAATGG
CGTACTGCTAAAAGGAATAACAAGATGAAGGTAGTTTCGCTGATAAGAGACAAGACTGGAT
ATGTTGTCAGCCCAGATGCGATGTTTGATGTGCAGGTATATTTTGGACTAGAAACATGTGT
CTCCTGTTAAATATGGTTTAACTCGGATCTTGGTACTTGACACTTAGGTGAAAAGAATACAT
GAATATAAGCGGCAGCTGCTAAATATCCTTGGTATCGTTTACCGATACAAGAAGATGAAAG
AAATGAGTGCCAAAGATAGAATAAAGAGCTTTGTTCCAAGGGTATGCATATTTGGTGGGAA
AGCTTTTGCCACTTACGTACAGGCAAAGAGGATTGTGAAGTTTATCACAGATGTCGCAGCT
ACTGTAAATTATGACCCTGATGTTGGAGATTTACTGAAGGTAAATTTGTGTCTTCAATGGCA
CTTATCATTCTAAATTTTCATGATCTGTCACTAGCAATTTGACTCCGACTAGGGGTTACTGA
AAAGCACTGAATGTTCCGAGTTTCAAAAGAAAACAGAGCAGTGAATGTTCTTAATGTCGGT
GCTTGTAGAACTGAGAAACTGAATAGCCACAGTTTTTTTTTAGAGAACAATAGCCACAATTG
TTCTGATGCTGATTTTGCTGATAACTAGATGAGGTAAATACAAGGTGACAGGTTAAGTTCTG
AGGATTTTTGTTTGTTATCCAGGTTGTATTTGTCCCAGATTATAATGTTAGCGTTGCTGAGA
AGCTCATTCCTGCCAGTGAATTGTCCCAGCATATCAGGTAATATTCTGGCTACTCTTATATA
GTGCTCCCTCCGTCCCAAAATTCTTGTCTTAGATTTGTCCAAATACGGATGTATCTAGTTAC
ATTTTAGTGTTAGATACATCCGTATCTAGACAAATGTAAGAGAAGAATTTTGGGACGGAGG
GAGTATGTGAATACAAAGAAAAGGAGTCTTATCTGGAGGTGACTGATGTGGAAATTTTCAT
GTGCCTTTTTGTTTGCTACTCCCTCCGTCCCATAATATAAGAGCGTTTTTGACAGTACACTA
TTTTCAAAAATGCTCTTATAATATGGGACGGAGGGAGTATAATATATACCTGGAGTCTTGCT
GGGATAGTGACATCCAGAAGTGCCACGCCAGCTTCTATTTCTGTACAAGACGTGCATTTTT
AACACTATTACTATCCATTCAGTACCGCTGGAATGGAAGCTAGTGGAACCAGCAACATGAA
GTTTGCAATGAATGGTTGTATTCTTATTGGAACTTTGGATGGTGCGAATGTGGAGATCAGA
GAAGAGGTTGGAGAGGAGAACTTTTTCCTCTTTGGTGCACATGCACCTGAAATCGCTGGTT
TGAGGCAAGAGAGGGCTGAAGGAAAGGTACCACACTTGTTTATATAGCTGTGCCTACTATC
AAACTAGTAAAGATATCTCCCATATGTGTGCTGTCCAAATAGTTTAGCTGAAGGAAAATCAT
AACTTTATCATTTTCCTTTGCACTTGTAGTTTGTGCCTGACCCGAGATTTGAAGAGGTTAAG
GAATACGTCCGCAGTGGCGTCTTTGGGACTAGCAACTATGATGAATTGATGGGTTCTTTGG
AAGGAAATGAAGGTTATGGACGTGCAGATTATTTTCTTGTTGGCAAGGATTTTCCCAGTTAC
ATTGAATGCCAGCAAAAAGTTGATGAAGCATACCGAGATCAGAAGGTAAGTTGTATAACTG
ACGCTGTTTGTTTTCTGGCTGAAATGCATACGGTGATTATGCAAACTTTCTGATATGTGATG
GCTGACATCAGTGTTTCACTGTTAATGACTTTACTGATTCGTAGGAAGGAATATTTCTTAATT
CAAAGTTTCAAACCGGAGTATCTATCGATGTGTTGGAACTTGGAAGATCATAAACAATCAGA
GAATATAGTTGCTAGATGTGCTATACTACTCATCTGTTGGCTGTTGAGTGTTGATACATGAG
CTTTGATAAACCTAGCTAGACCTTCAGGAAAGTGCTTCTATGCATTCAATAAAAAAATAACTT
TATAACTTAACAAAATAATTAGGTTGAACCTCGCCAGGGGGGCCCTTTTGTAATCTGTTTTC
TGAAATGAAAATCAGAAGCAGGGAGCTCCTCGATTCTAAAATGAAAAATAAAATCAGGGAT
AACTGAGGGTCTGGTTTCCCTCTGTTGGAGCCCCAGAGAATTCTGCGGTCTTATTTTTGTA
AGAGTATAAGATGCACTGCTCCGTTAAGTTTCTCCTTCATCCATTTGTTTTCTTTGCTTCGTG
ACATTTGGTGTAACAGAGCATCCCATTTGTACACATTATTTTGCTATCGTGGATATTTCCACT
CTGTTTCATGTGCGACATTTTAAATTCGTTACATCGTTACTAACTACACTTAGTCTGATAGAT
TTTCAGAGTTTTGGTCCTCCTAATAACATAATATCCGTTTCTACCTGCAGCTATGGACAAGA
ATGTCTATCCTCAACACAGCGGGTTCCCCCAAGTTCAGCAGTGACCGGACGATCCATGAG
TACGCCAAGGACATATGGGATATCAGCCCCGTCATCATGCCCTGAATAATTCAAGCAGCTT
CCAGGAACCTTGAGAGTCTATGAGGCGCGATTGGTCGCATATTTGCCTCGATTCAGCGAG
AATGCATTGCTCCCCCCCAGTTTTGTATAGGCATATGCAAGTTCTGACATGGACCTGAGTG
CTGAGAGCATGCAGGGTAGTTAATCCTTAGCAATCTTGCGTGTCAGTTGGCCAGTATTACC
CAGGCTTATGGTGTGTGTATTTCGCCGAATAAAGTTGGAGCAGAATCATCAAACAAACATA
CTTTGGTGTATCTGTAATGATAAATAAATGGTAAATGACTGAACAAAAAGTGTCGTTGGCGC
ACACATGTAGTAGAAGCATGTTTCTTCAGAGAGGGTGCAAAGCTGAAAGCACCCCAACTTT
GAAGTGAGAATGCAGAAGTCACGAAAACATTTGTTTACATCCAAAGTGCAATGGGATTCGT
CAAGAGTTCAGAATCTTCCCTATAATAGAGTAGTAGCAGTGGTCCCTCACATTTTGA
SEQ ID NO: 18 >Wheat (hexaploid) PHS1 5B (Cadenza) CDS
ATGGCGACCGCCTCGCCGCCGCTCGCCACCGCCTTCCGCCCGCTCGCCGCCGCCGGCG
GCGCCGGAGGAGGAGGAGCGCACGCCGTGGGCGCCGCGGGCCGGGTCGCGCCGAGGC
GGGGGCGCCGGGGCTTCGTGGTGCGGAGCGTGGCGAGCGATCGGGAGGTGCGGGGGC
CCGCCTCGACCGAGGAAGAGCTTTCAGCCGTGCTAACTTCCATTGATTCTTCCGCCATCGC
GTCAAATATCCAGCACCATGCAGACTTCACACCGTTGTTCTCACCAGAGCACTCTTCACCC
CTAAAGGCTTACCATGCAACTGCTAAAAGTGTTTTTGATTCTCTGATCATCAATTGGAATGC
AACATATGACTATTACAACAAAGTGAATGCAAAGCAAGCTTATTACCTGTCCATGGAGTTTT
TGCAGGGAAGAGCTCTCACAAATGCTATTGGCAATCTAGAGCTAACAGGACAATATGCAGA
AGCGTTAAAACAACTTGGACAGAACCTAGAGGATGTTGCTAGTCAGGAACCAGATCCTGCC
CTTGGCAATGGTGGTCTAGGCCGTCTAGCGTCCTGTTTTCTGGATTCTATGGCAACCCTAA
ATTATCCAGCATGGGGATATGGACTTCGGTACAGATATGGCCTCTTTAAGCAAATCATAGC
AAAGGATGGTCAGGAGGAGGTAGCTGAGAATTGGCTAGAGATGGGAAATCCATGGGAGAT
CGTAAGAAATGATGTCTCTTATCCTGTGAAATTCTATGGCAAAGTGGTTGAAGGCACTGAT
GGGAGAAAGCACTGGATTGGAGGAGAGAATATCAAGGCTGTGGCACATGATGTTCCTATT
CCTGGCTACAAGACTAAAACTACTAATAATCTTCGTCTTTGGTCAACAACAGTACCATCACA
AAATTTTGATTTGGGTGCCTTTAATGCTGGGGATCATGCCAAGGCCAATGAAGCACATCTA
AATGCTGAAAAGATATGCCACGTATTGTATCCAGGGGACGAATCATCAGAGGGGAAAATTC
TTCGCTTGAAACAACAATATACATTATGCTCGGCCTCCCTGCAGGACATTATTTCTCGTTTT
GAGTCGAGGGCTGGTGATTCTCTCAACTGGGAGGACTTCCCCTCTAAAGTTGCAGTTCAG
ATGAATGACACTCATCCAACACTGTGCATTCCGGAGTTAATGAGAATACTGATGGATATAAA
GGGATTGAGCTGGAATGAGGCTTGGAGTATCACAGAAAGAACCGTTGCATACACTAACCA
CACGGTACTTCCTGAAGCTCTGGAGAAGTGGAGCTTGGACATTATGCAGAAACTTCTACCC
CGACATGTTGAGATCATAGAAACAATAGATGAGAAGCTGATGAACAACATCGTCTCAAAATA
TGGAACGGCAGATATTTCACTGTTAAAACAGAAGCTTAAAGATATGAGGATCTTAGACAAC
GTTGATCTTCCAGCCTCTGTTGCTAAACTGTTTATTAAACCTAAAGAGAAAACGGGGAAGTT
GCTTGTTCAATCTTTGGAGTCTATTGCTGAAGGTGACGAGAAAACTGAGTCACAAGAGGAG
GAAAACATTCTATCCGAGACAGCAGAGAAAAAGGGCGGATCTGACTCTGAAGAAGCTCCT
GATGCAGAAAAGGAGGATCCTGTGTATGAGTTAGATCCATTTGCAAAATACGATCCTCAGT
TACCTAGAGTTGTTCGAATGGCAAATCTCTGTGTTGTTGGTGGACATTCAGTTAATGGTGTG
GCTGAGATTCACAGTGAAATTGTTAAGCAAGATGTGTTCAATAGCTTTTATGAGATGTGGCC
TACTAAGTTCCAAAACAAAACAAACGGAGTAACTCCCAGGCGTTGGATCCGGTTTTGTAAT
CCTGAATTAAGTGCAATCATTTCAAAATGGATAGGCTCTGATGACTGGATTCTGAACACTGA
TAAACTTGCAGGACTGAAGAAGTTTGCTGATGATGAGGATCTGCAATCAGAATGGCGTACT
GCTAAAAGGAATAACAAGATGAAGGTAGTTTCGCTGATAAGAGACAAGACTGGATATGTTG
TCAGCCCAGATGCGATGTTTGATGTGCAGGTGAAAAGAATACATGAATATAAGCGGCAGCT
GCTAAATATCCTTGGTATCGTTTACCGATACAAGAAGATGAAAGAAATGAGTGCCAAAGATA
GAATAAAGAGCTTTGTTCCAAGGGTATGCATATTTGGTGGGAAAGCTTTTGCCACTTACGTA
CAGGCAAAGAGGATTGTGAAGTTTATCACAGATGTCGCAGCTACTGTAAATTATGACCCTG
ATGTTGGAGATTTACTGAAGGTTGTATTTGTCCCAGATTATAATGTTAGCGTTGCTGAGAAG
CTCATTCCTGCCAGTGAATTGTCCCAGCATATCAGTACCGCTGGAATGGAAGCTAGTGGAA
CCAGCAACATGAAGTTTGCAATGAATGGTTGTATTCTTATTGGAACTTTGGATGGTGCGAAT
GTGGAGATCAGAGAAGAGGTTGGAGAGGAGAACTTTTTCCTCTTTGGTGCACATGCACCT
GAAATCGCTGGTTTGAGGCAAGAGAGGGCTGAAGGAAAGTTTGTGCCTGACCCGAGATTT
GAAGAGGTTAAGGAATACGTCCGCAGTGGCGTCTTTGGGACTAGCAACTATGATGAATTGA
TGGGTTCTTTGGAAGGAAATGAAGGTTATGGACGTGCAGATTATTTTCTTGTTGGCAAGGA
TTTTCCCAGTTACATTGAATGCCAGCAAAAAGTTGATGAAGCATACCGAGATCAGAAGCTAT
GGACAAGAATGTCTATCCTCAACACAGCGGGTTCCCCCAAGTTCAGCAGTGACCGGACGA
TCCATGAGTACGCCAAGGACATATGGGATATCAGCCCCGTCATCATGCCCTGA
SEQ ID NO: 19 >Wheat (hexaploid) PHS1 5B (Cadenza) protein
MATASPPLATAFRPLAAAGGAGGGGAHAVGAAGRVAPRRGRRGFVVRSVASDREVRGPAST
EEELSAVLTSIDSSAIASNIQHHADFTPLFSPEHSSPLKAYHATAKSVFDSLIINWNATYDYYNKV
NAKQAYYLSMEFLQGRALTNAIGNLELTGQYAEALKQLGQNLEDVASQEPDPALGNGGLGRLA
SCFLDSMATLNYPAWGYGLRYRYGLFKQIIAKDGQEEVAENWLEMGNPWEIVRNDVSYPVKF
YGKVVEGTDGRKHWIGGENIKAVAHDVPIPGYKTKTTNNLRLWSTTVPSQNFDLGAFNAGDHA
KANEAHLNAEKICHVLYPGDESSEGKILRLKQQYTLCSASLQDIISRFESRAGDSLNWEDFPSK
VAVQMNDTHPTLCIPELMRILMDIKGLSWNEAWSITERTVAYTNHTVLPEALEKWSLDIMQKLLP
RHVEIIETIDEKLMNNIVSKYGTADISLLKQKLKDMRILDNVDLPASVAKLFIKPKEKTGKLLVQSL
ESIAEGDEKTESQEEENILSETAEKKGGSDSEEAPDAEKEDPVYELDPFAKYDPQLPRVVRMA
NLCVVGGHSVNGVAEIHSEIVKQDVFNSFYEMWPTKFQNKTNGVTPRRWIRFCNPELSAIISK
WIGSDDWILNTDKLAGLKKFADDEDLQSEWRTAKRNNKMKVVSLIRDKTGYVVSPDAMFDVQ
VKRIHEYKRQLLNILGIVYRYKKMKEMSAKDRIKSFVPRVCIFGGKAFATYVQAKRIVKFITDVAA
TVNYDPDVGDLLKVVFVPDYNVSVAEKLIPASELSQHISTAGMEASGTSNMKFAMNGCILIGTL
DGANVEIREEVGEENFFLFGAHAPEIAGLRQERAEGKFVPDPRFEEVKEYVRSGVFGTSNYDE
LMGSLEGNEGYGRADYFLVGKDFPSYIECQQKVDEAYRDQKLWTRMSILNTAGSPKFSSDRTI
HEYAKDIWDISPVIMP*
SEQ ID NO: 20 >Wheat (hexaploid) PHS1 5B (Cadenza) promoter (−2 kb upstream of ATG)
ATGCATTACCGAGTGGGCCCAGAGATACCTCTCCGTCATACGGAGTGACAAATCCCAGTCT
CGATCCGTGTCAACCCAACAGACACTTTCGGAGATACCCGTAGTATACCTTTATAGTCACC
CAGTTATGTTGTGACGTTTGGTACACCCAAAGCACTCCTACGGTGTCCGGGAGTTACACGA
TCTCATGGTCTAAGGAAAAGATACTTGACATTGGAAAAGCTCTAGCAAACGAACTACACGA
TCTTGTGCTATGCTTAGGATTGGGTCTTGTCCATCACATCATTCTCCTAATGATGTGATCCC
GTTATCAACGACATCCAATGTCCATAGTCAAGAAACCATGACTATCATTTGATCAACGAGCT
AGTCAACTAGAGGCTTACTAGGGACATGTTGTGGTCTATGTATTCACACATGTATTACGATT
TCCGGATAACACAATTATAACATGAATAACAGACGATTATCATGAACAAGGAAATATAATAA
TAATCTTTTATTATTGCCTCTAGGGCATATTTCCAACAATTGCGGTGAGGCGCGTGGAGGTT
GGCGTGTCCCCAAATCTAGTTATCTGGGTTGGGGTTTTTCTTTGGAAGGTTTATCTCAGTG
GTAGTGTCCCGTGAAGCGGATGACACGGCTATGTGTCTTGGTCCTCCATGCTCTAGTTTTG
ACATGTGAAGGTTGGCCAACTTCGGCGTTGTCAGGGAGCATATGAGATTTGTGTTCCTTTA
CTTCGCCTGGTCAGACCTACGGTATTATATAAGCCCTCTCATGTGTTTTCTTCTCTTGGGCA
GCAGTCAGTTACGCAAATCGTGGTCGCTGACATCTTCTAGTCCACATCGGCGACTTCCCGA
CCGCCGCTTCTACAAGCTCCTTATTTTTCAACAAGTTTTCTCCACCGAGATGGAAGTCAAGA
GGCGGCAACGGCTTTGCTCATGGCGCACTGCGGATGGATGCAGGAGAAGGAAGACATTC
CACACCCCTCAAACTATGAAGATGTAATTTTTAGTTTTTGTAAGTATTTTTGTGAGGATTTGT
GCTACTTTTAATATATATGGCCTTTTGCCTTTTTCCAGGAAAGGAAAAACTAAACCATTACAT
GAGGCCATTCCCATCCGTCCAATAAATACGACAAAACGCTTTACAGCACGGGGGCAATTCC
CAGCCGTCGAAATGCGCTGCAACGGAACTAGCTCTCCCAGACAGACACAGCGGCACATGA
GCCACGCCTCCCATCGCAACCGTGCACCGGCGGTGGGCCCACCGGCCAGCGCCCCCAC
ACGTGTACCGGCGGCCAGCATTGCACGGCACGGCGACGGAGAGATATTTTTAACGCCTTC
CAATTCAGAAGCTCCCCATCACTCGCTCGGGTATCAGAGCGGGCCGCGTGACTCTCGCGT
CGTCGCATCGCGCGCGCTGCACACCACCGTCAGCGCGACCACTGGCGCCA
SEQ ID NO: 21 >Wheat (hexaploid) PHS1 5D (Cadenza) genomic DNA
AGTGGTAGTGTCTCGTGAAAGCGGATGACGCGACTATGTGTCTTGGTCCTCCACGCTCTA
GTTTCGACAGGTGAAGGTTGGCCAGCTTCGGCCTCGTCGGGGAGCGTATGAGATTTGTGT
TCCTTTACTTCGTCTGGCCAGACTTCCCGACCGCCGCTTCTACAAGCTCCTTATTTCAAGC
AAGTTTTCTTCACCGAGATGGAAGTCCAGAGGCGGCAACAGCTTTGCTCACGGCGCAGTG
GCGGTGGATGCAGGAGAAAGAAGACATTCAACACCCCTAAACTATGAAGATGTAATTTTTT
ATTTTTGTAAGTGCTTTTGTGAGGACTTGTGCTACTTTTAATATGTGGCCTTTTGCCTTTTTC
CAGAAAAAAAAAACTAAACCATTACACGAGGAGAAGAGCCATTCCCAGCCGTCCAATAAAC
ACGACAAAACGCTTTACAGCACGGGGGCAGTTCCCAGCCGTCGAAATGCGCTGCAACGGA
ACTAGTAGCTCTCCCTGACACGGCGGCACATGAGCCACGCCTTGCGCCGTGCACCGGCG
GTGGGCCCACCGGCCAGTGCCCCCACACGTGTACCGGCGGCCAGCATTGCACGGCACG
GCGACGGAGAGATATTTTTAACGCCTTCCAATTCAGAAGCTCCCCCATCACTCACTCGGGT
ACAGAGCGGGCCGCGTGACTCTCGCGTCGTCGCATCGCGCGCGCTGCACACCACCGTCA
GCGCGACCACTGCCACCAATGGCGACCGCCTCGCCGCCGCTCGCCACCGCCTTCCGCCC
GCTCGCCGCCGGCGGCGGCACCGGAGGAGGAGGAGCGCACGCCGCGGGCTGGGTCGC
GCCGAGGCGGGGGCGCCGGGGGTTCGTGGTGCGGAGCGTGGCGAGCGATCGGGAGGT
GCGGGGGCCGGCCTCGACCGAGGAAGGTAAGCGCCGCGCGCTCCCGTTGGCTACGACG
ACCCGGAATGCTGGTCGGCTGTAATGTGGTTCGGATTTTCGCTGGTGCGACCTGAATTTGT
GATCCTCTGACTGACGGTCTCTGTGTTCCCCGGTTTCGGGCTATTGATTTTGTGCGCGCGC
ATTGCTAATCTACTGCTCTAGGATTTGTTAGGGTTCAGAGGAATCTTAAATTCGTGCATAAA
CTTCGACGAATGTATTCTGAATTTACTGAAACTGGCTTAGTTTGGCGCATCAAGTTAGAATG
ATGTATTTATGGTACAGTAGCAAATTCTGAGAAATTCTTCTGTGCCTGCACAGCCACCCCTT
GTGGATGATGCAGCAAGTGTCAACATACTGTCGCTACTATCTGTAGCTGTAACAAAATCTTA
CGCCTTGTTTTCTGGTGGTACAGTAGTATTATCATTTTACAAAGAGTAGCTGTAACAAAATC
TTACGCCTTGTTTTCTGGTGGCTTGCCATTGGGGAGTAGAATGCAAATGGTGATACAGTAT
ATTATCTACGATTTTGTGCAAGAATTTTCACACGAAAACAATGTCCCATGCAGTTTTTAGTTT
TGACTCTTGACTGTTTAATACCTACTCTTTGTAAGATTTCGGATCCTAATATTGTTGTTCTTG
CAACTCAGAGCTTTCAGCCGTGCTAACTTCCATTGATTCTTCCGCCATCGCGTCAAATATCC
AGCACCATGCAGACTTCACACCGTTGTTCTCGCCAGAGCACTCTTCACCCCTAAAGGCTTA
CCATGCAACTGCTAAAAGTGTTTTTGATTCTCTGATCATCAATTGGAATGCAACATATGACT
ATTACAACAAAGTGAATGCAAAGCAAGCTTATTACCTGTCCATGGAGTTTTTGCAGGTCTCA
CAATATACTTCCTTTAGCCTTATCTCAATACTTTTTGGTAACTGCGTTATGATGTTATATGGT
TATTATCTGTGTACTGCAGGGAAGAGCTCTCACAAATGCTATTGGCAATCTAGAGCTAACA
GGACAATATGCAGAAGCGTTAAAACAACTTGGACATAACCTAGAGGATGTTGCTAGTCAGG
TAAGTGATTTTTGTCAGAGATTGGTTTTTAGTCTGTATCAAAATTATCGCAGAGCCATTTTTT
TTTTCTAAAAAACACATTTTATTATTAATCATCAAGTTCCTTTTGTTTAAAAACTATCTGAAAA
GAAGAAGTATTTTCCCCTGGGGGTTCCAGCATCTGACTTATATTCTTTATGCAGTTTTCAGA
GTGAGTGACTATGAAATTACGCCAAAACACGAGCTTATTGATGCCTAGAGTGCCCTGTAGT
TTAGAAATCTATTATTGGATATGTTACTTTTCTATTACAATGAAACAGGAACCAGATCCTGCC
CTTGGCAATGGTGGTCTAGGCCGTCTAGCGTCCTGTTTTCTGGATTCTATGGCAACCCTAA
ATTATCCAGCATGGGGATATGGACTTCGGTACAGATATGGCCTCTTTAAGCAAATCATAACA
AAGGATGGTCAGGAGGAGGTAGCTGAGAATTGGCTAGAGGTAGTGATATGTCTTCTACTTG
TTTACTGTTTCGAAGTGTTGACTATGTTGGTTTATGTTTTAATGACCTGCATGTTAGCTTCTT
TTGCTGTTATAACTGATGCTGTATCTCCAGATGGGAAATCCATGGGAGATTGTAAGAAATGA
TGTCTCTTATCCTGTGAAATTCTACGGCAAAGTGGTTGAAGGGACTGATGGGAGAAAACAC
TGGATTGGAGGAGAGAATATTAAGGCTGTGGCACATGATGTTCCTATTCCTGGCTACAAGA
CTAAAACTACCAATAATCTTCGTCTTTGGTCAACAACAGTACCATCACAAAATTTTGATCTG
GGAGCTTTCAATGCTGGGGATCATGCCAAGGCCAATGAAGCACATCTAAATGCTGAAAAG
GTATTCTTCCGTCTACATTTATTTTGAACGTAAACCATTAACTCTCAACAACTATTGATTCAA
AGGCTAAACACTGTCTTGCTAGTAGTTACTGTGTTTTCTCTTAGATGTCATTTTAGAATGTG
CGCGGTCAATCGTTTGATAACTTTGATAGTAATAATATACTGGTATACACAAACCTATTTGG
GTTTCTCATGTAAAAATGTCGTCACCAAATTTTTCATGAAAATACTTATTGTATTTTCATGTAA
ATAGAAAAGCTGATGGTCAGGTGTTTTGTGTGTCAAATGACAAGTAAAAATGAACAGAGTTT
GTGCACCAATAACTTCTTCACCTGGTGTTTGTAATTCTTTCGGTTGTATTTATGTGTTTTCTT
ATTCTGCCTTAATTAACTCAGCATATGCATCAGGTTACATTACTCCTCTGTGTCAAGAAAAA
AGAAGTCTGATGAGATGCAGTCCAGTAGTCTGTAGATGGCACATTGTAGAAGAACTTTTCA
CATGGAAACGTTACTCTGTGATTTTTGTTATGCCTTTTGAGTGTTTGTGCATTGTGCACATAT
ACCATTAAGTATAAGTGTTCAAAAGTGGTTCTCTCATACTGTGCAAACTAGCTAAACAACTT
GACTGGACAAGATAAATTGTACTCACATCTGAAGCTGCCGGGTTGGACAGATTTGTCCGAA
CCATCTTTGAACATCCCTACTAGTGTGCATGGGTTAAATTGTTGACTTTTCCTCGTTTGTTC
AGTAGTAGGACTATCTAAATATATTTATATTCAAGTTTTCTTTGTGTGTTACGTTATAAACTTT
TAGTCATAATTACATTTGTGACCATTGTGGACAAAATTTATCATGTCTAACTTAAGTTTGTGA
CTGGTCTTCTCATCATGCCAGATATGCCACGTATTGTATCCAGGGGACGAATCATCAGAGG
GGAAAATTCTTCGCTTGAAACAACAATATACATTATGCTCGGCCTCCCTGCAGGACATTATT
TCTCGTTTTGAGTCGAGGGCTGGTGATTCTCTCAACTGGGAGGACTTCCCCTCTAAAGTTG
CAGTTCAGATGAATGACACTCATCCAACACTGTGCATTCCCGAGTTAATGAGAATACTGAT
GGATATAAAGGGATTGAGCTGGAATGAGGCTTGGAGTATCACAGAAAGGTACCACAGAAG
GGTACTGCAGTTTGATGTGTTCTGCTGTTCCTCCAGTTTCAATTAACTAATTTTATCCCTCAT
ATGTCTTCCATTCAATAATAAACTGATTGTCTGATATCATTTGTAGAACCGTTGCATACACTA
ACCACACGGTACTTCCTGAAGCCCTGGAGAAGTGGAGCTTGGACATTATGCAGAAACTTCT
ACCTCGACATGTTGAGATCATAGAAACAATAGATGAGGAGGTAACAAATGTGCAGATAATA
TTATACAATTAATTTCATGCTCTGTAGGTCAACTTATGGTTTACTTATCTTCATCGTTGATTTT
AAAACAGCTGATGAACAACATCGTCTCAAAATATGGAACGGCAGATATTTCACTGTTAAAAC
AGAAGCTTAAAGATATGAGGATCTTAGACAACGTTGATCTTCCAGCCTCTGTGGCTAAACT
GTTTGTTAAAACAAAAATGAAAAAGGGGAAGTTGCTTGTTGAATCTTTGGAGTCTATTGCTG
AAGCTGACGAGAAAACTGAGCCAGAAGAAGAGGAAAACATTCTATCCGAGACAACAGAGA
AGAAGGGCGAGTCTGACTCTGAAGAAGCTCCTGATGCAGAAAAGGAGGATCCTGAGTATG
AGTTAGATCCATTTGCAAAATACGATCCTCAGTTACCTAGAGTTGTTCGAATGGCAAACCTC
TGTGTTGTTGGTGGACATTCAGTTAATGGTGTGGCTGAGATTCACAGTGAAATTGTTAAGC
AAGATGTGTTCAATAGCTTTTATGAGGTATTTGCACGATGATTTGGAAACCATGATCTTATC
TTCTAACGGCTGCTATAATCGCCAGGTAATATGGTATTCACCATCTGCCATTTTCTTGTGTT
CTTTGCCAGATGTGGCCTACTAAGTTCCAAAACAAAACAAACGGAGTAACTCCCAGGCGTT
GGATCCGGTTTTGTAATCCTGAATTAAGTGCAATCATTTCAAAATGGATAGGCTCTGATGAC
TGGATTCTGAACACTGATAAACTTGCAGGACTGAAGAAGGTACTTCCAGTAATTTTAATTTC
CAATAATCAGTAATCTAATGCTTCCTTCACTTCCATGTTACCCGTGCCTTGTTTTTACATTGA
TATGTGTTCATCCCGTAATAGTTTGCTGATGATGAGGATCTGCAATCAGAATGGCGTACTG
CTAAAAGGAATAACAAGATGAAAGTAGTTTCACTGATAAGAGATAAGACTGGATATGTTGTC
AGCCCAGATGCGATGTTTGATGTGCAGGTATATTTTGGACAAGAAACATCTGTCTTCTGTTA
AATATGGTTTAACTCGGATCTTGGTACTTGACACTTAGGTGAAAAGAATACATGAATATAAG
CGGCAGCTGCTAAATATCCTTGGTATCGTTTACCGGTACAAGAAGATGAAAGAAATGAGTG
CAAAAGATAGAATAAAGAGCTTTGTTCCAAGGGTATGCATATTTGGTGGGAAAGCTTTTGC
CACTTATGTACAGGCAAAGAGGATTGTGAAGTTTATCACAGATGTCGCAGCTACTGTAAATT
ATGATCCTGATATTGGAGATTTACTGAAGGTAAATTTGTGTCTTCAATGGCACTTATTATTCT
TAATTTTCATGTTCTGTCACTGGGAATTTGACTCCGACTAGGGGTTACTGAAAAGCAGTGAA
TGTTCCGAGTTTCAAAAGAAAACAGAGCAGTGAATGTTCTTAATGTCGGTGCTTGTAGAAC
CGAGAAACTGAATAGCCACAGTATTTTTTAGAGAACAATAGCCACAATTGTTTTGACCCTTC
TATTGCTATCAAGCATCTGATGCCGATTTTACTGATAACTAGATGAGGTAAATACAAGGTGA
CAGGTTAAGTTCTGAGGATTTTTGTTTGTTATACAGGTTGTATTTGTCCCAGATTATAATGTT
AGCGTTGCTGAGAAGCTCATTCCTGCCAGTGAATTGTCTCAGCATATCAGGTAATATTCTG
GCTACTCTTATATAGTATGTGAATACATAGAAAAGGAGTCTTACCTGGAGTTGGCTGATGTG
GAAATTTTCATGTGCCGTTTTGTTTGCTATAATATATACCTGGAGTCTTGCTGGGATAGTGC
CATCCAGAAGTGCCACGCCAGCTTCTATTTCTGTACAGGATGTGCATTTTTAACACTATTAC
TATCCATTCAGTACCGCTGGAATGGAGGCTAGTGGAACCAGCAACATGAAGTTTGCAATGA
ATGGTTGTATTCTTATTGGAACTTTGGATGGTGCGAATGTGGAGATCAGAGAAGAGGTTGG
AGAGGAGAACTTTTTCCTCTTTGGTGCACATGCACCTGAAATCGCTGGTTTGAGGCAAGAG
AGGGCTGAAGGAAAGGTACCATCACTTTGCAAACATCTCCAAATTCGTTCCTCCAGTATGT
GAAACAAATATCCACTTGTTTATATAGCTGTGCCTACTATCAAACTACTCCCTCCGTCCATA
ATATAAGAGTGTTTTTTACACTACACTAGTGTCAAAAACGCTCTTATATTATGGGACGGAGG
GAGTAGTAAAGATCTCTCCCAAATGTGTGCTGTCCAAATAGTTTAGCTGAAGGAAAATTATA
AATCATTTTCCTTTGCACTTGTAGTTTGTGCCTGACCCGAGATTTGAAGAGGTTAAGGAATA
CGTCCGCAGTGGCGTCTTTGGGACTAGCAACTATGATGAATTGATGGGTTCTTTGGAAGGA
AATGAAGGTTATGGACGTGCAGATTATTTTCTTGTTGGCAAGGATTTTCCCAGTTACATTGA
ATGCCAGGAAAAAGTTGATGAAGCATACCGAGATCAGAAGGTAAGTTGTATAACTGACTCT
GTTTGTTTTCTGGCTGAAATGCACACAGTGATTATGCAAACTTTCTGATATGTGATGGCTGA
CATCAGTGTTTCACTGTGAACGACTTCACTGATTCGTAGGAAGGAATATTTCTTAGTTCAAA
GTTTCAAACTGAAGTATCTATCGGTGTGTTGGAAGATCATAAACAATCAGAGAATATAGTTG
CTCGATGTGCTATATTACTCATCTGTTGGCTGTTGAGTGTTGATACATGAGCTTTGATGAAC
CTAGCTAGACCTTCGGGAAAGTGCTTCTATGCATTCCAATAAAAAAAACTTATATCTTAACA
AAACAATTAGGTTGAACCTCGCCAGGGGGCTTTTTGTTATCTGTTTCCTGAAATGAAAATCA
GAAGCGGGGAGCTCCTCAATTTGAAAAAAAAATCAAGGGTAACTGAGGGTCTGGTTTCCCT
CTGTTGGAGCCCCCAAGAATTCTGTGGTCTTATTTTTGTAATAGTATAAGATGCACTGCTCC
GTTAAGTTTCTCCTTCATCCATTTGCTTTTATTGCTTCGTGACATTTCGTGTAACAGCTCAGA
TTACCATGCATCCCATTACACATTATTTTGCTACTGTGGATATTTCCACTCTGTTTCATGCTT
GACATTTTTAATTCGTTACATCGTGACCAACTAAACATAGTCTGATAGATTTTCAGAGTTTTG
ATCCTCCTAATCACATAATATCCGTTTCTACCTGCAGCTATGGACAAGAATGTCTATCCTCA
ACACAGCGGGTTCCCCCAAGTTCAGCAGTGACCGGACGATCCACGAGTACGCCAAGGACA
TATGGGATATCAGCCCTGTCATCATGCCCTGAATAATTCAAGCAGCTTCCAGGAACCTTGA
GAGTCTATGAGGCGCGATTGGTCGCAGATTTGCCTCGATTCAGCGAGAATGTATTGCTCCC
CCCAGTTTTGTATAGGCATATGCAAGTTCTGGCATGGACCTGAGTGCTGAGAGCATGCAG
GGTAGTTCATCCTTAGCAATCTTGCATGTCAGTTGGCCAGTATACCCAGGCTTATGGTGTG
TATTTCGCCGAATAAAGTTGGAGCAGAATCATCAAACAAACATACTTTGGTGTATCTGTAAT
GATAAATAAAGGGTAAATGACTGAACAAAAAGTGTCGTTGGCGCACACATGTAGTAGAAGC
ATGTTTCTTCAGAGAGGGTGCAAAGCTGAAAGCACCCCCAGCTTTGAAATGAGAATGCAGA
AGTTGGGAAAACATTTGTTTACATCCAAAGTGCAACGGGATTCGTCAAGAGTTCAGAGTCT
TCCATCACGGCCATAGAGCATGTCCAATAGCAGTCCTATAATAGAGCAGTAGCAGTGGTCC
CTCATATTTTGAACCAAAAACGCAGCTCCAACGGCTGCCCTATTTGTAAAATTTACTTCAAA
ATTTGTTGGACAGCCCGAAAATGCAACACCAGCTGATGTAAATGTACAACACTTCGGCTGC
TGCCCTAAAATTTGCTTCAAGTGGCGAACCACTCAAGACCACAAGAAAACAGTTGTTTTAC
GTTACTTTGTGCCAAAAACGTTGCTCCAACAACTGCGCTATAATAGGCCACCCTAATGTATT
TTAGGGCTGTTGTTGGAGATACATATTTTCGGGTGCCTAAAAATAAAATCATTTTTTGGTGC
CTTACGTTTTACGCCAACCACTCTTTTGTGCCCTATTATAGGGCTGCTATTGGAGATACTCT
AACAGTTCCATTTTTCCATAAAAGTTTCTGATTTGGAGGGAGTTGAGCAAAAAACAAAGTTT
TTGTAAACGTGAATTTTTAGTGGCCTTTATAGTTAGCCCAACTCCCCCTCCACCTAA
SEQ ID NO: 22 >Wheat (hexaploid) PHS1 5D (Cadenza) CDS
ATGGCGACCGCCTCGCCGCCGCTCGCCACCGCCTTCCGCCCGCTCGCCGCCGGCGGCG
GCACCGGAGGAGGAGGAGCGCACGCCGCGGGCTGGGTCGCGCCGAGGCGGGGGCGCC
GGGGGTTCGTGGTGCGGAGCGTGGCGAGCGATCGGGAGGTGCGGGGGCCGGCCTCGAC
CGAGGAAGAGCTTTCAGCCGTGCTAACTTCCATTGATTCTTCCGCCATCGCGTCAAATATC
CAGCACCATGCAGACTTCACACCGTTGTTCTCGCCAGAGCACTCTTCACCCCTAAAGGCTT
ACCATGCAACTGCTAAAAGTGTTTTTGATTCTCTGATCATCAATTGGAATGCAACATATGAC
TATTACAACAAAGTGAATGCAAAGCAAGCTTATTACCTGTCCATGGAGTTTTTGCAGGGAAG
AGCTCTCACAAATGCTATTGGCAATCTAGAGCTAACAGGACAATATGCAGAAGCGTTAAAA
CAACTTGGACATAACCTAGAGGATGTTGCTAGTCAGGAACCAGATCCTGCCCTTGGCAATG
GTGGTCTAGGCCGTCTAGCGTCCTGTTTTCTGGATTCTATGGCAACCCTAAATTATCCAGC
ATGGGGATATGGACTTCGGTACAGATATGGCCTCTTTAAGCAAATCATAACAAAGGATGGT
CAGGAGGAGGTAGCTGAGAATTGGCTAGAGATGGGAAATCCATGGGAGATTGTAAGAAAT
GATGTCTCTTATCCTGTGAAATTCTACGGCAAAGTGGTTGAAGGGACTGATGGGAGAAAAC
ACTGGATTGGAGGAGAGAATATTAAGGCTGTGGCACATGATGTTCCTATTCCTGGCTACAA
GACTAAAACTACCAATAATCTTCGTCTTTGGTCAACAACAGTACCATCACAAAATTTTGATCT
GGGAGCTTTCAATGCTGGGGATCATGCCAAGGCCAATGAAGCACATCTAAATGCTGAAAA
GATATGCCACGTATTGTATCCAGGGGACGAATCATCAGAGGGGAAAATTCTTCGCTTGAAA
CAACAATATACATTATGCTCGGCCTCCCTGCAGGACATTATTTCTCGTTTTGAGTCGAGGG
CTGGTGATTCTCTCAACTGGGAGGACTTCCCCTCTAAAGTTGCAGTTCAGATGAATGACAC
TCATCCAACACTGTGCATTCCCGAGTTAATGAGAATACTGATGGATATAAAGGGATTGAGC
TGGAATGAGGCTTGGAGTATCACAGAAAGAACCGTTGCATACACTAACCACACGGTACTTC
CTGAAGCCCTGGAGAAGTGGAGCTTGGACATTATGCAGAAACTTCTACCTCGACATGTTGA
GATCATAGAAACAATAGATGAGGAGCTGATGAACAACATCGTCTCAAAATATGGAACGGCA
GATATTTCACTGTTAAAACAGAAGCTTAAAGATATGAGGATCTTAGACAACGTTGATCTTCC
AGCCTCTGTGGCTAAACTGTTTGTTAAAACAAAAATGAAAAAGGGGAAGTTGCTTGTTGAAT
CTTTGGAGTCTATTGCTGAAGCTGACGAGAAAACTGAGCCAGAAGAAGAGGAAAACATTCT
ATCCGAGACAACAGAGAAGAAGGGCGAGTCTGACTCTGAAGAAGCTCCTGATGCAGAAAA
GGAGGATCCTGAGTATGAGTTAGATCCATTTGCAAAATACGATCCTCAGTTACCTAGAGTT
GTTCGAATGGCAAACCTCTGTGTTGTTGGTGGACATTCAGTTAATGGTGTGGCTGAGATTC
ACAGTGAAATTGTTAAGCAAGATGTGTTCAATAGCTTTTATGAGATGTGGCCTACTAAGTTC
CAAAACAAAACAAACGGAGTAACTCCCAGGCGTTGGATCCGGTTTTGTAATCCTGAATTAA
GTGCAATCATTTCAAAATGGATAGGCTCTGATGACTGGATTCTGAACACTGATAAACTTGCA
GGACTGAAGAAGTTTGCTGATGATGAGGATCTGCAATCAGAATGGCGTACTGCTAAAAGGA
ATAACAAGATGAAAGTAGTTTCACTGATAAGAGATAAGACTGGATATGTTGTCAGCCCAGAT
GCGATGTTTGATGTGCAGGTGAAAAGAATACATGAATATAAGCGGCAGCTGCTAAATATCC
TTGGTATCGTTTACCGGTACAAGAAGATGAAAGAAATGAGTGCAAAAGATAGAATAAAGAG
CTTTGTTCCAAGGGTATGCATATTTGGTGGGAAAGCTTTTGCCACTTATGTACAGGCAAAG
AGGATTGTGAAGTTTATCACAGATGTCGCAGCTACTGTAAATTATGATCCTGATATTGGAGA
TTTACTGAAGGTTGTATTTGTCCCAGATTATAATGTTAGCGTTGCTGAGAAGCTCATTCCTG
CCAGTGAATTGTCTCAGCATATCAGTACCGCTGGAATGGAGGCTAGTGGAACCAGCAACAT
GAAGTTTGCAATGAATGGTTGTATTCTTATTGGAACTTTGGATGGTGCGAATGTGGAGATCA
GAGAAGAGGTTGGAGAGGAGAACTTTTTCCTCTTTGGTGCACATGCACCTGAAATCGCTGG
TTTGAGGCAAGAGAGGGCTGAAGGAAAGTTTGTGCCTGACCCGAGATTTGAAGAGGTTAA
GGAATACGTCCGCAGTGGCGTCTTTGGGACTAGCAACTATGATGAATTGATGGGTTCTTTG
GAAGGAAATGAAGGTTATGGACGTGCAGATTATTTTCTTGTTGGCAAGGATTTTCCCAGTTA
CATTGAATGCCAGGAAAAAGTTGATGAAGCATACCGAGATCAGAAGCTATGGACAAGAATG
TCTATCCTCAACACAGCGGGTTCCCCCAAGTTCAGCAGTGACCGGACGATCCACGAGTAC
GCCAAGGACATATGGGATATCAGCCCTGTCATCATGCCCTGA
SEQ ID NO: 23 >Wheat (hexaploid) PHS1 5D (Cadenza) protein
MATASPPLATAFRPLAAGGGTGGGGAHAAGWVAPRRGRRGFVVRSVASDREVRGPASTEEE
LSAVLTSIDSSAIASNIQHHADFTPLFSPEHSSPLKAYHATAKSVFDSLIINWNATYDYYNKVNAK
QAYYLSMEFLQGRALTNAIGNLELTGQYAEALKQLGHNLEDVASQEPDPALGNGGLGRLASCF
LDSMATLNYPAWGYGLRYRYGLFKQIITKDGQEEVAENWLEMGNPWEIVRNDVSYPVKFYGK
WVEGTDGRKHWIGGENIKAVAHDVPIPGYKTKTTNNLRLWSTTVPSQNFDLGAFNAGDHAKAN
EAHLNAEKICHVLYPGDESSEGKILRLKQQYTLCSASLQDIISRFESRAGDSLNWEDFPSKVAV
QMNDTHPTLCIPELMRILMDIKGLSWNEAWSITERTVAYTNHTVLPEALEKWSLDIMQKLLPRH
VEIIETIDEELMNNIVSKYGTADISLLKQKLKDMRILDNVDLPASVAKLFVKTKMKKGKLLVESLES
IAEADEKTEPEEEENILSETTEKKGESDSEEAPDAEKEDPEYELDPFAKYDPQLPRVVRMANLC
VVGGHSVNGVAEIHSEIVKQDVFNSFYEMWPTKFQNKTNGVTPRRWIRFCNPELSAIISKWIGS
DDWILNTDKLAGLKKFADDEDLQSEWRTAKRNNKMKVVSLIRDKTGYVVSPDAMFDVQVKRIH
EYKRQLLNILGIVYRYKKMKEMSAKDRIKSFVPRVCIFGGKAFATYVQAKRIVKFITDVAATVNYD
PDIGDLLKVVFVPDYNVSVAEKLIPASELSQHISTAGMEASGTSNMKFAMNGCILIGTLDGANVE
IREEVGEENFFLFGAHAPEIAGLRQERAEGKFVPDPRFEEVKEYVRSGVFGTSNYDELMGSLE
GNEGYGRADYFLVGKDFPSYIECQEKVDEAYRDQKLWTRMSILNTAGSPKFSSDRTIHEYAKDI
WDISPVIMP*
SEQ ID NO: 24 >Wheat (hexaploid) PHS1 5D (Cadenza) promoter (−2 kb upstream of ATG)
TATGTTAGTCCAAATAAATATCATCTAAAATTGTGCCTAAACCATATACAAATGATGTAAATG
TGGACATAATACTTCATAAAGTATAGATATGTTTGAGACGTATCAGGAGCGTCGACCTTTTT
GGGAAGCCAAGTGAGCAAAGCCTCATCAAGTCTTGGGAATCCACGGTCATTGAGGGAGAA
AAATTTATCAAAGGTCTCACACATACCCCCTTTGATGATGGACCAGCAGGACCGCAGTAAT
TTGGCCGTGAAGCCATCTAGCCCCGTCAACTTGCCGGAAGGGAACCTCTTGACCGCCTCC
TAAATTTCATCCTCAGTGAAGGGTTGCTCAAGTGCAGGGAGGTCAAAGTGATGCGGGTCG
GTACTATGGAGATCAAGGGAGAGAGTCAGTGCTCAAAAGTGTCAAGAATGGAAGAGAAGT
GATCATATGCCGCCCTGGCCATATCGGGCTCGCCAGAATGTGCCCACCATCAGACTGCAC
CCCTAGGATTATTAACTTCTGTCGTCGGTGCACAGAATGGATCTTGAAGAAGGTGGAATTT
GTGTCGCCCTCACGTAGCCAGGAGAACCTGGCAGGCAGATGTGCAATCGACCTCTCCACC
GAAGCAAACCCTAGGTAGGCGCGCTTCAGCTCGCGGAGTAACCATACTTCTGGAGGAGCA
AGCAGGCGGTGATCTTGAGCCGCATCGAGATGAGCGGTCAACTCCCGAGCGAGAAGCAT
CTGAGTGGAGATGTTACCAAGCGTTCTAGACCTCCAACTCTACAGTCAGCGCATTGTCAGC
TTGAGCTTGGCGTAGATCCCCTGAAAATGATTGGGGTCATGCACGCCAGAATTCCAGGTGT
CAGAGACCACCTAATGAAAACAATCGAGCTTTGGCCAAAATAGCTCAAAATGGAATCTCCG
ATCACCTTGGGAGTGAAAATGTTACTAGTTTCGTTGGAGGCCAAGGTAGTTATGTATTTGTG
TGTGCAATTACTAATCATTGGCCCTTAGCAAACTCCTATTGGTTCAAACCGTTTCACTTTGA
AAAAAATGCTACTTGCTACAAACCGTTTCACTTGGAGGGGCGTCTTTTCTAGTTGAGAGGG
AGCAAAAGGCGTATCAAGACGACAACGCCACCCTCGGCAATGTACAAGTCCTTCCCCGTC
CTCGTTTCGATGCTGCTTTTTGTCATCGCAGCGAGGCGTGTAGAGGGTGGTGTGTCCCCA
GATCTAGTTATCTGGGTTGGGGTTTTTCTTTGGCAGGTTTATCTCAGTGGTAGTGTCTCGTG
AAAGCGGATGACGCGACTATGTGTCTTGGTCCTCCACGCTCTAGTTTCGACAGGTGAAGG
TTGGCCAGCTTCGGCCTCGTCGGGGAGCGTATGAGATTTGTGTTCCTTTACTTCGTCTGGC
CAGACTTCCCGACCGCCGCTTCTACAAGCTCCTTATTTCAAGCAAGTTTTCTTCACCGAGAT
GGAAGTCCAGAGGCGGCAACAGCTTTGCTCACGGCGCAGTGGCGGTGGATGCAGGAGAA
AGAAGACATTCAACACCCCTAAACTATGAAGATGTAATTTTTTATTTTTGTAAGTGCTTTTGT
GAGGACTTGTGCTACTTTTAATATGTGGCCTTTTGCCTTTTTCCAGAAAAAAAAAACTAAAC
CATTACACGAGGAGAAGAGCCATTCCCAGCCGTCCAATAAACACGACAAAACGCTTTACAG
CACGGGGGCAGTTCCCAGCCGTCGAAATGCGCTGCAACGGAACTAGTAGCTCTCCCTGAC
ACGGCGGCACATGAGCCACGCCTTGCGCCGTGCACCGGCGGTGGGCCCACCGGCCAGT
GCCCCCACACGTGTACCGGCGGCCAGCATTGCACGGCACGGCGACGGAGAGATATTTTTA
ACGCCTTCCAATTCAGAAGCTCCCCCATCACTCACTCGGGTACAGAGCGGGCCGCGTGAC
TCTCGCGTCGTCGCATCGCGCGCGCTGCACACCACCGTCAGCGCGACCACTGCCACCA
SEQ ID NO: 25 >Wheat (tetraploid) PHS1 5A (Svevo reference genome) genomic DNA
CGAGGGCGGCGGCGAAGGGATCTGGTGTGGTGTCGCTCGAGGGCACCGGACTACCGGA
GGCTTCGCTCGCTCGACCGCACGTTGCGAGTCCAAGACGATTTTTTTTCGCTGGTTTTCTT
TCGTCGGTTTAATTTCGTTCGATCTACTTTGCCTGCGAGGGGAGTGCCTGCAGGCATCGCT
CCCTGATAACGTAGGTTTGTTCGGTTTTTTTAACGTACGAGGGGATGTGGGAGGAAATACC
AAAAAAGTATCAAAAAAGACCGGGTGAGGTGGGACGAAAATAAAACCTGGAATGCGACCTA
CCAACTGAGATATTAGGAGTAGAGATATATGTTTTGTATCTCCTTTTTTTGGTATAATTTTTTT
TGGTGAAACGCCGTTTCTTTTGTAAGCGAAGATCCATATCTCCGATCAGGCAGATTCTGCA
CATTCTTTTCTTATTTTAGGATTGCTCTTGCAAAAAGAAGATCATCAATCAACTTAGGGATCC
TCCACGCAGACCCACCACGCAGCCTAGGGGCACCGTCTGGCCATCTGGAGCCCCGGTGG
CTCCCTTGGACCCCATCTTCTCGCTAGTGGGGCCTTGGTCCAAAATAATCCATGTAATTTTT
CATGTTTTCCGGCTTCGTATTTTTTGGTAAAAACCCAAAAACAATAGAAAAGAAGAATTGGC
CTCTGACACTAGATTGATAGGTTAGTCCAAATCAATATTATCAAAAATTGTGCCCAAACCAT
ATACAAATGATATAAATATAACTAGAATACTTCATAAATTTAGATACGTTTGATAAAGTGCCA
AGAATGGAAGAGAAGTGATCATATGCCGCCCTGTCCATATCGGGCTCATCAGAATGTTCCC
ACCATCAGACTCCAGCCCCAGGATTATTAACTTATGTCGGCGGTGCGCGGAATGGATCTTG
AAGAAAGCGGAATTTGTGTCGCCCTCACGTAACCAGGAGAACTTGGCAGGTTGACGCGCA
ATCGACCTCTCCAGCAAAGCAAGTCCTAGGTAGGCGCGCTTCAGCTCGCAGAGTAACCGT
ACTTCTGGAGGAGCAAGCGGGTGGTGATCTTGAGCCGCCGAGACGAGCAATCAATTACCG
AGCGAGAAGCATCTGAGTGGAGATGTTACCGAGCGTTCTGGAGCTCCAACTCTGCCGTCA
GTGCATTGTCAGCTTGTGCTTTGCGTAGATTCGCCGGAAAGGATCAGGATCATGCACGCC
AGAATTCCAGGTGTCAGAGACCACCTAATGAAAACCATCGAGCTTTGATCAAAATAGCTCA
AGATGGAATCTCCGATCACCTTGGGAGCGAAAATCTTACTAGTTTCGTTCAAGGCCAAGGT
AGTTATATATTTGTGTGTGCAATTATTAATCATTGGCCGCTTAGCAAACTCTTATTGGTTCGA
TAAACATTAAAGTCATCCCCACTTTGAGAAAAAATGCTACTTGCTACAAACCCTNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNGCAAGTAATAAATCCTTGCCGCCTCGTCCTTGTTTGGATGCTGCTTCTTGTCATC
TCAAGGAGGCGTGTGGAGGGTGGTGTGTCCCCGGATCTTGTTCTCTCGGTTGGGTTTTTT
CTATGGCAGGTTTATCTCAGTGGTAGTGTCACGTGAAGCAGATGACGCGACTATGCGTCTT
GGTCCTCTGCGCTCTAATTCCGGCAGATGAAGGTTGACCAGCTTCAGCCTCGTCGGGGAG
CGTATGAGATTTGTGTTTTTTGACTCCATCTGGTCGGACCTGTGGCATTATGTAGACCCTCT
CATGTATTCTTTTTCTCCTGGACAGTAGTCTGTTACACAAATCGTGGTCGTCGGATCTTCTA
GTCCACATCGGCGACTTTTCCGACCGCCACTTCTACAAACTCCTTATTTTCAACAAGTCTCC
TTCATTGAGATGGAAGTCCAGGCAATGGCTTTGCTCACGGCACACTGCCGGCGGATGCAA
GAGAAAGAAGACATTCGACACCCCTAAAACTATGAAGATGTAATTTTTAATCTTTGTAAGTG
TTTTTGTGAGGATTTGTGCTACTTTTAATATATTGCCTTTTGCCTTTTGCAAGGGAAAAAAAA
CTAAACCATTACACGAAGAGAAGAGCCATTCCCAGCCGTCCAATAAACACGACAAAACGCT
TTACAGCACGGGGGCAATTCCCAGCCGTCGAAATGCGCTGCAACGGAACCAGCGCTCCC
CGACACGGCGGCACATGAGCCACAACTCGTACCATGCACCGTGCACCGGCGGTGGGCCC
ACTGGCCAGCGTCCCCACACGTGTACCGGCGGCCACAATTACACGGCACGGCACGGCGA
CGGAGAGATATTTTAACGCCTTCCAATTCAGAAGCTCCCCCATCACTCACTCGGGTACAGA
GCGCAGCGGGCCGCGTGACTCTCGCGTCGTCGCATCGCGCGCGCTGCACACCACCGTCA
GCGCGACCACTGCCGCCAATGGCGACCGCCTCGCCGCCGCTCGCCACCCCCTTCCGCCC
GCTCGCCGTCGCCGGAGGTAGCGGCGGACTCGTCGTGGGCGCCAGGGCCGTGGTACCG
CCGAGGCGGGGGGGGCGGGGGTTCGTGGTGCGGAGCGTGGCGAGCGATCGGGAGGTG
CGGGGGCCCGCCCCGACCGAGGAAGGTAAGCGGCCGCGCGCTCCCATTGGCTGCCACG
ACCTGGAATGCTGGTCGGCTGTAATGTGGTTCGGATTTTCGCTGGTGCGACCTGAATTTGT
GATCCTCTGACTGACGGCCTATGTGTTCCCCGGGTTTCGGGCTGTTGATTTTGTGCGCGC
GCATTGCTAATCTACTTCTCTAGGATTTGTTAGGGCTCAGAGCAATCTTAAATTCGCGAATA
CACTTCGACGAATGTAGTCTGAATTTACTGAAACTAGCTTAGTTTGGCGCGTCAAGATAGA
CTGATGTATTTATGGTAGCAAATTCTGAAAACTTCTTCTGTGCGTGGACAGCCACCCCTCGT
GGATGATGCAGCAAGTGCTAACACAGTGTTTGCTAGTACTTGCTAGTATCTGTAGCTGTAA
CAAAATCCTTCGAATCTGAACTAGTGAGATAAATCGTTTGAGATTTTTGCTGAAGATTACAC
AGTATCAATATTTCTGAAGGTGATTGGAACAGTATAGCATTTCAAACTGGCAACCAAGCAG
GATCAGTATACTGACGCAATTTTTTTCTGAAGGTGGTTGGAATACCGGTATGAATTTTATAA
TCATTGAATTAAGGCGTAAGATTTGGTAACAAAAACTGCTTTTCACGGGAAACGATGAAACA
TGGCACAATTCAACCTGACTTTCTTTTATGAAAAGGCTCTCAAGCTGCCTTGTACTCCCTCC
GTCCCATAATATAAGAACGTTTTTGACATTAGTGTAGTACCAAAAACGTTCTTATATTATGGG
ACAGAGGGAGTATTTATTAATGGATAAAAAGAGAGGTACAGGGTTCTAGTAGTAACATATG
CAAATGGTGATACAGTATATTATCTACGATTTTGTGCAAGAATTTTCACACGAAAACAATATT
CCATGCAGTTTTTAGTTTTGACTTTTTTTTTTGAATTTTCACGCGCGCGGGGGGTGGGGGG
GGGGGGAGAGAACTCCCCCACCTGAATATATTTCTCGAACCCAACATACAGTACAATAGTA
CAAATGCCGGGGCGGCAGCAGAGAGAGAGGCAGGGGGCGACCCCCCCATGCTATGTTAC
ATGCAGCTTTGTTCTACCCACGCAGAGCCTGTAGCCAGTTTTGACTCTTTTTGAATTGTTTG
GATTGCTTACCACATGTGTCACGTGGTTTTAGTTTTGACTCTTGACTGTTTAATACCTACTCT
TTTTAAGATTTTGGATTCTAATGTCATCTGTCTTGCAACTCAGAGCTTTCAGCCGTGCTAAC
TTCCATTGATTCATCCGCCATCGCATCAAACATCCAGCACCATGCAGACTTCACACCGTTAT
TCTCGCCAGAGCACTCTTCACCCCTAAAGGCTTACCATGCAACTGCCAAAAGTGTTTTTGA
TTCTCTGATATTGAATTGGAATGCAACATATGACTATTACAACAAAGTGAATGCAAAGCAAG
CTTATTACCTATCCATGGAATTTTTGCAGGTCTCGCAATATACTTTCTTCAGCCTTATCTCAT
ACCTTTTGGTAACTGCGTTATGATGTTATATGTTTTTTTTTGTACTGCAGGGAAGAGCTCTTA
CAAATGCTATTGGCAACCTAGAGCTAACAGGACAATATGCAGAAGCGTTAAAACAACTTGG
ACAGAACCTAGAGGATGTTGCTAGTCAGGTAAGCCATTTTTGTCAGAGAATGGTTTTTAGTC
CATATCAAAATTATTGCCGAGCCATTTTTCTAAATAACATTTTATTATTATTCATGAACTTCCA
TATTTTTTTCAAATCTGCAAAAGGAGAAGTTTTTTTCCTGGGGGTTCCACCATCTGACTTATA
TTTGTTATGCAGTTTTCAGAGTGATTGACTATGAAATTACGCCAAAACACGAGCATTGCCTA
GAGTGCCCTGTAGTTTTAGAAATGTTAAGATTGGATATGTTACTTTTCTATTACAATGAAACA
GGAACCAGATCCTGCCCTTGGCAATGGTGGACTAGGCCGTCTAGCGTCCTGTTTTCTGGA
TTCTATGGCAACCCTAAATTATCCAGCATGGGGATATGGACTTCGGTACAGATATGGCCTC
TTTAAGCAAATCATAACAAAGGATGGTCAGGAGGAGGTAGCTGAGAATTGGCTAGAGGTAG
TGATATGTCTTCTACTTGTTTACTGTTTGGAAGCGTTGATTATGTTGGTTTCTGTTTTAATGA
TCTGCATGTTAGCTTCTTTTGCCATTTATAACTGATGCTGTATCTCCAGATGGGAAATCCAT
GGGAGATTGTAAGAAATGATGTCTCTTATCCTCTGAAATTCTATGGCAAAGTGGTTGAAGG
CACTGATGGGAGAAAACACTGGATTGGAGGAGAGAATATCAAGGCTGTGGCACATGATGT
TCCTATTCCTGGCTACAAGACTAAAACTACTAATAATCTTCGTCTTTGGTCAACAACAGTAC
CATCACAAAACTTTGATTTGGGAGCTTTTAATGCTGGGGATCATGTCAAGGCCAACGAAGC
ACATCTAAATGCTGAAAAGGTATTCCTTTGTCTGCATTTATTTTGAATATAAATCATTAAATCT
CAATAACTATTGATTTAAATGGTAAACACTAAACATTGTCTAGCTAGTAGTCACTGTGGTGT
CCCTCAATGTCATTTTAGAATGTGCACAGTCAAATGTTTGATAACTTTGATTGTAATAATATG
ATAATATACAGAAACTTTGATTGACACTAGATTTTTCATAAACATACTTATTGTATTTCCATGT
AAATAGAAAAGCTGATGTTCAGAGGTTTGTGTGTCAAAATGAACAGAGTTTGTGCACACATA
ATATCTTCATCTGGTGTTTGCAGTTCTCTAGGTTGTGTTTGCGTTTTCTCCTGCTCTGCCTT
AACTAATCTCAGCATATGCATCAGGCTACATTGCTCCTCTGTGTCTATAAAAAATAAGTCTG
ATGATATATGGTTGCAGTCCAGTAGTTCGTAGATGGCACAGCATGGAAGAACTTTTCACAT
GGAAGCCTTACCCTTATAATTTTAATTATACCTTTTGAGTGTTTGTGCATTGTGTACATACAC
CATTAAGTATGGGTGCTCAAAAATGGTTTATCCCAAACTGTGCAAAAGCCGCCACTGTTTGT
GTATTAATTTTTTTGAACTAGCTAAACAACTTGACCGAACAAGATACATACAATTGCACTCAC
TTCTGAAGCAGCTCAGTTGGACGGATTTGTTCCAATCCCGTGCATGGGTTAAATTGTCGAG
TTGTCCTCGCTTGTTCAGTAGTAGGAGTACTATCTATTATATTTATACTAAAGTTTTCTTTTT
GTGATACAGTACAAACTTTTAGTAATACTTACATTTGCGACCACTGTGGACAAAATTTATTAT
GTCTAACTTAACTTTATCACTGGTCTTCTCATCATGCCAGATATGCCACGTATTGTATCCAG
GGGACGAATCATCAGAGGGGAAAATTCTTCGCCTGAAACAACAATATACACTATGCTCGGC
CTCCCTGCAGGACATTATTTCTCGTTTTGAGTCGAGGGCTGGTGATTCTCTCAACTGGGAG
GACTTCCCCTCTAAAGTTGCAGTTCAGATGAATGACACCCATCCAACACTGTGCATTCCCG
AGTTAATGAGAATACTGATGGATGTTAAGGGATTGAGCTGGAATGAGTCTTGGAGTATCAC
GGAAAGGTATCACAGAAGGGTACTGCAGTTTGATGTGTTATGCTGTTCCTCCAGTTCCAGT
TAACTAATTTTATTTCCTCATCTGTCTTCCATTCAATCATGTACTGATTTTCTGATATCCACAT
CATTTGTAGAACCGTTGCATACACTAACCACACAGTACTTCCTGAAGCTCTGGAGAAGTGG
AGCTTGGACATTATGCAGAAACTTCTACCTCGACATGTTGAGATTATAGAAAGAATAGATGA
GGAGGTATCAAATGTGCAGATAATATTATACAATTAATTTCATGCTCTGTAGTTCAACTTATG
GTTTACTTGTCTTCATCGTTGATTTTAAACAGCTGATGAACACCATCGTCTCAAAATATGGA
ACGGCAGATATTTCACTGTTAAAACAGAAGCTTAAAGATATGAGGATCTTAGACAACGTTGA
TCTTCCAGCCTCTGTTGCTAAACTGTTTATTAAACCAAAAGAGAAAAAGGGGAAGTTGCTTG
TTGAATCTTTGGAGTCTATTGCTGAAGCTGACGAGAAAACTGAGTCAGAAGAGGAGGAAAA
CATTCTATCCGAGACAACAGAGAAAAAGGGCGAATCTGACTCTGAAGAAGCTCCTGATGCA
GAAAAGGAGGATCCTGAGTATGAGTTAGATCCATTTACAAAATACGATCCTCAGTTACCTAG
AGTTGTTCGAATGGCGAACCTCTGCGTTGTTGGTGGACATTCAGTTAATGGTGTGGCTGAG
ATTCACAGTGAAATTGTTAAGCAAGATGTGTTCAATAGCTTTTATGAGGTATTTGCATGATG
ATTTGGAAACCGTGATCTTATTTTCCAATGGCTGCTATAATCGTCAGGTGATATGGTATTCA
CCATCTGCCATTTTCTTGTGTTCTTTGCCAGATGTGGCCTACTAAGTTCCAAAACAAAACAA
ATGGAGTGACCCCCAGGCGTTGGATCCGGTTTTGTAATCCTGAATTAAGTACAATAATTTCA
AAATGGATAGGCTCTGATGACTGGATTCTGAACACTGATAAACTTGCAGGACTGAAGAAGG
TACTTCCAGTAGTTTTATTCCCCAATAATCAGTAATCTAATGCTTCCTTCACTTCAATGTTAA
CTGTGCATTGTTTTTACATTGATGTGTGTTCATCCTGTAACAGTTTGCTGATGATGAGGATC
TGCAATCAGAATGGCGTACTGCTAAAAGGAATAACAAGATGAAGGTAGTTTCGCTGATAAG
AGACAAGACTGGATATGTTGTCAGCCCAGATGCAATGTTTGATGTGCAGGTATATTTTGTAC
TAGAAAACATGTGTCTTCTTATAAATATGGTTTAACTCAGCTCTTGGTACTGGACACTTAGG
TGAAAAGAATACATGAATATAAGCGGCAGCTTCTAAATATCCTTGGTATCGTTTACCGCTAC
AAGAAGATGAAAGAAATGAGTGCAAAAGATAGAATAAAGAGCTTTGTTCCAAGGGTATGCA
TATTTGGCGGGAAAGCTTTTGCCACTTACGTACAGGCAAAGAGGATTGTGAAGTTTATCAC
AGATGTCGCAGCTACTGTAAATTATGATCCTGATATTGGAGATCTACTGAAGGTAAATTTGT
GTCTTTGATGCCACTTATCAGTCTTAATTTTCATGTTCTGTCACTGGCAAGTTGACTCCGAC
TGAAAAGCATTGAATGTTCAGAGTTTCAAAGGAAAACAGAGCAGTGAATGTTCTTAATGTCG
GTGCTTGTAAAACTGAGCAACTGAATAGCCACAGGTTTTTTAGAGAACAATATAGCCACAAA
TTTTTTTGACACTTTTATTGCTATCAGCATCTGATGCTGATATTACTGATAACTAGATGAGGT
AAATATAAGGTGACAGGTTAAGTCTGAGGATTTTTGTTTGTTATCCAGGTTGTATTTGTCCC
AGATTATAATGTTAGCGTTGCTGAGACGCTCATTCCTGCCAGTGAATTGTCCCAGCATATCA
GGTAATATTCTGCCTACTCTTATATAGTATGTGAATACATAAAAAAAGAATATTACCTGGAGT
TGGCTGACATAGAATTTTTCATGTGCCTTTTTGTTTGCTATAATATGTACCTGGAGTCTTACT
GGGATAGTGCCATCCAGAAGTGCCACCCCAGTTTCTATTTCTGTATAAGACGTGCATTTTTA
ACAGTATTACTATCCATTCAGTACCGCTGGAATGGAAGCTAGTGGAACCAGCAACATGAAG
TTTGCAATGAATGGCTGTATTCTTATTGGAACTTTGGATGGTGCGAATGTGGAGATCAGAG
AAGAGGTTGGAGAGGAGAACTTTTTCCTCTTTGGTGCACATGCACCTGAAATCGCTGGTTT
GAGGCAAGAGAGAGCTGAAGGGAAGGTACCACACTTGTTTATATAGCTGTGCCTACTATCA
AACTAGTAAAGATCTCTCCCATATGTGCTGTCCAAATAGTTTAGCTGAAGGAAAATCATAAC
TTTACAAGTTTCCTTTGCACTTGTAGTTTGTGCCTGACCCGAGATTTGAAGAGGTTAAGGAA
TACGTCCGCAGTGGCATCTTTGGGACTGGCAACTATGATGAATTGATGGGTTCTTTGGAAG
GAAATGAAGGTTATGGACGTGCAGATTATTTTCTTGTTGGCAAGGATTTTCCCAGTTACATT
GAATGCCAGGAAAAAGTTGATGAAGCATACCGAGATCAGAAGGTAAGTTGTATAACTGACG
CTGTTTGTTTTCTGGCTGAAATGCTCACAGTGATTATGCAAACTTTCTGATATGTGATGGCT
GACATCAGTGTTTCACTGTGAACGACTTCATTGATTCGTAGGAAGGAATATTTCTTAGTTTA
AAGTTTCAAACTGAAGTATCTATCGGTGTGTTGGAAGATCATAAACAATCAGAGAATATAGT
TGCTAGATGTGCTATATTACTCATCTGTTGGCTGTTGAGTGTTGATACATGAGCTTTGATGT
AGCTAGACCTTCAGGAAAGTGCTTCTGTGCATTCCAATAAAAAAATAACTTATAACTTAACA
AAACAAAACAATTAGGTTGAACCTCACCAGGGGGCCTTTTGTTATCTGTTTTCTGAAATGAA
AATCAGAAGTGGGGAGCTCCTCAATTCGAAAAAGAAAAAGAAAATCAGGGGGTAACTGAG
GGTCTGGTTTCCCTCTGTTGGAGCCCCCAAGAATTCTGTGGTCTTATTTTTGTAAGAGTATA
AGATGCACTGCTCCGTTAAGTTTCTCCTTCACCCATTTGTTTTCATTGCTTCGTGACATTTC
GTGTGACAGCTCAGATCACCATATGCATCCCATTTGTACACATTATTTTGCTACCGTGGATA
TTTCCACTCTGTTTCATGTGTGACATTTTTAATTCGTTACATTGTTACTCTAAACATTAGTCTA
ATAGATTTTCAGAGTTTTGACCCTCCTAATCACATAATATCCGTTTCGACCTGCAGCTATGG
ACAAGAATGTCTATCCTCAACACAGCGGGTTCCCCCAAGTTCAGCAGTGACCGGACGATC
CACGAGTATGCCAAGGACATATGGGGTATCAGCCCCGTCATCATGCCCTGAATAATTCAAG
CAGCTTCCAGGAACCTTGAGAGTCTATGAGGCGCGATTGGTCGCTGATTTGCCTTGATTCA
GCGAGAATGTATTGCTCCCCCCAGTTTTGTATAGGCATATGCAAGTTCTGACATGGACCTG
AGTGCTGAGAGCATGCAGGGTAGTTCATCCTTAGCAATATTGCATGTCAGTTAGCCAGTAT
TACCCAGGCTTATGGTGTGTGTATTTCGCTGAATAAAGTTGGAGCAGAGTCATCAAACAAA
CATACTTTGGTGTATCTGTAATGATAAATAAATGGTACATGTGACTGAACAAAAAGTGTCGT
TGGCGTGCACATGTAGTAGAAGCATGTTTCTTCAGAGAGGGTGCAAAGCTGAAAGCACCC
CAGCTTTGGAGTGAGAATGCAGAAGTTACGAAAACATTTGTTTACATCCAAAGTGCAACGG
GATTCGTCAAGAGTTCAGAGTCTTCCATCACGGTCATAGAGCATGTCCAATAGCAGCCCTA
TAAACCTATAACAGAGCAGTAGCAGTGGTCCCTCACATTTTGAACCAAAAATGCAGCTCCA
ACGGCTGCCCTATTTGTAAAATTTACTTCAAAATTTGTTGG
SEQ ID NO: 26 >Wheat (tetraploid) PHS1 5A (Svevo reference genome) CDS
ATGGCGACCGCCTCGCCGCCGCTCGCCACCCCCTTCCGCCCGCTCGCCGTCGCCGGAG
GTAGCGGCGGACTCGTCGTGGGCGCCAGGGCCGTGGTACCGCCGAGGCGGGGGGGGC
GGGGGTTCGTGGTGCGGAGCGTGGCGAGCGATCGGGAGGTGCGGGGGCCCGCCCCGA
CCGAGGAAGAGCTTTCAGCCGTGCTAACTTCCATTGATTCATCCGCCATCGCATCAAACAT
CCAGCACCATGCAGACTTCACACCGTTATTCTCGCCAGAGCACTCTTCACCCCTAAAGGCT
TACCATGCAACTGCCAAAAGTGTTTTTGATTCTCTGATATTGAATTGGAATGCAACATATGA
CTATTACAACAAAGTGAATGCAAAGCAAGCTTATTACCTATCCATGGAATTTTTGCAGGGAA
GAGCTCTTACAAATGCTATTGGCAACCTAGAGCTAACAGGACAATATGCAGAAGCGTTAAA
ACAACTTGGACAGAACCTAGAGGATGTTGCTAGTCAGGAACCAGATCCTGCCCTTGGCAAT
GGTGGACTAGGCCGTCTAGCGTCCTGTTTTCTGGATTCTATGGCAACCCTAAATTATCCAG
CATGGGGATATGGACTTCGGTACAGATATGGCCTCTTTAAGCAAATCATAACAAAGGATGG
TCAGGAGGAGGTAGCTGAGAATTGGCTAGAGATGGGAAATCCATGGGAGATTGTAAGAAA
TGATGTCTCTTATCCTCTGAAATTCTATGGCAAAGTGGTTGAAGGCACTGATGGGAGAAAA
CACTGGATTGGAGGAGAGAATATCAAGGCTGTGGCACATGATGTTCCTATTCCTGGCTACA
AGACTAAAACTACTAATAATCTTCGTCTTTGGTCAACAACAGTACCATCACAAAACTTTGATT
TGGGAGCTTTTAATGCTGGGGATCATGTCAAGGCCAACGAAGCACATCTAAATGCTGAAAA
GATATGCCACGTATTGTATCCAGGGGACGAATCATCAGAGGGGAAAATTCTTCGCCTGAAA
CAACAATATACACTATGCTCGGCCTCCCTGCAGGACATTATTTCTCGTTTTGAGTCGAGGG
CTGGTGATTCTCTCAACTGGGAGGACTTCCCCTCTAAAGTTGCAGTTCAGATGAATGACAC
CCATCCAACACTGTGCATTCCCGAGTTAATGAGAATACTGATGGATGTTAAGGGATTGAGC
TGGAATGAGTCTTGGAGTATCACGGAAAGAACCGTTGCATACACTAACCACACAGTACTTC
CTGAAGCTCTGGAGAAGTGGAGCTTGGACATTATGCAGAAACTTCTACCTCGACATGTTGA
GATTATAGAAAGAATAGATGAGGAGCTGATGAACACCATCGTCTCAAAATATGGAACGGCA
GATATTTCACTGTTAAAACAGAAGCTTAAAGATATGAGGATCTTAGACAACGTTGATCTTCC
AGCCTCTGTTGCTAAACTGTTTATTAAACCAAAAGAGAAAAAGGGGAAGTTGCTTGTTGAAT
CTTTGGAGTCTATTGCTGAAGCTGACGAGAAAACTGAGTCAGAAGAGGAGGAAAACATTCT
ATCCGAGACAACAGAGAAAAAGGGCGAATCTGACTCTGAAGAAGCTCCTGATGCAGAAAA
GGAGGATCCTGAGTATGAGTTAGATCCATTTACAAAATACGATCCTCAGTTACCTAGAGTTG
TTCGAATGGCGAACCTCTGCGTTGTTGGTGGACATTCAGTTAATGGTGTGGCTGAGATTCA
CAGTGAAATTGTTAAGCAAGATGTGTTCAATAGCTTTTATGAGATGTGGCCTACTAAGTTCC
AAAACAAAACAAATGGAGTGACCCCCAGGCGTTGGATCCGGTTTTGTAATCCTGAATTAAG
TACAATAATTTCAAAATGGATAGGCTCTGATGACTGGATTCTGAACACTGATAAACTTGCAG
GACTGAAGAAGTTTGCTGATGATGAGGATCTGCAATCAGAATGGCGTACTGCTAAAAGGAA
TAACAAGATGAAGGTAGTTTCGCTGATAAGAGACAAGACTGGATATGTTGTCAGCCCAGAT
GCAATGTTTGATGTGCAGGTGAAAAGAATACATGAATATAAGCGGCAGCTTCTAAATATCCT
TGGTATCGTTTACCGCTACAAGAAGATGAAAGAAATGAGTGCAAAAGATAGAATAAAGAGC
TTTGTTCCAAGGGTATGCATATTTGGCGGGAAAGCTTTTGCCACTTACGTACAGGCAAAGA
GGATTGTGAAGTTTATCACAGATGTCGCAGCTACTGTAAATTATGATCCTGATATTGGAGAT
CTACTGAAGGTTGTATTTGTCCCAGATTATAATGTTAGCGTTGCTGAGACGCTCATTCCTGC
CAGTGAATTGTCCCAGCATATCAGTACCGCTGGAATGGAAGCTAGTGGAACCAGCAACAT
GAAGTTTGCAATGAATGGCTGTATTCTTATTGGAACTTTGGATGGTGCGAATGTGGAGATC
AGAGAAGAGGTTGGAGAGGAGAACTTTTTCCTCTTTGGTGCACATGCACCTGAAATCGCTG
GTTTGAGGCAAGAGAGAGCTGAAGGGAAGTTTGTGCCTGACCCGAGATTTGAAGAGGTTA
AGGAATACGTCCGCAGTGGCATCTTTGGGACTGGCAACTATGATGAATTGATGGGTTCTTT
GGAAGGAAATGAAGGTTATGGACGTGCAGATTATTTTCTTGTTGGCAAGGATTTTCCCAGT
TACATTGAATGCCAGGAAAAAGTTGATGAAGCATACCGAGATCAGAAGCTATGGACAAGAA
TGTCTATCCTCAACACAGCGGGTTCCCCCAAGTTCAGCAGTGACCGGACGATCCACGAGT
ATGCCAAGGACATATGGGGTATCAGCCCCGTCATCATGCCCTGA
SEQ ID NO: 27 >Wheat (tetraploid) PHS1 5A (Svevo reference genome) protein
MATASPPLATPFRPLAVAGGSGGLVVGARAVVPPRRGRRGFVVRSVASDREVRGPAPTEEEL
SAVLTSIDSSAIASNIQHHADFTPLFSPEHSSPLKAYHATAKSVFDSLILNWNATYDYYNKVNAK
QAYYLSMEFLQGRALTNAIGNLELTGQYAEALKQLGQNLEDVASQEPDPALGNGGLGRLASCF
LDSMATLNYPAWGYGLRYRYGLFKQIITKDGQEEVAENWLEMGNPWEIVRNDVSYPLKFYGK
VVEGTDGRKHWIGGENIKAVAHDVPIPGYKTKTTNNLRLWSTTVPSQNFDLGAFNAGDHVKAN
EAHLNAEKICHVLYPGDESSEGKILRLKQQYTLCSASLQDIISRFESRAGDSLNWEDFPSKVAV
QMNDTHPTLCIPELMRILMDVKGLSWNESWSITERTVAYTNHTVLPEALEKWSLDIMQKLLPRH
VEIIERIDEELMNTIVSKYGTADISLLKQKLKDMRILDNVDLPASVAKLFIKPKEKKGKLLVESLESI
AEADEKTESEEEENILSETTEKKGESDSEEAPDAEKEDPEYELDPFTKYDPQLPRVVRMANLC
VVGGHSVNGVAEIHSEIVKQDVFNSFYEMWPTKFQNKTNGVTPRRWIRFCNPELSTIISKWIGS
DDWILNTDKLAGLKKFADDEDLQSEWRTAKRNNKMKVVSLIRDKTGYVVSPDAMFDVQVKRIH
EYKRQLLNILGIVYRYKKMKEMSAKDRIKSFVPRVCIFGGKAFATYVQAKRIVKFITDVAATVNYD
PDIGDLLKVVFVPDYNVSVAETLIPASELSQHISTAGMEASGTSNMKFAMNGCILIGTLDGANVEI
REEVGEENFFLFGAHAPEIAGLRQERAEGKFVPDPRFEEVKEYVRSGIFGTGNYDELMGSLEG
NEGYGRADYFLVGKDFPSYIECQEKVDEAYRDQKLWTRMSILNTAGSPKFSSDRTIHEYAKDI
WGISPVIMP
SEQ ID NO: 28 >Wheat (tetraploid) PHS1 5A (Svevo reference genome) promoter (−2 kb
upstream of ATG)
CTAGTGGGGCCTTGGTCCAAAATAATCCATGTAATTTTTCATGTTTTCCGGCTTCGTATTTTT
TGGTAAAAACCCAAAAACAATAGAAAAGAAGAATTGGCCTCTGACACTAGATTGATAGGTTA
GTCCAAATCAATATTATCAAAAATTGTGCCCAAACCATATACAAATGATATAAATATAACTAG
AATACTTCATAAATTTAGATACGTTTGATAAAGTGCCAAGAATGGAAGAGAAGTGATCATAT
GCCGCCCTGTCCATATCGGGCTCATCAGAATGTTCCCACCATCAGACTCCAGCCCCAGGA
TTATTAACTTATGTCGGCGGTGCGCGGAATGGATCTTGAAGAAAGCGGAATTTGTGTCGCC
CTCACGTAACCAGGAGAACTTGGCAGGTTGACGCGCAATCGACCTCTCCAGCAAAGCAAG
TCCTAGGTAGGCGCGCTTCAGCTCGCAGAGTAACCGTACTTCTGGAGGAGCAAGCGGGTG
GTGATCTTGAGCCGCCGAGACGAGCAATCAATTACCGAGCGAGAAGCATCTGAGTGGAGA
TGTTACCGAGCGTTCTGGAGCTCCAACTCTGCCGTCAGTGCATTGTCAGCTTGTGCTTTGC
GTAGATTCGCCGGAAAGGATCAGGATCATGCACGCCAGAATTCCAGGTGTCAGAGACCAC
CTAATGAAAACCATCGAGCTTTGATCAAAATAGCTCAAGATGGAATCTCCGATCACCTTGG
GAGCGAAAATCTTACTAGTTTCGTTCAAGGCCAAGGTAGTTATATATTTGTGTGTGCAATTA
TTAATCATTGGCCGCTTAGCAAACTCTTATTGGTTCGATAAACATTAAAGTCATCCCCACTTT
GAGAAAAAATGCTACTTGCTACAAACCCTNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNGCAAGTAATAAATCCTTG
CCGCCTCGTCCTTGTTTGGATGCTGCTTCTTGTCATCTCAAGGAGGCGTGTGGAGGGTGG
TGTGTCCCCGGATCTTGTTCTCTCGGTTGGGTTTTTTCTATGGCAGGTTTATCTCAGTGGTA
GTGTCACGTGAAGCAGATGACGCGACTATGCGTCTTGGTCCTCTGCGCTCTAATTCCGGC
AGATGAAGGTTGACCAGCTTCAGCCTCGTCGGGGAGCGTATGAGATTTGTGTTTTTTGACT
CCATCTGGTCGGACCTGTGGCATTATGTAGACCCTCTCATGTATTCTTTTTCTCCTGGACAG
TAGTCTGTTACACAAATCGTGGTCGTCGGATCTTCTAGTCCACATCGGCGACTTTTCCGAC
CGCCACTTCTACAAACTCCTTATTTTCAACAAGTCTCCTTCATTGAGATGGAAGTCCAGGCA
ATGGCTTTGCTCACGGCACACTGCCGGCGGATGCAAGAGAAAGAAGACATTCGACACCCC
TAAAACTATGAAGATGTAATTTTTAATCTTTGTAAGTGTTTTTGTGAGGATTTGTGCTACTTTT
AATATATTGCCTTTTGCCTTTTGCAAGGGAAAAAAAACTAAACCATTACACGAAGAGAAGAG
CCATTCCCAGCCGTCCAATAAACACGACAAAACGCTTTACAGCACGGGGGCAATTCCCAG
CCGTCGAAATGCGCTGCAACGGAACCAGCGCTCCCCGACACGGCGGCACATGAGCCACA
ACTCGTACCATGCACCGTGCACCGGCGGTGGGCCCACTGGCCAGCGTCCCCACACGTGT
ACCGGCGGCCACAATTACACGGCACGGCACGGCGACGGAGAGATATTTTAACGCCTTCCA
ATTCAGAAGCTCCCCCATCACTCACTCGGGTACAGAGCGCAGCGGGCCGCGTGACTCTCG
CGTCGTCGCATCGCGCGCGCTGCACACCACCGTCAGCGCGACCACTGCCGCCA
SEQ ID NO: 29 >Wheat (tetraploid) PHS1 5B (Svevo reference genome) genomic DNA
AACTAAACCATTACATGAGGCCATTCCCATCCGTCCAATAAATACGACAAAACGCTTTACAG
CACGGGGGCAATTCCCAGCCGTCGAAATGCGCTGCAACGGAACTAGCTCTCCCAGACAGA
CACAGCGGCACATGAGCCACGCCTCCCATCGCAACCGTGCACCGGCGGTGGGCCCACCG
GCCAGCGCCCCCACACGTGTACCGGCGCCCAGCATTGCACGGCACGGCGACGGAGAGAT
ATTTTTAACGCCTTCCAATTCAGAAGCTCCCCCATCACTCACTCGGGTACAGAGCGCAGCG
GGCCGCGTGACTCTCGCGTCGTCGCATCGCGCGCGCTGCACACCACCGTCAGCGCGACC
ACTGCCACCAATGGCGACCGCCTCGCCGCCGCTCGCCACCGCCTTCCGCCCGCTCGCAG
CCGGCGGCGGCGCCGGAGGAGGAGGAGCGCACGGCGTGGGCGCCGCGGGCCGGGTCG
CGCCGAGGCGGGGGCGCCGGGGCTTCGTGGTGCGGAGCGTGGCGAGCGATCGGGAGG
TGCGGGGCCCCGCCTCGACCGAGGAAGGTAAGCCGTCGCGCGCTCCCGTTGGCTGCGA
CGACCCGGAATGCTGGTCGGCTGTAATGTGGTTCGGATTTTCGCTGGTGCGACCTGAATT
TCTGATCCTCTGACTAATGGTCTCTCTGTTCCCGGGTTTCGGGCTGTTGATTTTGTGCGCG
CGCATTGCTAATCTACTGCTCTAGGATTTGTTAGGGTTCAGAGGAATCTTAAATTCACGAAT
AAACTTCGACGAGTGTATTCTGAATTTACTGAAACTGGCTTAGTTTGGGGCGTCAAGTTAGA
ATGGTGTATTTATGGCACAGTAGCAAATTCTGAGAAATGCTTCTGTGCGTGCACAGCCACC
CCTTGTGGATGATGCAGCAAGTGTCAACATACCGTCGCTACTATCTGTAGCTGTAACAAAA
TCTTACGCCTTGTTTTCTGGTGGTACAGTAATATTATCATTTTACAAAGAGTGGCTGTAACA
AAATCTTACGCCTTGTTTTCTGGTGGCTTGCCATTGAGGAGTAGAATGCAAATGGCGATAC
AGTATATTATCTACGATTTTGTGCAAGAATTTTCACACGTATCCCATGCAATTTTTAGTTTTG
ACTCTCGACTGTTTATACCTACTCTTTGTAAAATTTTGGATCCTAATATTGTTGTTCTTGCAA
CTCAGAGCTTTCAGCCGTGCTAACTTCCATTGATTCTTCCGCCATCGCGTCAAATATCCAG
CACCATGCAGACTTCACACCGTTGTTCTCGCCAGAGCACTCTTCACCCCTAAAGGCTTACC
ATGCAACTGCTAAAAGTGTTTTTGATTCTCTGATCATCAATTGGAATGCAACATATGACTATT
ACAACAAAGTGAATGCAAAGCAAGCTTATTACCTGTCCATGGAGTTTTTGCAGGTCTCACAA
TATACTTCCTTTAGCCTTACCTCAATACTTTTTGGTAACTGCGTTATGATGTTATATGGTTGT
TATCTGTGTACTGCAGGGAAGAGCTCTCACAAATGCTATTGGCAATCTAGAGCTAACAGGA
CAATATGCAGAAGCGTTAAAACAACTTGGACAGAACCTAGAGGATGTTGCTAGTCAGGTAA
GTGATTTTTGTCGGAGATTGGTTTTTAGTCCGTATCAAAATTATCGCAGGGCCATTATTTTTA
TTTTGAAAACGCATTTTATTATTAATCATCAAGTTCCCTTTGTTTAAAAACAATCTGAAAAGAA
GAAGTTTTTTTCCTGAGGGTTCCACCGTCTGACTTATGATCTTTATGCAGTTTTCAGAGTGA
CTATGAAATTACGCCAAAACACGATCTTATTGATGCCTAGAGTGCCCTGTAGTTTAGAAATC
TAAGATTGGATATGTTACTTTTCTATTACAATGAAACAGGAACCAGATCCTGCCCTTGGCAA
TGGTGGTCTAGGCCGTCTAGCGTCCTGTTTTCTGGATTCTATGGCAACCCTAAATTATCCA
GCATGGGGATATGGACTTCGGTACAGATACGGCCTCTTTAAGCAAATCATAACAAAGGATG
GTCAGGAGGAGGTAGCTGAGAATTGGCTAGAGGTAGTGATGTGTCTTCTACTTGTTTACTG
TTTGGAAGTGTTGATTATGTTGGTTTATTTATGTTCTAATGACCTGCATGTTAGCTTCTTTTC
TATTTATAACTGATGCTGTATCTCCAGATGGGAAATCCATGGGAGATCGTAAGAAATGATGT
CTCTTATCCTGTGAAATTCTATGGCAAAGTGGTTGAAGGCACTGATGGGAGAAAACACTGG
ATTGGAGGAGAGAATATCAAGGCTGTGGCACATGATGTTCCTATTCCTGGCTACAAGACTA
AAACTGCTAATAATCTTCGTCTTTGGTCAACAACAGTACCATCACAAAATTTTGATTTGGGT
GCCTTTAATGCTGGGGATCATGCCAAGGCCAATGAAGCACATCTAAATGCTGAAAAGGTAT
TCTTTCGTCTACTTTATTTTGAATATAAACCATTAACTCTCAACAACTACTGATTCAAAGGCT
AAACAGTGCTTTGCTAGTAGTTACTGTGTTTTCTCTTAGATGTCATTTCATAATGTGTGCAGT
CAATCGTTTGATATACACAAACCTATTTGGATTTCTCCTGTAAAAATGTTGTCACTAGATTTT
TCATGAAAAAACTTATTGTATATGTAAATAGAAAAGCTGATGCTCAGGTGCTTTGTGTTCAA
ATGACAAGTAAAAATGAACAGAGTTTGTGCACCCATAACTTCTTCACCTGGTGTGTGCAATT
CGTTCGGTTGTATTTATGCGTGTTCTTATTCTGCCTGAATGATTCTCAGCATATGCATCAGG
CTACATTTCTCCTCTGTGTCAAGAAAAAATAAGTCTGACCAGATGCAGTCCAGTAGTCTTTA
GATCTTTTCACATGGAAGCGTTACTCTATGATTTTTATTATACCTTTAGAGTGTTTGTGCATT
GTGCACATATACCATGAAGTATGGGTGTTCAAATGTGGTTCTCTCAAACTGTGCTAAACCCA
CCACTGTTTGCCAGTATTCATTTTTCTGAACTAGCAACAACTTGACTGGACAAGATACATTG
TACTCACATCTGAAGCTGCTGGGTTGGACAGATTTGTCCGACCCGTCTTTGAACATCTCTA
CTATTTAGTGTGCATGGGTTAAATTGTTGACTTTTCCTCGTTTGTTCAGTAGTAGGAGCACT
ATCTAATTATATTTATATTCAAGTTTTCTCTGTGTGTTACGTTATAAACTTTTAGTCATACTTA
CATTTGTGGACAAAAGTTATCATGTCTAACTTAAGTTTGTGACTGATCTTCTCATCATGCCA
GATATGCCACGTATTGTATCCAGGGGACGAATCATCAGAGGGGAAAATTCTTCGCTTGAAA
CAACAATATACATTATGCTCGGCCTCCCTGCAGGACATTATTTCTCGTTTTGAGTCGAGGG
CTGGTGATTCTCTCAACTGGGAGGACTTCCCCTCTAAAGTTGCAGTTCAGATGAATGACAC
TCATCCAACACTGTGCATTCCGGAGTTAATGAGAATACTGATGGATATAAAGGGATTGAGC
TGGAATGAGGCTTGGAGTATCACAGAAAGGTACCACAGAAGGGTACTGCAGTTTGATGTGT
TATGCTGTTCCTCCAGCTCCAATTAACTAATTGTATTGCCTCATCTGTCTTCCATTCAATTAT
GTACTGATTCTCTGATATCATTTGTAGAACCGTTGCATACACTAACCACACGGTACTTCCTG
AAGCTCTGGAGAAGTGGAGCTTGGACATTATGCAGAAACTTCTACCCCGACATGTTGAGAT
CATAGAAACAATAGATGAGAAGGTAACAAATGTGCAGATAATATTATGCAATAAATTTCATT
CTCTGTAGTTCAACTTATGGTTTACTTATCAACATCGTTGATTTTAAAACAGCTGATGAACAA
CATCGTCTCAAAATATGGAACGGCAGATATTTCACTGTTAAAACAGAAGCTTAAAGATATGA
GGATCTTAGACAACGTTGATCTTCCAGCCTCTGTTGCTAAACTGTTTATTAAACCTAAAGAG
AAAAAGGGGAAGTTGCTTGTTGAATCTTTGGAGTCTATTGCTGAAGGTGACGAGAAAACTG
AGTCACAAGAGGAGGAAAACATTCTATCCGAGACAGCAGAGAAAAAGGGCGGATCTGACT
CTGAAGAAGCTCCTGATGCAGAAAAGGAGGATACTGTGTATGAGTTAGATCCATTTGCAAA
ATACGATCCTCAGTTACCTAGAGTTGTTCGAATGGCAAATCTCTGTGTTGTTGGTGGACATT
CAGTTAATGGTGTGGCTGAGATTCACAGTGAAATTGTTAAGCAAGATGTGTTCAATAGCTTT
TATGAGGTATTTGCACGATGATTTGGAAACCATGATCTTATTTTCTAACGGCTGCTATAATC
GCCAGGTAATATGGTATCACCATCTGCCATTTTCTTATGTTATTTGCCAGATGTGGCCTACT
AAGTTCCAAAACAAAACAAACGGAGTAACTCCCAGGCGTTGGATCCGGTTTTGTAATCCTG
AATTAAGTGCAATCATTTCAAAATGGATAGGCTCTGATGACTGGATTCTGAACACTGATAAA
CTTGCAGGACTGAAGAAGGTACTTCCAGTAATTTTAATTTCCAAATATCAGTAATCTAATGC
TTCCTTCACTTCCATGTTACCTGTGCCTTGTTTTTACATTGATGTGTGTTCATCCCGTAATAG
TTTGCTGATGATGAGGATCTGCAATCAGAATGGCGTACTGCTAAAAGGAATAACAAGATGA
AGGTAGTTTCGCTGATAAGAGACAAGACTGGATATGTTGTCAGCCCAGATGCAATGTTTGA
TGTGCAGGTATATTTTGGACTAGAAACATGTGTCTCCTGTTAAATATGGTTTAACTCGGATC
TTGGTACTTGACACTTAGGTGAAAAGAATACATGAATATAAGCGGCAGCTGCTAAATATCCT
TGGTATCGTTTACCGATACAAGAAGATGAAAGAAATGAGTGCCAAAGATAGAATAAAGAGC
TTTGTTCCAAGGGTATGCATATTTGGTGGGAAAGCTTTTGCCACTTACGTACAGGCAAAGA
GGATTGTGAAGTTTATCACAGATGTCGCAGCTACTGTAAATTATGACCCTGATGTTGGAGAT
TTACTGAAGGTAAATTTGTGTCTTCAATGGCACTTATCATTCTAAATTTTCATGATCTGTCAC
TAGCAATTTGACTCCGACTAGGGGTTACTGAAAAGCACTGAATGTTCCGAGTTTCAAAAGA
AAACAGAGCAGTGAATGTTCTTAATGTCGGTGCTTGTAGAACTGAGAAACTGAATAGCCAC
AGTTTTTTTTAGAGAACAATAGCCACAATTGTTCTGATGCTGATTTTGCTGATAACTAGATGA
GGTAAATACAAGGTGACAGGTTAAGTTCTGAGGATTTTTGTTTGTTATCCAGGTTGTATTTG
TCCCAGATTATAATGTTAGCATTGCTGAGAAGCTCATTCCTGCCAGTGAATTGTCCCAGCAT
ATCAGGTAATATTCTGGCTACTCTTATATAGTATGTGAATACAAAGAAAAGGAGTCTTATCT
GGAGGTGACTGATGTGGAAATTTTCATGTGCCTTTTTGTTTGCTACTCCCTCTGTCCCATAA
TATAAGAGCGCTTTTGACAGTACACTAGTGTCAAAAACGCTCTTATAATATGGGACGGAGG
GAGTATAATATATACCTGGAGTCTTGCTGGGATAGTGACATCCAGAAGTGCCACGCCAGCT
TCTATTTCTGTACAAGACGTGCATTTTTAACACTATTACTATCCATTCAGTACCGCTGGAATG
GAAGCTAGTGGAACAAGCAACATGAAGTTTGCAATGAATGGTTGTATTCTTATTGGAACTTT
GGATGGTGCGAATGTGGAGATCAGAGAAGAGGTTGGAGAGGAGAACTTTTTCCTCTTTGG
TGCACATGCACCTGAAATCGCTGGTTTGAGGCAAGAGAGAGCTGAAGGAAAGGTACCACA
CTTGTTTATAGAACTGTGCCTAACTATGAAACTAGTAAAGATCTCTCCCAAATGTGTGTTGT
CCAAATAGTTTAAAGGAAGAAAAATTATAACTTTACCATTTTCCTTTGCACTTGTAGTTTGTG
CCTGACCCGAGATTTGAAGAGGTTAAGGAATACGTCCGCAGTGGCATCTTTGGGACTAGC
AACTATGATGAATTGATGGGTTCTTTGGAAGGAAATGAAGGTTATGGACGTGCAGATTATTT
TCTTGTTGGCAAGGATTTTCCCAGTTACATTGAATGCCAGCAAAAAGTTGATGAAGCATACC
GAGATCAGAAGGTAAGTTGTATAACTGACGCTGTTTGTTTTCTGGCTGAAATGCATACAGT
GATTATGCAAACTTTCTGATATGTGATGGCTGCCATCAGTGTTTCACTGTGAACGACTTCAT
TGATTCGTAGGAAGGAATATTTCTTAGTTCAAAGTTTCAAACTGGATTATCTATAGGTGTGTT
GGAAGATCATAAACAATCAGAGAATATAGTTGCTAGATGTGCTATACTATATGTTGGCTGTT
GAGTGTTGATACATGAGCTTTGATGAACCTAGCTAGACCTTCAGGAAAGTGCTTCTATGCA
TTCCAATAAAAAAAACTTATAACTTAACAAAACAATTAGGTTGAACCTCGCCAGGGAGCGTT
TTGTTATCTGTTTTCTGAAATGAAAATCAGAAGCGGGGAGCTCCTCGATTCTAAAATGAAAA
AAAAATCCAGGGATAACTGAGGGCCTGGTTTCCCTCTGTTGGAGCCCCAAAGAATTCTGTG
GCCTTATTTTTGTAAGAGTATAAGATGCACTGCTCCGTTAAGTTTCTCCTTCATCCATTTGTT
TTCATTGCTTCGTGACATTTCGTGTAACAGAGCATCCCATTTGTACACATTATTTTGCTATCG
TGGATATTTCCACTCTGTTTCATGTGTAACATTTTTAATTCGTTACATCCTTGCTAACTACAC
ATAGTCTGATAGATTTTCAGAGTTTTGGTCCTCCTAATAACATAATATCCGTTTCTACCTGCA
GCTATGGACAAGAATGTCCATCCTCAACACAGTTGGTTCCCCCAAGTTCAGCAGTGACCGG
ACGATCCACGAGTATGCCAAGGACATATGGGATATCAGCCCCGTCATCATGCCCTGAATTA
TTCAAGCAGCTTTCAGGAACATTCAGAGTCTATGAGGCGCGATTGGTCGCAATGTATTGCT
CCCCCCAGTTTTGTATAGGCACATATGCAAGTTCTGACATGGACCTGAGTGCTGAGAGCAT
GCAGGGTAGTTCATCCTTAGCAATATTGCATGTCACTTAGCCCGTATTACCCAGGCTTATG
GTGTGTGTATTTCGCCGAATAAAGTTGGAGCAGAATCATCAAACAAACATACTTTGGTGTAT
CTGTAATGATAAATAAATGGTAAATGACTGAACAAAAAGTGTCGTTGGCGCACACATGTAGT
AGAAGCATGTTTCTTCAGAGAGGCTGCAAAACTGAAAGCACCCTAGCTTTGAAGTGAGAAT
GAAGAAGTTACGGAAACATTTGTTTACATCCAAAGTGCAACGGGATTCGTCAAGACTCAAG
AGTTCAGAGTCTTCCGTCACGGCCATAGGGCATGTCCAACAGCAGCCCTATAATAGAGCA
GTAGCAGTGGTCTCTCACATTTTGAACCAAAAACGCAGCTCCAACGGCTGCCCTATTTGTA
AAATTTACTTCAAAATTTGTTGGACAGCCCGAAAATGCAACACCAG
SEQ ID NO: 30 >Wheat (tetraploid) PHS1 5B (Svevo reference genome) CDS
ATGGCGACCGCCTCGCCGCCGCTCGCCACCGCCTTCCGCCCGCTCGCAGCCGGCGGCG
GCGCCGGAGGAGGAGGAGCGCACGGCGTGGGCGCCGCGGGCCGGGTCGCGCCGAGGC
GGGGGCGCCGGGGCTTCGTGGTGCGGAGCGTGGCGAGCGATCGGGAGGTGCGGGGCC
CCGCCTCGACCGAGGAAGAGCTTTCAGCCGTGCTAACTTCCATTGATTCTTCCGCCATCGC
GTCAAATATCCAGCACCATGCAGACTTCACACCGTTGTTCTCGCCAGAGCACTCTTCACCC
CTAAAGGCTTACCATGCAACTGCTAAAAGTGTTTTTGATTCTCTGATCATCAATTGGAATGC
AACATATGACTATTACAACAAAGTGAATGCAAAGCAAGCTTATTACCTGTCCATGGAGTTTT
TGCAGGGAAGAGCTCTCACAAATGCTATTGGCAATCTAGAGCTAACAGGACAATATGCAGA
AGCGTTAAAACAACTTGGACAGAACCTAGAGGATGTTGCTAGTCAGGAACCAGATCCTGCC
CTTGGCAATGGTGGTCTAGGCCGTCTAGCGTCCTGTTTTCTGGATTCTATGGCAACCCTAA
ATTATCCAGCATGGGGATATGGACTTCGGTACAGATACGGCCTCTTTAAGCAAATCATAAC
AAAGGATGGTCAGGAGGAGGTAGCTGAGAATTGGCTAGAGATGGGAAATCCATGGGAGAT
CGTAAGAAATGATGTCTCTTATCCTGTGAAATTCTATGGCAAAGTGGTTGAAGGCACTGAT
GGGAGAAAACACTGGATTGGAGGAGAGAATATCAAGGCTGTGGCACATGATGTTCCTATTC
CTGGCTACAAGACTAAAACTGCTAATAATCTTCGTCTTTGGTCAACAACAGTACCATCACAA
AATTTTGATTTGGGTGCCTTTAATGCTGGGGATCATGCCAAGGCCAATGAAGCACATCTAA
ATGCTGAAAAGATATGCCACGTATTGTATCCAGGGGACGAATCATCAGAGGGGAAAATTCT
TCGCTTGAAACAACAATATACATTATGCTCGGCCTCCCTGCAGGACATTATTTCTCGTTTTG
AGTCGAGGGCTGGTGATTCTCTCAACTGGGAGGACTTCCCCTCTAAAGTTGCAGTTCAGAT
GAATGACACTCATCCAACACTGTGCATTCCGGAGTTAATGAGAATACTGATGGATATAAAG
GGATTGAGCTGGAATGAGGCTTGGAGTATCACAGAAAGAACCGTTGCATACACTAACCACA
CGGTACTTCCTGAAGCTCTGGAGAAGTGGAGCTTGGACATTATGCAGAAACTTCTACCCCG
ACATGTTGAGATCATAGAAACAATAGATGAGAAGCTGATGAACAACATCGTCTCAAAATATG
GAACGGCAGATATTTCACTGTTAAAACAGAAGCTTAAAGATATGAGGATCTTAGACAACGTT
GATCTTCCAGCCTCTGTTGCTAAACTGTTTATTAAACCTAAAGAGAAAAAGGGGAAGTTGCT
TGTTGAATCTTTGGAGTCTATTGCTGAAGGTGACGAGAAAACTGAGTCACAAGAGGAGGAA
AACATTCTATCCGAGACAGCAGAGAAAAAGGGCGGATCTGACTCTGAAGAAGCTCCTGAT
GCAGAAAAGGAGGATACTGTGTATGAGTTAGATCCATTTGCAAAATACGATCCTCAGTTAC
CTAGAGTTGTTCGAATGGCAAATCTCTGTGTTGTTGGTGGACATTCAGTTAATGGTGTGGC
TGAGATTCACAGTGAAATTGTTAAGCAAGATGTGTTCAATAGCTTTTATGAGATGTGGCCTA
CTAAGTTCCAAAACAAAACAAACGGAGTAACTCCCAGGCGTTGGATCCGGTTTTGTAATCC
TGAATTAAGTGCAATCATTTCAAAATGGATAGGCTCTGATGACTGGATTCTGAACACTGATA
AACTTGCAGGACTGAAGAAGTTTGCTGATGATGAGGATCTGCAATCAGAATGGCGTACTGC
TAAAAGGAATAACAAGATGAAGGTAGTTTCGCTGATAAGAGACAAGACTGGATATGTTGTC
AGCCCAGATGCAATGTTTGATGTGCAGGTGAAAAGAATACATGAATATAAGCGGCAGCTGC
TAAATATCCTTGGTATCGTTTACCGATACAAGAAGATGAAAGAAATGAGTGCCAAAGATAGA
ATAAAGAGCTTTGTTCCAAGGGTATGCATATTTGGTGGGAAAGCTTTTGCCACTTACGTACA
GGCAAAGAGGATTGTGAAGTTTATCACAGATGTCGCAGCTACTGTAAATTATGACCCTGAT
GTTGGAGATTTACTGAAGGTTGTATTTGTCCCAGATTATAATGTTAGCATTGCTGAGAAGCT
CATTCCTGCCAGTGAATTGTCCCAGCATATCAGTACCGCTGGAATGGAAGCTAGTGGAACA
AGCAACATGAAGTTTGCAATGAATGGTTGTATTCTTATTGGAACTTTGGATGGTGCGAATGT
GGAGATCAGAGAAGAGGTTGGAGAGGAGAACTTTTTCCTCTTTGGTGCACATGCACCTGAA
ATCGCTGGTTTGAGGCAAGAGAGAGCTGAAGGAAAGTTTGTGCCTGACCCGAGATTTGAA
GAGGTTAAGGAATACGTCCGCAGTGGCATCTTTGGGACTAGCAACTATGATGAATTGATGG
GTTCTTTGGAAGGAAATGAAGGTTATGGACGTGCAGATTATTTTCTTGTTGGCAAGGATTTT
CCCAGTTACATTGAATGCCAGCAAAAAGTTGATGAAGCATACCGAGATCAGAAGCTATGGA
CAAGAATGTCCATCCTCAACACAGTTGGTTCCCCCAAGTTCAGCAGTGACCGGACGATCCA
CGAGTATGCCAAGGACATATGGGATATCAGCCCCGTCATCATGCCCTGA
SEQ ID NO: 31 >Wheat (tetraploid) PHS1 5B (Svevo reference genome) protein
MATASPPLATAFRPLAAGGGAGGGGAHGVGAAGRVAPRRGRRGFVVRSVASDREVRGPAST
EEELSAVLTSIDSSAIASNIQHHADFTPLFSPEHSSPLKAYHATAKSVFDSLIINWNATYDYYNKV
NAKQAYYLSMEFLQGRALTNAIGNLELTGQYAEALKQLGQNLEDVASQEPDPALGNGGLGRLA
SCFLDSMATLNYPAWGYGLRYRYGLFKQIITKDGQEEVAENWLEMGNPWEIVRNDVSYPVKF
YGKVVEGTDGRKHWIGGENIKAVAHDVPIPGYKTKTANNLRLWSTTVPSQNFDLGAFNAGDHA
KANEAHLNAEKICHVLYPGDESSEGKILRLKQQYTLCSASLQDIISRFESRAGDSLNWEDFPSK
VAVQMNDTHPTLCIPELMRILMDIKGLSWNEAWSITERTVAYTNHTVLPEALEKWSLDIMQKLLP
RHVEIIETIDEKLMNNIVSKYGTADISLLKQKLKDMRILDNVDLPASVAKLFIKPKEKKGKLLVESL
ESIAEGDEKTESQEEENILSETAEKKGGSDSEEAPDAEKEDTVYELDPFAKYDPQLPRVVRMA
NLCVVGGHSVNGVAEIHSEIVKQDVFNSFYEMWPTKFQNKTNGVTPRRWIRFCNPELSAIISK
WIGSDDWILNTDKLAGLKKFADDEDLQSEWRTAKRNNKMKVVSLIRDKTGYVVSPDAMFDVQ
VKRIHEYKRQLLNILGIVYRYKKMKEMSAKDRIKSFVPRVCIFGGKAFATYVQAKRIVKFITDVAA
TVNYDPDVGDLLKVVFVPDYNVSIAEKLIPASELSQHISTAGMEASGTSNMKFAMNGCILIGTLD
GANVEIREEVGEENFFLFGAHAPEIAGLRQERAEGKFVPDPRFEEVKEYVRSGIFGTSNYDELM
GSLEGNEGYGRADYFLVGKDFPSYIECQQKVDEAYRDQKLWTRMSILNTVGSPKFSSDRTIHE
YAKDIWDISPVIMP*
SEQ ID NO: 32 >Wheat (tetraploid) PHS1 5B (Svevo reference genome) promoter (−2 kb
upstream of ATG)
GTTGGCTCCCACATGCTCCTCGATGATCTTCATCGGATGAACCACGATGTCGAGGATTCAA
GCAACCCCGTATACAATTCCCTTTGTCAATCGATACGTTACTTGCCCGAGATTCGATCGTC
GGTATCCCAATACCTCGCTCAATCTCATTACCGGCAAGTCACTTTACTCGTACCGTAATGCA
TGATCCCGTGACCAAACACTTGGTCACTTTGAGCTCATTATGATGATGCATTACCGAGTGG
GCCCAGAGATACCTCTCCGTCATACGGAGTGACAAATCCCAGTCTCGATCCGTGTCAACC
CAACAGACACTTTCGGAGATACCCGTAGTATACCTTTATAGTCACCCAGTTATGTTGTGACG
TTTGGTACACCCAAAGCACTCCTACGGTGTCCGGGAGTTACACGATCTCATGGTCTAAGGA
AAAGATACTTGACATTGGAAAAGCTCTAGCAAACGAACTACACGATCTTGTGCTATGCTTAG
GATTGGGTCTTGTCCATCACATCATTCTCCTAATGATGTGATCCCGTTATCAACGACATCCA
ATGTCCATAGTCAAGAAACCATGACTATCATTTGATCAACGAGCTAGTCAACTAGAGGCTTA
CTAGGGACATGTTGTGGTCTATGTATTCACACATGTATTACGATTTCCGGATAACACAATTA
TAACATGAATAACAGACGATTATCATGAACAAGGAAATATAATAATAATCTTTTATTATTGCC
TCTAGGGCATATTTCCAACAATTGCGGTGAGGCGCGTGGAGGTTGGCGTGTCCCCAAATC
TAGTTATCTGGGTCAGGTTTTTTCTTTGGTAGGTTTATCTCAGTGGTAGTGTCCCGTGAAGC
GGATGACACGACTATGTGTCTTGGTCCTCCATGCTCTAGTTGGGATGGAATCTCCGACCAC
CTATCGAAAACCATCAAGCTTTCGCTAAAATTAGTCAAGATGGAATCTCCGATCACCTTGGG
AGCGAAAATTTCATTGGAGGCCAAGGTAGTTATGTATTTGTGTGTGCAATTACCGACCATTG
GCCGCTTAGCAAACTCCTATTGGCTCGGTAAAGCNNNNNNNNNNGTTGGCGTGTCCCCAA
ATCTAGTTATCTGGGTTGGGGTTTTTCTTTGGAAGGTTTATCTCAGTGGTAGTGTCCCGTGA
AGCGGATGACACGGCTATGTGTCTTGGTCCTCCATGCTCTAGTTTTGACATGTGAAGGTTG
GCCAACTTCGGCGTTGTCAGGGAGCATATGAGATTTGTGTTCCTTTACTTCGCCTGGTCAG
ACCTACGGTATTATATAAGCCCTCTCATGTGTTTTCTTCTCTTGGGCAGCAGTCAGTTACGC
AAATCGTGGTCGCTGACATCTTCTAGTCCACATCGGCGACTTCCCGACCGCCGCTTCTACA
AGCTCCTTATTTTTCAACAAGTTTTCTCCACCGAGATGGAAGTCAAGAGGCGGCAACGGCT
TTGCTCATGGCGCACTGCGGATGGATGCAGGAGAAGGAAGACATTCCACACCCCTCAAAC
TATGAAGATGTAATTTTTAGTTTTTGTAAGTATTTTTGTGAGGATTTGTGCTACTTTTAATATA
TATGGCCTTTTGCCTTTTTCCAGGAAAGGAAAAACTAAACCATTACATGAGGCCATTCCCAT
CCGTCCAATAAATACGACAAAACGCTTTACAGCACGGGGGCAATTCCCAGCCGTCGAAAT
GCGCTGCAACGGAACTAGCTCTCCCAGACAGACACAGCGGCACATGAGCCACGCCTCCC
ATCGCAACCGTGCACCGGCGGTGGGCCCACCGGCCAGCGCCCCCACACGTGTACCGGC
GCCCAGCATTGCACGGCACGGCGACGGAGAGATATTTTTAACGCCTTCCAATTCAGAAGC
TCCCCCATCACTCACTCGGGTACAGAGCGCAGCGGGCCGCGTGACTCTCGCGTCGTCGC
ATCGCGCGCGCTGCACACCACCGTCAGCGCGACCACTGCCACCA
SEQ ID NO: 33 >Wheat (tetraploid) PHS1 5A (Kronos) genomic DNA
GGCACGGCGACGGAGAGATATTTTAACGCCTTCCAATTCAGAAGCTCCCCCATCACTCACT
CGGGTACAGAGCGCAGCGGGCCGCGTGACTCTCGCGTCGTCGCATCGCGCGCGCTGCA
CACCACCGTCAGCGCGACCACTGCCGCCAATGGCGACCGCCTCGCCGCCGCTCGCCACC
CCCTTCCGCCCGCTCGCCGTCGCCGGAGGTAGCGGCGGACTCGTCGTGGGCGCCAGGG
CCGTGGTACCGCCGAGGCGGGGGGGGGGGGTTCGTGGTGCGGAGCGTGGCGAGCG
ATCGGGAGGTGCGGGGGCCCGCCCCGACCGAGGAAGGTAAGCGGCCGCGCGCTCCCAT
TGGCTGCCACGACCTGGAATGCTGGTCGGCTGTAATGTGGTTCGGATTTTCGCTGGTGCG
ACCTGAATTTGTGATCCTCTGACTGACGGCCTATGTGTTCCCCGGGTTTCGGGCTGTTGAT
TTTGTGCGCGCGCATTGCTAATCTACTTCTCTAGGATTTGTTAGGGCTCAGAGCAATCTTAA
ATTCGCGAATACACTTCGACGAATGTAGTCTGAATTTACTGAAACTAGCTTAGTTTGGCGCG
TCAAGATAGACTGATGTATTTATGGTAGCAAATTCTGAAAACTTCTTCTGTGCGTGGACAGC
CACCCCTCGTGGATGATGCAGCAAGTGCTAACACAGTGTTTGCTAGTACTTGCTAGTATCT
GTAGCTGTAACAAAATCCTTCGAATCTGAACTAGTGAGATAAATCGTTTGAGATTTTTGCTG
AAGATTACACAGTATCAATATTTCTGAAGGTGATTGGAACAGTATAGCATTTCAAACTGGCA
ACCAAGCAGGATCAGTATACTGACGCAATTTTTTTCTGAAGGTGGTTGGAATACCGGTATG
AATTTTATAATCATTGAATTAAGGCGTAAGATTTGGTAACAAAAACTGCTTTTCACGGGAAA
CGATGAAACATGGCACAATTCAACCTGACTTTCTTTTATGAAAAGGCTCTCAAGCTGCCTTG
TACTCCCTCCGTCCCATAATATAAGAACGTTTTTGACATTAGTGTAGTACCAAAAACGTTCT
TATATTATGGGACAGAGGGAGTATTTATTAATGGATAAAAAGAGAGGTACAGGGTTCTAGTA
GTAACATATGCAAATGGTGATACAGTATATTATCTACGATTTTGTGCAAGAATTTTCACACG
AAAACAATATTCCATGCAGTTTTTAGTTTTGACTTTTTTTTTTGAATTTTCNACTTTTTTTTTTG
AATTTTCACGCGCGCGGGGGGTGGGGGGGGGGGGAGAGAACTCCCCCACCTGAATATAT
TTCTCGAACCCAACATACAGTACAATAGTACAAATGCCGGGGCGGCAGCAGAGAGAGAGG
CAGGGGGCGACCCCCCCATGCTATGTTACATGCAGCTTTGTTCTACCCACGCAGAGCCTG
TAGCCAGTTTTGACTCTTTTTGAATTGTTTGGATTGCTTACCACATGTGTCACGTGGTTTTA
GTTTTGACTCTTGACTGTTTAATACCTACTCTTTTTAAGATTTTGGATTCTAATGTCATCTGT
CTTGCAACTCAGAGCTTTCAGCCGTGCTAACTTCCATTGATTCATCCGCCATCGCATCAAA
CATCCAGCACCATGCAGACTTCACACCGTTATTCTCGCCAGAGCACTCTTCACCCCTAAAG
GCTTACCATGCAACTGCCAAAAGTGTTTTTGATTCTCTGATATTGAATTGGAATGCAACATA
TGACTATTACAACAAAGTGAATGCAAAGCAAGCTTATTACCTATCCATGGAATTTTTGCAGG
TCTCGCAATATACTTTCTTCAGCCTTATCTCATACCTTTTGGTAACTGCGTTATGATGTTATA
TGTTTTTTTTTGTACTGCAGGGAAGAGCTCTTACAAATGCTATTGGCAACCTAGAGCTAACA
GGACAATATGCAGAAGCGTTAAAACAACTTGGACAGAACCTAGAGGATGTTGCTAGTCAGG
TAAGCCATTTTTGTCAGAGAATGGTTTTTAGTCCATATCAAAATTATTGCCGAGCCATTTTTC
TAAATAACATTTTATTATTATTCATGAACTTCCATATTTTTTTCAAATCTGCAAAAGGAGAAGT
TTTTTTCCTGGGGGTTCCACCATCTGACTTATATTTGTTATGCAGTTTTCAGAGTGATTGACT
ATGAAATTACGCCAAAACACGAGCATTGCCTAGAGTGCCCTGTAGTTTTAGAAATGTTAAGA
TTGGATATGTTACTTTTCTATTACAATGAAACAGGAACCAGATCCTGCCCTTGGCAATGGTG
GACTAGGCCGTCTAGCGTCCTGTTTTCTGGATTCTATGGCAACCCTAAATTATCCAGCATG
GGGATATGGACTTCGGTACAGATATGGCCTCTTTAAGCAAATCATAACAAAGGATGGTCAG
GAGGAGGTAGCTGAGAATTGGCTAGAGGTAGTGATATGTCTTCTACTTGTTTACTGTTTGG
AAGCGTTGATTATGTTGGTTTCTGTTTTAATGATCTGCATGTTAGCTTCTTTTGCCATTTATA
ACTGATGCTGTATCTCCAGATGGGAAATCCATGGGAGATTGTAAGAAATGATGTCTCTTATC
CTCTGAAATTCTATGGCAAAGTGGTTGAAGGCACTGATGGGAGAAAACACTGGATTGGAG
GAGAGAATATCAAGGCTGTGGCACATGATGTTCCTATTCCTGGCTACAAGACTAAAACTAC
TAATAATCTTCGTCTTTGGTCAACAACAGTACCATCACAAAACTTTGATTTGGGAGCTTTTAA
TGCTGGGGATCATGTCAAGGCCAACGAAGCACATCTAAATGCTGAAAAGGTATTCCTTTGT
CTGCATTTATTTTGAATATAAATCATTAAATCTCAATAACTATTGATTTAAATGGTAAACACTA
AACATTGTCTAGCTAGTAGTCACTGTGGTGTCCCTCAATGTCATTTTAGAATGTGCACAGTC
AAATGTTTGATAACTTTGATTGTAATAATATGATAATATACAGAAACTTTGATTGACACTAGA
TTTTTCATAAACATACTTATTGTATTTCCATGTAAATAGAAAAGCTGATGTTCAGAGGTTTGT
GTGTCAAAATGAACAGAGTTTGTGCACACATAATATCTTCATCTGGTGTTTGCAGTTCTCTA
GGTTGTGTTTGCGTTTTCTCCTGCTCTGCCTTAACTAATCTCAGCATATGCATCAGGCTACA
TTGCTCCTCTGTGTCTATAAAAAATAAGTCTGATGATATATGGTTGCAGTCCAGTAGTTCGT
AGATGGCACAGCATGGAAGAACTTTTCACATGGAAGCCTTACCCTTATAATTTTAATTATAC
CTTTTGAGTGTTTGTGCATTGTGTACATACACCATTAAGTATGGGTGCTCAAAAATGGTTTA
TCCCAAACTGTGCAAAAGCCGCCACTGTTTGTGTATTAATTTTTTTGAACTAGCTAAACAAC
TTGACCGAACAAGATACATACAATTGCACTCACTTCTGAAGCAGCTCAGTTGGACGGATTT
GTTCCAATCCCGTGCATGGGTTAAATTGTCGAGTTGTCCTCGCTTGTTCAGTAGTAGGAGT
ACTATCTATTATATTTATACTAAAGTTTTCTTTTTGTGATACAGTACAAACTTTTAGTAATACT
TACATTTGCGACCACTGTGGACAAAATTTATTATGTCTAACTTAACTTTATCACTGGTCTTCT
CATCATGCCAGATATGCCACGTATTGTATCCAGGGGACGAATCATCAGAGGGGAAAATTCT
TCGCCTGAAACAACAATATACACTATGCTCGGCCTCCCTGCAGGACATTATTTCTCGTTTTG
AGTCGAGGGCTGGTGATTCTCTCAACTGGGAGGACTTCCCCTCTAAAGTTGCAGTTCAGAT
GAATGACACCCATCCAACACTGTGCATTCCCGAGTTAATGAGAATACTGATGGATGTTAAG
GGATTGAGCTGGAATGAGTCTTGGAGTATCACGGAAAGGTATCACAGAAGGGTACTGCAG
TTTGATGTGTTATGCTGTTCCTCCAGTTCCAGTTAACTAATTTTATTTCCTCATCTGTCTTCC
ATTCAATCATGTACTGATTTTCTGATATCCACATCATTTGTAGAACCGTTGCATACACTAACC
ACACAGTACTTCCTGAAGCTCTGGAGAAGTGGAGCTTGGACATTATGCAGAAACTTCTACC
TCGACATGTTGAGATTATAGAAAGAATAGATGAGGAGGTATCAAATGTGCAGATAATATTAT
ACAATTAATTTCATGCTCTGTAGTTCAACTTATGGTTTACTTGTCTTCATCGTTGATTTTAAA
CAGCTGATGAACACCATCGTCTCAAAATATGGAACGGCAGATATTTCACTGTTAAAACAGAA
GCTTAAAGATATGAGGATCTTAGACAACGTTGATCTTCCAGCCTCTGTTGCTAAACTGTTTA
TTAAACCAAAAGAGAAAAAGGGGAAGTTGCTTGTTGAATCTTTGGAGTCTATTGCTGAAGCT
GACGAGAAAACTGAGTCAGAAGAGGAGGAAAACATTCTATCCGAGACAACAGAGAAAAAG
GGCGAATCTGACTCTGAAGAAGCTCCTGATGCAGAAAAGGAGGATCCTGAGTATGAGTTA
GATCCATTTACAAAATACGATCCTCAGTTACCTAGAGTTGTTCGAATGGCGAACCTCTGCGT
TGTTGGTGGACATTCAGTTAATGGTGTGGCTGAGATTCACAGTGAAATTGTTAAGCAAGAT
GTGTTCAATAGCTTTTATGAGGTATTTGCATGATGATTTGGAAACCGTGATCTTATTTTCCAA
TGGCTGCTATAATCGTCAGGTGATATGGTATTCACCATCTGCCATTTTCTTGTGTTCTTTGC
CAGATGTGGCCTACTAAGTTCCAAAACAAAACAAATGGAGTGACCCCCAGGCGTTGGATCC
GGTTTTGTAATCCTGAATTAAGTACAATAATTTCAAAATGGATAGGCTCTGATGACTGGATT
CTGAACACTGATAAACTTGCAGGACTGAAGAAGGTACTTCCAGTAGTTTTATTCCCCAATAA
TCAGTAATCTAATGCTTCCTTCACTTCAATGTTAACTGTGCATTGTTTTTACATTGATGTGTG
TTCATCCTGTAACAGTTTGCTGATGATGAGGATCTGCAATCAGAATGGCGTACTGCTAAAA
GGAATAACAAGATGAAGGTAGTTTCGCTGATAAGAGACAAGACTGGATATGTTGTCAGCCC
AGATGCAATGTTTGATGTGCAGGTATATTTTGTACTAGAAAACATGTGTCTTCTTATAAATAT
GGTTTAACTCAGCTCTTGGTACTGGACACTTAGGTGAAAAGAATACATGAATATAAGCGGC
AGCTTCTAAATATCCTTGGTATCGTTTACCGCTACAAGAAGATGAAAGAAATGAGTGCAAAA
GATAGAATAAAGAGCTTTGTTCCAAGGGTATGCATATTTGGCGGGAAAGCTTTTGCCACTT
ACGTACAGGCAAAGAGGATTGTGAAGTTTATCACAGATGTCGCAGCTACTGTAAATTATGA
TCCTGATATTGGAGATCTACTGAAGGTAAATTTGTGTCTTTGATGCCACTTATCAGTCTTAAT
TTTCATGTTCTGTCACTGGCAAGTTGACTCCGACTGAAAAGCATTGAATGTTCAGAGTTTCA
AAGGAAAACAGAGCAGTGAATGTTCTTAATGTCGGTGCTTGTAAAACTGAGCAACTGAATA
GCCACAGGTTTTTTAGAGAACAATATAGCCACAAATTTTTTTGACACTTTTATTGCTATCAGC
ATCTGATGCTGATATTACTGATAACTAGATGAGGTAAATATAAGGTGACAGGTTAAGTCTGA
GGATTTTTGTTTGTTATCCAGGTTGTATTTGTCCCAGATTATAATGTTAGCGTTGCTGAGAC
GCTCATTCCTGCCAGTGAATTGTCCCAGCATATCAGGTAATATTCTGCCTACTCTTATATAG
TATGTGAATACATAAAAAAAGAATATTACCTGGAGTTGGCTGACATAGAATTTTTCATGTGC
CTTTTTGTTTGCTATAATATGTACCTGGAGTCTTACTGGGATAGTGCCATCCAGAAGTGCCA
CCCCAGTTTCTATTTCTGTATAAGACGTGCATTTTTAACAGTATTACTATCCATTCAGTACCG
CTGGAATGGAAGCTAGTGGAACCAGCAACATGAAGTTTGCAATGAATGGCTGTATTCTTAT
TGGAACTTTGGATGGTGCGAATGTGGAGATCAGAGAAGAGGTTGGAGAGGAGAACTTTTT
CCTCTTTGGTGCACATGCACCTGAAATCGCTGGTTTGAGGCAAGAGAGAGCTGAAGGGAA
GGTACCACACTTGTTTATATAGCTGTGCCTACTATCAAACTAGTAAAGATCTCTCCCATATG
TGCTGTCCAAATAGTTTAGCTGAAGGAAAATCATAACTTTACAAGTTTCCTTTGCACTTGTA
GTTTGTGCCTGACCCGAGATTTGAAGAGGTTAAGGAATACGTCCGCAGTGGCATCTTTGG
GACTGGCAACTATGATGAATTGATGGGTTCTTTGGAAGGAAATGAAGGTTATGGACGTGCA
GATTATTTTCTTGTTGGCAAGGATTTTCCCAGTTACATTGAATGCCAGGAAAAAGTTGATGA
AGCATACCGAGATCAGAAGGTAAGTTGTATAACTGACGCTGTTTGTTTTCTGGCTGAAATG
CTCACAGTGATTATGCAAACTTTCTGATATGTGATGGCTGACATCAGTGTTTCACTGTGAAC
GACTTCATTGATTCGTAGGAAGGAATATTTCTTAGTTTAAAGTTTCAAACTGAAGTATCTATC
GGTGTGTTGGAAGATCATAAACAATCAGAGAATATAGTTGCTAGATGTGCTATATTACTCAT
CTGTTGGCTGTTGAGTGTTGATACATGAGCTTTGATGTAGCTAGACCTTCAGGAAAGTGCT
TCTGTGCATTCCAATAAAAAAATAACTTATAACTTAACAAAACAAAACAATTAGGTTGAACCT
CACCAGGGGGCCTTTTGTTATCTGTTTTCTGAAATGAAAATCAGAAGTGGGGAGCTCCTCA
ATTCGAAAAAGAAAAAGAAAATCAGGGGGTAACTGAGGGTCTGGTTTCCCTCTGTTGGAGC
CCCCAAGAATTCTGTGGTCTTATTTTTGTAAGAGTATAAGATGCACTGCTCCGTTAAGTTTC
TCCTTCACCCATTTGTTTTCATTGCTTCGTGACATTTCGTGTGACAGCTCAGATCACCATAT
GCATCCCATTTGTACACATTATTTTGCTACCGTGGATATTTCCACTCTGTTTCATGTGTGAC
ATTTTTAATTCGTTACATTGTTACTCTAAACATTAGTCTAATAGATTTTCAGAGTTTTGACCCT
CCTAATCACATAATATCCGTTTCGACCTGCAGCTATGGACAAGAATGTCTATCCTCAACACA
GCGGGTTCCCCCAAGTTCAGCAGTGACCGGACGATCCACGAGTATGCCAAGGACATATGG
GGTATCAGCCCCGTCATCATGCCCTGAATAATTCAAGCAGCTTCCAGGAACCTTGAGAGTC
TATGAGGCGCGATTGGTCGCTGATTTGCCTTGATTCAGCGAGAATGTATTGCTCCCCCCAG
TTTTGTATAGGCATATGCAAGTTCTGACATGGACCTGAGTGCTGAGAGCATGCAGGGTAGT
TCATCCTTAGCAATATTGCATGTCAGTTAGCCAGTATTACCCAGGCTTATGGTGTGTGTATT
TCGCTGAATAAAGTTGGAGCAGAGTCATCAAACAAACATACTTTGGTGTATCTGTAATGATA
AATAAATGGTACATGTGACTGAACAAAAAGTGTCGTTGGCGTGCACATGTAGTAGAAGCAT
GTTTCTTCAGAGAGGGTGCAAAGCTGAAAGCACCCCAGCTTTGGAGTGAGAATGCAGAAG
TTACGAAAACATTTGTTTACATCCAAAGTGCAACGGGATTCGTCAAGAGTTCAGAGTCTTCC
ATCACGGTCATAGAGCATGTCCAATAGCAGCCCTATAAACCTATAACAGAGCAGTAGCAGT
GGTCCCTCACATTTTGAACCAAAAATGCAGCTCCAACGGCTGCCCTATTTGTAAAATTTACT
TCAAAATTTGTTGGACAGCCCTAAAATGCAACACCAACTGATGTAAATGTACAACTATTCGG
CTGCTGCCCTAAAATTTGCTCCAAGTGGCGAACCACTCAGGACCACAAGAAAACAGTTGTT
TTACGTTGTGCCAAAAATGTTGCTTCAACTACTGC
SEQ ID NO: 34 >Wheat (tetraploid) PHS1 5A (Kronos) CDS
ATGGCGACCGCCTCGCCGCCGCTCGCCACCCCCTTCCGCCCGCTCGCCGTCGCCGGAG
GTAGCGGCGGACTCGTCGTGGGCGCCAGGGCCGTGGTACCGCCGAGGGGGGGGCGGC
GGGGGTTCGTGGTGCGGAGCGTGGCGAGCGATCGGGAGGTGCGGGGGCCCGCCCCGA
CCGAGGAAGAGCTTTCAGCCGTGCTAACTTCCATTGATTCATCCGCCATCGCATCAAACAT
CCAGCACCATGCAGACTTCACACCGTTATTCTCGCCAGAGCACTCTTCACCCCTAAAGGCT
TACCATGCAACTGCCAAAAGTGTTTTTGATTCTCTGATATTGAATTGGAATGCAACATATGA
CTATTACAACAAAGTGAATGCAAAGCAAGCTTATTACCTATCCATGGAATTTTTGCAGGGAA
GAGCTCTTACAAATGCTATTGGCAACCTAGAGCTAACAGGACAATATGCAGAAGCGTTAAA
ACAACTTGGACAGAACCTAGAGGATGTTGCTAGTCAGGAACCAGATCCTGCCCTTGGCAAT
GGTGGACTAGGCCGTCTAGCGTCCTGTTTTCTGGATTCTATGGCAACCCTAAATTATCCAG
CATGGGGATATGGACTTCGGTACAGATATGGCCTCTTTAAGCAAATCATAACAAAGGATGG
TCAGGAGGAGGTAGCTGAGAATTGGCTAGAGATGGGAAATCCATGGGAGATTGTAAGAAA
TGATGTCTCTTATCCTCTGAAATTCTATGGCAAAGTGGTTGAAGGCACTGATGGGAGAAAA
CACTGGATTGGAGGAGAGAATATCAAGGCTGTGGCACATGATGTTCCTATTCCTGGCTACA
AGACTAAAACTACTAATAATCTTCGTCTTTGGTCAACAACAGTACCATCACAAAACTTTGATT
TGGGAGCTTTTAATGCTGGGGATCATGTCAAGGCCAACGAAGCACATCTAAATGCTGAAAA
GATATGCCACGTATTGTATCCAGGGGACGAATCATCAGAGGGGAAAATTCTTCGCCTGAAA
CAACAATATACACTATGCTCGGCCTCCCTGCAGGACATTATTTCTCGTTTTGAGTCGAGGG
CTGGTGATTCTCTCAACTGGGAGGACTTCCCCTCTAAAGTTGCAGTTCAGATGAATGACAC
CCATCCAACACTGTGCATTCCCGAGTTAATGAGAATACTGATGGATGTTAAGGGATTGAGC
TGGAATGAGTCTTGGAGTATCACGGAAAGAACCGTTGCATACACTAACCACACAGTACTTC
CTGAAGCTCTGGAGAAGTGGAGCTTGGACATTATGCAGAAACTTCTACCTCGACATGTTGA
GATTATAGAAAGAATAGATGAGGAGCTGATGAACACCATCGTCTCAAAATATGGAACGGCA
GATATTTCACTGTTAAAACAGAAGCTTAAAGATATGAGGATCTTAGACAACGTTGATCTTCC
AGCCTCTGTTGCTAAACTGTTTATTAAACCAAAAGAGAAAAAGGGGAAGTTGCTTGTTGAAT
CTTTGGAGTCTATTGCTGAAGCTGACGAGAAAACTGAGTCAGAAGAGGAGGAAAACATTCT
ATCCGAGACAACAGAGAAAAAGGGCGAATCTGACTCTGAAGAAGCTCCTGATGCAGAAAA
GGAGGATCCTGAGTATGAGTTAGATCCATTTACAAAATACGATCCTCAGTTACCTAGAGTTG
TTCGAATGGCGAACCTCTGCGTTGTTGGTGGACATTCAGTTAATGGTGTGGCTGAGATTCA
CAGTGAAATTGTTAAGCAAGATGTGTTCAATAGCTTTTATGAGATGTGGCCTACTAAGTTCC
AAAACAAAACAAATGGAGTGACCCCCAGGCGTTGGATCCGGTTTTGTAATCCTGAATTAAG
TACAATAATTTCAAAATGGATAGGCTCTGATGACTGGATTCTGAACACTGATAAACTTGCAG
GACTGAAGAAGTTTGCTGATGATGAGGATCTGCAATCAGAATGGCGTACTGCTAAAAGGAA
TAACAAGATGAAGGTAGTTTCGCTGATAAGAGACAAGACTGGATATGTTGTCAGCCCAGAT
GCAATGTTTGATGTGCAGGTGAAAAGAATACATGAATATAAGCGGCAGCTTCTAAATATCCT
TGGTATCGTTTACCGCTACAAGAAGATGAAAGAAATGAGTGCAAAAGATAGAATAAAGAGC
TTTGTTCCAAGGGTATGCATATTTGGCGGGAAAGCTTTTGCCACTTACGTACAGGCAAAGA
GGATTGTGAAGTTTATCACAGATGTCGCAGCTACTGTAAATTATGATCCTGATATTGGAGAT
CTACTGAAGGTTGTATTTGTCCCAGATTATAATGTTAGCGTTGCTGAGACGCTCATTCCTGC
CAGTGAATTGTCCCAGCATATCAGTACCGCTGGAATGGAAGCTAGTGGAACCAGCAACAT
GAAGTTTGCAATGAATGGCTGTATTCTTATTGGAACTTTGGATGGTGCGAATGTGGAGATC
AGAGAAGAGGTTGGAGAGGAGAACTTTTTCCTCTTTGGTGCACATGCACCTGAAATCGCTG
GTTTGAGGCAAGAGAGAGCTGAAGGGAAGTTTGTGCCTGACCCGAGATTTGAAGAGGTTA
AGGAATACGTCCGCAGTGGCATCTTTGGGACTGGCAACTATGATGAATTGATGGGTTCTTT
GGAAGGAAATGAAGGTTATGGACGTGCAGATTATTTTCTTGTTGGCAAGGATTTTCCCAGT
TACATTGAATGCCAGGAAAAAGTTGATGAAGCATACCGAGATCAGAAGCTATGGACAAGAA
TGTCTATCCTCAACACAGCGGGTTCCCCCAAGTTCAGCAGTGACCGGACGATCCACGAGT
ATGCCAAGGACATATGGGGTATCAGCCCCGTCATCATGCCCTGA
SEQ ID NO: 35 >Wheat (tetraploid) PHS1 5A (Kronos) protein
MATASPPLATPFRPLAVAGGSGGLVVGARAVVPPRRGRRGFVVRSVASDREVRGPAPTEEEL
SAVLTSIDSSAIASNIQHHADFTPLFSPEHSSPLKAYHATAKSVFDSLILNWNATYDYYNKVNAK
QAYYLSMEFLQGRALTNAIGNLELTGQYAEALKQLGQNLEDVASQEPDPALGNGGLGRLASCF
LDSMATLNYPAWGYGLRYRYGLFKQIITKDGQEEVAENWLEMGNPWEIVRNDVSYPLKFYGK
VVEGTDGRKHWIGGENIKAVAHDVPIPGYKTKTTNNLRLWSTTVPSQNFDLGAFNAGDHVKAN
EAHLNAEKICHVLYPGDESSEGKILRLKQQYTLCSASLQDIISRFESRAGDSLNWEDFPSKVAV
QMNDTHPTLCIPELMRILMDVKGLSWNESWSITERTVAYTNHTVLPEALEKWSLDIMQKLLPRH
VEIIERIDEELMNTIVSKYGTADISLLKQKLKDMRILDNVDLPASVAKLFIKPKEKKGKLLVESLESI
AEADEKTESEEEENILSETTEKKGESDSEEAPDAEKEDPEYELDPFTKYDPQLPRVVRMANLC
VVGGHSVNGVAEIHSEIVKQDVFNSFYEMWPTKFQNKTNGVTPRRWIRFCNPELSTIISKWIGS
DDWILNTDKLAGLKKFADDEDLQSEWRTAKRNNKMKVVSLIRDKTGYVVSPDAMFDVQVKRIH
EYKRQLLNILGIVYRYKKMKEMSAKDRIKSFVPRVCIFGGKAFATYVQAKRIVKFITDVAATVNYD
PDIGDLLKVVFVPDYNVSVAETLIPASELSQHISTAGMEASGTSNMKFAMNGCILIGTLDGANVEI
REEVGEENFFLFGAHAPEIAGLRQERAEGKFVPDPRFEEVKEYVRSGIFGTGNYDELMGSLEG
NEGYGRADYFLVGKDFPSYIECQEKVDEAYRDQKLWTRMSILNTAGSPKFSSDRTIHEYAKDI
WGISPVIMP*
SEQ ID NO: 36 >Wheat (tetraploid) PHS1 5A (Kronos) promoter (−2 kb upstream of ATG)
ATAAATATAACTAGAATACTTCATAAATTTAGATACGTTTGATAAAGTGCCAAGAATGGAAGA
GAAGTGATCATATGCCGCCCTGTCCATATCGGGCTCATCAGAATGTTCCCACCATCAGACT
CCAGCCCCAGGATTATTAACTTATGTCGGCGGTGCGCGGAATGGATCTTGAAGAAAGCGG
AATTTGTGTCGCCCTCACGTAACCAGGAGAACTTGGCAGGTTGACGCGCAATCGACCTCT
CCAGCAAAGCAAGTCCTAGGTAGGCGCGCTTCAGCTCGCAGAGTAACCGTACTTCTGGAG
GAGCAAGCGGGTGGTGATCTTGAGCCGCCGAGACGAGCAATCAATTACCGAGCGAGAAG
CATCTGAGTGGAGATGTTACCGAGCGTTCTGGAGCTCCAACTCTGCCGTCAGTGCATTGTC
AGCTTGTGCTTTGCGTAGATTCGCCGGAAAGGATCAGGATCATGCACGCCAGAATTCCAG
GTGTCAGAGACCACCTAATGAAAACCATCGAGCTTTGATCAAAATAGCTCAAGATGGAATC
TCCGATCACCTTGGGAGCGAAAATCTTACTAGTTTCGTTCAAGGCCAAGGTAGTTATATATT
TGTGTGTGCAATTATTAATCATTGGCCGCTTAGCAAACTCTTATTGGTTCGATAAACATTAAA
GTCATCCCCACTTTGAGAAAANNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNNNNNNNNNNNNNNNTGGGGGGGGGGGGGGGGGGGGGGGGGCAATGCCATCCTTGG
CAAGTAATAAATCCTTGCCGCCTCGTCCTTGTTTGGATGCTGCTTCTTGTCATCTCAAGGAG
GCGTGTGGAGGGTGGTGTGTCCCCGGATCTTGTTCTCTCGGTTGGGTTTTTTCTATGGCA
GGTTTATCTCAGTGGTAGTGTCACGTGAAGCAGATGACGCGACTATGCGTCTTGGTCCTCT
GCGCTCTAATTCCGGCAGATGAAGGTTGACCAGCTTCAGCCTCGTCGGGGAGCGTATGAG
ATTTGTGTTTTTTGACTCCATCTGGTCGGACCTGTGGCATTATGTAGACCCTCTCATGTATT
CTTTTTCTCCTGGACAGTAGTCTGTTACACAAATCGTGGTCGTCGGATCTTCTAGTCCACAT
CGGCGACTTTTCCGACCGCCACTTCTACAAACTCCTTATTTTCAACAAGTCTCCTTCATTGA
GATGGAAGTCCAGGCAATGGCTTTGCTCACGGCACACTGCCGGCGGATGCAAGAGAAAGA
AGACATTCGACACCCCTAAAACTATGAAGATGTAATTTTTAATCTTTGTAAGTGTTTTTGTGA
GGATTTGTGCTACTTTTAATATATTGCCTTTTGCCTTTTGCAAGGGAAAAAAAACTAAACCAT
TACACGAAGAGAAGAGCCATTCCCAGCCGTCCAATAAACACGACAAAACGCTTTACAGCAC
GGGGGCAATTCCCAGCCGTCGAAATGCGCTGCAACGGAACCAGCGCTCCCCGACACGGC
GGCACATGAGCCACAACTCGTACCATGCACCGTGCACCGGCGGTGGGCCCACTGGCCAG
CGTCCCCACACGTGTACCGGCGGCCACAATTACACGGCACGGCACGGCGACGGAGAGAT
ATTTTAACGCCTTCCAATTCAGAAGCTCCCCCATCACTCACTCGGGTACAGAGCGCAGCGG
GCCGCGTGACTCTCGCGTCGTCGCATCGCGCGCGCTGCACACCACCGTCAGCGCGACCA
CTGCCGCCA
SEQ ID NO: 37 >Wheat (tetraploid) PHS1 5B (Kronos) genomic DNA
TTGCACGGCACGGCGACGGAGAGATATTTTTAACGCCTTCCAATTCAGAAGCTCCCCCATC
ACTCACTCGGGTACAGAGCGCAGCGGGCCGCGTGACTCTCGCGTCGTCGCATCGCGCGC
GCTGCACACCACCGTCAGCGCGACCACTGCCACCAATGGCGACCGCCTCGCCGCCGCTC
GCCACCGCCTTCCGCCCGCTCGCAGCCGGCGGCGGCGCCGGAGGAGGAGGAGCGCACG
GCGTGGGCGCCGCGGGCCGGGTCGCGCCGAGGCGGGGGCGCCGGGGCTTCGTGGTGC
GGAGCGTGGCGAGCGATCGGGAGGTGCGGGGCCCCGCCTCGACCGAGGAAGGTAAGCC
GTCGCGCGCTCCCGTTGGCTGCGACGACCCGGAATGCTGGTCGGCTGTAATGTGGTTCG
GATTTTCGCTGGTGCGACCTGAATTTCTGATCCTCTGACTAATGGTCTCTCTGTTCCCGGG
TTTCGGGCTGTTGATTTTGTGCGCGCGCATTGCTAATCTACTGCTCTAGGATTTGTTAGGG
TTCAGAGGAATCTTAAATTCACGAATAAACTTCGACGAGTGTATTCTGAATTTACTGAAACT
GGCTTAGTTTGGGGCGTCAAGTTAGAATGGTGTATTTATGGCACAGTAGCAAATTCTGAGA
AATGCTTCTGTGCGTGCACAGCCACCCCTTGTGGATGATGCAGCAAGTGTCAACATACCGT
CGCTACTATCTGTAGCTGTAACAAAATCTTACGCCTTGTTTTCTGGTGGTACAGTAATATTA
TCATTTTACAAAGAGTGGCTGTAACAAAATCTTACGCCTTGTTTTCTGGTGGCTTGCCATTG
AGGAGTAGAATGCAAATGGCGATACAGTATATTATCTACGATTTTGTGCAAGAATTTTCACA
CGTATCCCATGCAATTTTTAGTTTTGACTCTCGACTGTTTATACCTACTCTTTGTAAAATTTT
GGATCCTAATATTGTTGTTCTTGCAACTCAGAGCTTTCAGCCGTGCTAACTTCCATTGATTC
TTCCGCCATCGCGTCAAATATCCAGCACCATGCAGACTTCACACCGTTGTTCTCGCCAGAG
CACTCTTCACCCCTAAAGGCTTACCATGCAACTGCTAAAAGTGTTTTTGATTCTCTGATCAT
CAATTGGAATGCAACATATGACTATTACAACAAAGTGAATGCAAAGCAAGCTTATTACCTGT
CCATGGAGTTTTTGCAGGTCTCACAATATACTTCCTTTAGCCTTACCTCAATACTTTTTGGTA
ACTGCGTTATGATGTTATATGGTTGTTATCTGTGTACTGCAGGGAAGAGCTCTCACAAATGC
TATTGGCAATCTAGAGCTAACAGGACAATATGCAGAAGCGTTAAAACAACTTGGACAGAAC
CTAGAGGATGTTGCTAGTCAGGTAAGTGATTTTTGTCGGAGATTGGTTTTTAGTCCGTATCA
AAATTATCGCAGGGCCATTATTTTTATTTTGAAAACGCATTTTATTATTAATCATCAAGTTCC
CTTTGTTTAAAAACAATCTGAAAAGAAGAAGTTTTTTTCCTGAGGGTTCCACCGTCTGACTT
ATGATCTTTATGCAGTTTTCAGAGTGACTATGAAATTACGCCAAAACACGATCTTATTGATG
CCTAGAGTGCCCTGTAGTTTAGAAATCTAAGATTGGATATGTTACTTTTCTATTACAATGAAA
CAGGAACCAGATCCTGCCCTTGGCAATGGTGGTCTAGGCCGTCTAGCGTCCTGTTTTCTG
GATTCTATGGCAACCCTAAATTATCCAGCATGGGGATATGGACTTCGGTACAGATACGGCC
TCTTTAAGCAAATCATAACAAAGGATGGTCAGGAGGAGGTAGCTGAGAATTGGCTAGAGGT
AGTGATGTGTCTTCTACTTGTTTACTGTTTGGAAGTGTTGATTATGTTGGTTTATTTATGTTC
TAATGACCTGCATGTTAGCTTCTTTTCTATTTATAACTGATGCTGTATCTCCAGATGGGAAAT
CCATGGGAGATCGTAAGAAATGATGTCTCTTATCCTGTGAAATTCTATGGCAAAGTGGTTG
AAGGCACTGATGGGAGAAAGCACTGGATTGGAGGAGAGAATATCAAGGCTGTGGCACATG
ATGTTCCTATTCCTGGCTACAAGACTAAAACTACTAATAATCTTCGTCTTTGGTCAACAACA
GTACCATCACAAAATTTTGATTTGGGTGCCTTTAATGCTGGGGATCATGCCAAGGCCAATG
AAGCACATCTAAATGCTGAAAAGGTATTCTTTCGTCTACTTTATTTTGAATATAAACCATTAA
CTCTCAACAACTACTGATTCAAAGGCTAAACAGTGCTTTGCTAGTAGTTACTGTGTTTTCTC
TTAGATGTCATTTCATAATGTGTGCAGTCAATCGTTTGATATACACAAACCTATTTGGATTTC
TCCTGTAAAAATGTTGTCACTAGATTTTTCATGAAAAAACTTATTGTATATGTAAATAGAAAA
GCTGATGCTCAGGTGCTTTGTGTTCAAATGACAAGTAAAAATGAACAGAGTTTGTGCACCC
ATAACTTCTTCACCTGGTGTGTGCAATTCGTTCGGTTGTATTTATGCGTGTTCTTATTCTGC
CTGAATGATTCTCAGCATATGCATCAGGCTACATTTCTCCTCTGTGTCAAGAAAAAATAAGT
CTGACCAGATGCAGTCCAGTAGTCTTTAGATCTTTTCACATGGAAGCGTTACTCTATGATTT
TTATTATACCTTTAGAGTGTTTGTGCATTGTGCACATATACCATGAAGTATGGGTGTTCAAAT
GTGGTTCTCTCAAACTGTGCTAAACCCACCACTGTTTGCCAGTATTCATTTTTCTGAACTAG
CAACAACTTGACTGGACAAGATACATTGTACTCACATCTGAAGCTGCTGGGTTGGACAGAT
TTGTCCGACCCGTCTTTGAACATCTCTACTATTTAGTGTGCATGGGTTAAATTGTTGACTTTT
CCTCGTTTGTTCAGTAGTAGGAGCACTATCTAATTATATTTATATTCAAGTTTTCTCTGTGTG
TTACGTTATAAACTTTTAGTCATACTTACATTTGTGGACAAAAGTTATCATGTCTAACTTAAG
TTTGTGACTGATCTTCTCATCATGCCAGATATGCCACGTATTGTATCCAGGGGACGAATCAT
CAGAGGGGAAAATTCTTCGCTTGAAACAACAATATACATTATGCTCGGCCTCCCTGCAGGA
CATTATTTCTCGTTTTGAGTCGAGGGCTGGTGATTCTCTCAACTGGGAGGACTTCCCCTCT
AAAGTTGCAGTTCAGATGAATGACACTCATCCAACACTGTGCATTCCGGAGTTAATGAGAA
TACTGATGGATATAAAGGGATTGAGCTGGAATGAGGCTTGGAGTATCACAGAAAGGTACCA
CAGAAGGGTACTGCAGTTTGATGTGTTATGCTGTTCCTCCAGCTCCAATTAACTAATTGTAT
TGCCTCATCTGTCTTCCATTCAATTATGTACTGATTCTCTGATATCATTTGTAGAACCGTTGC
ATACACTAACCACACGGTACTTCCTGAAGCTCTGGAGAAGTGGAGCTTGGACATTATGCAG
AAACTTCTACCCCGACATGTTGAGATCATAGAAACAATAGATGAGAAGGTAACAAATGTGC
AGATAATATTATGCAATAAATTTCATTCTCTGTAGTTCAACTTATGGTTTACTTATCAACATC
GTTGATTTTAAAACAGCTGATGAACAACATCGTCTCAAAATATGGAACGGCAGATATTTCAC
TGTTAAAACAGAAGCTTAAAGATATGAGGATCTTAGACAACGTTGATCTTCCAGCCTCTGTT
GCTAAACTGTTTATTAAACCTAAAGAGAAAAAGGGGAAGTTGCTTGTTGAATCTTTGGAGTC
TATTGCTGAAGGTGACGAGAAAACTGAGTCACAAGAGGAGGAAAACATTCTATCCGAGACA
GCAGAGAAAAAGGGCGGATCTGACTCTGAAGAAGCTCCTGATGCAGAAAAGGAGGATACT
GTGTATGAGTTAGATCCATTTGCAAAATACGATCCTCAGTTACCTAGAGTTGTTCGAATGGC
AAATCTCTGTGTTGTTGGTGGACATTCAGTTAATGGTGTGGCTGAGATTCACAGTGAAATTG
TTAAGCAAGATGTGTTCAATAGCTTTTATGAGGTATTTGCACGATGATTTGGAAACCATGAT
CTTATTTTCTAACGGCTGCTATAATCGCCAGGTAATATGGTATCACCATCTGCCATTTTCTTA
TGTTATTTGCCAGATGTGGCCTACTAAGTTCCAAAAAAAACAAACGGAGTAACTCCCAGG
CGTTGGATCCGGTTTTGTAATCCTGAATTAAGTGCAATCATTTCAAAATGGATAGGCTCTGA
TGACTGGATTCTGAACACTGATAAACTTGCAGGACTGAAGAAGGTACTTCCAGTAATTTTAA
TTTCCAAATATCAGTAATCTAATGCTTCCTTCACTTCCATGTTACCTGTGCCTTGTTTTTACA
TTGATGTGTGTTCATCCCGTAATAGTTTGCTGATGATGAGGATCTGCAATCAGAATGGCGT
ACTGCTAAAAGGAATAACAAGATGAAGGTAGTTTCGCTGATAAGAGACAAGACTGGATATG
TTGTCAGCCCAGATGCAATGTTTGATGTGCAGGTATATTTTGGACTAGAAACATGTGTCTCC
TGTTAAATATGGTTTAACTCGGATCTTGGTACTTGACACTTAGGTGAAAAGAATACATGAAT
ATAAGCGGCAGCTGCTAAATATCCTTGGTATCGTTTACCGATACAAGAAGATGAAAGAAAT
GAGTGCCAAAGATAGAATAAAGAGCTTTGTTCCAAGGGTATGCATATTTGGTGGGAAAGCT
TTTGCCACTTACGTACAGGCAAAGAGGATTGTGAAGTTTATCACAGATGTCGCAGCTACTG
TAAATTATGACCCTGATGTTGGAGATTTACTGAAGGTAAATTTGTGTCTTCAATGGCACTTA
TCATTCTAAATTTTCATGATCTGTCACTAGCAATTTGACTCCGACTAGGGGTTACTGAAAAG
CACTGAATGTTCCGAGTTTCAAAAGAAAACAGAGCAGTGAATGTTCTTAATGTCGGTGCTT
GTAGAACTGAGAAACTGAATAGCCACAGTTTTTTTTAGAGAACAATAGCCACAATTGTTCTG
ATGCTGATTTTGCTGATAACTAGATGAGGTAAATACAAGGTGACAGGTTAAGTTCTGAGGAT
TTTTGTTTGTTATCCAGGTTGTATTTGTCCCAGATTATAATGTTAGCATTGCTGAGAAGCTCA
TTCCTGCCAGTGAATTGTCCCAGCATATCAGGTAATATTCTGGCTACTCTTATATAGTATGT
GAATACAAAGAAAAGGAGTCTTATCTGGAGGTGACTGATGTGGAAATTTTCATGTGCCTTTT
TGTTTGCTACTCCCTCTGTCCCATAATATAAGAGCGCTTTTGACAGTACACTAGTGTCAAAA
ACGCTCTTATAATATGGGACGGAGGGAGTATAATATATACCTGGAGTCTTGCTGGGATAGT
GACATCCAGAAGTGCCACGCCAGCTTCTATTTCTGTACAAGACGTGCATTTTTAACACTATT
ACTATCCATTCAGTACCGCTGGAATGGAAGCTAGTGGAACAAGCAACATGAAGTTTGCAAT
GAATGGTTGTATTCTTATTGGAACTTTGGATGGTGCGAATGTGGAGATCAGAGAAGAGGTT
GGAGAGGAGAACTTTTTCCTCTTTGGTGCACATGCACCTGAAATCGCTGGTTTGAGGCAAG
AGAGAGCTGAAGGAAAGGTACCACACTTGTTTATAGAACTGTGCCTAACTATGAAACTAGT
AAAGATCTCTCCCAAATGTGTGTTGTCCAAATAGTTTAAAGGAAGAAAAATTATAACTTTACC
ATTTTCCTTTGCACTTGTAGTTTGTGCCTGACCCGAGATTTGAAGAGGTTAAGGAATACGTC
CGCAGTGGCATCTTTGGGACTAGCAACTATGATGAATTGATGGGTTCTTTGGAAGGAAATG
AAGGTTATGGACGTGCAGATTATTTTCTTGTTGGCAAGGATTTTCCCAGTTACATTGAATGC
CAGCAAAAAGTTGATGAAGCATACCGAGATCAGAAGGTAAGTTGTATAACTGACGCTGTTT
GTTTTCTGGCTGAAATGCATACAGTGATTATGCAAACTTTCTGATATGTGATGGCTGCCATC
AGTGTTTCACTGTGAACGACTTCATTGATTCGTAGGAAGGAATATTTCTTAGTTCAAAGTTT
CAAACTGGATTATCTATAGGTGTGTTGGAAGATCATAAACAATCAGAGAATATAGTTGCTAG
ATGTGCTATACTATATGTTGGCTGTTGAGTGTTGATACATGAGCTTTGATGAACCTAGCTAG
ACCTTCAGGAAAGTGCTTCTATGCATTCCAATAAAAAAAACTTATAACTTAACAAAACAATTA
GGTTGAACCTCGCCAGGGAGCGTTTTGTTATCTGTTTTCTGAAATGAAAATCAGAAGCGGG
GAGCTCCTCGATTCTAAAATGAAAAAAAAATCCAGGGATAACTGAGGGCCTGGTTTCCCTC
TGTTGGAGCCCCAAAGAATTCTGTGGCCTTATTTTTGTAAGAGTATAAGATGCACTGCTCC
GTTAAGTTTCTCCTTCATCCATTTGTTTTCATTGCTTCGTGACATTTCGTGTAACAGAGCATC
CCATTTGTACACATTATTTTGCTATCGTGGATATTTCCACTCTGTTTCATGTGTAACATTTTT
AATTCGTTACATCCTTGCTAACTACACATAGTCTGATAGATTTTCAGAGTTTTGGTCCTCCTA
ATAACATAATATCCGTTTCTACCTGCAGCTATGGACAAGAATGTCCATCCTCAACACAGTTG
GTTCCCCCAAGTTCAGCAGTGACCGGACGATCCACGAGTATGCCAAGGACATATGGGATA
TCAGCCCCGTCATCATGCCCTGAATTATTCAAGCAGCTTTCAGGAACATTCAGAGTCTATG
AGGCGCGATTGGTCGCAATGTATTGCTCCCCCCAGTTTTGTATAGGCACATATGCAAGTTC
TGACATGGACCTGAGTGCTGAGAGCATGCAGGGTAGTTCATCCTTAGCAATATTGCATGTC
ACTTAGCCCGTATTACCCAGGCTTATGGTGTGTGTATTTCGCCGAATAAAGTTGGAGCAGA
ATCATCAAACAAACATACTTTGGTGTATCTGTAATGATAAATAAATGGTAAATGACTGAACAA
AAAGTGTCGTTGGCGCACACATGTAGTAGAAGCATGTTTCTTCAGAGAGGCTGCAAAACTG
AAAGCACCCTAGCTTTGAAGTGAGAATGAAGAAGTTACGGAAACATTTGTTTACATCCAAAG
TGCAACGGGATTCGTCAAGA
SEQ ID NO: 38 >Wheat (tetraploid) PHS1 5B (Kronos) CDS
ATGGCGACCGCCTCGCCGCCGCTCGCCACCGCCTTCCGCCCGCTCGCAGCCGGCGGCG
GCGCCGGAGGAGGAGGAGCGCACGGCGTGGGCGCCGCGGGCCGGGTCGCGCCGAGGC
GGGGGCGCCGGGGCTTCGTGGTGCGGAGCGTGGCGAGCGATCGGGAGGTGCGGGGCC
CCGCCTCGACCGAGGAAGAGCTTTCAGCCGTGCTAACTTCCATTGATTCTTCCGCCATCGC
GTCAAATATCCAGCACCATGCAGACTTCACACCGTTGTTCTCGCCAGAGCACTCTTCACCC
CTAAAGGCTTACCATGCAACTGCTAAAAGTGTTTTTGATTCTCTGATCATCAATTGGAATGC
AACATATGACTATTACAACAAAGTGAATGCAAAGCAAGCTTATTACCTGTCCATGGAGTTTT
TGCAGGGAAGAGCTCTCACAAATGCTATTGGCAATCTAGAGCTAACAGGACAATATGCAGA
AGCGTTAAAACAACTTGGACAGAACCTAGAGGATGTTGCTAGTCAGGAACCAGATCCTGCC
CTTGGCAATGGTGGTCTAGGCCGTCTAGCGTCCTGTTTTCTGGATTCTATGGCAACCCTAA
ATTATCCAGCATGGGGATATGGACTTCGGTACAGATACGGCCTCTTTAAGCAAATCATAAC
AAAGGATGGTCAGGAGGAGGTAGCTGAGAATTGGCTAGAGATGGGAAATCCATGGGAGAT
CGTAAGAAATGATGTCTCTTATCCTGTGAAATTCTATGGCAAAGTGGTTGAAGGCACTGAT
GGGAGAAAGCACTGGATTGGAGGAGAGAATATCAAGGCTGTGGCACATGATGTTCCTATT
CCTGGCTACAAGACTAAAACTACTAATAATCTTCGTCTTTGGTCAACAACAGTACCATCACA
AAATTTTGATTTGGGTGCCTTTAATGCTGGGGATCATGCCAAGGCCAATGAAGCACATCTA
AATGCTGAAAAGATATGCCACGTATTGTATCCAGGGGACGAATCATCAGAGGGGAAAATTC
TTCGCTTGAAACAACAATATACATTATGCTCGGCCTCCCTGCAGGACATTATTTCTCGTTTT
GAGTCGAGGGCTGGTGATTCTCTCAACTGGGAGGACTTCCCCTCTAAAGTTGCAGTTCAG
ATGAATGACACTCATCCAACACTGTGCATTCCGGAGTTAATGAGAATACTGATGGATATAAA
GGGATTGAGCTGGAATGAGGCTTGGAGTATCACAGAAAGAACCGTTGCATACACTAACCA
CACGGTACTTCCTGAAGCTCTGGAGAAGTGGAGCTTGGACATTATGCAGAAACTTCTACCC
CGACATGTTGAGATCATAGAAACAATAGATGAGAAGCTGATGAACAACATCGTCTCAAAATA
TGGAACGGCAGATATTTCACTGTTAAAACAGAAGCTTAAAGATATGAGGATCTTAGACAAC
GTTGATCTTCCAGCCTCTGTTGCTAAACTGTTTATTAAACCTAAAGAGAAAAAGGGGAAGTT
GCTTGTTGAATCTTTGGAGTCTATTGCTGAAGGTGACGAGAAAACTGAGTCACAAGAGGAG
GAAAACATTCTATCCGAGACAGCAGAGAAAAAGGGCGGATCTGACTCTGAAGAAGCTCCT
GATGCAGAAAAGGAGGATACTGTGTATGAGTTAGATCCATTTGCAAAATACGATCCTCAGT
TACCTAGAGTTGTTCGAATGGCAAATCTCTGTGTTGTTGGTGGACATTCAGTTAATGGTGTG
GCTGAGATTCACAGTGAAATTGTTAAGCAAGATGTGTTCAATAGCTTTTATGAGATGTGGCC
TACTAAGTTCCAAAAAAAACAAACGGAGTAACTCCCAGGCGTTGGATCCGGTTTTGTAAT
CCTGAATTAAGTGCAATCATTTCAAAATGGATAGGCTCTGATGACTGGATTCTGAACACTGA
TAAACTTGCAGGACTGAAGAAGTTTGCTGATGATGAGGATCTGCAATCAGAATGGCGTACT
GCTAAAAGGAATAACAAGATGAAGGTAGTTTCGCTGATAAGAGACAAGACTGGATATGTTG
TCAGCCCAGATGCAATGTTTGATGTGCAGGTGAAAAGAATACATGAATATAAGCGGCAGCT
GCTAAATATCCTTGGTATCGTTTACCGATACAAGAAGATGAAAGAAATGAGTGCCAAAGATA
GAATAAAGAGCTTTGTTCCAAGGGTATGCATATTTGGTGGGAAAGCTTTTGCCACTTACGTA
CAGGCAAAGAGGATTGTGAAGTTTATCACAGATGTCGCAGCTACTGTAAATTATGACCCTG
ATGTTGGAGATTTACTGAAGGTTGTATTTGTCCCAGATTATAATGTTAGCATTGCTGAGAAG
CTCATTCCTGCCAGTGAATTGTCCCAGCATATCAGTACCGCTGGAATGGAAGCTAGTGGAA
CAAGCAACATGAAGTTTGCAATGAATGGTTGTATTCTTATTGGAACTTTGGATGGTGCGAAT
GTGGAGATCAGAGAAGAGGTTGGAGAGGAGAACTTTTTCCTCTTTGGTGCACATGCACCT
GAAATCGCTGGTTTGAGGCAAGAGAGAGCTGAAGGAAAGTTTGTGCCTGACCCGAGATTT
GAAGAGGTTAAGGAATACGTCCGCAGTGGCATCTTTGGGACTAGCAACTATGATGAATTGA
TGGGTTCTTTGGAAGGAAATGAAGGTTATGGACGTGCAGATTATTTTCTTGTTGGCAAGGA
TTTTCCCAGTTACATTGAATGCCAGCAAAAAGTTGATGAAGCATACCGAGATCAGAAGCTAT
GGACAAGAATGTCCATCCTCAACACAGTTGGTTCCCCCAAGTTCAGCAGTGACCGGACGA
TCCACGAGTATGCCAAGGACATATGGGATATCAGCCCCGTCATCATGCCCTGA
SEQ ID NO: 39 >Wheat (tetraploid) PHS1 5B (Kronos) protein
MATASPPLATAFRPLAAGGGAGGGGAHGVGAAGRVAPRRGRRGFVVRSVASDREVRGPAST
EEELSAVLTSIDSSAIASNIQHHADFTPLFSPEHSSPLKAYHATAKSVFDSLIINWNATYDYYNKV
NAKQAYYLSMEFLQGRALTNAIGNLELTGQYAEALKQLGQNLEDVASQEPDPALGNGGLGRLA
SCFLDSMATLNYPAWGYGLRYRYGLFKQIITKDGQEEVAENWLEMGNPWEIVRNDVSYPVKF
YGKVVEGTDGRKHWIGGENIKAVAHDVPIPGYKTKTTNNLRLWSTTVPSQNFDLGAFNAGDHA
KANEAHLNAEKICHVLYPGDESSEGKILRLKQQYTLCSASLQDIISRFESRAGDSLNWEDFPSK
VAVQMNDTHPTLCIPELMRILMDIKGLSWNEAWSITERTVAYTNHTVLPEALEKWSLDIMQKLLP
RHVEIIETIDEKLMNNIVSKYGTADISLLKQKLKDMRILDNVDLPASVAKLFIKPKEKKGKLLVESL
ESIAEGDEKTESQEEENILSETAEKKGGSDSEEAPDAEKEDTVYELDPFAKYDPQLPRVVRMA
NLCVVGGHSVNGVAEIHSEIVKQDVFNSFYEMWPTKFQNKTNGVTPRRWIRFCNPELSAIISK
WIGSDDWILNTDKLAGLKKFADDEDLQSEWRTAKRNNKMKVVSLIRDKTGYVVSPDAMFDVQ
VKRIHEYKRQLLNILGIVYRYKKMKEMSAKDRIKSFVPRVCIFGGKAFATYVQAKRIVKFITDVAA
TVNYDPDVGDLLKVVFVPDYNVSIAEKLIPASELSQHISTAGMEASGTSNMKFAMNGCILIGTLD
GANVEIREEVGEENFFLFGAHAPEIAGLRQERAEGKFVPDPRFEEVKEYVRSGIFGTSNYDELM
GSLEGNEGYGRADYFLVGKDFPSYIECQQKVDEAYRDQKLWTRMSILNTVGSPKFSSDRTIHE
YAKDIWDISPVIMP*
SEQ ID NO: 40 >Wheat (tetraploid) PHS1 5B (Kronos) promoter (−2 kb upstream of ATG)
CCCACCCGGTGGACCCCCGGGACCCTTCCGGTGGTTCCAGTACAATACCGGTGACCCCC
GAAACTTTCCCGATGGCCGAAACCTGACTTCCTATATATAATTCTTTACCTCCAAACCATTTT
GGAACTCCTCGAACGTCCGGGATCTCATCCGGGACTCCGAACAACTTTCGGTTTACTGCAT
ACTCATATCTCTACAACCCTAGCGTCACCGAACCTTAAGTGTGTAGACCCTACGGATTCGG
GAGACACGCAGACATGACCGAGACGGCTCTCCGGTCAATAACCAACAGCGGGATCTAGAT
ACCCATGTTGGCTCCCACATGCTCCTCGATGATCTTCATCGGATGAACCACGATGTCGAGG
ATTCAAGCAACCCCGTATACAATTCCCTTTGTCAATCGATACGTTACTTGCCCGAGATTCGA
TCGTCGGTATCCCAATACCTCGCTCAATCTCATTACCGGCAAGTCACTTTACTCGTACCGTA
ATGCATGATCCCGTGACCAAACACTTGGTCACTTTGAGCTCATTATGATGATGCATTACCGA
GTGGGCCCAGAGATACCTCTCCGTCATACGGAGTGACAAATCCCAGTCTCGATCCGTGTC
AACCCAACAGACACTTTCGGAGATACCCGTAGTATACCTTTATAGTCACCCAGTTATGTTGT
GACGTTTGGTACACCCAAAGCACTCCTACGGTGTCCGGGAGTTACACGATCTCATGGTCTA
AGGAAAAGATACTTGACATTGGAAAAGCTCTAGCAAACGAACTACACGATCTTGTGCTATG
CTTAGGATTGGGTCTTGTCCATCACATCATTCTCCTAATGATGTGATCCCGTTATCAACGAC
ATCCAATGTCCATAGTCAAGAAACCATGACTATCATTTGATCAACGAGCTAGTCAACTAGAG
GCTTACTAGGGACATGTTGTGGTCTATGTATTCACACATGTATTACGATTTCCGGATAACAC
AATTATAACATGAATAACAGACGATTATCATGAACAAGGAAATATAATAATAATCTTTTATTAT
TGCCTCTAGGGCATATTTCCAACAATTGCGGTGAGGCGCGTGGAGGTTGGCGTGTCCCCA
AATCTAGTTATCTGGGTCAGGTTTTTTCTTTGGTAGGTTTATCTCAGTGGTAGTGTCCCGTG
AAGCGGATGACACGGCTATGTGTCTTGGTCCTCCATGCTCTAGTTTTGACATGTGAAGGTT
GGCCAACTTCGGCGTTGTCAGGGAGCATATGAGATTTGTGTTCCTTTACTTCGCCTGGTCA
GACCTACGGTATTATATAAGCCCTCTCATGTGTTTTCTTCTCTTGGGCAGCAGTCAGTTACG
CAAATCGTGGTCGCTGACATCTTCTAGTCCACATCGGCGACTTCCCGACCGCCGCTTCTAC
AAGCTCCTTATTTTTCAACAAGTTTTCTCCACCGAGATGGAAGTCAAGAGGCGGCAACGGC
TTTGCTCATGGCGCACTGCGGATGGATGCAGGAGAAGGAAGACATTCCACACCCCTCAAA
CTATGAAGATGTAATTTTTAGTTTTTGTAAGTATTTTTGTGAGGATTTGTGCTACTTTTAATAT
ATATGGCCTTTTGCCTTTTTCCAGGAAAGGAAAAACTAAACCATTACATGAGGCCATTCCCA
TCCGTCCAATAAATACGACAAAACGCTTTACAGCACGGGGGCAATTCCCAGCCGTCGAAAT
GCGCTGCAACGGAACTAGCTCTCCCAGACAGACACAGCGGCACATGAGCCACGCCTCCC
ATCGCAACCGTGCACCGGCGGTGGGCCCACCGGCCAGCGCCCCCACACGTGTACCGGC
GCCCAGCATTGCACGGCACGGCGACGGAGAGATATTTTTAACGCCTTCCAATTCAGAAGC
TCCCCCATCACTCACTCGGGTACAGAGCGCAGCGGGCCGCGTGACTCTCGCGTCGTCGC
ATCGCGCGCGCTGCACACCACCGTCAGCGCGACCACTGCCACCA
SEQ ID NO: 41 >Barley PHS1 (Morex reference genome) genomic DNA
AAAAAAAGTGCTAAGACTTCTTAAAGAAACAGGGCGTGAGATGGAAGTGCAGAGGCGGCA
GCGAATTAGCTCACGGGGCACTACCGGTGGATGTAGGAAAAAGAAGGCATTTGACACCTC
TCAAAACTATGAAGATGTAAATTTTAATTTCTAAGTGTTTTTGTGAGGATTTGTGCTATTTAAT
ACTCCCTCAATTCCAAAATAAATGTTTTAATTTTGTATTATCTCTAATACAAAGTTATACTAAC
TTTAAGATACCTATTTTAAAACAGAGGAAATATATGCATTTTGCCTTTCTCACAAAAAAAACC
CCTAAACCATCACACGAGGGGAAGAGCCATTCCCAGCCGTCCAATAAACACGACAAAAAG
CTGTACAGCACGGGAGCAATTCCCAGCCGTCGAAATGCGCTGCAACGGAAGTAGCGCTCC
CTGACACAGCGGCACATGAGCCACGCCTCGCACCACCGCACCGTGCACCGGCGCGGGG
CCCACCGGCCAGCGTCCCCACACGCGTACCGGCGGCCAGCATTGCGCGGCACGGCGAC
GGAGAGATATTTTTAACGCCTTCCAGTTCGGAAGCGCCCCCCATCACTCACTCGGGTACAG
GGCGGGCCGCGTGAGTCTCGCGTCGTCGCATCGCGCGCGCTGCACACCCACCGTCGTCA
GCGCGGACCACCGGCGCCCCACCCAATGGCGACCGCCTCGCCGCCGCTCGCCACCGCC
TTCCGCCCGCTCGCCGCCGCCGGAGGCAGCGGGGGGCTCGTCGTGGGCGCCGGGGCC
GGGGCGGCGCCGAGGCGGGGGGGGCGGGGGCTCGTGGTGCGGAGCGTGGCGAGCGAT
CGGGAGGTGCGGGGCCCCGCCTCCACCGAGGAAGGTAAGCGGCCGCGCGCTCCCGTTG
GCAGCGACGACACGGAATGCCGGTCGGCTGTAATGTGGTTTGGGTTCCCGCTGGTGCGG
CCCGTGAATTCGTGATCCTCTGACTGATTGTGCTCCCGGGTTTCGGTGGCGCGGGAGCTT
TGGGTGGGCGCGCGCATTGCTAATGTACTGCTCTAGGATTTGTTAGGGTTCAGAGGAAAC
TTAAATTCGCGAATAAACTTCAACGAATGTATTCTGAGTTTCTGAAGCTGGCTTAGTTTGGC
GCGTCAAGTTAGAATGTGTATTTATGGTAGCAAATTCTGAGAAATTCTTCTATGCGTGGAGA
GCCACCCCCCTGTGGATGCTGCAGCAAGCGCTAATATACTGTTTGCTAGTATCTGTAGCTG
TAACAAAATCCTTCAAATCTGAACTAGCGAAATAAATATGAGATTTTTGCTGAAGATTATACA
GTGTCAATATTTCTGAAGGTGGTTGGGACAGTATAGCACTTCAAACCAGCAACCAGCAGGA
TCAGTATACCGACTCATCTTTTTTTTCTGAAGGTGGTTGGAATACCGGTATCGTTTTTATAAT
TATTAAATTAAGGCGTAAGATTTGGTAGCAAAAACTATTTTCACCAGAAACGATGAAACATG
GCACAATTCAACCTGAGTACCTGACTACCTTTTTTGATGAAAAGGCTATGGGAGCTCCCTTT
CATTTATTAATTAATGGATAAAAAGAGAGGTGGAGGGTTCTAGTAGAATGCAAATGGTGATA
CAGTATATTATCTACTATTTTGTACAAGATTTTTCACACGAAAACAATATCCCATGCAGTTTT
TTGTTTCGACTCTTGACTGTTTAATACCTACTCTTTATAAGGTTTCGGATCCTAATATTGCTG
TTCTTGCAACTCAGAGCTTTCAGCCGTGCTAACTTCCATTGATTCTTCCGCCATCGCGTCAA
ATATCCAGCACCATGCAGACTTCACACCGTTGTTCTCGCCAGAGCACTCTTCACCCTTAAA
GGCTTACCATGCAACTGCTAAAAGTGTTTTTGATTCTCTGATAATGAATTGGAATGCAACAT
ATGACTATTACAACAAAGTGAATGCAAAGCAAGCTTATTACCTGTCCATGGAGTTTTTGCAG
GTCTCGCAATATACTTCCTTTAGCTTTATCTCATACCTTTTGGTAACTGCGTTACGATGTTAT
ATGGTTATTTTTTGTGTACTGCAGGGAAGAGCTCTTACAAATGCTATTGGCAACCTAGAACT
AACAGGACAATATGCAGAAGCATTAAAACAACTTGGACATAACCTAGAGGATGTTGCTAGT
CAGGTAAGCGTTTTTTGTCAGAGATTGGTTTTTAGTCCGTATATAAATTATCATTGAACCATT
ATCTAAAAAACACATTTTATTATTAATCATCAAGTTCCTTTTGTTTAAAAACTATCTGAAAAGA
AGAAGTTTTTTTCCTGGGGGCCACCATCTGACTTATATTCTTTATGCGTTTTCCAGAGTGAG
TGACTATAAAATTACGCCAAAACACGAGCTTATTGATGCCTAGAGTGCCCTGTAGTTTATAA
ATCTTAAGATTGGATATGATACTTTTCTATTACAATGAAACAGGAACCAGATCCTGCCCTTG
GCAATGGTGGTCTAGGCCGTCTAGCGTCCTGTTTCCTGGATTCTTTGGCAACCCTAAATTA
TCCAGCATGGGGATATGGACTTCGGTACAGATATGGCCTCTTTAAGCAAATCATAACAAAG
GATGGTCAGGAGGAGGTAGCTGAGAATTGGCTAGAGGTAGTGATATGTCTTCTACTTGTTT
ACTGTTTGGAATTGTTGATTATGTTGGTTTATGTTTTAATGACCTGCATGTTAGCTTCTTTTG
CCATTAACTGATGCTGTATCTCCAGATGGGAAATCCATGGGAGATTGTAAGAAATGATGTC
TCTTATCCTGTGAAATTCTATGGCAAAGTGGTTGAAGGCACTGATGGGAGAAAACACTGGA
TTGGAGGAGAGAATATCAAGGCTGTGGCACATGACGTTCCTATTCCTGGCTACAAGACTAA
AACTACTAACAATCTTCGTCTTTGGTCGACAACAGTACCATCACAAAATTTTGATTTGGGAG
CTTTTAATGCTGGGGACCATGCCAAGGCCAATGAAGCGCATCTAAATGCTGAAAAGGTATT
CTTTCGTCTACATTTTTTTTTGAATATAAACCATTAACTCTCAATAACTATCGATTCAAAGGCT
AAACAGTGTTTTGCTAGTAGTTACTGTGTTTTCATTTTAGAATGTGTGCAGTCAATCTTTTAA
TAACTTTGATTGTAATAATATACTGATATACACAAACCTATTTGAATTTCTCGTGTACAAATG
TCGTCACTAGATTTTTCATGAAAATACTTATTGTATTTTCATGTAAATAGAAAAGCTGATGGT
TAGATGTTTTGTGTCAAGTGACAAATGAAAATGAACAGTGTTTGTACACCCATAACTTCTTC
ACATGGTGTTTGCAATTCTTTCGGTTGTATTTATGTGTTTTCTTATTCTGAGTTAATTAATCTC
AGCATATGCATCAGGCTACATTACTCCTGTGTGTCAAGAAAAAATAAGTCTGACGAGGTGC
AGTCCAGTAGTGTGTAGATGGCACATTGTAGAATAGCTTTTCTCATGGAAGCGTTACTTTAT
GATTTTTGTTATACCTTTTGAGTGTTTGTGCATTGTGCACATATACCATTAAGTATGAGTGTT
CAAAAGTGGTTCTCTCAAACTGTGCAAAACCCGCCACTGTTTGCGAGTATTCATTTATCTGA
ACTAGTTAAACAACTTGACTGGACAAGATGCATTGTACTGACATCTGAAGCTGCCGGGTTG
GACAGATTTGTGCTCACATCTATTTAGCGTACATGGATTAAATTGTTCAATTTTCCTCATTTG
TTTAGCTTGAGGAGTACTATCTAATTATATTTATATTCAAATTTTCTTTGTGTGTTACGTTATA
AACTTCTAGTCATACTTACATTTGTGGCCATTGTGGACAAAATTTATCATGTCTAACTTAAGT
TTGTGATTGGTCTTCTCATCATGCCAGATATGCCACGTATTGTATCCAGGTGACGAATCATC
AGAGGGGAAAATTCTTCGCTTGAAACAACAATATACATTATGCTCGGCCTCCCTGCAGGAC
ATCATTTCTCGTTTTGAGTCGAGGGCTGGTGATTCTCTCAACTGGGAGGACTTCCCCTCTA
AAGTTGCAGTCCAGATGAATGACACTCATCCAACACTGTGCATTCCCGAGTTAATGAGAAT
ACTGATGGATATAAAGGGATTGAGCTGGAATGAGGCTTGGAGTATCACAGAAAGGTACCAC
AGAAGGGTACTGCAGTTTGATGTGTTATATGCTGTTCCTCCAGTTCTATTTACCTAATTTTAT
TTCCTCATCTGTCTTCCATTCAATTATGTACTGATTTTCTGATATCATTTGTAGAACCGTTGC
ATACACTAACCACACGGTATTGCCTGAAGCTCTAGAGAAGTGGAGCTTGGACATTATGCAG
AAACTTCTACCTCGACATGTTGAGATCATAGAAACAATAGATGAGGAGGTAACAAATGTGC
AAATAATATTATACAATTAATTTCATGCTCTGTAGTTCAACTTATGGTTTACTTATCTTAATCA
TTGATTTAAAAACAGCTGATGAACAACATCGTCTCAAAATATGGAACGGCAGATATTTCACT
GTTAAAGCAGAAGCTTAAAGATATGAGGATCTTAGACAACGTTGATCTTCCAGCCTCTGTTG
CTAAACTGTTTATTAAACCTAAAGAGAAAAGGGGAAAGTTGCTTGTCGAATCTTTGGAGTCT
ATTGCTGAAGCTGACGAGAAAACTGAGTCACAAGAGGTGGAAAACATTCTATCTGAGACAA
CAGAGAAAAAGGCTGAATCTGACTCTGAAGAAGCTCCTGATGCAGAAAAGGAGGATCCTG
AGTATGAGTTAGATCCATTTGCAAAATACGATCCTCAGTTTCCTAGAGTTGTTCGAATGGCA
AACCTCTGTGTTGTTGGTGGACATTCAGTTAATGGTGTGGCTGAAATTCACAGTGAAATTGT
TAAGCAAGATGTGTTCAATAGCTTTTATGAGGTATTTGCACGATGATTTGGAAACTGTGATC
TTATTTTCCAATGGCTGCTATAATCGTCAGGTGGTATGGTATTCACCATCTGCCATTTTGTT
GTGTTCTTTGCCAGATGTGGCCTACTAAGTTCCAAAACAAAACAAATGGAGTGACCCCCAG
GCGTTGGATCCGATTTTGTAATCCTGAATTAAGTACAATCATTTCAAAATGGATAGGCTCTG
ATGACTGGATCCTGAACACTGATAAACTTGCAGGACTGAAGAAGGTACTTCCAGTAGTTTT
ATTTTCCATTAATCAGTAATCTAATGCTTCCTTCACTTCTATGTTACCTGTGCCTTGTTACAT
TGATGTGTGTTCATCCTGTAACAGTTTGCTGATGATGAGGATCTACAATCAGAATGGCGTA
CTGCTAAAAGGAATAACAAGATGAAGGTAGTTTCGCTGATAAGAGACAAGACTGGATATAT
TGTCAGCCCAGATGCGATGTTTGATGTGCAGGTATATTTTATACTAGAAAACATGTGTTTTC
TTATAAATATGGTTTAGCTCAGCTCTTGGTACTGGACACTTAGGTGAAAAGAATACATGAGT
ATAAGCGGCAGCTGCTAAATATCCTTGGTATCGTTTACCGCTACAAGAAGATGAAAGAAAT
GAGTGCAAAAGATAGAAGAAAGAGCTTTGTTCCAAGGGTATGCATATTTGGTGGGAAAGCT
TTTGCCACTTACGTGCAGGCAAAGAGGATTGTGAAGTTTATCACAGATGTTGCAGCTACTG
TAAATTATGATCCTGATATTGGAGATTTACTGAAGGTAAATTTGTGTCTTTGATGTCACTTGT
CGTTCTTAATTCTCATGTTCTGTCACTGGCAATTTGACTCCGACTAGAGTTACTGAAAAGCA
GTGAATGTTCAGAGTTTCAAAAGAAAACAGAGCAGTGAATGTTCGTAATGTCGGTGCTTGC
AAAACTGAGAAACTGAATAGCCACAGATTTTTATAGAGAACAATATAGCCACAATTGTTTTG
ACCCTTTTATTGCTATCAAGCATCTGATGCTTATATTACTGATAACTAGATGAGGTAAATACA
AGGAGACACGCTAAGTTCTGAGGATTTTTGTTTGTTATCCAGGTTGTATTTGTCCCAGATTA
TAATGTTAGCGTTGCTGAGACGCTCATTCCTGCCAGTGAATTGTCCCAGCATATCAGGTAA
TATTCTGGCTACTCTTTTTCATAGCATGTGAATACATAGAGAAAGAATCTTAGCTGGAGTTG
GCTGATGTGGAAATTTTCATGTGCCTTTTTGTTTGCTCTAATATATACCTGGAGTCTTACCA
GGATAGTGCCACCCCAGTTTCTATTTCTGTACAAGACGTGCATTTTTAACACTATTACTATC
CATTCAGTACTGCTGGAATGGAAGCTAGTGGAACCAGCAACATGAAGTTTGCAATGAATGG
TTGTCTTCTTATTGGAACTTTGGATGGTGCGAATGTGGAGATCAGAGAAGAGGTTGGAGAG
GAGAATTTTTTCCTCTTTGGTGCACATGCACCTGAAATCGCTGGTTTGAGGCAAGAGAGAG
CTGAAGGAAAGGTAGGTACCACACTTGTTTATATAGCCGTGCCCACGATCAAACTAGCAAA
GATCTCTCCCAAATGTGTGCCGCCCAAGTAGTTTATCTGAAGGAAAATCATAACTTTACCAT
CTTCCTTTGCACTTGTAGTTTGTGCCTGACCTGAGATTTGAAGAGGTTAAGGAATACGTCC
GCAGTGGCGTCTTTGGGACTAGCAACTATGATGAATTAATGGGTTCTTTGGAAGGAAATGA
AGGTTATGGACGTGCAGATTATTTTCTTGTTGGCAAGGATTTTCCCAGTTACATTGAATGCC
AGGAAAAAGTTGATGAAGCATACCGAGATCAGAAGGTAAGTTGTATAACTGACGCCTTTTC
TTTTATGACTGAAATGCACACAGTGATTGTGTGACTTCCTGGTATGTGATGGCTGACATCAG
TGTTTCACTATGAACGATTTCACTGATTTGTAGGAATATTTCTTAATTCAAACTGAAGTATCT
ATTGATCTATTAGTATGTTGGAATAACATCATAAACAATCAGAGAATACGGTTGCTAGATGT
GATATATGAGCTTTAAGGAACCTAGCTAGACATTCAGGGAAGTGTTTCTATGCATTTCAATC
CAAACATAAACTGACGGAACAATTAAGTTTAAACTCACCGCGGGGCTTTGTGTTATTGGGTT
TTCTGAAATAAAATCTGGAGCGAGGAGCTCCTCAATTCGAAAAGAAGGCCGAAACAATTCA
GTGTAACTGGGGACCTGGTTCCCTCTGTTGGAGTCTCCCAAGAATTCTGCAGTCTTGTTTC
TGGGAGAGTATAAAATGCACCACCCTGTTCAGATTCCCCTTCACCCACTTGGTTTTCTTGCT
TGGCGACATTTTATGAGACGGCTTAGATTATCATTTGTACACTATGGATATTTCCACTCTGT
TTCAAGTGTGATTAACAGTGTTAATTTGCTACTAACCAGACATAGACTGATAGATAGTTGGA
GTGTTTAATCCTCCTAATCACATGATATCCGTCTCTACCTGCAGCTATGGACAAGAATGTCT
ATCCTCAACACAGCGGGTTCCCCCAAGTTCAGCAGTGACCGGACGATCCACGAGTACGCC
AAGGACATATGGGATATCAGCCCCGTCATCATGCCCTGAGTAATTCAAGCAGTTTCCAGGA
ACCTCGAGTCTGTGAGGCGTCACAGATTTGCCTCACTCCTCAGTGAGAGGGTCTTGTTGG
GCCCAGTTTTGTATAGGCATATGCAAGCTCTGACATGAACCTGAGAGCTGAGAGCATGCAG
GATAGTTCATCTTTAACAAACTTGCATGCCGGTTAGCCAGTGTCACCCGGGCTTATAGTGT
GCGAATTTCGCCGAATAATGTTGGAGCAGAATCATCAAACAAACAAACTTTGGTGTATCTGT
GATGATAAATAAATGGCAAATGATTGAATGAGAAGTGTCACTGGCGCACACATGTAGAAGC
ATGTTTCTTCGGAGAGGGTGCAAAGCGGAAACACCCCAGCTTTGAAGTGAGAATGCAGAA
ATTACTGCCACGTTTGGTACCATGCATCATCAGGGCAGTCAGGCATACTAAATGCAGTTTT
GGCCTGTTTGGTTGACCGAGTTGCATCAAAATCCTGGTCCGCATGAATATTACTCCCTCCG
TTTTCTAAATATAAATCTTTTTAAAGATTTCACTAAGAGACTACATACATATGTATATGGATAT
GCTTTAGAAAGTAGATTCATTCATTTTACTTTGTATGTAGTCCTTTAATCAAATCTCTAAAAA
GACTTATATTTAGGAACCGAGGGAGTAAAACACCTCTGGTCAAGAGATGTTTGAATCGGCC
GTTTTTTCACAGCCAGGCTCAAGCCATGCACCTCGAGGCATGTGGGCCATACCTGGCCAA
ACGGGCCGGGCCGGGCCGGGCCGGGCCGGCCTGGCCCGGCCATAAACAGGCCGACAA
GACCCGCCCAGACCCGAATATTAGCCGGACCGTGCCGTGCCGGCTCATGTACGCAGCCTT
TCGGCCCAGGCACGACACGGGA
SEQ ID NO: 42 >Barley PHS1 (Morex reference genome) CDS
ATGGCGACCGCCTCGCCGCCGCTCGCCACCGCCTTCCGCCCGCTCGCCGCCGCCGGAG
GCAGCGGGGGGCTCGTCGTGGGCGCCGGGGCCGGGGCGGCGCCGAGGCGGGGGGGG
CGGGGGCTCGTGGTGCGGAGCGTGGCGAGCGATCGGGAGGTGCGGGGCCCCGCCTCCA
CCGAGGAAGAGCTTTCAGCCGTGCTAACTTCCATTGATTCTTCCGCCATCGCGTCAAATAT
CCAGCACCATGCAGACTTCACACCGTTGTTCTCGCCAGAGCACTCTTCACCCTTAAAGGCT
TACCATGCAACTGCTAAAAGTGTTTTTGATTCTCTGATAATGAATTGGAATGCAACATATGA
CTATTACAACAAAGTGAATGCAAAGCAAGCTTATTACCTGTCCATGGAGTTTTTGCAGGGAA
GAGCTCTTACAAATGCTATTGGCAACCTAGAACTAACAGGACAATATGCAGAAGCATTAAAA
CAACTTGGACATAACCTAGAGGATGTTGCTAGTCAGGAACCAGATCCTGCCCTTGGCAATG
GTGGTCTAGGCCGTCTAGCGTCCTGTTTCCTGGATTCTTTGGCAACCCTAAATTATCCAGC
ATGGGGATATGGACTTCGGTACAGATATGGCCTCTTTAAGCAAATCATAACAAAGGATGGT
CAGGAGGAGGTAGCTGAGAATTGGCTAGAGATGGGAAATCCATGGGAGATTGTAAGAAAT
GATGTCTCTTATCCTGTGAAATTCTATGGCAAAGTGGTTGAAGGCACTGATGGGAGAAAAC
ACTGGATTGGAGGAGAGAATATCAAGGCTGTGGCACATGACGTTCCTATTCCTGGCTACAA
GACTAAAACTACTAACAATCTTCGTCTTTGGTCGACAACAGTACCATCACAAAATTTTGATTT
GGGAGCTTTTAATGCTGGGGACCATGCCAAGGCCAATGAAGCGCATCTAAATGCTGAAAA
GATATGCCACGTATTGTATCCAGGTGACGAATCATCAGAGGGGAAAATTCTTCGCTTGAAA
CAACAATATACATTATGCTCGGCCTCCCTGCAGGACATCATTTCTCGTTTTGAGTCGAGGG
CTGGTGATTCTCTCAACTGGGAGGACTTCCCCTCTAAAGTTGCAGTCCAGATGAATGACAC
TCATCCAACACTGTGCATTCCCGAGTTAATGAGAATACTGATGGATATAAAGGGATTGAGC
TGGAATGAGGCTTGGAGTATCACAGAAAGAACCGTTGCATACACTAACCACACGGTATTGC
CTGAAGCTCTAGAGAAGTGGAGCTTGGACATTATGCAGAAACTTCTACCTCGACATGTTGA
GATCATAGAAACAATAGATGAGGAGCTGATGAACAACATCGTCTCAAAATATGGAACGGCA
GATATTTCACTGTTAAAGCAGAAGCTTAAAGATATGAGGATCTTAGACAACGTTGATCTTCC
AGCCTCTGTTGCTAAACTGTTTATTAAACCTAAAGAGAAAAGGGGAAAGTTGCTTGTCGAAT
CTTTGGAGTCTATTGCTGAAGCTGACGAGAAAACTGAGTCACAAGAGGTGGAAAACATTCT
ATCTGAGACAACAGAGAAAAAGGCTGAATCTGACTCTGAAGAAGCTCCTGATGCAGAAAAG
GAGGATCCTGAGTATGAGTTAGATCCATTTGCAAAATACGATCCTCAGTTTCCTAGAGTTGT
TCGAATGGCAAACCTCTGTGTTGTTGGTGGACATTCAGTTAATGGTGTGGCTGAAATTCAC
AGTGAAATTGTTAAGCAAGATGTGTTCAATAGCTTTTATGAGATGTGGCCTACTAAGTTCCA
AAACAAAACAAATGGAGTGACCCCCAGGCGTTGGATCCGATTTTGTAATCCTGAATTAAGT
ACAATCATTTCAAAATGGATAGGCTCTGATGACTGGATCCTGAACACTGATAAACTTGCAG
GACTGAAGAAGTTTGCTGATGATGAGGATTACAATCAGAATGGCGTACTGCTAAAAGGAA
TAACAAGATGAAGGTAGTTTCGCTGATAAGAGACAAGACTGGATATATTGTCAGCCCAGAT
GCGATGTTTGATGTGCAGGTGAAAAGAATACATGAGTATAAGCGGCAGCTGCTAAATATCC
TTGGTATCGTTTACCGCTACAAGAAGATGAAAGAAATGAGTGCAAAAGATAGAAGAAAGAG
CTTTGTTCCAAGGGTATGCATATTTGGTGGGAAAGCTTTTGCCACTTACGTGCAGGCAAAG
AGGATTGTGAAGTTTATCACAGATGTTGCAGCTACTGTAAATTATGATCCTGATATTGGAGA
TTTACTGAAGGTTGTATTTGTCCCAGATTATAATGTTAGCGTTGCTGAGACGCTCATTCCTG
CCAGTGAATTGTCCCAGCATATCAGTACTGCTGGAATGGAAGCTAGTGGAACCAGCAACAT
GAAGTTTGCAATGAATGGTTGTCTTCTTATTGGAACTTTGGATGGTGCGAATGTGGAGATC
AGAGAAGAGGTTGGAGAGGAGAATTTTTTCCTCTTTGGTGCACATGCACCTGAAATCGCTG
GTTTGAGGCAAGAGAGAGCTGAAGGAAAGTTTGTGCCTGACCTGAGATTTGAAGAGGTTAA
GGAATACGTCCGCAGTGGCGTCTTTGGGACTAGCAACTATGATGAATTAATGGGTTCTTTG
GAAGGAAATGAAGGTTATGGACGTGCAGATTATTTTCTTGTTGGCAAGGATTTTCCCAGTTA
CATTGAATGCCAGGAAAAAGTTGATGAAGCATACCGAGATCAGAAGCTATGGACAAGAATG
TCTATCCTCAACACAGCGGGTTCCCCCAAGTTCAGCAGTGACCGGACGATCCACGAGTAC
GCCAAGGACATATGGGATATCAGCCCCGTCATCATGCCCTGA
SEQ ID NO: 43 >Barley PHS1 (Morex reference genome) protein
MATASPPLATAFRPLAAAGGSGGLVVGAGAGAAPRRGRRGLVVRSVASDREVRGPASTEEEL
SAVLTSIDSSAIASNIQHHADFTPLFSPEHSSPLKAYHATAKSVFDSLIMNWNATYDYYNKVNAK
QAYYLSMEFLQGRALTNAIGNLELTGQYAEALKQLGHNLEDVASQEPDPALGNGGLGRLASCF
LDSLATLNYPAWGYGLRYRYGLFKQIITKDGQEEVAENWLEMGNPWEIVRNDVSYPVKFYGKV
VEGTDGRKHWIGGENIKAVAHDVPIPGYKTKTTNNLRLWSTTVPSQNFDLGAFNAGDHAKANE
AHLNAEKICHVLYPGDESSEGKILRLKQQYTLCSASLQDIISRFESRAGDSLNWEDFPSKVAVQ
MNDTHPTLCIPELMRILMDIKGLSWNEAWSITERTVAYTNHTVLPEALEKWSLDIMQKLLPRHVE
IIETIDEELMNNIVSKYGTADISLLKQKLKDMRILDNVDLPASVAKLFIKPKEKRGKLLVESLESIAE
ADEKTESQEVENILSETTEKKAESDSEEAPDAEKEDPEYELDPFAKYDPQFPRVVRMANLCVV
GGHSVNGVAEIHSEIVKQDVFNSFYEMWPTKFQNKTNGVTPRRWIRFCNPELSTIISKWIGSDD
WILNTDKLAGLKKFADDEDLQSEWRTAKRNNKMKVVSLIRDKTGYIVSPDAMFDVQVKRIHEYK
RQLLNILGIVYRYKKMKEMSAKDRRKSFVPRVCIFGGKAFATYVQAKRIVKFITDVAATVNYDPD
IGDLLKVVFVPDYNVSVAETLIPASELSQHISTAGMEASGTSNMKFAMNGCLLIGTLDGANVEIR
EEVGEENFFLFGAHAPEIAGLRQERAEGKFVPDLRFEEVKEYVRSGVFGTSNYDELMGSLEGN
EGYGRADYFLVGKDFPSYIECQEKVDEAYRDQKLWTRMSILNTAGSPKFSSDRTIHEYAKDIW
DISPVIMP
SEQ ID NO: 44 >Barley PHS1 (Morex reference genome) promoter (−2 kb upstream of ATG)
TCGAGAACTATGTAGACATGACCCGAGAGATTCCTCGGTCAATATCCAATAGCGGGACCTG
GATGCCCATATTGGATCCTACGTATTCTACGAAGATCTTATCGTTTGAACCTCAGTGCCAAG
GATTCATATAATCCCGTATGTCATTCCCTTTGTCCTTCGGTATGTTACTTGCCCGAGATTCG
ATCGTCAGTATCCGTATACGTATTTCAATCTCGTTTACCGGCAAGTCTCTTTACTCGTTCCG
TAATACAAGATCCCGCAACTTACACTAAGTCACATTGCTTGCAAGGCTTGTGTGTGATGTTG
TATTACCGAGTGGGCCCCGAGATACCTCTCCGTCACACGGAGTGACAAATCCCAGTCTCG
ATCCATACTAACTCAATGAACACCTTCGGAGATACCTGTAGAGCATCTTTATAGTCACCCAG
TTACGTTGCGACGTTTGATACACACAAAGTATTCCTCCGGTGTCAGTGAGTTATATGATCTC
ATGGTCATAGGAACAAATACTTGACACGCAGAAAACTGTAGCAACAAAATGACACGATCAA
CATGCTACGTCTATTAGTTTGGGTCTAGTCCATCACGTGATTCTCCTAATGACGTGATCCAG
TTATCAAGCAACAACACCTTGTTTATAATCAGAAGACACTGACTATCTTTGATCAACTGGCT
AGCCAACTAGAGGCTTGCTAGGGACAGTGTTTTGTCTATGTATCCACACATGTATATAAGTC
TTCATTCAATACAATTATAGCATGGATAATAAACGATTATCTTGATACAGGAATTATAATAAT
AACTATATTATTATTGCCTCTAGGACATAATTCCAACAATATATGCCATCTTCCGTGTTTTAT
TCTTCTGGGCAGCGGTCTGTTATGCAAAACGTGGTCGCCGATATCTTCTAGTCTATATCGA
TGACTTTTCGACAACTGCATCTGCAAGCTCCTTAGTTTCAACAAGTTTATTCCACTGAGGGG
GTGTTTCTTTACAGGGACTTTTTGCTGTAGGGACTAAAAAACGTCCCTTTTAGTCCCATGTG
AAAGAAACAGGAGAGACTTTTAGGGACTAAAAGGGGGTATTTGGAACTAAAGGAAGAAGTC
CCTATGGGAGGGACTTTTTGAGACTTTTTTGACACTTTTTACAACAGTGCCCCTACTTTTAT
CATGTCATTTAATAACTTCTAGTACATTACTAGGGGTAACATGGTCTTTTTACATGTCATTTA
ATGACTTCCAATCCCTATTTAGTCCCTGGAAAGAAACGGGTAGGGACTAGAGACTTTTTAG
TTGGTATTAAAAAAAAAGTGCTAAGACTTCTTAAAGAAACAGGGCGTGAGATGGAAGTGCA
GAGGCGGCAGCGAATTAGCTCACGGGGCACTACCGGTGGATGTAGGAAAAAGAAGGCAT
TTGACACCTCTCAAAACTATGAAGATGTAAATTTTAATTTCTAAGTGTTTTTGTGAGGATTTG
TGCTATTTAATACTCCCTCAATTCCAAAATAAATGTTTTAATTTTGTATTATCTCTAATACAAA
GTTATACTAACTTTAAGATACCTATTTTAAAACAGAGGAAATATATGCATTTTGCCTTTCTCA
CAAAAAAAACCCCTAAACCATCACACGAGGGGAAGAGCCATTCCCAGCCGTCCAATAAACA
CGACAAAAAGCTGTACAGCACGGGAGCAATTCCCAGCCGTCGAAATGCGCTGCAACGGAA
GTAGCGCTCCCTGACACAGCGGCACATGAGCCACGCCTCGCACCACCGCACCGTGCACC
GGCGCGGGGCCCACCGGCCAGCGTCCCCACACGCGTACCGGCGGCCAGCATTGCGCGG
CACGGCGACGGAGAGATATTTTTAACGCCTTCCAGTTCGGAAGCGCCCCCCATCACTCAC
TCGGGTACAGGGCGGGCCGCGTGAGTCTCGCGTCGTCGCATCGCGCGCGCTGCACACCC
ACCGTCGTCAGCGCGGACCACCGGCGCCCCACCCA
SEQ ID NO: 45 >Rye PHS1 genomic DNA
CATCGATGACTTCCAGACCGCCGCTTCTACAAGCTCCTTATTTTCAAGTCGCTTCTACAAGC
TCCATATTTTCAACAAGTTTCCTTGGCAACGGCTTTGCTCATGGCGCACTGCCCGTGGTTG
CAAGAGAAAGAATACATTCGACACCCCTAAAACCATGCAGATGTAATTTCTTTTTGTAAATG
TTTTTGTGACCTTTTGCCTTTTTCCAAAAAAACAAACAAACTAAACCATCACACGAGGAGAA
GAGCCATTCCCAGCCGTCCAATAAACACGACAAAACGCTTTACAGCACGGGGGCAACTCC
CAGCCGTCGAAAATGCATTGCAACGGAACTACTAGCTCTCCCTGACACAGCGGCACATGA
GCCACGCCTCGCACCGCAACCGTGCACCGGCGGTGGGCCCACCGGCCAGTGTCCCCCA
CACGTGTACCGGCGGCCAGCATTGCACGGCACGGCGACGGAGAGATATTTTAACGCCTTC
CAGTTCAGAAGCACCCCCCATCACTCACTCGGGTACAGAGCGGGGCCGCGTGACTCTCG
CGTCGTCGCATCGCGCGCGCTGCACACCACCGTCGTCAGTGCGACCACTGGCACCAATG
GCGACCGCCTCGCCGCCGCTCGCCACCGCCTTCCGCCCGCTCGCCGCCGGCAGCGGCG
GCACCCGGGGAGGAGGAGAAGGAGCGCACGCCGCGGGCCGGGTCGCGCCGAGGCGGG
GGCGCCGGGGGTTCGTGGTGCGGAGCGTGGCGAGCGATCGAGAGGTGCGGGGGCCCG
CCTCGACCGAGGAAGGTAAGCGGCCGCGCGCTCCCGTTGGCTTCGACAACCCGGATGCT
GGCCGGCTGTAATGTGGTTTGGATTTCCGCTGGTGCGACCCGTGAATTTATGATCCTCTGA
CTGATGGTCTCTGTGTTCCCGGGTTTCGGGCTATTGGTTTTGTGCGCGCGCATTGCTAATC
TACTGCTCTAGGATTTGTTAGGGTTCAGAGGAATCTTAATTCGCGAATAAACTTTCGAGGAA
TGTGTTCTGAATTTACTGAAACTGGTTTAGTTTGGTGCGTCAAGTTAGAATGATGTATTTAT
GGTACAGTAGCAAACTCTGAGTAATTCTACTGTGCGTGGACAGCCACCCCTGGTGGATGAT
GCAGCAAGGGTCAACATACTGTCGCTACTATCTGTAGCTGCAACAAAATACTTGGAATCTG
AACTAGTGAAATAAATCATTTAAGATTTTTGCTGAAGATTACACAGTATCAATGTTTCTGAAG
GTGATTGGAACAGTATAGCACTTCAAAATGGCAACCAAGCAGGATCAGTATACTGACACAT
CTTTTTTCTAAAGGTGGTTCGAATACCGGTATGAAGTTTATAATTATTTAATTAAGGCGTAAG
ATTTGGTAGCAAAAACTATTTTTACGGGAAATGATGAAACATGGCACAAAAACTCTAGGAGC
TGCCTTTCATTTATTAATCGATAAAAAGAGAGGTGCAGGGTTCTAGTAGAATGCAAATGGTG
ATACAGTATATTATCTACCATTTTGTGCAAGCATTTTCACACGAAAACAATATCCCATGCAGT
TTTTAGTTTTGACTCTCGACTGTTTAATACCTACTCTTTGTAAGATTTCAGATCCTAATATTG
CTGTTCTTGCAACTCAGAGCTTTCAGCCGTGCTAACTTCCATTGATTCTTCCGCCATCGCGT
CAAATATCCAGCACCATGCAGACTTCACACCGTTGTTCTCGCCAGAGCACTCTTCACCCCT
AAAGGCTTACCATGCAACTGCTAAAAGTGTTTTTGATTCTCTGATCATGAATTGGAATGCAA
CGTATGACTATTACAACAAAGTGAATGCAAAGCAAGCTTATTACCTGTCCATGGAGTTTTTG
CAGGTCTCGCAACATACTTCCTTTAGCCTTATCTCATACGTTTTGGTAACTGCGTTATGATG
TTATATGTTTTTTTTTTTTTTTGTACTGCAGGGAAGAGCTCTTACAAATGCTATTGGCAACCT
AGAGCTAACAGGACAATATGCAGAAGCGTTAAAACAACTTGGACAGAATCTAGAGGATGTT
GCTAGTCAGGTAAGCGATTTCTGTCAAAGAATAGTTTTTAGTCCATATCAAAATTATTGCCG
AGCCATTTTTTTTTGAAAAAGTAACATTTTATTATTATTCATCAACTTCCATTTTATTCAAAAC
TATCTGCAAAAGGAGAAAATTTTCTCCTGGGGGTTCCACCATCTGTCTTATATTTGTTATGC
AGTTTTTAGAGTGATTGACTATGAAATTACGCCAAAACATGAGCTTTGCCTAGAGTGCCCTG
TAGTTTAGAAATCTTAAGATTGGATATGTTACTTTTCTATTACAATGAAACAGGAACCAGATC
CTGCCCTTGGCAATGGTGGTCTAGGCCGTCTAGCGTCCTGTTTTCTGGATTCTATGGCTAC
CCTAAATTATCCAGCATGGGGATATGGACTTCGGTACAGATATGGCCTTTTTAAGCAAATCA
TAACGAAGGATGGTCAGGAGGAGGTAGCTGAGAATTGGCTAGAGGTAGTGATATGTTTTCT
ACTTGTTTACTGTTTGGAAGTGTTGATTATGTTGGTTTCAGTTTTAATGACCTGCACGTTAG
CTTCTTTTGCCATTTATAACTGTTGCTGTATCTCCAGATGGGAAATCCATGGGAGATTGTAA
GAAATGATGTCTCTTATCCTGTGAAATTCTATGGCAAAGTGGTTGAAGGCACTGATGGGAG
AAAACACTGGATTGGAGGAGAGAATATCAAGGCTGTGGCACATGATGTTCCTATTCCTGGC
TACAAGACTAAAACTACTAATAATCTTCGCCTTTGGTCAACAACAGTACCATCACAAAACTTT
GATTTGGGAGCTTTTAATGCTGGGGATCATGCCAAGGCCAATGAAGCACACCTAAATGCTG
AAAAGGTATTCTTTTGTCTGCATTTATTTTGAATATAAATCATTAAATCTCAATAACTATTGAT
TTGAATGGTAAACACTAAACAATGTCTAGCTAGTAGTCACCACGGTGTCCCTTGATGTCATT
TTAGAATGTGCGCAGTCAAATGTTTGATAACTTTGATTGTAATAATATACAAATATTCAGAAA
CTTTGATTGACACTAGATTTTTCATAAACATACTTATTGTATTTCCATGTAAATAGAAAAGCT
GATGTTCAGAGGTTTGTGTGTCAAAATGACAAGTAAATATGAACAGAGTTTGTGCACACATA
ACCTCTTCATCTGGTGTTTGCAGTTCTCTAGGTTGTGTTTGTGTTTTCTCCTGCTCTACCTTA
ACTAATCTCAGCATATGCATCAGGTACATTGCTCCTCTGTGTCTATAAAAAATAAGTCTGAT
GAGATATGGTTGCAGTCCAGTAGTTCGTAGTTGGCACAGCATGGAAGAACTTTTCACATGG
AAGCCTTACCCTTATGATTTTAATTATACCTTCGGAGTGTTTGTGCATCGTGTACATAGACC
ATTAAGTATGGGTGTTCAAAAATAGTTTTCCCAAACTGTGTAAAAGCCACCACTGTTTGTGT
ATTCATTTTTTCGAACTAGCTAAACAACTTGACCGGAGAAGATACATACAATTGTACTCACA
TCTGAAGCTGCTCAGTTGGACAGATTTGTTCCAATCCCGTGCATGGGTTAAATTGTCGACG
TTTCCTCACTTGTTCAGTAGTAGGAGTACTATCTATTATATTTATACTAAAGTTTTCTTTTTGT
GATATGGTACAAACTTTTAATAATACTTACATTTGCAAACACTGTGGACAAAATTTATTATGT
CTAACTTAACTTTATGACTGGTCTTCGCATCATGCCAGATATGCCACGTATTGTATCCAGGG
GACGAATCGTCAGAGGGGAAAATTCTTCGCCTGAAACAACAATATACACTATGCTCGGCCT
CCCTGCAGGACATTATTTCTCGTTTTGAGTCGAGGGCTGGTGATTCTCTCAACTGGCAGGA
CTTCCCCTCTAAAGTTGCAGTTCAGATGAATGACACCCATCCAACACTGTGCATTCCCGAG
TTAATGAGAATACTGATGGATGTTAAGGGATTGAGCTGGACTGAGTCTTGGAGTATCACGG
AAAGGTATCACAGAAGGGTACTGCAGTTTGATGTGTTATGCTGTTCCTCCTGTTCTAACTAA
TTTTATTTCCTCATCAGTGTTCCATTCAATCATGTACTGATTTTCTGACACCCACATCATTTG
TAGAACCGTTGCATACACTAACCACACAGTACTTCCTGAAGCTCTGGAGAAGTGGAGCTTG
GACATTATGCAGAAACTTCTACCTCGACATGTTGAGATTATAGAAAGAATAGATGAGGAGG
TATCAAATGTGCAGATAATATTATACGATTAATTTCATTCTCTGTATTTCAACTTATGGTTTAC
TTGTCTTCATCGTTGATTTTAAACAGCTAATGAACACCATCATCTCAAAATATGGAACGGCA
GATATTTCACTGTTAAAACAGAAGCTTAAAGATATGAGGATCTTAGACAACGTTGATCTTCC
AGCCTCTGTTGCTAAACTGTTTATTAAACCTAAAGAGAAAAAGGGGAAGTTGCTTGTTGAAT
CTTTGGAGTCTATTGCTGAAGCTGACGAGAAAAGTGAGTCACAAGAGGAGGAAAACATTCA
ATCTGAGACAACAGAGAAAAAGGGTGAATCTGACTCTGAAGAAGCCGCTGATGCAGAAAA
GGAGGATCCTGAGTATGAGTTAGATCCATTTGCAAAATACGATCCTCAGTTACCTAGAGTT
GTTCGTATGGCAAACCTCTGTGTTGTTGGTGGACATTCAGTTAATGGTGTTGCCGAGATTC
ACAGTGAAATTGTTAAGCAAGATGTGTTCAATAGCTTTTATGAGGTATTTGCACAATGATTT
GGAAACCGTGATCTTATTTTTCAATGACTGCTATAATCGTCAGGTGATATGCTATTCACCAT
CTGCCATTTTCTTGTGTTCTTTGCCAGATGTGGCCTGCTAAGTTCCAAAACAAAACAAATGG
AGTGACCCCCAGGCGTTGGATCCGGTTTTGTAATCCTGAATTAAGTACAATCATTTCAAAAT
GGATAGGCTCCGATGACTGGATTCTGAACACTGATAAACTTGCAGGACTGAAGAAGGTACT
TCCAGTAGCTTTATTCCCCAATAATCAGTAATCTAATGCTTCCTTCACTTCAATGTTAACTGT
ACCTTGTTTTTAAATTGATGTGTTTTCATCCTGTAACAGTTTGCTGATGATGAGGATCTGCAA
TCAGAATGGCGTACTGCTAAAAGGAATAACAAGATGAAGGTAGTTTCGCTGATAAGAGACA
AGACTGGATATGTTGTCAGCCCAGATGCAATGTTTGATGTGCAGGTATATTTTGTACTAGAA
AACATGCGTCTTTTTATAAATATGGTTTGACTCAGCTCCTGGTACTGGACACTTAGGTGAAA
AGAATACATGAATATAAACGGCAGCTGCTAAATATCCTTGGTATCGTTTACCGCTACAAGAA
GATGAAAGAAATGAGTGCAAAAGATAGAATAAAGAGCTTTGTTCCAAGGGTATGCATATTTG
GCGGGAAAGCTTTTGCCACTTACGTACAGGCAAAGAGGATTGTGAAGTTTATCACAGATGT
CGCAGCTACTGTAAATTATGATCCTGATATTGGAGATTTACTGAAGGTAAATTTGTGTCTTT
GATGCCACTTATCGATTTTAATTTTCATGTTCTGTCATTGGCAAGTTGACTCAGACTGAAAA
GCAGTGAATGTTCAGAGTTTCAAAAGAAAACAGAGCAGTGAATGTTCTTAATGCCGGTGCT
TGTACAACTGAGAAACTGAATAACCGCAGTTCTTTTGGAGAACAATATAGCCACAATTGTTT
TGACCCTTTTATTGCTATCAAGCATCTGATGCTGATATTATTGATAACTAGATGAGGTAAATA
CAAGGCGACAGGTTAAGTCTGAGGATTTTTGTTTGTTATCCAGGTTGTATTTGTCCCAGATT
ATAATGTTAGCGTTGCTGAGACGCTCATTCCTGCCAGTGAATTGTCCCAGCATATCAGGTA
ATATTCTGCCTATTCTTATATAGTACTCCCTCTGTAAACAAATATAAGAGCATTTAGATCACT
ATTTTAGTGATCTAAACGCTCTTATGTTTGTTTACACAGGGAGTATGTGAGTACATCAAAAA
AGAATATTACCTTTTCGTTTGCTATAATATATACCTAGAGTCTTACTGGGATAGTGCCACCC
CAGTTTCTATTTCTGTACAAGATGTGCATTTTTAACAGTTACTATCCATTCAGTACCGCTGGA
ATGGAAGCTAGTGGAACCAGCAACATGAAGTTTGCAATGAATGGTTGTATTCTTATTGGAA
CTTTGGATGGTGCGAATGTGGAGATCAGAGAAGAGGTTGGAGAGGAGAACTTTTTCCTCTT
TGGTGCACATGCACCTGAAATCGCTGGTTTGCGGCAAGAGAGGGCTGAAGGAAAGGTACC
ACACTTGTTTATATAGCTGTGCCTACTATCAAACTAGTAAAGATATCTCCCATATGTGTGCT
GTCCAAATAGTTTAGCTGAGGGAAAATCATAACTTTACCATTTTCCTTTGCACTTGTAGTTTG
TGCCTGACCCGAGATTTGAAGAGGTTAAGGAATACGTCCGCAGTGGCGTCTTTGGGACTA
GTAACTATGATGAATTGATGGGTTCTTTGGAAGGAAATGAAGGTTATGGACGTGCAGATTA
TTTTCTTGTTGGCAAGGATTTTCCCAGTTACATTGAATGCCAGGAAAAAGTTGATGAAGCAT
ACCGAGATCAGAAGGTAAGTTGTATAACTGACGCTGTTTGTTTTCTGGCTGAAATGCACAT
GGTGATTATGCAAACTTTCTGATATGTGATGGCTGACATCAGTGTTTCGCTGTGAACGACTT
CACTGATTCGTAGGAAGGAATATTTCTTAGTTCAAAGTTTCAAACCGGAGTATCTATCGATG
TGTTGGAACTTGGAAGATCATAAACAATCAGAGAATATAGTTGCTAGATGTGCTATACTACT
CATCTGTTGGCTGTTGAGTGTTGATACATGAGCTTTGATGAACCTAGCTAGACCTTCATGAA
AATGCTTCTATGCATTCCAATAAAAAAAATAACTTTATAACTTAACAAAACAATTAGGTTGAA
CCTCGCCAGGGGGCCTTTTGTAATCTGTTTTCTGAAATGAAAATCAGAAGCAGGGAGCTCC
TCGATTCTAAAATGAAAAATAAAATCAGGGATAACTGAGGATCTGGTTTTCCTCTGTTGGAG
CCCCAAAGAATTCTGTGGTCTTATTATTGTAAGAGTATAAGATGCACTGCTCCGATAAGTTT
CTCCTTCATCCATTTGTTTTTATTGCTTTGTGACATTTCGTGTGACAGCTCAGATCACCATG
CATCCCAGTTGTACACATTATTTTGCTATCGTGGATATTTCCACTCTGTTTCATGTGCGACA
TTTTTAATTTGTTACATTGTTACTAACTACACATACTCTGATAGATTTTCAGAGTTTTGGTCCT
CCTAATCACATAATATCTGTCTCTACCTGCAGCTATGGACAAGAATGTCTATCCTCAACACA
GCTGGTTCCCCCAAGTTCAGCAGTGACCGGACGATCCACGAGTACGCCAAGGACATATGG
GACATCAGCCCCGTCATCATGCCCTGAATAATTCAAGCAGCTTCCTGGAACCTTGAGAGTC
TATGAGGCGCGATTGGTCGCAGATTTGCCTCCATTCAGCGAGAATGTATTGCGCCCCCCA
GTTTTGTATAGGCATATGCAAGTTCTGACATGGACCTGAGTGCTGAGAGCATGCAGGGTAG
TTCATCCTTAGCAATATTGCATGTCAGTTAGCCAGTAAGTATTACCCAGGCTTATGGTGTGT
GTATTTCGCCGAATAAAGTTGGAGCAGAATCATCAAACAAACATACTTTGGTGTATCTGTAA
TGATAAATAAATGGTAAATGACTGAACAAAAAGTGTCGTTGGCGCACACATGTAGTAGAAG
CATGTTTCTTCCGGAGAGGGTGCAAAGCTGAAAGCACCCCAGTTTTGAAGTGAGAATGCA
GAAGTTACAAAAACATTTGTTTACATGCAAAGTGCAACGGGATTCGTCAAGAGTTCAGGGC
TCCTTTGATTCAAAGGATTTGCATAGGAATTTTGGAGGATTAGAGTCCTTAGGAATTTTTTCT
ACGTTGAGGCCTCATTTGATTTATAGGATAGAAATATCATAGGAATAGGAAAATCATAGTAA
GTGAGATGATATGC
SEQ ID NO: 46 >Rye PHS1 CDS
ATGGCGACCGCCTCGCCGCCGCTCGCCACCGCCTTCCGCCCGCTCGCCGCCGGCAGCG
GCGGCACCCGGGGAGGAGGAGAAGGAGCGCACGCCGCGGGCCGGGTCGCGCCGAGGC
GGGGGCGCCGGGGGTTCGTGGTGCGGAGCGTGGCGAGCGATCGAGAGGTGCGGGGGC
CCGCCTCGACCGAGGAAGAGCTTTCAGCCGTGCTAACTTCCATTGATTCTTCCGCCATCGC
GTCAAATATCCAGCACCATGCAGACTTCACACCGTTGTTCTCGCCAGAGCACTCTTCACCC
CTAAAGGCTTACCATGCAACTGCTAAAAGTGTTTTTGATTCTCTGATCATGAATTGGAATGC
AACGTATGACTATTACAACAAAGTGAATGCAAAGCAAGCTTATTACCTGTCCATGGAGTTTT
TGCAGGGAAGAGCTCTTACAAATGCTATTGGCAACCTAGAGCTAACAGGACAATATGCAGA
AGCGTTAAAACAACTTGGACAGAATCTAGAGGATGTTGCTAGTCAGGAACCAGATCCTGCC
CTTGGCAATGGTGGTCTAGGCCGTCTAGCGTCCTGTTTTCTGGATTCTATGGCTACCCTAA
ATTATCCAGCATGGGGATATGGACTTCGGTACAGATATGGCCTTTTTAAGCAAATCATAACG
AAGGATGGTCAGGAGGAGGTAGCTGAGAATTGGCTAGAGATGGGAAATCCATGGGAGATT
GTAAGAAATGATGTCTCTTATCCTGTGAAATTCTATGGCAAAGTGGTTGAAGGCACTGATG
GGAGAAAACACTGGATTGGAGGAGAGAATATCAAGGCTGTGGCACATGATGTTCCTATTCC
TGGCTACAAGACTAAAACTACTAATAATCTTCGCCTTTGGTCAACAACAGTACCATCACAAA
ACTTTGATTTGGGAGCTTTTAATGCTGGGGATCATGCCAAGGCCAATGAAGCACACCTAAA
TGCTGAAAAGATATGCCACGTATTGTATCCAGGGGACGAATCGTCAGAGGGGAAAATTCTT
CGCCTGAAACAACAATATACACTATGCTCGGCCTCCCTGCAGGACATTATTTCTCGTTTTGA
GTCGAGGGCTGGTGATTCTCTCAACTGGCAGGACTTCCCCTCTAAAGTTGCAGTTCAGATG
AATGACACCCATCCAACACTGTGCATTCCCGAGTTAATGAGAATACTGATGGATGTTAAGG
GATTGAGCTGGACTGAGTCTTGGAGTATCACGGAAAGAACCGTTGCATACACTAACCACAC
AGTACTTCCTGAAGCTCTGGAGAAGTGGAGCTTGGACATTATGCAGAAACTTCTACCTCGA
CATGTTGAGATTATAGAAAGAATAGATGAGGAGCTAATGAACACCATCATCTCAAAATATGG
AACGGCAGATATTTCACTGTTAAAACAGAAGCTTAAAGATATGAGGATCTTAGACAACGTTG
ATCTTCCAGCCTCTGTTGCTAAACTGTTTATTAAACCTAAAGAGAAAAAGGGGAAGTTGCTT
GTTGAATCTTTGGAGTCTATTGCTGAAGCTGACGAGAAAAGTGAGTCACAAGAGGAGGAAA
ACATTCAATCTGAGACAACAGAGAAAAAGGGTGAATCTGACTCTGAAGAAGCCGCTGATGC
AGAAAAGGAGGATCCTGAGTATGAGTTAGATCCATTTGCAAAATACGATCCTCAGTTACCTA
GAGTTGTTCGTATGGCAAACCTCTGTGTTGTTGGTGGACATTCAGTTAATGGTGTTGCCGA
GATTCACAGTGAAATTGTTAAGCAAGATGTGTTCAATAGCTTTTATGAGATGTGGCCTGCTA
AGTTCCAAAACAAAACAAATGGAGTGACCCCCAGGCGTTGGATCCGGTTTTGTAATCCTGA
ATTAAGTACAATCATTTCAAAATGGATAGGCTCCGATGACTGGATTCTGAACACTGATAAAC
TTGCAGGACTGAAGAAGTTTGCTGATGATGAGGATCTGCAATCAGAATGGCGTACTGCTAA
AAGGAATAACAAGATGAAGGTAGTTTCGCTGATAAGAGACAAGACTGGATATGTTGTCAGC
CCAGATGCAATGTTTGATGTGCAGGTGAAAAGAATACATGAATATAAACGGCAGCTGCTAA
ATATCCTTGGTATCGTTTACCGCTACAAGAAGATGAAAGAAATGAGTGCAAAAGATAGAATA
AAGAGCTTTGTTCCAAGGGTATGCATATTTGGCGGGAAAGCTTTTGCCACTTACGTACAGG
CAAAGAGGATTGTGAAGTTTATCACAGATGTCGCAGCTACTGTAAATTATGATCCTGATATT
GGAGATTTACTGAAGGTTGTATTTGTCCCAGATTATAATGTTAGCGTTGCTGAGACGCTCAT
TCCTGCCAGTGAATTGTCCCAGCATATCAGTACCGCTGGAATGGAAGCTAGTGGAACCAG
CAACATGAAGTTTGCAATGAATGGTTGTATTCTTATTGGAACTTTGGATGGTGCGAATGTGG
AGATCAGAGAAGAGGTTGGAGAGGAGAACTTTTTCCTCTTTGGTGCACATGCACCTGAAAT
CGCTGGTTTGCGGCAAGAGAGGGCTGAAGGAAAGTTTGTGCCTGACCCGAGATTTGAAGA
GGTTAAGGAATACGTCCGCAGTGGCGTCTTTGGGACTAGTAACTATGATGAATTGATGGGT
TCTTTGGAAGGAAATGAAGGTTATGGACGTGCAGATTATTTTCTTGTTGGCAAGGATTTTCC
CAGTTACATTGAATGCCAGGAAAAAGTTGATGAAGCATACCGAGATCAGAAGCTATGGACA
AGAATGTCTATCCTCAACACAGCTGGTTCCCCCAAGTTCAGCAGTGACCGGACGATCCAC
GAGTACGCCAAGGACATATGGGACATCAGCCCCGTCATCATGCCCTGA
SEQ ID NO: 47 >Rye PHS1 protein
MATASPPLATAFRPLAAGSGGTRGGGEGAHAAGRVAPRRGRRGFVVRSVASDREVRGPAST
EEELSAVLTSIDSSAIASNIQHHADFTPLFSPEHSSPLKAYHATAKSVFDSLIMNWNATYDYYNK
VNAKQAYYLSMEFLQGRALTNAIGNLELTGQYAEALKQLGQNLEDVASQEPDPALGNGGLGRL
ASCFLDSMATLNYPAWGYGLRYRYGLFKQIITKDGQEEVAENWLEMGNPWEIVRNDVSYPVK
FYGKVVEGTDGRKHWIGGENIKAVAHDVPIPGYKTKTTNNLRLWSTTVPSQNFDLGAFNAGDH
AKANEAHLNAEKICHVLYPGDESSEGKILRLKQQYTLCSASLQDIISRFESRAGDSLNWQDFPS
KVAVQMNDTHPTLCIPELMRILMDVKGLSWTESWSITERTVAYTNHTVLPEALEKWSLDIMQKL
LPRHVEIIERIDEELMNTIISKYGTADISLLKQKLKDMRILDNVDLPASVAKLFIKPKEKKGKLLVES
LESIAEADEKSESQEEENIQSETTEKKGESDSEEAADAEKEDPEYELDPFAKYDPQLPRVVRMA
NLCVVGGHSVNGVAEIHSEIVKQDVFNSFYEMWPAKFQNKTNGVTPRRWIRFCNPELSTIISK
WIGSDDWILNTDKLAGLKKFADDEDLQSEWRTAKRNNKMKVVSLIRDKTGYVVSPDAMFDVQ
VKRIHEYKRQLLNILGIVYRYKKMKEMSAKDRIKSFVPRVCIFGGKAFATYVQAKRIVKFITDVAA
TVNYDPDIGDLLKVVFVPDYNVSVAETLIPASELSQHISTAGMEASGTSNMKFAMNGCILIGTLD
GANVEIREEVGEENFFLFGAHAPEIAGLRQERAEGKFVPDPRFEEVKEYVRSGVFGTSNYDEL
MGSLEGNEGYGRADYFLVGKDFPSYIECQEKVDEAYRDQKLWTRMSILNTAGSPKFSSDRTIH
EYAKDIWDISPVIMP
SEQ ID NO: 48 >Rye PHS1 promoter (−2 kb upstream of ATG)
TTGCTCGATCGATAGAGGGGCGCGCTATACTCGGGACGGTCTTCACGGGGCCTGCGGAG
GCGCGTGCAACCGACGGGCCAGGCCGGTCGCACCGCGTAGATGAGGCAGATCGCGGCG
ACGGGCGGGTGACCAATTCGATCGCGCGCGAGGTCGGGGACACTGCTCGGTGGAGGTG
GGGTGGCGGCGGAGTCCGAGATGAAGCCGGCGATGACAAACGTCGTGGGACATCGTGCG
GACGAGCGCGTCAGCACCGGCGCCCGGGCAACGAGGCCCGAGCGACCAAGGTAGCAGC
GGCGCCCGAGCCCGACGCAACCGACGAGCCTGACGCAGCCGGCCCGACACAGCCGAGG
ACGAGGCCGACGAGGTAACACGCAGGGTCGCCGTGTAGACGCGACGAAGACGGATGGC
GTGAATCGATTGACGTGCCGGCACGCAAGCGACGGCTATGATTGGCCGCCGCATGGCGC
GAGGCCGCCGGCTGGGGGCGATGCAGGTGCCCCGGTCGGAGCAGGGCGGTGACGGCC
GGCGCGGGGCGACGGGCGTGCCGGCGCGCGAACGACGGCCTTGAGGGGCCGCCTGTC
GTGCGAGGCCGCCGGGTTGGTGAAGTAGCCGGCCAGTCGCGTCGGTGTTAGAGTAGAGG
CGCGCGGATGTCAACGGCGGCGGGTGGACGACGCAAACCGATCTTAGATCGGAAAACCA
AAAAGTAAACGCCGATCAAAGCGACCGGTGAGAGAGAGAAAAAACTGGAGCAAAGGCTCG
GGAAAAGACTCTCTAGGGAAGTCGGTCAACACAACTGACGGACGAACCCTAGGTATGGGT
GACGCGGCCCCCAGCGGCGGTCATGGAGGCCGACCGCTCCGGGGGGGGCGCGCGGGT
GCGGAAGCCGCGGCGGTGGCTAGGGTTTAGGATCGATTAGGCTGATACCATGTCAGAAG
GGAGATAGAGGTTTGGCGGAATAGTTGTTGTATTGCTTGAGCCTCGTGGGCATATATATAT
AGAAGTACATGGTCATCTTGAAGTACAAGGCAAGGTAAAATAATATCCTATGCTATCCTAGC
TTTCCTAATAATCACGATACTCAACATTGTCATTGTAGTGAGGCATGTGAAGGGTGGTTTGT
CCCAAATCTAGTTAGATGGGTTGGGGTTTTTCTTTGGCAGGTTTATCTCAGTGGTAGTGTC
CCGTGAAGCGGATGACACGACTAGGTGTCTTGGTCCTTCACGCTCTAGTTTTGGCAGGTG
AAGGTTGGTCAGCTTCGCCTCGTCGGGGAGCTTATGAGATTTGTGTATCTTTACTTCGTCC
GGTCAAACCTGTGGCATTATATAAGCACTCTCATGTGTTTTCTTCTTTTGGACAGCAGTCTG
TTGTGTAAATCGTAGTCGTCGACATCTTCTAGTCCACATCGATGACTTCCAGACCGCCGCT
TCTACAAGCTCCTTATTTTCAAGTCGCTTCTACAAGCTCCATATTTTCAACAAGTTTCCTTGG
CAACGGCTTTGCTCATGGCGCACTGCCCGTGGTTGCAAGAGAAAGAATACATTCGACACC
CCTAAAACCATGCAGATGTAATTTCTTTTTGTAAATGTTTTTGTGACCTTTTGCCTTTTTCCA
AAAAAACAAACAAACTAAACCATCACACGAGGAGAAGAGCCATTCCCAGCCGTCCAATAAA
CACGACAAAACGCTTTACAGCACGGGGGCAACTCCCAGCCGTCGAAAATGCATTGCAACG
GAACTACTAGCTCTCCCTGACACAGCGGCACATGAGCCACGCCTCGCACCGCAACCGTGC
ACCGGCGGTGGGCCCACCGGCCAGTGTCCCCCACACGTGTACCGGCGGCCAGCATTGCA
CGGCACGGCGACGGAGAGATATTTTAACGCCTTCCAGTTCAGAAGCACCCCCCATCACTC
ACTCGGGTACAGAGCGGGGCCGCGTGACTCTCGCGTCGTCGCATCGCGCGCGCTGCACA
CCACCGTCGTCAGTGCGACCACTGGCACCA

As a full knockout of PHS1 gene function is most desirable in wheat, the most ideal target sequences are those that are common to all three homeologs of PHS1 (i.e., so that the PHS1 5A, 5B and 5D copies can be edited simultaneously). Suitable target sequences can be found using resources such a the Wheat CRISPR tool https://crispr.bioinfo.nrc.ca/WheatCrispr/ (Cram et al., 2019. BMC Plant Biology 19:474). Examples of four ideal target sequences are given below, where the PAM sequence (NGG) is underlined. Moreover, the first two target sequences can also target PHS1 in barley, and the first three target sequences can also target PHS1 in rye.

CRISPR constructs

SEQ ID NO: 49 Wheat PHS1 target sequence 1 (also works in barley, rye)
ACCCTAAATTATCCAGCATGGGG
SEQ ID NO: 50 Wheat PHS1 target sequence 2 (also works in barley, rye)
CAGGAACCAGATCCTGCCCTTGG
SEQ ID NO: 51 Wheat PHS1 target sequence 3 (also works in rye)
GTTGCATGGTAAGCCTTTAGGG
SEQ ID NO: 52 Wheat PHS1 target sequence 4
CAGGGGACGAATCATCAGAGGGG
SEQ ID NO: 53 Wheat PHS1 protospacer sequence 1
ACCCTAAATTATCCAGCATG
SEQ ID NO: 54 Wheat PHS1 protospacer sequence 2
CAGGAACCAGATCCTGCCCT
SEQ ID NO: 55 Wheat PHS1 protospacer sequence 3
GTTGCATGGTAAGCCTTTA
SEQ ID NO: 56 Wheat PHS1 protospacer sequence 4
CAGGGGACGAATCATCAGAG
SEQ ID NO: 57 tracrRNA sequence
GUUUUAGAGCUAGAAAUAGCAAGUUAAAAUAAGGCUAGUCCGUUAUCAACUUGAAAAAG
UGGCACCGAGUCGGUGCUUUUUUU
SEQ ID NO: 58 Wheat PHS1 complete sgRNA-encoding nucleic acid sequence 1
ACCCTAAATTATCCAGCATGGTTTTAGAGCTAGAAATAGCAAGTTAAAATAAGGCTAGTCCG
TTATCAACTTGAAAAAGTGGCACCGAGTCGGTGCTTTTTTT
SEQ ID NO: 59 Wheat PHS1 complete sgRNA-encoding nucleic acid sequence 2
CAGGAACCAGATCCTGCCCTGTTTTAGAGCTAGAAATAGCAAGTTAAAATAAGGCTAGTCC
GTTATCAACTTGAAAAAGTGGCACCGAGTCGGTGCTTTTTTT
SEQ ID NO: 60 Wheat PHS1 complete sgRNA-encoding nucleic acid sequence 3
GTTGCATGGTAAGCCTTTAGTTTTAGAGCTAGAAATAGCAAGTTAAAATAAGGCTAGTCCGT
TATCAACTTGAAAAAGTGGCACCGAGTCGGTGCTTTTTTT
SEQ ID NO: 61 Wheat PHS1 complete sgRNA-encoding nucleic acid sequence 4
CAGGGGACGAATCATCAGAGGTTTTAGAGCTAGAAATAGCAAGTTAAAATAAGGCTAGTCC
GTTATCAACTTGAAAAAGTGGCACCGAGTCGGTGCTTTTTTT
SEQ ID NO: 62 Wheat PHS1 complete sgRNA RNA sequence 1
ACCCUAAAUUAUCCAGCAUGGUUUUAGAGCUAGAAAUAGCAAGUUAAAAUAAGGCUAGU
CCGUUAUCAACUUGAAAAAGUGGCACCGAGUCGGUGCUUUUUUU
SEQ ID NO: 63 Wheat PHS1 complete sgRNA RNA sequence 2
CAGGAACCAGAUCCUGCCCUGUUUUAGAGCUAGAAAUAGCAAGUUAAAAUAAGGCUAGU
CCGUUAUCAACUUGAAAAAGUGGCACCGAGUCGGUGCUUUUUUU
SEQ ID NO: 64 Wheat PHS1 complete sgRNA RNA sequence 3
GUUGCAUGGUAAGCCUUUAGUUUUAGAGCUAGAAAUAGCAAGUUAAAAUAAGGCUAGUC
CGUUAUCAACUUGAAAAAGUGGCACCGAGUCGGUGCUUUUUUU
SEQ ID NO: 65 Wheat PHS1 complete sgRNA RNA sequence 4
CAGGGGACGAAUCAUCAGAGGUUUUAGAGCUAGAAAUAGCAAGUUAAAAUAAGGCUAGU
CCGUUAUCAACUUGAAAAAGUGGCACCGAGUCGGUGCUUUUUUU
SEQ ID NO: 66 Cas9 sequence
ATGGCTCCTAAGAAGAAGCGGAAGGTTGGTATTCACGGGGTGCCTGCGGCTGACAAGAAG
TACTCCATCGGCCTCGACATCGGCACCAACAGCGTCGGCTGGGCGGTGATCACCGACGA
GTACAAGGTCCCGTCCAAGAAGTTCAAGGTCCTGGGCAACACCGACCGCCACTCCATCAA
GAAGAACCTCATCGGCGCCCTCCTCTTCGACTCCGGCGAGACGGCGGAGGCGACCCGCC
TCAAGCGCACCGCCCGCCGCCGCTACACCCGCCGCAAGAACCGCATCTGCTACCTCCAG
GAGATCTTCTCCAACGAGATGGCGAAGGTCGACGACTCCTTCTTCCACCGCCTCGAGGAG
TCCTTCCTCGTGGAGGAGGACAAGAAGCACGAGCGCCACCCCATCTTCGGCAACATCGTC
GACGAGGTCGCCTACCACGAGAAGTACCCCACTATCTACCACCTTCGTAAGAAGCTTGTTG
ACTCTACTGATAAGGCTGATCTTCGTCTCATCTACCTTGCTCTCGCTCACATGATCAAGTTC
CGTGGTCACTTCCTTATCGAGGGTGACCTTAACCCTGATAACTCCGACGTGGACAAGCTCT
TCATCCAGCTCGTCCAGACCTACAACCAGCTCTTCGAGGAGAACCCTATCAACGCTTCCGG
TGTCGACGCTAAGGCGATCCTTTCCGCTAGGCTCTCCAAGTCCAGGCGTCTCGAGAACCT
CATCGCCCAGCTCCCTGGTGAGAAGAAGAACGGTCTTTTCGGTAACCTCATCGCTCTCTCC
CTCGGTCTGACCCCTAACTTCAAGTCCAACTTCGACCTCGCTGAGGACGCTAAGCTTCAGC
TCTCCAAGGATACCTACGACGATGATCTCGACAACCTCCTCGCTCAGATTGGAGATCAGTA
CGCTGATCTCTTCCTTGCTGCTAAGAACCTCTCCGATGCTATCCTCCTTTCGGATATCCTTA
GGGTTAACACTGAGATCACTAAGGCTCCTCTTTCTGCTTCCATGATCAAGCGCTACGACGA
GCACCACCAGGACCTCACCCTCCTCAAGGCTCTTGTTCGTCAGCAGCTCCCCGAGAAGTA
CAAGGAGATCTTCTTCGACCAGTCCAAGAACGGCTACGCCGGTTACATTGACGGTGGAGC
TAGCCAGGAGGAGTTCTACAAGTTCATCAAGCCAATCCTTGAGAAGATGGATGGTACTGAG
GAGCTTCTCGTTAAGCTTAACCGTGAGGACCTCCTTAGGAAGCAGAGGACTTTCGATAACG
GCTCTATCCCTCACCAGATCCACCTTGGTGAGCTTCACGCCATCCTTCGTAGGCAGGAGG
ACTTCTACCCTTTCCTCAAGGACAACCGTGAGAAGATCGAGAAGATCCTTACTTTCCGTATT
CCTTACTACGTTGGTCCTCTTGCTCGTGGTAACTCCCGTTTCGCTTGGATGACTAGGAAGT
CCGAGGAGACTATCACCCCTTGGAACTTCGAGGAGGTTGTTGACAAGGGTGCTTCCGCCC
AGTCCTTCATCGAGCGCATGACCAACTTCGACAAGAACCTCCCCAACGAGAAGGTCCTCC
CCAAGCACTCCCTCCTCTACGAGTACTTCACGGTCTACAACGAGCTCACCAAGGTCAAGTA
CGTCACCGAGGGTATGCGCAAGCCTGCCTTCCTCTCCGGCGAGCAGAAGAAGGCTATCGT
TGACCTCCTCTTCAAGACCAACCGCAAGGTCACCGTCAAGCAGCTCAAGGAGGACTACTT
CAAGAAGATCGAGTGCTTCGACTCCGTCGAGATCAGCGGCGTTGAGGACCGTTTCAACGC
TTCTCTCGGTACCTACCACGATCTCCTCAAGATCATCAAGGACAAGGACTTCCTCGACAAC
GAGGAGAACGAGGACATCCTCGAGGACATCGTCCTCACTCTTACTCTCTTCGAGGATAGG
GAGATGATCGAGGAGAGGCTCAAGACTTACGCTCATCTCTTCGATGACAAGGTTATGAAGC
AGCTCAAGCGTCGCCGTTACACCGGTTGGGGTAGGCTCTCCCGCAAGCTCATCAACGGTA
TCAGGGATAAGCAGAGCGGCAAGACTATCCTCGACTTCCTCAAGTCTGATGGTTTCGCTAA
CAGGAACTTCATGCAGCTCATCCACGATGACTCTCTTACCTTCAAGGAGGATATTCAGAAG
GCTCAGGTGTCCGGTCAGGGCGACTCTCTCCACGAGCACATTGCTAACCTTGCTGGTTCC
CCTGCTATCAAGAAGGGCATCCTTCAGACTGTTAAGGTTGTCGATGAGCTTGTCAAGGTTA
TGGGTCGTCACAAGCCTGAGAACATCGTCATCGAGATGGCTCGTGAGAACCAGACTACCC
AGAAGGGTCAGAAGAACTCGAGGGAGCGCATGAAGAGGATTGAGGAGGGTATCAAGGAG
CTTGGTTCTCAGATCCTTAAGGAGCACCCTGTCGAGAACACCCAGCTCCAGAACGAGAAG
CTCTACCTCTACTACCTCCAGAACGGTAGGGATATGTACGTTGACCAGGAGCTCGACATCA
ACAGGCTTTCTGACTACGACGTCGACCACATTGTTCCTCAGTCTTTCCTTAAGGATGACTC
CATCGACAACAAGGTCCTCACGAGGTCCGACAAGAACAGGGGTAAGTCGGACAACGTCCC
TTCCGAGGAGGTTGTCAAGAAGATGAAGAACTACTGGAGGCAGCTTCTCAACGCTAAGCT
CATTACCCAGAGGAAGTTCGACAACCTCACGAAGGCTGAGAGGGGTGGCCTTTCCGAGCT
TGACAAGGCTGGTTTCATCAAGAGGCAGCTTGTTGAGACGAGGCAGATTACCAAGCACGT
TGCTCAGATCCTCGATTCTAGGATGAACACCAAGTACGACGAGAACGACAAGCTCATCCGC
GAGGTCAAGGTGATCACCCTCAAGTCCAAGCTCGTCTCCGACTTCCGCAAGGACTTCCAG
TTCTACAAGGTCCGCGAGATCAACAACTACCACCACGCTCACGATGCTTACCTTAACGCTG
TCGTTGGTACCGCTCTTATCAAGAAGTACCCTAAGCTTGAGTCCGAGTTCGTCTACGGTGA
CTACAAGGTCTACGACGTTCGTAAGATGATCGCCAAGTCCGAGCAGGAGATCGGCAAGGC
CACCGCCAAGTACTTCTTCTACTCCAACATCATGAACTTCTTCAAGACCGAGATCACCCTC
GCCAACGGCGAGATCCGCAAGCGCCCTCTTATCGAGACGAACGGTGAGACTGGTGAGAT
CGTTTGGGACAAGGGTCGCGACTTCGCTACTGTTCGCAAGGTCCTTTCTATGCCTCAGGTT
AACATCGTCAAGAAGACCGAGGTCCAGACCGGTGGCTTCTCCAAGGAGTCTATCCTTCCA
AAGAGAAACTCGGACAAGCTCATCGCTAGGAAGAAGGATTGGGACCCTAAGAAGTACGGT
GGTTTCGACTCCCCTACTGTCGCCTACTCCGTCCTCGTGGTCGCCAAGGTGGAGAAGGGT
AAGTCGAAGAAGCTCAAGTCCGTCAAGGAGCTCCTCGGCATCACCATCATGGAGCGCTCC
TCCTTCGAGAAGAACCCGATCGACTTCCTCGAGGCCAAGGGCTACAAGGAGGTCAAGAAG
GACCTCATCATCAAGCTCCCCAAGTACTCTCTTTTCGAGCTCGAGAACGGTCGTAAGAGGA
TGCTGGCTTCCGCTGGTGAGCTCCAGAAGGGTAACGAGCTTGCTCTTCCTTCCAAGTACG
TGAACTTCCTCTACCTCGCCTCCCACTACGAGAAGCTCAAGGGTTCCCCTGAGGATAACGA
GCAGAAGCAGCTCTTCGTGGAGCAGCACAAGCACTACCTCGACGAGATCATCGAGCAGAT
CTCCGAGTTCTCCAAGCGCGTCATCCTCGCTGACGCTAACCTCGACAAGGTCCTCTCCGC
CTACAACAAGCACCGCGACAAGCCCATCCGCGAGCAGGCCGAGAACATCATCCACCTCTT
CACGCTCACGAACCTCGGCGCCCCTGCTGCTTTCAAGTACTTCGACACCACCATCGACAG
GAAGCGTTACACGTCCACCAAGGAGGTTCTCGACGCTACTCTCATCCACCAGTCCATCACC
GGTCTTTACGAGACTCGTATCGACCTTTCCCAGCTTGGTGGTGATAAGCGTCCTGCTGCCA
CCAAAAAGGCCGGACAGGCTAAGAAAAAGAAGTAG
SEQ ID NO: 67 Csy4 endoribonuclease nucleic acid sequence
ATGGACCACTACCTCGACATCAGGCTCAGGCCAGACCCAGAGTTCCCACCAGCCCAGCTC
ATGTCCGTCCTCTTCGGCAAGCTCCACCAGGCCCTCGTGGCCCAGGGCGGCGACAGGAT
CGGCGTGTCCTTCCCAGACCTCGACGAGTCCAGGTCCAGGCTCGGCGAGAGGCTCCGCA
TCCACGCCTCCGCCGACGACCTCAGGGCCCTCCTCGCCAGGCCGTGGCTGGAGGGCCTC
AGGGACCACCTCCAGTTCGGCGAGCCAGCCGTGGTGCCACACCCAACCCCATACAGGCA
AGTGTCCAGGGTGCAAGCCAAGTCCAACCCAGAGAGGCTCAGGAGGAGGCTCATGAGGA
GGCACGACCTCTCCGAGGAAGAGGCCAGGAAGCGCATCCCAGACACCGTGGCCAGGGCC
CTCGACCTCCCATTCGTGACCCTCAGGTCCCAGTCCACCGGCCAGCACTTCCGCCTCTTC
ATCAGGCACGGCCCACTCCAGGTGACCGCCGAGGAGGGGGCTTTACCTGCTACGGCCT
CTCCAAGGGCGGCTTCGTGCCGTGGTTC
SEQ ID NO: 68 Wheat U6 promoter
GACCAAGCCCGTTATTCTGACAGTTCTGGTGCTCAACACATTTATATTTATCAAGGAGCACA
TTGTTACTCACTGCTAGGAGGGAATCGAACTAGGAATATTGATCAGAGGAACTACGAGAGA
GCTGAAGATAACTGCCCTCTAGCTCTCACTGATCTGGGTCGCATAGTGAGATGCAGCCCA
CGTGAGTTCAGCAACGGTCTAGCGCTGGGCTTTTAGGCCCGCATGATCGGGCTTTTGTCG
GGTGGTCGACGTGTTCACGATTGGGGAGAGCAACGCAGCAGTTCCTCTTAGTTTAGTCCC
ACCTCGCCTGTCCAGCAGAGTTCTGACCGGTTTATAAACTCGCTTGCTGCATCAGACTTG
SEQ ID NO: 69 Maize Ubiquitin1 promoter
TGCAGTGCAGCGTGACCCGGTCGTGCCCCTCTCTAGAGATAATGAGCATTGCATGTCTAA
GTTATAAAAAATTACCACATATTTTTTTTGTCACACTTGTTTGAAGTGCAGTTTATCTATCTTT
ATACATATATTTAAACTTTACTCTACGAATAATATAATCTATAGTACTACAATAATATCAGTGT
TTTAGAGAATCATATAAATGAACAGTTAGACATGGTCTAAAGGACAATTGAGTATTTTGACA
ACAGGACTCTACAGTTTTATCTTTTTAGTGTGCATGTGTTCTCCTTTTTTTTTGCAAATAGCT
TCACCTATATAATACTTCATCCATTTTATTAGTACATCCATTTAGGGTTTAGGGTTAATGGTT
TTTATAGACTAATTTTTTTAGTACATCTATTTTATTCTATTTTAGCCTCTAAATTAAGAAAACTA
AAACTCTATTTTAGTTTTTTTATTTAATAATTTAGATATAAAATAGAATAAAATAAAGTGACTA
AAAATTAAACAAATACCCTTTAAGAAATTAAAAAAACTAAGGAAACATTTTTCTTGTTTCGAG
TAGATAATGCCAGCCTGTTAAACGCCGTCGACGAGTCTAACGGACACCAACCAGCGAACC
AGCAGCGTCGCGTCGGGCCAAGCGAAGCAGACGGCACGGCATCTCTGTCGCTGCCTCTG
GACCCCTCTCGAGAGTTCCGCTCCACCGTTGGACTTGCTCCGCTGTCGGCATCCAGAAAT
TGCGTGGCGGAGCGGCAGACGTGAGCCGGCACGGCAGGCGGCCTCCTCCTCCTCTCAC
GGCACCGGCAGCTACGGGGGATTCCTTTCCCACCGCTCCTTCGCTTTCCCTTCCTCGCCC
GCCGTAATAAATAGACACCCCCTCCACACCCTCTTTCCCCAACCTCGTGTTGTTCGGAGCG
CACACACACACAACCAGATCTCCCCCAAATCCACCCGTCGGCACCTCCGCTTCAAG
SEQ ID NO: 84 Barley PHS1 target sequence
CGGAAGGCGGTGGCGAGCGGCGG
SEQ ID NO: 85 Barley PHS1 target sequence 2
GAGCGTGGCGAGCGATCGGGAGG
SEQ ID NO: 86 Barley PHS1 protospacer sequence 1
CGGAAGGCGGTGGCGAGCGG
SEQ ID NO: 87 Barley PHS1 protospacer sequence 2
GAGCGTGGCGAGCGATCGGG
SEQ ID NO: 88 Barley PHS1 complete sgRNA-encoding nucleic acid sequence 1
CGGAAGGCGGTGGCGAGCGGGTTTTAGAGCTAGAAATAGCAAGTTAAAATAAGGCTAGTC
CGTTATCAACTTGAAAAAGTGGCACCGAGTCGGTGCTTTTTTT
SEQ ID NO: 89 Barley PHS1 complete sgRNA-encoding nucleic acid sequence 2
GAGCGTGGCGAGCGATCGGGGTTTTAGAGCTAGAAATAGCAAGTTAAAATAAGGC
TAGTCCGTTATCAACTTGAAAAAGTGGCACCGAGTCGGTGCTTTTTTT

1. A genetically altered plant, part thereof or plant cell comprising reduced or abolished expression or activity of at least one PHS1 (plastidial α-glucan phosphorylase) gene.

2. The genetically altered plant, part thereof or plant cell of claim 1, wherein the plant comprises a mutation in at least one gene encoding PHS1 and/or the PHS1 promoter wherein the mutation in at least one gene encoding PHS1 is a loss of function or partial loss of function mutation, wherein preferably the mutation reduces or abolished the phosphorylase activity of PHS1.

3. (canceled)

4. The genetically altered plant, part thereof or plant cell of claim 1, wherein the plant comprises an RNAi interference construct that reduces or abolishes the expression of at least one PHS1 gene.

5. The genetically altered plant, part thereof or plant cell of claim 1, wherein the PHS1 gene encodes a PHS1 polypeptide comprising SEQ ID NO: 3, 7, 11, 15, 19, 23, 27, 31, 35, 39, 43 or 47 or a functional variant or homologue thereof, wherein optionally the functional variant or homologue has at least 60% overall sequence identity to SEQ ID NO: 3, 7, 11, 15, 19, 23, 27, 31, 35, 39, 43 or 47.

6. (canceled)

7. The genetically altered plant, part thereof or plant cell of claim 1, wherein the plant is selected from wheat, barley, rye, maize, potato and sorghum.

8. A method of altering the starch granule characteristics of a plant, the method comprising reducing the expression or activity of at least one PHS1 (plastidial α-glucan phosphorylase) gene.

9. (canceled)

10. The method of claim 8, wherein the plant has a bimodal starch granule composition comprising a first population of starch granules of a first granule size range and at least a second population of starch granules of a second, smaller size range, and wherein the method comprises reducing the number of granules in the second population and/or increasing the size of starch granules in the second population.

11. The method of claim 8, wherein the plant comprises A-type and B-type starch granules, and wherein the method comprises reducing the number and/or increasing the size of B-type starch granules.

12. The method of claim 8, wherein the method comprises introducing at least one mutation into at least one gene encoding PHS1 and/or the PHS1 promoter.

13. The method of claim 8, wherein the mutation is a loss of function or partial loss of function mutation, wherein preferably the mutation reduces or abolishes the phosphorylase activity of PHS1.

14. The method of claim 8, wherein the method comprises introducing and expressing in the plant an RNAi interference construct that reduces or abolishes the expression of at least one PHS1 gene.

15. The method of claim 8, wherein the PHS1 gene encodes a PHS1 polypeptide comprising SEQ ID NO: 3, 7, 11, 15, 19, 23, 27, 31, 35, 39, 43 or 47 or a functional variant or homologue thereof, wherein optionally the functional variant or homologue has at least 60% overall sequence identity to SEQ ID NO: 3, 7, 11, 15, 19, 23, 27, 31, 35, 39, 43 or 47.

16. (canceled)

17. The method of claim 8, wherein the plant is selected from wheat, barley, rye, maize, potato and sorghum.

18. A plant, plant part or plant cell obtained or obtainable by the method of claim 8.

19. Grain derived from the genetically altered plant of claim 1, wherein the grain is characterised by reduced or abolished expression of a PHS1 gene and/or reduced or abolished activity of a PHS1 polypeptide.

20. The grain of claim 19, wherein the grain comprises at least one mutation in a PHS1 gene and/or promoter.

21. Starch obtained or obtainable from at least one plant cell of the genetically altered plant of claim 1.

22. A food or feed composition prepared from the grain of claim 19.

23. (canceled)

24. A nucleic acid construct comprising a nucleic acid sequence encoding at least one DNA-binding domain or protospacer element that can bind to at least one target sequence in a PHS1 gene and/or promoter, wherein preferably the target sequence is selected from SEQ ID NO: 49 to 52, 84 or 85 or a variant thereof, wherein preferably, the sequence of the protospacer element is selected from SEQ ID Nos 53 to 56 or 86 or 87 or a variant thereof.

25. The nucleic acid construct of claim 24, wherein said construct encodes at least one single-guide RNA (sgRNA), wherein said sgRNA comprises the tracrRNA sequence and the crRNA or protospacer sequence, wherein the sgRNA comprises or consists of a sequence selected from SEQ ID Nos 58 to 61 or 88 or 89 or a functional variant thereof.

26. (canceled)
